SEQUENCE LISTING (29 sequences)

SEQ ID NO: 1 - protein, 118 aa, synthetic construct
  source 1..118: note = VH region
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSS

SEQ ID NO: 2 - protein, 112 aa, synthetic construct
  source 1..112: note = VL region
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 3 - protein, 5 aa, synthetic construct
  source 1..5: note = VH CDR 1
EYYMY

SEQ ID NO: 4 - protein, 17 aa, synthetic construct
  source 1..17: note = VH CDR 2
RIDPEDGSIDYVEKFKK

SEQ ID NO: 5 - protein, 9 aa, synthetic construct
  source 1..9: note = VH CDR 3
GKFNYRFAY

SEQ ID NO: 6 - protein, 16 aa, synthetic construct
  source 1..16: note = VL CDR 1
RSSQSLLHSSGNTYLN

SEQ ID NO: 7 - protein, 7 aa, synthetic construct
  source 1..7: note = VL CDR 2
LVSKLES

SEQ ID NO: 8 - protein, 9 aa, synthetic construct
  source 1..9: note = VL CDR 3
MQFTHYPYT

SEQ ID NO: 9 - protein, 330 aa, synthetic construct
  source 1..330: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 330 aa, synthetic construct
  source 1..330: note = Heavy chain constant region
ASTKGPSVFPLAPSSKSTSGGTAALGCLVKDYFPEPVTVSWNSGALTSGVHTFPAVLQSSGLHSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 330 aa, synthetic construct
  source 1..330: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 12 - protein, 330 aa, synthetic construct [sequence omitted]

SEQ ID NO: 13 - protein, 330 aa, synthetic construct
  source 1..330: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLSGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 107 aa, synthetic construct
  source 1..107: note = Light chain constant region
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 15 - protein, 107 aa, synthetic construct [sequence omitted]

SEQ ID NO: 16 - DNA, 1350 bp, synthetic construct
  source 1..1350: mol_type = other DNA; note = Optimized Heavy chain sequence
caagtgcagctggtgcagagcggagctgaggtgcagagacccggcgccagcgtcaaggtgagctgtaaggccagcggctacatcttcacagaatactacatgtactgggtgaggcaagcccccggccaaggactggagctggtgggcagaatcgatccagaggatggcagcatcgactacgtggagaagttcaagaagaaggtgactctgacagccgacacaagcagcagcactgcttacatggagctgagctctctgactagcgatgacactgccgtgtactactgtgctaggggcaagttcaactataggttcgcctactggggccaaggcactctggtgacagtcagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatga

SEQ ID NO: 17 - DNA, 663 bp, synthetic construct
  source 1..663: mol_type = other DNA; note = Optimized Light chain sequence
gacgtggtgatgactcagagccctccttctctgctggtgactctgggccagccagccagcatcagctgtaggagcagccagtctctgctgcactccagcggcaacacttatctgaactggctgctgcagagacccggccagagccctcagcctctgatctacctcgtgagcaagctggagagcggcgtgccagataggtttagcggcagcggaagcggcactgacttcactctgaagatcagcggcgtggaagctgaggatgtgggcgtctactactgcatgcagttcacacactacccatacactttcggccaaggcacaaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatga

SEQ ID NO: 18 - protein, 102 aa, synthetic construct
  source 1..102: note = CH2 domain
SVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTK

SEQ ID NO: 19 - protein, 102 aa, synthetic construct
  source 1..102: note = CH2 domain
SVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAK

SEQ ID NO: 20 - protein, 107 aa, synthetic construct
  source 1..107: note = CH3 domain
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 107 aa, synthetic construct
  source 1..107: note = CH3 domain
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 6 aa, synthetic construct
  source 1..6: note = tocilizumab heavy chain variable region CDR 1
SDHAWS

SEQ ID NO: 23 - protein, 16 aa, synthetic construct
  source 1..16: note = tocilizumab heavy chain variable region CDR 2
YISYSGITTYNPSLKS

SEQ ID NO: 24 - protein, 10 aa, synthetic construct
  source 1..10: note = tocilizumab heavy chain variable region CDR 3
SLARTTAMDY

SEQ ID NO: 25 - protein, 11 aa, synthetic construct
  source 1..11: note = tocilizumab light chain variable region CDR 1
RASQDISSYLN

SEQ ID NO: 26 - protein, 7 aa, synthetic construct
  source 1..7: note = tocilizumab light chain variable region CDR 2
YTSRLHS

SEQ ID NO: 27 - protein, 9 aa, synthetic construct
  source 1..9: note = tocilizumab light chain variable region CDR 3
QQGNTLPYT

SEQ ID NO: 28 - protein, 119 aa, synthetic construct [sequence omitted]

SEQ ID NO: 29 - protein, 107 aa, synthetic construct [sequence omitted]

(19) United States
(12) Patent Application Publication
(10) Pub. No.: US 2025/0276061 A1
(43) Pub. Date: Sep. 4, 2025

(54) METHOD AND COMPOSITION FOR INDUCING TOLERANCE

(71) Applicant: ZELARION MALTA LIMITED, Birkirkara (MT)

(72) Inventors: David Berglund, New York, NY (US); Erik Berglund, New York, NY (US)

(73) Assignee: ZELARION MALTA LIMITED, Birkirkara (MT)

(21) Appl. No.: 19/210,482

(22) Filed: May 16, 2025

Related U.S. Application Data
(63) Continuation of application No. 18/424,381, filed on Jan. 26, 2024.
Application No. 18/424,381 is a continuation of application No. PCT/EP2022/074646, filed on Sep. 5, 2022.
(60) Provisional application No. 63/241,358, filed on Sep. 7, 2021.

(51) Int. Cl. A61K 39/395; A61K 31/436; A61K 31/573; A61K 31/675; A61K 31/7076; A61K 35/12; A61K 35/28; A61K 39/00; C07K 16/28

(52) U.S. Cl. CPC A61K 39/3955; A61K 31/436; A61K 31/573; A61K 31/675; A61K 31/7076; A61K 35/28; C07K 16/2803; C07K 16/2887; A61K 2035/122; A61K 2039/507; A61K 2039/577

(57) ABSTRACT

Provided herein are methods for inducing tolerance to an organ or tissue transplant in a patient. Specifically, provided herein are methods for inducing temporary mixed chimerism in a transplant-eligible patient, wherein the method comprises administering an anti-CD2 antibody or antigen binding fragment thereof to the patient, transplanting bone marrow from a donor to the patient; and administering at least one other agent prior to and/or after transplant to the patient.

### CROSS-REFERENCE TO RELATED APPLICATIONS

The present application claims the benefit of priority to U.S. Ser. No. 63/241,358 filed Sep. 7, 2021, the disclosure of which is incorporated herein by reference in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14526-007-228_SEQ_LISTING.xml”, was created on Aug. 31, 2022 and is 36,184 bytes in size.

### 1. INTRODUCTION

Provided herein are methods for inducing tolerance to an organ or tissue transplant in a patient. Compositions for use with these methods and kits are also disclosed.

### 2. BACKGROUND

Long-term results of organ transplantation remain unsatisfactory. Chronic rejection of the donor graft and health issues associated with long-term immunosuppressant use continue to be serious complications of the procedure. These complications, however, could be overcome by inducing tolerance to the donor graft by the recipient's immune system. Tolerance avoids a destructive immune response following transplantation without the need for systemic immunosuppression.

Combined kidney and bone marrow transplantation has been described. See, e.g., Kawai et al. 2014, American Journal of Transplantation 14:1599-1611. This study showed long-term stable kidney allograft survival without maintenance immunosuppression. However, revisions to the conditioning regimen are needed for optimized clinical outcome. The methods disclosed herein describes the refined treatment regimens to provide consistent tolerance to organ or tissue transplantation.

Siplizumab is a humanized IgG1κ monoclonal antibody directed against the human CD2 glycoprotein, a receptor expressed on virtually all mature human T cells and on the vast majority of thymocytes (Bierer B E, Burakoff S J., Immunol Rev. 1989; 111:267-294). CD2 promotes the adhesion of T cells to antigen-presenting cells through its interaction with the ligand LFA-3 (CD58) (van der Merwe P A, Barclay A N, Mason D W, et al. Biochemistry. 1994; 33(33):10149-10160). Binding of CD2 to LFA-3 also leads to a cascade of intracellular signals necessary for T-cell activation, conferring an important costimulatory function to this molecule (Bockenstedt L K. et al., J Immunol. 1988; 141(6):1904-1911; Binder, Christian, et al., (2020). CD2 Immunobiology. Front. Immunol. 11:1090. doi: 10.3389/fimmu.2020.01090).

Ligation of either siplizumab or its parent antibody (BTI-322/Lo-CD2a) to human CD2 promotes T-cell depletion through antibody-dependent cell-mediated cytotoxicity (ADCC), an effect that is notably more pronounced on activated T cells (Branco L. et al., Transplantation. 1999; 68(10):1588-1596; Nizet Y, et al. Transplantation. 2000; 69(7):1420-1428). In addition, these drugs efficiently inhibit the in vitro allogeneic mixed-lymphocyte reaction (MLR), with subsequent hyporesponsiveness upon allogeneic restimulation, while preserving reactivity to non-specific stimulation. This effect was found to be mediated, at least in part, by specific activation-associated T-cell depletion (Latinne D. et al., Int Immunol. 1996; 8(7):1113-1119; Xu Y. et al., Clin Exp Immunol. 2004; 138(3):476-483; Podestà MA, Binder C, Sellberg F, et al. Siplizumab selectively depletes effector memory T cells and promotes a relative expansion of alloreactive regulatory T cells in vitro. Am J Transplant. 2019; 20:88-100. https://doi.org/10.1111/ajt.15533; Binder Christian, et al. (2020) Siplizumab, an Anti-CD2 Monoclonal Antibody, Induces a Unique Set of Immune Modulatory Effects Compared to Alemtuzumab and Rabbit AntiThymocyte Globulin In Vitro. Front. Immunol. 11:592553. doi: 10.3389/fimmu.2020.592553).

Siplizumab has been used in conditioning regimens for hematopoietic cell transplantation and tolerance induction with combined kidney-bone marrow transplantation (Dey, Bimalangshu, et al. Anti-tumour response despite loss of donor chimaerism in patients treated with non-myeloablative conditioning and allogeneic stem cell transplantation. British Journal of Haematology, 2005, 128, 351-359, doi: 10.1111/j. 1365-2141.2004.05328.x; Shaffer, Juanita, et al. Regulatory T-cell recovery in recipients of haploidentical nonmyeloablative hematopoietic cell transplantation with a humanized anti-CD2 mAb, MEDI-507, with or without fludarabine. Exp Hematol. 2007 July; 35(7):1140-52. doi: 10.1016/j.exphem.2007.03.018).

Siplizumab depletes T cells globally while enriching regulatory T cells (Tregs) early following transplantation. In recipients of hematopoietic cell transplantation to treat malignancies (Shaffer J. et al., Exp Hematol. 2007; 35(7):1140-1152) and CKBMT recipients (Andreola G. et al., Am J Transplant. 2011; 11(6):1236-1247), a marked early enrichment in regulatory T cells (Tregs) was observed during the T-cell reconstitution phase (Savage, Thomas, et al., Early expansion of donor-specific Tregs in tolerant kidney transplant recipients. JCI Insight. 2018 Nov. 15; 3(22): e124086. doi: 10.1172/jci.insight. 124086; Sprangers, Ben, et al. Origin of Enriched regulatory t cells in patients receiving combined kidney/bone marrow transplantation to induce transplantation tolerance. Am J Transplant. 2017 August; 17(8): 2020-2032. doi: 10.1111/ajt. 14251; LoCascio, Samuel, et al. Mixed Chimerism, Lymphocyte Recovery and Evidence for Early Donor-Specific Unresponsiveness in Patients Receiving CKBMT to Induce Tolerance. Transplantation. 2010 Dec. 27; 90(12): 1607-1615. doi: 10.1097/TP.0b013e3181ffbaff).

Previous studies have shown that anti-IL6R treatment restores the physiological Treg/Th17 balance which may be important for the clinical response observed in patients with rheumatoid arthritis when administered humanized IL6R antibodies (Schinnerling, et al., Clin Exp Immunol. 2017 189(1):12-20). Anti-IL6R treatment has been shown to promote the expansion of protective Treg cells; Tada, BMC Musculoskelet Disord. 2016 17:290). Additionally, the use of an anti-IL6R blockade is efficient in treating cytokine release syndrome (CRS) (Brudno J N. and Kochenderfer J N. Blood. 2016 127(26):3321-30; Hay, K. Br J Haematol. 183(3):364-374. 2018). The use of an anti-IL6R antibody to block IL6 signaling is FDA approved as a therapeutic for CRS in CAR-T cell patients, (Le, et al., Oncologist. 2018 (8):943-947. 2018; Highlights of Prescribing Information; ACTEMRA® (tocilizumab) injection, for intravenous or subcutaneous use. Genentech, Inc. 2017).

### 3. SUMMARY

Provided herein are methods for inducing transient mixed chimerism in a transplant-eligible patient, wherein the methods comprise administering an anti-CD2 antibody or antigen binding fragment thereof to the patient, subjecting the patient to an organ or tissue transplant, infusing bone marrow cells from a donor to the patient; and administering one or more than one agent(s) to the subject prior to and/or after transplant. In certain embodiments, the chimerism can persist for several weeks to months after the transplant surgery. In certain embodiments, the methods include a conditioning regimen and a postoperative regimen. In certain embodiments, the methods induce tolerance in the recipient towards the transplanted organ or tissue. This tolerance can be long-lasting or permanent and minimized risk of Graft versus Host Disease.

Without being bound by theory, hematopoeitic cells, obtained from the bone marrow of the donor, can be infused into the recipient to create a state in which donor and recipient hematopoeitic cells coexist. In combination with non-myeloablative conditioning, bone marrow transplant results in chimerism of the recipient, leading to the induction of donor-specific tolerance. Advantages of this approach include the avoidance of ablating the recipient's immune system and inhibiting the development of Graft versus Host diseases without the need for long-term systemic immunosuppressive use.

In one aspect provided herein is a method of transplanting an organ or a tissue from a donor to a subject, wherein the method comprises: a) administering a B-cell depleting antibody to the subject; b) administering an anti-CD2 antibody or antigen binding fragment thereof to the subject; c) administering fludarabine to the subject; d) transplanting the organ or the tissue into the subject; and e) infusing bone marrow cells from the donor to the subject.

In one aspect provided herein is a method of transplanting an organ or a tissue from a donor to a subject, wherein the method comprises: a) administering a B-cell depleting antibody to the subject; b) administering an anti-CD2 antibody or antigen binding fragment thereof to the subject; c) transplanting the organ or the tissue into the subject; and d) infusing bone marrow cells from the donor to the subject; wherein the B-cell depleting antibody is administered to the subject more than 7 days prior to the transplanting.

In one aspect provided herein is a method of transplanting an organ or a tissue from a donor to a subject, wherein the method comprises: a) administering a B-cell depleting antibody to the subject; b) administering an anti-CD2 antibody or antigen binding fragment thereof to the subject; c) transplanting the organ or the tissue into the subject; and d) infusing bone marrow cells from the donor to the subject; wherein a first dose of the anti-CD2 antibody or antigen binding fragment thereof is 1 day prior to the transplanting.

In one aspect provided herein is a method of transplanting an organ or a tissue from a donor to a subject, wherein the method comprises: a) administering a B-cell depleting antibody to the subject; b) administering an anti-CD2 antibody or antigen binding fragment thereof to the subject; c) transplanting the organ or the tissue into the subject; and d) infusing bone marrow cells from the donor to the subject; wherein the anti-CD2 antibody or antigen binding fragment thereof is administered to the subject more than 2 days prior to the transplanting.

In one aspect provided herein is a method of minimizing chimeric transition syndrome in a subject in need of an organ or a tissue transplant, wherein the method comprises: a) administering a B-cell depleting antibody to the subject; b) administering an anti-CD2 antibody or antigen binding fragment thereof to the subject; c) administering fludarabine to the subject; d) transplanting the organ or the tissue into the subject; and e) infusing bone marrow cells from the donor to the subject.

In one aspect provided herein is a method of minimizing chimeric transition syndrome in a subject in need of an organ or a tissue transplant, wherein the method comprises: a) administering a B-cell depleting antibody to the subject; b) administering an anti-CD2 antibody or antigen binding fragment thereof to the subject; c) transplanting the organ or the tissue into the subject; and d) infusing bone marrow cells from the donor to the subject; wherein the B-cell depleting antibody is administered to the subject more than 7 days prior to the transplanting.

In one aspect provided herein is a method of minimizing chimeric transition syndrome in a subject in need of an organ or a tissue transplant, wherein the method comprises: a) administering a B-cell depleting antibody to the subject; b) administering an anti-CD2 antibody or antigen binding fragment thereof to the subject; c) transplanting the organ or the tissue into the subject; and d) infusing bone marrow cells from the donor to the subject; wherein a first dose of the anti-CD2 antibody or antigen binding fragment thereof is 1 day prior to the transplanting.

In one aspect provided herein is a method of minimizing chimeric transition syndrome in a subject in need of an organ or a tissue transplant, wherein the method comprises: a) administering a B-cell depleting antibody to the subject; b) administering an anti-CD2 antibody or antigen binding fragment thereof to the subject; c) transplanting the organ or the tissue into the subject; and d) infusing bone marrow cells from the donor to the subject; wherein the anti-CD2 antibody or antigen binding fragment thereof is administered to the subject more than 2 days prior to the transplanting.

In some aspects, the B-cell depleting antibody is administered to the subject 9 days prior to the transplanting. In some aspects, the B-cell depleting antibody is administered to the subject 9 days prior to and 2 days prior to the transplanting. In some aspects, the B-cell depleting antibody is administered to the subject 9 days prior to and 2 days prior to the transplanting and 5 days post and 12 days post the transplanting. In some aspects, the B-cell depleting antibody is rituximab. In some aspects, the B-cell depleting antibody is administered to the subject at a dose of about 375 mg/m². In some aspects, the B-cell depleting antibody is not administered 7 days prior to the transplanting.

In some aspects, the anti-CD2 antibody or antigen binding fragment thereof is administered to the subject 1 day prior to the transplanting, on the day of the transplanting, and 1 day post the transplanting. In some aspects, the anti-CD2 antibody or antigen binding fragment thereof is administered to the subject 6 days prior to the transplanting, 1 day prior to the transplanting, on the day of the transplanting, and 1 day post the transplanting. In some aspects, the anti-CD2 antibody or antigen binding fragment thereof is administered to the subject 6 days prior to the transplanting, 1 day prior to the transplanting, on the day of the transplanting, 1 day post the transplanting, and 6 days post the transplanting. In some aspects, the anti-CD2 antibody or antigen binding fragment thereof is siplizumab. In some aspects, the anti-CD2 antibody or antigen binding fragment thereof is administered to the subject at a dose of about 0.6 mg/kg. In some aspects, the anti-CD2 antibody or antigen binding fragment thereof comprises: a) a heavy chain variable region CDR 1 of SEQ ID NO:3; and b) a heavy chain variable region CDR 2 of SEQ ID NO:4; and c) a heavy chain variable region CDR 3 of SEQ ID NO:5; and d) a light chain variable region CDR 1 of SEQ ID NO:6; and e) a light chain variable region CDR 2 of SEQ ID NO:7; and f) a light chain variable region CDR 3 of SEQ ID NO:8. In some aspects, the anti-CD2 antibody or antigen binding fragment thereof is a humanized antibody. In some aspects, the anti-CD2 antibody or antigen binding fragment thereof is administered to the subject at a dose of more than about 0.1 mg/kg. In some aspects, the anti-CD2 antibody or antigen binding fragment thereof is administered to the subject at a dose of about 0.6 mg/kg. In some aspects, each dose of the anti-CD2 antibody or antigen binding fragment thereof that is administered to the subject comprises the same dose.

In some aspects, the method provided herein further comprises administering a non-myeloablative conditioning agent to the subject. In some aspects, the non-myeloablative conditioning agent comprises an immunosuppressive agent. In some aspects, the immunosuppressive agent is cyclophosphamide. In some aspects, the immunosuppressive agent is administered to the subject 5 days prior to and 4 days prior to the transplanting. In some aspects, the immunosuppressive agent is administered to the subject at a dose of about 60 mg/kg. In some aspects, the immunosuppressive agent is administered to the subject at a dose of about 22.5 mg/kg. In some aspects, the non-myeloablative conditioning agent comprises an anti-neoplastic agent. In some aspects, the anti-neoplastic agent is a chemotherapy drug. In some aspects, the anti-neoplastic agent is fludarabine. In some aspects, the anti-neoplastic agent is administered to the subject 6 days prior to, 5 days prior to, 4 days prior to, and 3 days prior to the transplanting. In some aspects, fludarabine is administered to the subject 6 days prior to, 5 days prior to, 4 days prior to, and 3 days prior to the transplanting. In some aspects, the anti-neoplastic agent is administered to the subject at a dose of about 10 mg/m². In some aspects, the fludarabine is administered to the subject at a dose of about 10 mg/m².

In some aspects, the infusing bone marrow cells is on the same day as the transplanting. In some aspects, the method further comprises providing thymus irradiation to the subject. In some aspects, the thymus irradiation is provided to the subject 1 day prior to the transplanting. In some aspects, the thymus irradiation is provided to the subject at about 7 Gy.

In some aspects, the method provided herein further comprises administering an anti-IL6R antibody to the subject. In some aspects, the anti-IL6R antibody is tocilizumab. In some aspects, the anti-IL6R antibody is administered to the subject at a dose of about 8 mg/kg. In some aspects, the anti-IL6R antibody is administered to the subject 7 days and 14 days post the transplanting. In some aspects, the anti-IL6R antibody is administered to the subject after the transplanting. In some aspects, the anti-IL6R antibody is administered to the subject after about or after at least about 1 month, 2 months, 3 months, 4 months, 5 months, 6 months, 7 months, 8 months, 9 months, 10 months, 11 months, 12 months, or more than 12 months after the transplanting. In some aspects, the anti-IL6R antibody is administered to the subject after about 3-4 months after the transplanting.

In some aspects, the method provided herein further comprises administering a steroid to the subject. In some aspects, the steroid is administered to the subject on the day of the transplanting. In some aspects, the steroid is administered to the subject for about 20 days after the transplanting. In some aspects, the steroid is administered to the subject for about 6 months after the transplanting. In some aspects, the steroid is corticosteroid. In some aspects, the steroid is administered to the subject after the transplanting. In some aspects, the corticosteroid is administered to the subject after the transplanting. In some aspects, the steroid is administered to the subject after about or after at least about 1 month, 2 months, 3 months, 4 months, 5 months, 6 months, 7 months, 8 months, 9 months, 10 months, 11 months, 12 months, or more than 12 months after the transplanting. In some aspects, the corticosteroid is administered to the subject after about or after at least about 1 month, 2 months, 3 months, 4 months, 5 months, 6 months, 7 months, 8 months, 9 months, 10 months, 11 months, 12 months, or more than 12 months after the transplanting. In some aspects, the steroid is administered to the subject after about 3-4 months after the transplanting. In some aspects, the corticosteroid is administered to the subject after about 3-4 months after the transplanting.

In some aspects, the method provided herein further comprises administering tacrolimus to the subject. In some aspects, the tacrolimus is administered to the subject at a dose of about 4-11 ng/mL. In some aspects, the tacrolimus is administered to the subject at a dose of about 8-10 ng/mL. In some aspects, the tacrolimus is administered to the subject on the day of the transplanting. In some aspects, the tacrolimus is administered to the subject for about 9-12 months post the transplanting. In some aspects, the tacrolimus is administered to the subject for about 1 month post the transplanting. In some aspects, the tacrolimus is administered to the subject after the transplanting. In some aspects, the tacrolimus is administered to the subject after about or after at least about 1 month, 2 months, 3 months, 4 months, 5 months, 6 months, 7 months, 8 months, 9 months, 10 months, 11 months, 12 months, or more than 12 months after the transplanting. In some aspects, the tacrolimus is administered to the subject after about 3-4 months after the transplanting.

In some aspects, the method provided herein further comprises administering mycophenolate mofetil to the subject. In some aspects, the mycophenolate mofetil is administered to the subject at a dose of about 2 g/day. In some aspects, the mycophenolate mofetil is administered to the subject on the day of the transplanting. In some aspects, the mycophenolate mofetil is administered to the subject for about 2 months post the transplanting. In some aspects, the mycophenolate mofetil is administered to the subject after the transplanting. In some aspects, the mycophenolate mofetil is administered to the subject after about or after at least about 1 month, 2 months, 3 months, 4 months, 5 months, 6 months, 7 months, 8 months, 9 months, 10 months, 11 months, 12 months, or more than 12 months after the transplanting. In some aspects, the mycophenolate mofetil is administered to the subject after about 3-4 months after the transplanting.

In some aspects, the method provided herein further comprises administering sirolimus to the subject. In some aspects, the sirolimus is administered to the subject at a dose of about 5-8 ng/mL. In some aspects, the sirolimus is administered to the subject at about or after about 1 month post the transplanting. In some aspects, the sirolimus is administered to the subject for up to about 12 months after the transplanting. In some aspects, the sirolimus is administered to the subject after the transplanting. In some aspects, the sirolimus is administered to the subject after about or after at least about 1 month, 2 months, 3 months, 4 months, 5 months, 6 months, 7 months, 8 months, 9 months, 10 months, 11 months, 12 months, or more than 12 months after the transplanting. In some aspects, the sirolimus is administered to the subject after about 3-4 months after the transplanting.

In some aspects, the method provided herein induces mixed chimerism in the subject. In some aspects, the mixed chimerism is characterized by a percentage of donor cells in the lymphohematopoietic system of the subject of at least about 5%, 10%, 15%, 20%, 25%, 30%, 35%, 40%, 45%, 50%, 55%, 60%, 65%, 70%, 75%, 80%, 85%, or at least about 90%. In some aspects, the mixed chimerism persists for about or at most about 1 month, 2 months, 3 months, 4 months, 5 months, 6 months, 7 months, 8 months, 9 months, 10 months, 11 months, or at most 12 months. In some aspects, the ratio of FoxP3+ T cells to CD4+ T cells is increased in the subject relative to the ratio of FoxP3+ T cells to CD4+ T cells in the absence of the anti-CD2 antibody or antigen binding fragment thereof. In some aspects, the FoxP3+ expression persists in actively proliferating T cells after the last administration of the anti-CD2 antibody or antigen binding fragment thereof.

In some aspects, the method provided herein reduces the risk of Graft versus Host Disease in the subject. In some aspects, the organ or the tissue is kidney. In some aspects, the subject is a human. In some aspects, the donor is a human. In some aspects, the method povided herein induces tolerance in the subject to the transplanted organ or tissue.

### 4. BRIEF DESCRIPTION OF THE FIGURES

The foregoing and other objects, features and advantages will be apparent from the following description of particular embodiments of the invention, as illustrated in the accompanying drawings. The drawings are not necessarily to scale, emphasis instead being placed upon illustrating the principles of various embodiments of the invention.

FIG. 1. Graph showing tolerance overview.

FIG. 2. Table showing schedule of assessment.

FIG. 3. Graph showing tolerance study overview.

FIG. 4. Table showing schedule of assessment.

FIG. 5. Graph showing IL-6 mAb (e.g., tocilizumab) treatment for chimeric transition syndrome (CTS). Graph shows administration of anti-CD2 antibody or antigen binding fragment thereof (e.g., siplizumab) on days −6, −1, 0, and 1 (where day 0 is day of transplant) and administration of IL-6 mAb (e.g., tocilizumab) only if chimeric transition syndrome (CTS) occurs.

FIG. 6. Graph showing prophylactic IL-6 mAb (e.g., tocilizumab) treatment on days 7 and 14 and administration of anti-CD2 antibody or antigen binding fragment thereof (e.g., siplizumab) on days −6, −1, 0, and 1 (where day 0 is day of transplant).

FIG. 7. Graph showing anti-CD2 antibody or antigen binding fragment thereof (e.g., siplizumab) administered 6 days prior to transplant, 1 day prior to transplant, on the same day as transplant, 1 day post transplant, and 6 days post transplant, and prophylactic anti-IL-6R mAb (e.g., tocilizumab) treatment on days 7 and 14.

### 5. DETAILED DESCRIPTION OF THE INVENTION

Described herein are methods for the induction of tolerance of an organ or tissue transplant recipient's immune system towards the transplanted organ or tissue without the need for ongoing immunosuppressive therapy. Provided herein are methods for the combined organ/tissue and bone marrow transplantation preceded by specific conditioning treatment regimen and followed by a specific post-operative treatment regimen. The regimen can be a non-myeloablative procedure including administration of antibodies, immunosuppressors, and radiation that depletes the immune cells of the recipient. In certain embodiments, the components of the regimen can be modified as described herein to achieve mixed chimerism in the recipient. In certain embodiments, the components of the regimen can be modified as described herein to achieve induction of tolerance of an organ/tissue transplant recipient's immune system towards the transplanted organ/tissue without the need for ongoing immunosuppressive therapy.

Organ or tissue transplants and infusion of bone marrow for use with the methods provided herein are described in Section 5.2. Initial conditioning regimen and/or post operative treatment for use with the current methods are described in Section 5.3. Transplantation and bone marrow infusion are described in Section 5.4 and Section 5.5. Methods to assess clinical outcomes are described in Section 5.6. Pharmaceutical compositions are described in Section 5.7. Kits are described in Section 5.8. Examples of the methods provided herein are described in Section 6.

In a specific embodiment, an individual who has been selected to receive or has received an organ or tissue transplant may be treated using the methods described herein. Without being bound by theory, following the treatment regimen induces a state of transient, mixed chimerism. In a specific embodiment, a treatment regimen comprises:
a. Administering rituximab (375 mg/m²) to the recipient at 9 days prior to, 2 days prior to, 5 days post, and 12 days post transplant surgery;
b. Administering anti-CD2 antibody or antigen binding fragment thereof (e.g., siplizumab at about 0.6 mg/kg) 6 days prior to, 1 day prior to, on the same day, and 1 day post transplant surgery, OR administering anti-CD2 antibody or antigen binding fragment thereof (e.g., siplizumab at about 0.6 mg/kg) 6 days prior to, 1 day prior to, on the same day, 1 day post, and 6 days post transplant surgery;
c. Administering cyclophosphamide (60 mg/kg) at 5 days prior and 4 days prior to the transplant surgery;
d. Administering thymus irradiation (e.g., 7 Gy) 1 day prior to transplant surgery; and
e. Administering tocilizumab (e.g., 8 mg/kg) if chimeric transition syndrome (CTS) occurs or on days 7 and 14 post transplant surgery.

In some embodiments, a treatment regimen further comprises administering tacrolimus (e.g., after the transplant (e.g., at a dose of about 4-11 ng/mL), after about or after at least about 1 month, 2 months, 3 months, 4 months, 5 months, 6 months, 7 months, 8 months, 9 months, 10 months, 11 months, 12 months, or more than 12 months after the transplant, and/or 4-11 ng/mL on the day of transplant and for 9-12 months post transplant). In some embodiments, a treatment regimen further comprises administering mycophenolate mofetil (MMF) (e.g., after the transplant (e.g., at a dose of about 2 g/day), after about or after at least about 1 month, 2 months, 3 months, 4 months, 5 months, 6 months, 7 months, 8 months, 9 months, 10 months, 11 months, 12 months, or more than 12 months after the transplant, and/or 2 g/day on the day of transplant and for 2 months post transplant). In some embodiments, a treatment regimen further comprises administering steroid or corticosteroids (e.g., after the transplant, after about or after at least about 1 month, 2 months, 3 months, 4 months, 5 months, 6 months, 7 months, 8 months, 9 months, 10 months, 11 months, 12 months, or more than 12 months after the transplant, and/or on the day of transplant and for 20 days post transplant).

In a specific embodiment, a treatment regimen comprises:
a. Administering rituximab (375 mg/m²) to the recipient at 9 days prior to, 2 days prior to, 5 days post, and 12 days post transplant surgery;
b. Administering anti-CD2 antibody or antigen binding fragment thereof (e.g., siplizumab at about 0.6 mg/kg) 6 days prior to, 1 day prior to, on the same day, and 1 day post transplant surgery;
c. Administering cyclophosphamide (60 mg/kg) at 5 days prior to and 4 days prior to the transplant surgery;
d. Administering thymus irradiation (e.g., 7 Gy) 1 day prior to transplant surgery;
e. Subjecting a patient to organ or tissue (e.g., kidney) transplant and bone marrow cell infusion;
f. Administering tocilizumab (e.g., 8 mg/kg) only if CTS occurs;
g. Administering tacrolimus (e.g., 4-11 ng/ml) on the day of transplant and for 9-12 months post transplant;
h. Administering MMF (e.g., 2 g/day) on the day of transplant and for 2 months post transplant; and
i. Administering corticosteroids on the day of transplant and for 20 days post transplant.

In a specific embodiment, a treatment regimen comprises:
a. Administering rituximab (375 mg/m²) to the recipient at 9 days prior to, 2 days prior to, 5 days post, and 12 days post transplant surgery;
b. Administering anti-CD2 antibody or antigen binding fragment thereof (e.g., siplizumab at about 0.6 mg/kg) 6 days prior to, 1 day prior to, on the same day, and 1 day post transplant surgery;
c. Administering cyclophosphamide (60 mg/kg) at 5 days prior to and 4 days prior to the transplant surgery;
d. Administering thymus irradiation (e.g., 7 Gy) 1 day prior to transplant surgery; and
e. Subjecting a patient to organ or tissue (e.g., kidney) transplant and bone marrow cell infusion.

In some embodiments, tocilizumab (e.g., 8 mg/kg) is administered after the organ or tissue transplant. In some embodiments, tacrolimus (e.g., 4-11 ng/ml) is administered after the organ or tissue transplant. In some embodiments, MMF (e.g., 2 g/day) is administered after the organ or tissue transplant. In some embodiments, steroid or corticosteroid is administered after the organ or tissue transplant. In some embodiments, tocilizumab, tacrolimus, MMF, steroid, and/or corticosteroid is/are administered to a subject after about or after at least about 1 month, 2 months, 3 months, 4 months, 5 months, 6 months, 7 months, 8 months, 9 months, 10 months, 11 months, 12 months, or more than 12 months after the transplant.

In a specific embodiment, a treatment regimen comprises:
a Administering rituximab (375 mg/m²) to the recipient at 9 days prior to, 2 days prior to, 5 days post, and 12 days post transplant surgery;
b. Administering anti-CD2 antibody or antigen binding fragment thereof (e.g., siplizumab at about 0.6 mg/kg) 6 days prior to, 1 day prior to, on the same day, and 1 day post transplant surgery;
c. Administering cyclophosphamide (60 mg/kg) at 5 days prior to and 4 days prior to the transplant surgery;
d. Administering thymus irradiation (e.g., 7 Gy) 1 day prior to transplant surgery;
e. Subjecting a patient to organ or tissue (e.g., kidney) transplant and bone marrow cell infusion;
f. Administering tocilizumab (e.g., 8 mg/kg) at 7 days post and 14 days post transplant;
g. Administering tacrolimus (e.g., 4-11 ng/ml) on the day of transplant and for 9-12 months post transplant;
h. Administering MMF (e.g., 2 g/day) on the day of transplant and for 2 months post transplant; and
i. Administering corticosteroids on the day of transplant and for 20 days post transplant.

In a specific embodiment, a treatment regimen comprises:
a. Administering rituximab (375 mg/m²) to the recipient at 9 days prior to, 2 days prior to, 5 days post, and 12 days post transplant surgery;
b. Administering anti-CD2 antibody or antigen binding fragment thereof (e.g., siplizumab at about 0.6 mg/kg) 6 days prior to, 1 day prior to, on the same day, 1 day post, and 6 days post transplant surgery;
c. Administering cyclophosphamide (60 mg/kg) at 5 days prior to and 4 days prior to the transplant surgery;
d. Administering thymus irradiation (e.g., 7 Gy) 1 day prior to transplant surgery;
e. Subjecting a patient to organ or tissue (e.g., kidney) transplant and bone marrow cell infusion;
f. Administering tocilizumab (e.g., 8 mg/kg) at 7 days post and 14 days post transplant;
g. Administering tacrolimus (e.g., 4-11 ng/ml) on the day of transplant and for 9-12 months post transplant;
h. Administering MMF (e.g., 2 g/day) on the day of transplant and for 2 months post transplant; and
i. Administering corticosteroids on the day of transplant and for 20 days post transplant.

In a specific embodiment, a treatment regimen comprises:
a. Administering rituximab (375 mg/m²) to the recipient at 9 days prior to and 2 days prior to transplant surgery;
b. Administering anti-CD2 antibody or antigen binding fragment thereof (e.g., siplizumab at about 0.6 mg/kg) 1 day prior to, on the same day, and 1 day post transplant surgery;
c. Administering cyclophosphamide (22.5 mg/kg) at 5 days prior and 4 days prior to the transplant surgery;
d. Administering fludarabine (e.g., 10 mg/m²) at 6 days prior to, 5 days prior to, 4 days prior to, and 3 days prior to transplant surgery; and
e. Administering thymus irradiation (e.g., 7 Gy) 1 day prior to transplant surgery.

In some embodiments, a treatment regimen further comprises administering tacrolimus (e.g., after the transplant (e.g., at a dose of about 8-10 ng/mL), after about or after at least about 1 month, 2 months, 3 months, 4 months, 5 months, 6 months, 7 months, 8 months, 9 months, 10 months, 11 months, 12 months, or more than 12 months after the transplant, and/or 8-10 ng/mL on the day of transplant and for 1 month post transplant). In some embodiments, a treatment regimen further comprises administering sirolimus or mTOR inhibitor (e.g., after the transplant (e.g., at a dose of about 5-8 ng/ml), after about or after at least about 1 month, 2 months, 3 months, 4 months, 5 months, 6 months, 7 months, 8 months, 9 months, 10 months, 11 months, 12 months, or more than 12 months after the transplant, and/or 5-8 ng/mL 1 month post transplant and until 12 months post transplant). In some embodiments, a treatment regimen further comprises administering corticosteroids (e.g., after the transplant, after about or after at least about 1 month, 2 months, 3 months, 4 months, 5 months, 6 months, 7 months, 8 months, 9 months, 10 months, 11 months, 12 months, or more than 12 months after the transplant, and/or on the day of transplant and for 6 months post transplant).

In a specific embodiment, a treatment regimen comprises:
a. Administering rituximab (375 mg/m²) to the recipient at 9 days prior to and 2 days prior to transplant surgery;
b. Administering anti-CD2 antibody or antigen binding fragment thereof (e.g., siplizumab at about 0.6 mg/kg) 1 day prior to, on the same day, and 1 day post transplant surgery;
c. Administering cyclophosphamide (22.5 mg/kg) at 5 days prior and 4 days prior to the transplant surgery;
d. Administering fludarabine (e.g., 10 mg/m²) at 6 days prior to, 5 days prior to, 4 days prior to, and 3 days prior to transplant surgery;
e. Administering thymus irradiation (e.g., 7 Gy) 1 day prior to transplant surgery;
f. Subjecting a patient to organ or tissue (e.g., kidney) transplant and bone marrow cell infusion;
g. Administering tacrolimus (e.g., 8-10 ng/ml) on the day of transplant and for 1 month post transplant;
h. Administering sirolimus (e.g., 5-8 ng/mL) or mTOR inhibitor for 1 month post transplant and until 12 months post transplant; and
i. Administering corticosteroids on the day of transplant and for 6 months post transplant.

In a specific embodiment, a treatment regimen comprises:
a. Administering rituximab (375 mg/m²) to the recipient at 9 days prior to and 2 days prior to transplant surgery;
b. Administering anti-CD2 antibody or antigen binding fragment thereof (e.g., siplizumab at about 0.6 mg/kg) 1 day prior to, on the same day, and 1 day post transplant surgery;
c. Administering cyclophosphamide (22.5 mg/kg) at 5 days prior and 4 days prior to the transplant surgery;
d. Administering fludarabine (e.g., 10 mg/m²) at 6 days prior to, 5 days prior to, 4 days prior to, and 3 days prior to transplant surgery;
e. Administering thymus irradiation (e.g., 7 Gy) 1 day prior to transplant surgery; and
f. Subjecting a patient to organ or tissue (e.g., kidney) transplant and bone marrow cell infusion.

In some embodiments, tacrolimus (e.g., 8-10 ng/mL) is administered to the subject after the transplant (e.g., after the transplant, after about or after at least about 1 month, 2 months, 3 months, 4 months, 5 months, 6 months, 7 months, 8 months, 9 months, 10 months, 11 months, 12 months, or more than 12 months after the transplant). In some embodiments, sirolimus (e.g., 5-8 ng/mL) or mTOR inhibitor is administered to the subject after the transplant (e.g., after the transplant, after about or after at least about 1 month, 2 months, 3 months, 4 months, 5 months, 6 months, 7 months, 8 months, 9 months, 10 months, 11 months, 12 months, or more than 12 months after the transplant). In some embodiments, steroid or corticosteroid is administered to the subject after the transplant (e.g., after the transplant, after about or after at least about 1 month, 2 months, 3 months, 4 months, 5 months, 6 months, 7 months, 8 months, 9 months, 10 months, 11 months, 12 months, or more than 12 months after the transplant).

In some embodiments, described herein is a method of transplanting an organ and/or a tissue from a donor to a subject or a method of minimizing chimeric transition syndrome in a subject in need of an organ or a tissue transplant, wherein the method comprises: a) administering a B-cell depleting antibody to the subject; b) administering an anti-CD2 antibody or antigen binding fragment thereof to the subject; c) transplanting the organ or the tissue into the subject; and d) infusing bone marrow cells from the donor to the subject. In some embodiments, the anti-CD2 antibody or antigen binding fragment thereof is administered to the subject more than 2 days prior to the organ and/or tissue transplant. In some embodiments, the anti-CD2 antibody or antigen binding fragment thereof is not administered to the subject 2 days prior to the organ and/or tissue transplant. In some embodiments, the B-cell depleting antibody is administered to the subject more than 7 days prior to the organ and/or tissue transplant. In some embodiments, a first dose of the anti-CD2 antibody or antigen binding fragment thereof is 1 day prior to the organ and/or tissue transplant.

In some embodiments, described herein is a method of transplanting an organ and/or a tissue from a donor to a subject or a method of minimizing chimeric transition syndrome in a subject in need of an organ or a tissue transplant, wherein the method comprises: a) administering a B-cell depleting antibody to the subject; b) administering an anti-CD2 antibody or antigen binding fragment thereof to the subject; c) administering fludarabine to the subject; d) transplanting the organ or the tissue into the subject; and e) infusing bone marrow cells from the donor to the subject.

In some embodiments, the treatment regimen as described herein can be modified. Specifically, route of administration, dose, and exact timing of the various active pharmaceutical ingredients can be modified to adjust to the specific circumstances of the transplantation to achieve induction of tolerance of an organ transplant recipient's immune system towards the transplanted organ without the need for ongoing immunosuppressive therapy. In some embodiments, the treatment regimen as described herein can be modified, for example, to administer a comparable agent instead of or in addition to an agent described herein (e.g., in some embodiments, a B-cell depleting antibody is administered to a subject instead of or in addition to rituximab; an anti-IL-6R antibody is administered to a subject instead of or in addition to tocilizumab; an anti-CD2 antibody or an antigen binding fragment thereof is administered to a subject instead of or in addition to siplizumab; an anti-neoplastic agent is administered to a subject instead of or in addition to fludarabine; an antiproliferative agent is administered to a subject instead of or in addition to MMF; an immunosuppressant agent is administered to a subject instead of or in addition to cyclophosphamide; an immunosuppressant agent is administered to a subject instead of or in addition to tacrolimus; an mTOR inhibitor is administered to a subject instead of or in addition to sirolimus; and/or a steroid is administered to a subject instead of or in addition to corticosteroid). Without being bound by theory, the treatment regimen is modified such that it still results in transient, mixed chimerism. In certain embodiments, transient mixed chimerism is characterized by a percentage of donor cells in the lymphohematopoietic system of at least 5%, 10%, 15%, 20%, 25%, 30%, 35%, 40%, 45%, 50%, 55%, 60%, 65%, 70%, 75%, 80%, 85%, or at least 90%. In certain embodiments, transient mixed chimerism persists for at most 1, 2, 3, 4, 5, 6, 7, 8, 9, 10, 11 or at most 12 months. The ratio of FoxP3+ T cells to CD4+ T cells can be increased in the transplant recipient relative to the ratio of FoxP3+ T cells to CD4+ T cells in the absence of anti-CD2 antibody. FoxP3+ expression can persist in actively proliferating T cells after the last administration of anti-CD2 antibody. Mixed chimerism can be assayed as described in e.g., Sections 5.6 and 6.

### 5.1 Recipients

The terms “subject,” “recipient,” and “patient” are used interchangeably herein. In some embodiments, the subject is a mammal. In some embodiments, a subject or a subject in need is a subject who has or is predicted of having or developing an immune related disorder or disease. In some embodiments, a subject or a subject in need is a subject who has or will undergo transplant surgery. In some embodiments, a subject or a subject in need is a subject who has chronic or acute inflammatory disorder of the immune system. In some embodiments, the subject is a primate. In certain embodiments, the subject is a human. In some embodiments, the subject is an adult human. In some embodiments, the subject is a child. In some embodiments, the subject is a pediatric patient. In some embodiments, the subject is a juvenile human. In some embodiments, the subject is at least 5 years old, 10 years old, 15 years old, 18 years old, 21 years old, 50 years old, 60 years old, 65 years old, 70 years old, or more than 70 years old. In some embodiments, the subject is at most 5 years old, 10 years old, 15 years old, 18 years old, or 21 years old. In some embodiments, the subject has not received prior treatment related to organ transplant or for an immune associated disorder or disease. In some embodiments, the subject is a treatment naïve subject. In some embodiments, the subject is treatment naïve to a treatment for an immune associated disorder or disease. In some embodiments, the subject has not received prior treatment with an anti-CD2 antibody or an antigen-binding fragment thereof and/or with an agent described herein.

Individuals who have been selected to receive an organ transplant may follow these methods described herein with the goal of inducing a state of mixed chimerism in which the recipient and donor hematopoietic cells coexist in the recipient. When the state of mixed chimerism is achieved, it reduces the need for long-term immunosuppressive therapies.

Subjects who have undergone or will undergo an organ and/or tissue transplant can be suitable for treatment with a method provided herein. The methods described herein can be used for a subject who has undergone or will undergo any organ and/or tissue transplant (e.g., lung, heart, kidney, liver, stomach, intestines, pancreas, skin, or spleen). Individuals whose organ has been damaged by means including injury, disease, or birth defect may meet the criteria to receive an organ (e.g., liver or kidney) transplant and may be treated in accordance with the methods described herein. A recipient treated in accordance with the methods described herein may have required an organ transplant for any reason. Generally, a patient suffering from end-stage organ disease whose life expectancy is predicted to be extended by an organ transplant beyond the life expectancy without the organ transplant may be considered for an organ transplant.

In specific embodiments, a recipient treated in accordance with the methods described herein has received an organ transplant necessitated by a genetic disease. In specific embodiments, the organ transplant is a living donor organ transplant. In specific embodiments, the organ transplant is a deceased donor organ transplant. In some embodiments, the organ transplant may be an ABO (major human blood group system type A, type B, type O) compatible transplant. In some embodiments, the organ transplant may be an ABO incompatible transplant. In some embodiments, a recipient treated in accordance with the methods described herein can have a Model for End-Stage Liver Disease (MELD) score of less than 30, less than 20, or less than 10. In some embodiments, a recipient treated in accordance with the methods described herein can be seropositive for Epstein-Barr Virus (EBV). In some embodiments, the recipient has undergone a splenectomy prior to receiving the transplant. In some embodiments, the recipient has not undergone a splenectomy prior to receiving the transplant. In some embodiments, a subject is in need or has received a liver transplant. Examples of indications of a liver transplant are described, for example, in the EASL Clinical Practice Guidelines: Liver transplantation (J Hepatol. 2016 February; 64(2):433-485. doi: 10.1016/j.jhep.2015.10.006).

In some embodiments, the recipient can have a highly sensitized immune system. The development of a highly sensitized immune system is caused by previous exposure to foreign tissues and the development of antibodies towards those tissues. Causes of this exposure can include blood transfusions, a previous transplant, or pregnancy. In the sensitized state, the immune system is hyper-vigilant and produces antibodies that will attack the transplanted organ. In certain embodiments, the recipient as described herein can have a sensitized or unsensitized immune system. Some recipients can be sensitized to rituximab. In such recipients, IdeS can be administered with the rituximab. IdeS, an enzyme that cleaves the heavy chain of the IgG, inhibits antibody dependent cellular cytotoxicity (ADCC) and complement dependent cytotoxicity (CDC). In certain embodiments, IdeS is administered in combination with rituximab or an anti-CD19 plasma cell-directed treatment. In certain embodiments, plasmapheresis can be used in combination with IdeS treatment and rituximab.

### 5.2 Transplant and Donors

### 5.2.1 Organ or Tissue Transplant

As used herein, the donor is the individual from which the organ or tissue to be transplanted is taken. In some embodiments, the donor is of the same species as the recipient and the donor can be alive or deceased. The donor can be related to the recipient or not related to the recipient. As used herein, the recipient is the individual that will receive or has received the transplanted organ or tissue. The recipient can be related or not related to the donor. The recipient can be HLA-matched or HLA-mismatched with the donor. In some embodiments, the organ or tissue to be transplanted is artificially made (e.g., not from a mammal), or made in vitro, or made outside of a body. In some embodiments, the organ or tissue to be transplanted is from a different species as the recipient (e.g., an organ or tissue from a different animal/mammal). In some embodiments, the organ or tissue to be transplanted is not from a human.

Organs that can be transplanted utilizing the methods provided herein can be any solid organ. In some embodiments, the organ or tissue can be or can be derived from a kidney, heart, intestine, liver, lung, pancreas or other organ or tissue that can be transplanted using the methods provided herein. In some embodiments, the organ or tissue can be a vascular-composite allograft including hands, feet, other limbs, faces, or other body parts that can be transplanted using the methods provided herein. In some embodiments, the transplanted organ or tissue may be whole organ, a part of an organ, or cells derived from an organ.

### 5.2.2 Bone Marrow

Hematopoietic stem cells can be derived from bone marrow of the donor. In some embodiments, the bone marrow cells, obtained from the organ or tissue donor, can be HLA-matched to the recipient. In certain embodiments, the bone marrow cells, obtained from the organ or tissue donor, can be HLA-mismatched. In certain embodiments, the bone marrow cell transplant can be combined with the organ or tissue transplant, can occur after the organ or tissue transplant, or can occur prior to the organ or tissue transplant.

In certain embodiments, the desired outcome can be to induce a state of chimerism in the recipient. By performing a combined transplant of a solid organ or tissue and an infusion of hematopoietic cells from unprocessed donor bone marrow, in combination with non-myeloablative conditioning, the recipient may develop persistent mixed chimerism. As used herein, “chimerism” refers to a state where both donor hematopoietic cells and recipient hematopoetic cells coexist. In certain embodiments, the recipient may be monitored to assess the presence of mixed chimerism. In certain embodiments, the recipient will have or has at least 1% circulating donor hematopoetic cells. In certain embodiments, the recipient will have about or at least about 5%, 10%, 15%, 20%, 25%, 30%, 35%, 40%, 45%, 50%, 55%, 60%, 65%, 70%, 75%, 80%, 85%, 90%, or 95% circulating donor hematopoetic cells. In certain embodiments, the recipient will have or has about 1-10%, 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, or 85-95% circulating donor hematopoetic cells. In certain embodiments, this state of chimerism can last for hematopoietic and/or immune cells, for a period of time, for example for 5 days. 10 days, 15 days, 25 days, 30 days, 1 month, 2 months, 3 months, 4 months, 5 months, 6 months, or longer than 6 months. In a specific embodiment, the chimerism persist in the recipient for at least 6 months. In certain embodiments, the components of the bone marrow transplant can be modified as described herein to achieve and/or maintained mixed chimerism in the recipient as described herein.

### 5.3 Conditioning Regimen and Postoperative Regimen

The conditioning regimen or use with the methods provided herein can include administration of one or more of the treatments below. In some embodiments, an anti-CD2 antibody or antigen binding fragment thereof is provided to a subject. In some embodiments, an anti-CD2 antibody or antigen binding fragment thereof (e.g., siplizumab) is provided to a subject prior to transplant or cell infusion (e.g., bone marrow cell infusion).

The conditioning regimen or use with the methods provided herein can include administration of one or more than one agent described herein (e.g., rituximab, cyclophosphamide, tocilizumab, corticosteroid, tacrolimus, MMF, antiproliferative agent, steroid, sirolimus, polyclonal rabbit anti-thymocyte globulin (rATG), fludarabine, B- or plasma cell depleting antibody, immunosuppressive agent, chemotherapeutic agent, antiproliferative agent, antineoplastic agent, anti-IL6R antibody, and/or irradiation) as described herein. In some embodiments, an agent or more than one agent as described herein is administered with, prior to, or after thymic (or thymus) irradiation, transplant, and/or cell (e.g., bone marrow) infusion. In some embodiments, an agent or more than one agent is administered prophylactically (e.g., tocilizumab).

In some embodiments, an agent or more than one agent is administered to a subject prior to transplant. In some embodiments, an agent or more than one agent is or comprises an anti-CD2 antibody or antigen binding fragment thereof and/or a non-myeloblative agent. In some embodiments, an anti-CD2 antibody or antigen binding fragment thereof is administered in combination with a non-myeloblative agent. In some embodiments, an anti-CD2 antibody or antigen binding fragment thereof is administered prior to, on the same day, or after a non-myeloblative agent. In some embodiments, a non-myeloblative agent includes one or more than one from a B-cell depleting antibody, rituximab, fludarabine, cyclophosphamide, chemotherapy agent, and/or irradiation (e.g., thymic irradiation). In some embodiments, a non-myeloblative agent includes fludarabine and cyclophosphamide. In some embodiments, a non-myeloblative agent includes fludarabine (or a comparable/replaceable agent or an agent from the same class as fludarabine). In some embodiments, a non-myeloblative agent includes fludarabine at about 10 mg/m². In some embodiments, a non-myeloblative agent includes cyclophosphamide (or a comparable/replaceable agent or an agent from the same class as cyclophosphamide). In some embodiments, a non-myeloblative agent includes cyclophosphamide at about 22.5 mg/kg. In some embodiments, a non-myeloblative agent includes cyclophosphamide at about 60 mg/kg. In some embodiments, a non-myeloblative agent includes a B-cell depleting antibody (e.g., rituximab (at e.g., about 375 mg/m²). In some embodiments, a non-myeloblative agent includes low exposure fludarabine and cyclophosphamide. In some embodiments, a non-myeloblative agent includes fludarabine at about 10 mg/m² and cyclophosphamide at about 22.5 mg/kg. In some embodiments, a non-myeloblative agent includes cell infusion (e.g., bone marrow cell infusion). In some embodiments, a non-myeloblative agent includes thymic irradiation. In some embodiments, a non-myeloblative agent includes rituximab, cyclophosphamide, and/or thymic irradiation.

In some embodiments, an agent or more than one agent is administered to a subject prior to cell infusion (e.g., bone marrow cell infusion). In some embodiments, an agent or more than one agent is administered to a subject after a transplant. In some embodiments, an agent or more than one agent is administered to a subject after cell infusion (e.g., bone marrow cell infusion). In some embodiments, an agent or more than one agent is administered to a subject on the same day as the day of the transplant. In some embodiments, an agent or more than one agent is administered to a subject on the same day as the day of cell infusion (e.g., bone marrow cell infusion). In some embodiments, an agent or more than one agent is administered to a subject about or at least about 20, 19, 18, 17, 16, 15, 14, 13, 12, 11, 10, 9, 8, 7, 6, 5, 4, 3, 2, and/or 1 day(s) prior to transplant. In some embodiments, an agent or more than one agent is administered to a subject about or at least about 20, 19, 18, 17, 16, 15, 14, 13, 12, 11, 10, 9, 8, 7, 6, 5, 4, 3, 2, and/or 1 day(s) prior to cell infusion. In some embodiments, an agent or more than one agent is administered to a subject about or at least about 20, 19, 18, 17, 16, 15, 14, 13, 12, 11, 10, 9, 8, 7, 6, 5, 4, 3, 2, and/or 1 day(s) after the transplant. In some embodiments, an agent or more than one agent is administered to a subject about or at least about 20, 19, 18, 17, 16, 15, 14, 13, 12, 11, 10, 9, 8, 7, 6, 5, 4, 3, 2, and/or 1 day(s) after cell infusion. In some embodiments, an agent or more than one agent is administered to a subject 9 days prior to transplant. In some embodiments, an agent or more than one agent is administered to a subject 9 days prior to cell infusion. In some embodiments, an agent or more than one agent is administered to a subject 6 days prior to transplant. In some embodiments, an agent or more than one agent is administered to a subject 6 days prior to cell infusion. In some embodiments, an agent or more than one agent is administered to a subject 5 days prior to transplant. In some embodiments, an agent or more than one agent is administered to a subject 5 days prior to cell infusion. In some embodiments, an agent or more than one agent is administered to a subject 4 days prior to transplant. In some embodiments, an agent or more than one agent is administered to a subject 4 days prior to cell infusion. In some embodiments, an agent or more than one agent is administered to a subject 3 days prior to transplant. In some embodiments, an agent or more than one agent is administered to a subject 3 days prior to cell infusion. In some embodiments, an agent or more than one agent is administered to a subject 2 days prior to transplant. In some embodiments, an agent or more than one agent is administered to a subject 2 days prior to cell infusion. In some embodiments, an agent or more than one agent is administered to a subject 1 day prior to transplant. In some embodiments, an agent or more than one agent is administered to a subject 1 day prior to cell infusion. In some embodiments, an agent or more than one agent is administered to a subject 1 day after the transplant. In some embodiments, an agent or more than one agent is administered to a subject 1 day after cell infusion. In some embodiments, an agent or more than one agent is administered to a subject at least one, at least two, at least three, at least four, at least five, at least six, at least seven, at least eight, at least nine, at least ten, or more than at least ten times prior to a transplant. In some embodiments, an agent or more than one agent is administered to a subject at least one, at least two, at least three, at least four, at least five, at least six, at least seven, at least eight, at least nine, at least ten, or more than at least ten times prior to cell infusion. In some embodiments, an agent or more than one agent is administered to a subject at least one, at least two, at least three, at least four, at least five, at least six, at least seven, at least eight, at least nine, at least ten, or more than at least ten days prior to a transplant. In some embodiments, an agent or more than one agent is administered to a subject at least one, at least two, at least three, at least four, at least five, at least six, at least seven, at least eight, at least nine, at least ten, or more than at least ten days prior to cell infusion. In some embodiments, an agent or more than one agent is administered to a subject at least one, at least two, at least three, at least four, at least five, at least six, at least seven, at least eight, at least nine, at least ten, or more than at least ten times after a transplant. In some embodiments, an agent or more than one agent is administered to a subject at least one, at least two, at least three, at least four, at least five, at least six, at least seven, at least eight, at least nine, at least ten, or more than at least ten times after cell infusion. In some embodiments, an agent or more than one agent is administered to a subject at least one, at least two, at least three, at least four, at least five, at least six, at least seven, at least eight, at least nine, at least ten, or more than at least ten days after a transplant. In some embodiments, an agent or more than one agent is administered to a subject at least one, at least two, at least three, at least four, at least five, at least six, at least seven, at least eight, at least nine, at least ten, or more than at least ten days after cell infusion.

In some embodiments, an agent or more than one agent (e.g., anti-CD2 antibody or antigen binding fragment thereof or an agent or a non-myeloblative agent) is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) prior to transplant and/or cell infusion. In some embodiments, an agent or more than one agent (e.g., anti-CD2 antibody or antigen binding fragment thereof or an agent or a non-myeloblative agent) is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) after the transplant and/or cell infusion.

In some embodiments, an agent or more than one agent is administered to a subject for a continuous amount of time/days prior to a transplant. In some embodiments, an agent or more than one agent administered to a subject for a continuous amount of time/days prior to cell infusion. In some embodiments, an agent or more than one agent is administered to a subject for a continuous amount of time/days after a transplant. In some embodiments, an agent or more than one agent is administered to a subject for a continuous amount of time/days after cell infusion. In some embodiments, an agent or more than one agent is administered to a subject for about, at least about, or at most about 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 20 months, 21 months, 22 months, 23 months, 24 months, or more than 24 months after a transplant. In some embodiments, an agent or more than one agent is administered to a subject for about or at most about 12 months. In some embodiments, an agent or more than one agent is administered to a subject for about or at most about 18 months. In some embodiments, an agent or more than one agent is administered to a subject for about, at least about, or at most about 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 12 months, or more than 12 months after cell infusion.

In some embodiments, an agent or more than one agent is administered to a subject 9 and/or 2 days prior to transplant. In some embodiments, an agent or more than one agent is administered to a subject 9 and/or 2 days prior to cell infusion. In some embodiments, an agent or more than one agent is administered to a subject 6, 5, 4 and/or 3 days prior to transplant. In some embodiments, an agent or more than one agent is administered to a subject 6, 5, 4 and/or 3 days prior to cell infusion. In some embodiments, an agent or more than one agent is administered to a subject one day prior to transplant, on the same day as the transplant, and/or on the day after the transplant. In some embodiments, an agent or more than one agent is administered to a subject one day prior to cell infusion, on the same day as cell infusion, and/or on the day after cell infusion. In some embodiments, an agent or more than one agent is administered to a subject six days prior to transplant, one day prior to transplant, on the same day as the transplant, on the day after the transplant, and/or six days after the transplant. In some embodiments, an agent or more than one agent is administered to a subject six days prior to cell infusion, one day prior to cell infusion, on the same day as cell infusion, on the day after cell infusion, and/or six days after cell infusion. In some embodiments, an agent or more than one agent is administered to a subject on the same day as the transplant. In some embodiments, an agent or more than one agent is administered to a subject on the same day as cell infusion. In some embodiments, an agent or more than one agent is administered to a subject one day after the transplant. In some embodiments, an agent or more than one agent is administered to a subject one day after cell infusion.

In some embodiments, an agent or more than one agent is or comprises a B-cell depleting antibody (e.g., rituximab at, e.g., 375 mg/m²) and is administered to a subject (e.g., on days −9 and/or −2 prior to transplant/cell infusion). In some embodiments, an agent or more than one agent is or comprises a B-cell depleting antibody (e.g., rituximab at, e.g., 375 mg/m²) and is administered to a subject (e.g., on days −9 and/or −2 prior to transplant/cell infusion and/or five days after transplant/cell infusion, and/or twelve days after transplant/cell infusion). In some embodiments, an agent or more than one agent is or comprises an anti-neoplastic agent (e.g., fludarabine at, e.g., 10 mg/m²) and is administered to a subject (e.g., on days −6, −5, −4, and/or −3 prior to transplant/cell infusion). In some embodiments, an agent or more than one agent is or comprises an anti-CD2 antibody or an antigen-binding fragment thereof (e.g., siplizumab at 0.6 mg/kg) and is administered to a subject (e.g., on the day prior to transplant/cell infusion (day −1), on the day of the transplant/cell infusion (day 0), and/or on the day after the transplant/cell infusion (day 1)). In some embodiments, an agent or more than one agent is or comprises an anti-CD2 antibody or an antigen-binding fragment thereof (e.g., siplizumab at 0.6 mg/kg) and is administered to a subject (e.g., six days prior to transplant/cell infusion, on the day prior to transplant/cell infusion (day −1), on the day of the transplant/cell infusion (day 0), and/or on the day after the transplant/cell infusion (day 1)). In some embodiments, an agent or more than one agent is or comprises an anti-CD2 antibody or an antigen-binding fragment thereof (e.g., siplizumab at 0.6 mg/kg) and is administered to a subject (e.g., six days prior to transplant/cell infusion, on the day prior to transplant/cell infusion (day −1), on the day of the transplant/cell infusion (day 0), on the day after the transplant/cell infusion (day 1), and/or six days after the transplant (day 6)). In some embodiments, an agent or more than one agent is or comprises a chemotherapy agent or immunosuppressive agent (e.g., cyclophosphamide, e.g., at 22.5 mg/kg) and is administered to a subject (e.g., on days −5 and/or −4 prior to transplant). In some embodiments, irradiation (e.g., thymus irradiation, 7 Gy) is given to the subject on the day prior to transplant. In some embodiments, an agent or more than one agent is or comprises an immunosuppressive agent (e.g., tacrolimus, e.g., at 8-10 ng/ml) and is administered to the subject (e.g., on the day of transplant and/or for about 1 month). In some embodiments, an agent or more than one agent is or comprises an immunosuppressive agent (e.g., tacrolimus, e.g., at 4-11 ng/mL) and is administered to the subject (e.g., on the day of transplant and/or for about 9-12 months). In some embodiments, an agent or more than one agent is or comprises an antiproliferative agent (e.g., MMF, e.g., at about 1 g/day) and is administered to the subject (e.g., on the day of transplant and/or for about 1-2 months). In some embodiments, an agent or more than one agent is or comprises an immunosuppressive agent (e.g., corticosteroid) and is administered to the subject (e.g., on the day of transplant and/or for about 6 months). In some embodiments, an agent or more than one agent is or comprises an immunosuppressive agent (e.g., corticosteroid) and is administered to the subject (e.g., on the day of transplant and/or for about 20 days). In some embodiments, an agent or more than one agent is or comprises an immunosuppressive agent (e.g., sirolimus, e.g., initial trough 5-8 ng/ml) and is administered to the subject (e.g., one month after the day of transplant and/or for about 1-12 months). In some embodiments, an agent or more than one agent is or comprises an anti-IL6R antibody (e.g., tocilizumab) and is administered to a subject (e.g., if CTS occurs, seven days after transplant or cell infusion, and/or fourteen days after transplant or cell infusion).

### 5.3.1 Treatment of the Transplant

In certain embodiments, the conditioning regimen can include direct infusion of an anti-CD2 antibody or antigen binding fragment thereof (e.g., siplizumab) into the organ. In certain embodiments, the transplant can be directly injected with the anti-CD2 antibody or antigen binding fragment thereof. This injection can be administered at one site on the transplant or can be administered at multiple sites on the transplant. In certain embodiments, the transplant can be incubated in a bath containing the anti-CD2 antibody or antigen binding fragment thereof. This incubation can occur during transport to the surgery or at the surgical location. In some embodiments, incubation last for a duration of sufficient time to allow uptake of the anti-CD2 antibody or antigen binding fragment thereof into the transplant. This incubation can take place for about, at least about, or at most about: 15 minutes, 30 minutes, 45 minutes, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, 5.75 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours, or 24 hours. In certain embodiments, the dose of the anti-CD2 antibody or antigen binding fragment thereof for injection is about, at least about, or at most about: 0.01 mg/kg, 0.05 mg/kg, 0.1 mg/kg, 0.5 mg/kg, 0.6 mg/kg, 1 mg/kg, 5 mg/kg, 10 mg/kg, or 50 mg/kg.

In certain embodiments, the organ or tissue is incubated in a bath comprising anti-CD2 antibody or antigen binding fragment thereof (e.g., siplizumab). In some embodiments, the organ or tissue is maintained in a bath comprising anti-CD2 antibody or antigen binding fragment thereof from removing of the organ or tissue from the donor until implantation in the recipient. In certain embodiments, the concentration of the anti-CD2 antibody or antigen binding fragment thereof in the organ or tissue bath is about or at least about 0.01 mg/liter, about or at least about 0.05 mg/liter, about or at least about 0.1 mg/liter, about or at least about 0.5 mg/liter, about or at least about 0.6 mg/liter, about or at least about 1 mg/liter, about or at least about 5 mg/liter, about or at least about 10 mg/liter, or about or at least about 50 mg/liter.

In certain embodiments, the anti-CD2 antibody or antigen binding fragment thereof can be administered to the organ or tissue by way of the circulatory system. In some embodiments, a catheter can be inserted into an adjacent blood vessel and attached to a pump. The anti-CD2 antibody or antigen binding fragment thereof can be pumped through the blood vessel and delivered to the organ. In certain embodiments, the treatment of the transplant can be modified as described herein to achieve and/or maintained mixed chimerism in the recipient.

### 5.3.2 Anti-CD2 Antibody or Antigen Binding Fragment Thereof

In certain embodiments, the methods described herein include administering an anti-CD2 antibody or antigen binding fragment thereof to a subject (e.g., the recipient). As defined herein, an antibody refers to an immunoglobulin including IgG, IgM, IgE, IgA, and IgD. The antibody described herein can be a monoclonal antibody or a polyclonal antibody. In some embodiments the antibody can be a chimeric antibody. In some embodiments, the antibody can be a humanized antibody. In a specific embodiment, the antibody is a recombinant antibody. In a specific embodiment, the antibody is a humanized antibody. In a specific embodiment, an anti-CD2 antibody or antigen binding fragment thereof described herein is an IgG. In a certain embodiment, the IgG can be an IgG1, IgG2, IgG3, or an IgG4. In specific embodiments, an anti-CD2 antibody or antigen binding fragment thereof described herein is an IgG1, IgG2, or an IgG4. As described herein, an anti-CD2 antibody or antigen binding fragment thereof described herein can be an antigen-binding fragment. In certain embodiments, an antigen-binding fragment is, or can comprise, Fab, F(ab′)2, scFv (VH fused to a VL), or sdAb.

In some embodiments, an anti-CD2 antibody or antigen binding fragment thereof for use with the present methods and compositions has the CDR sequences of rat anti-CD2 monoclonal antibody BTI-322. In some embodiments, an anti-CD2 antibody or antigen binding fragment thereof can be a humanized IgG1 version of BTI-322 (siplizumab; MEDI-507). In some embodiments, an anti-CD2 antibody or antigen binding fragment thereof is an Fc-silent anti-CD2 antibody or an antigen-binding fragment thereof. In some embodiments, an anti-CD2 antibody or antigen binding fragment thereof is BTI-322 or an antigen-binding fragment thereof. In some embodiments, an anti-CD2 antibody or antigen binding fragment thereof is CB.219 or an antigen-binding fragment thereof. In some embodiments, an anti-CD2 antibody or antigen binding fragment thereof is LO-CD2b or an antigen-binding fragment thereof. In some embodiments, an anti-CD2 antibody or antigen binding fragment thereof is siplizumab or an antigen-binding fragment thereof. In some embodiments, an anti-CD2 antibody or antigen binding fragment thereof is anti-CD2 antibody 1 or an antigen-binding fragment thereof. In some embodiments, the anti-CD2 antibody 1 of the disclosure is siplizumab or an antigen-binding fragment thereof. In some embodiments, the methods described herein include administering at least one, at least two, at least three, or more than at least three anti-CD2 antibody or antigen binding fragment thereof. In some embodiments, the methods described herein include administering one or more than one anti-CD2 antibody or antigen binding fragment thereof. In certain embodiments, the methods described herein include administering an anti-CD2 antibody or antigen binding fragment thereof in combination with another agent to a subject. In certain embodiments, the methods described herein include administering an anti-CD2 antibody or antigen binding fragment thereof prior to, at the same time, and/or after another agent to a subject.

An antibody as described herein can be comprised of two heavy and two light chains connected by a disulfide bond. Each heavy chain can comprise a variable region (VH) and a constant region. Each light chain can comprise a variable region (VL) and a constant region. The variable region of both the heavy and the light chain dictates the binding of the antibody to the antigen. The complementarity determining regions (CDRs) are variable loops on the variable regions of the heavy and light chain. There are three CDRs on each heavy chain and three CDRs on each light chain. In certain embodiments, the antibody as described herein binds to CD2.

In certain embodiments, administration of an anti-CD2 antibody or antigen binding fragment thereof described herein does not result in target cell depletion. In certain embodiments, an anti-CD2 antibody or antigen binding fragment thereof described herein exhibits immunomodulatory activity. In a specific embodiment, the ability of an anti-CD2 antibody or antigen binding fragment thereof described herein to refrain from target cell depletion while retaining immunomodulatory activity is accomplished by eliminating glycosylation of Fc region.

In some embodiments, an anti-CD2 antibody or antigen-binding fragment thereof as described herein binds specifically to CD2 (also called T11, SRBC (sheep red blood cell receptor), and LFA-2). In a specific embodiment, an anti-CD2 antibody or antigen-binding fragment thereof described herein binds to human CD2 (GenBank Accession No. NM_001328609.1 (isoform 1); NM_001767.5 (isoform 2)). In certain embodiments, an anti-CD2 antibody or antigen-binding fragment thereof provided herein can bind competitively with siplizumab (MedImmune Inc.; International Publication No. WO 02/098370). Siplizumab (MEDI-507) is a humanized version of the CD2-specific rat antibody BTI-322 (MedImmune Inc.; International Publication No. WO 02/098370). Siplizumab is an IgG1 kappa class monoclonal antibody and binds to the CD2 found on human T cells and human NK cells. Siplizumab is composed of two heavy chains (˜50 kDa) and two light chains (˜25 kDa).

As defined herein, an epitope is the region of the antigen to which the antibody or the antigen-binding fragment binds. In some embodiments, the epitope can be linear. In other embodiments, the epitope can be conformational. In some embodiments, the epitope can be formed by contiguous amino acids. In other embodiments, the epitope can be formed by noncontiguous amino acids. In certain embodiments, the anti-CD2 antibody or antigen-binding fragment thereof described herein binds to an epitope on CD2. In certain embodiments, the anti-CD2 antibody or antigen-binding fragment thereof described herein binds to the same epitope of CD2 as siplizumab. In certain embodiments, an anti-CD2 antibody or antigen-binding fragment thereof provided herein can bind competitively with siplizumab in an appropriate in vitro competitive binding assay such as the one detailed by Clark et al., J Exp Med. 1988 Jun. 1; 167(6):1861-72. In certain embodiments, the anti-CD2 antibody or antigen-binding fragment thereof provided herein has the same IC₅₀ value as siplizumab, approximately 1 nM (Branco et al., Transplantation. 1999 Nov. 27; 68(10):1588-96). In certain embodiments, the anti-CD2 antibody or antigen-binding fragment thereof provided herein has a lower IC₅₀ value than siplizumab. In certain embodiments, the anti-CD2 antibody or antigen-binding fragment thereof provided herein has a higher IC₅₀ value than siplizumab. In certain embodiments, the anti-CD2 antibody or antigen-binding fragment thereof provided herein has an IC₅₀ value of about, at least about, or at most about: 0.5 nM, 0.6 nM, 0.7 nM, 0.8 nM, 0.9 nM, 1.0 nM, 1.1 nM, 1.2 nM, 1.3 nM, 1.4 nM, 1.5 nM, between 0.5 nM and 0.8 nM, between 0.6 nM and 0.9 nM, between 0.7 nM and 1.0 nM, between 0.8 nM and 1.1 nM, between 0.9 nM and 1.2 nM, between 1.0 nM and 1.3 nM, between 1.1 nM and 1.4 nM, or between 1.2 nM and 1.5 nM.

In certain embodiments, the sequence of the VH region of the anti-CD2 antibody or antigen binding fragment thereof described herein is 80%, 85%, 90%, 91%, 92%, 93%, 94%, 95%, 96%, 97%, 98%, 99%, or 100% identical to SEQ ID NO: 1. In certain embodiments, the sequence of the VL region of the anti-CD2 antibody or antigen binding fragment thereof described herein is 80%, 85%, 90%, 91%, 92%, 93%, 94%, 95%, 96%, 97%, 98%, 99%, or 100% identical to SEQ ID NO: 2. In certain embodiments, the sequence of the VH CDR1 of the anti-CD2 antibody or antigen binding fragment thereof described herein can be SEQ ID NO: 3. In certain embodiments, the sequence of the VH CDR2 of the anti-CD2 antibody or antigen binding fragment thereof described herein can be SEQ ID NO: 4. In certain embodiments, the sequence of the VH CDR3 of the anti-CD2 antibody or antigen binding fragment thereof described herein can be SEQ ID NO: 5. In certain embodiments, the sequence of the VL CDR1 of the anti-CD2 antibody or antigen binding fragment thereof described herein can be SEQ ID NO: 6. In certain embodiments, the sequence of the VL CDR2 of the anti-CD2 antibody or antigen binding fragment thereof described herein can be SEQ ID NO: 7. In certain embodiments, the sequence of the VL CDR3 of the anti-CD2 antibody or antigen binding fragment thereof described herein can be SEQ ID NO: 8. These sequences are shown in Table 1.

In certain embodiments, the anti-CD2 antibody or antigen binding fragment thereof for use with the methods provided herein has a heavy chain variable region comprising VH CDRs of SEQ ID NOS: 3-5, respectively, and a VL of SEQ ID NO: 2. In certain embodiments, the anti-CD2 antibody or antigen binding fragment thereof for use with the methods provided herein has a heavy chain variable region comprising VL CDRs of SEQ ID NOS: 6-8, respectively, and a VH of SEQ ID NO: 1.

In certain embodiments, the anti-CD2 antibody or antigen binding fragment thereof for use with the methods provided herein has a heavy chain variable region CDR 1 of SEQ ID NO: 3; a heavy chain variable region CDR 2 of SEQ ID NO: 4; a heavy chain variable region CDR 3 of SEQ ID NO: 5; a light chain variable region CDR 1 of SEQ ID NO: 6; a light chain variable region CDR 2 of SEQ ID NO: 7; and a light chain variable region CDR 3 of SEQ ID NO: 8. In certain embodiments, the anti-CD2 antibody or antigen binding fragment thereof for use with the methods provided herein has a heavy chain variable region CDR 1 comprising a sequence that is at least about or about 75%, 80%, 85%, 90%, 95%, 96%, 97%, 98%, 99%, or 100% identical to SEQ ID NO: 3; a heavy chain variable region CDR 2 comprising a sequence that is at least about or about 75%, 80%, 85%, 90%, 95%, 96%, 97%, 98%, 99%, or 100% identical to SEQ ID NO: 4; a heavy chain variable region CDR 3 comprising a sequence that is at least about or about 75%, 80%, 85%, 90%, 95%, 96%, 97%, 98%, 99%, or 100% identical to SEQ ID NO: 5; a light chain variable region CDR 1 comprising a sequence that is at least about or about 75%, 80%, 85%, 90%, 95%, 96%, 97%, 98%, 99%, or 100% identical to SEQ ID NO: 6; a light chain variable region CDR 2 comprising a sequence that is at least about or about 75%, 80%, 85%, 90%, 95%, 96%, 97%, 98%, 99%, or 100% identical to SEQ ID NO: 7; and/or a light chain variable region CDR 3 comprising a sequence that is at least about or about 75%, 80%, 85%, 90%, 95%, 96%, 97%, 98%, 99%, or 100% identical to SEQ ID NO: 8.

In certain embodiments, the anti-CD2 antibody or antigen binding fragment thereof for use with the methods provided herein has a heavy chain variable region CDR 1 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 3; a heavy chain variable region CDR 2 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 4; a heavy chain variable region CDR 3 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 5; a light chain variable region CDR 1 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 6; a light chain variable region CDR 2 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 7; and/or a light chain variable region CDR 3 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 8.

In some embodiments, an amino acid substitution is a conservative substitution. Illustrative examples for conserved amino acid exchanges are amino acid substitutions that maintain structural and/or functional properties of the amino acids' side-chains, e.g., an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In some embodiments, a conservative amino acid substitution is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In contrast, examples of non-conserved amino acid exchanges are amino acid substitutions that do not maintain structural and/or functional properties of the amino acids' side-chains, e.g., an aromatic amino acid is substituted for a basic, acidic, or aliphatic amino acid, an acidic amino acid is substituted for an aromatic, basic, or aliphatic amino acid, a basic amino acid is substituted for an acidic, aromatic or aliphatic amino acid, and an aliphatic amino acid is substituted for an aromatic, acidic or basic amino acid.

In certain embodiments, an anti-CD2 antibody or antigen binding fragment thereof for use with the present methods and compositions comprises 1, 2, or 3 of the heavy chain CDRs of BTI-322 or of siplizumab. In certain embodiments, an anti-CD2 antibody or antigen binding fragment thereof for use with the present methods and compositions comprises 1, 2, or 3 of the light chain CDRs of BTI-322 or of siplizumab. In certain embodiments, an anti-CD2 antibody or antigen binding fragment thereof for use with the present methods and compositions comprises 1, 2, 3, 4, 5, or all 6 of the CDRs of BTI-322 or of siplizumab. In certain embodiments, an anti-CD2 antibody or antigen binding fragment thereof for use with the methods described herein comprises 1, 2, 3, 4, 5, or all 6 of the CDRs set forth in this disclosure (e.g., in Table 1 and/or Table 15). In certain embodiments, 1, 2, 3, 4, 5, and/or all 6 of the CDRs of BTI-322 or of siplizumab have 1, 2, 3, 4, 5, 6, 7, 8, 9, or 10 amino acid substitutions. In a more specific embodiment, such an amino acid substitution is a conservative amino acid substitution

TABLE 1
Sequences of CD2-binding molecule (or an anti-CD2
antibody or an antigen binding fragment thereof)

| Description | Sequence |
|---|---|
| VH region | QVQLVQSGAEVQRPGASVKVSCKASGYIFTEYY |
| (SEQ ID | MYWVRQAPGQGLELVGRIDPEDGSIDYVEKFKK |
| NO: 1) | KVTLTADTSSSTAYMELSSLTSDDTAVYYCARG |
| | KFNYRFAYWGQGTLVTVSS |
| VL region | DVVMTQSPPSLLVTLGQPASISCRSSQSLLHSS |
| (SEQ ID | GNTYLNWLLQRPGQSPQPLIYLVSKLESGVPDR |
| NO: 2) | FSGSGSGTDFTLKISGVEAEDVGV |
| | YYCMQFTHYPYTFGQGTKLEIK |
| VH CDR 1 | EYYMY |
| (SEQ ID | |
| NO: 3) | |
| VH CDR 2 | RIDPEDGSIDYVEKFKK |
| (SEQ ID | |
| NO: 4) | |
| VH CDR 3 | GKFNYRFAY |
| (SEQ ID | |
| NO: 5) | |
| VL CDR 1 | RSSQSLLHSSGNTYLN |
| (SEQ ID | |
| NO: 6) | |
| VL CDR 2 | LVSKLES |
| (SEQ ID | |
| NO: 7) | |
| VL CDR 3 | MQFTHYPYT |
| (SEQ ID | |
| NO: 8) | |

In a certain embodiment, the anti-CD2 antibody or antigen-binding fragment thereof described herein has the same six CDR sequences as siplizumab. In a certain embodiment, the anti-CD2 antibody or antigen-binding fragment thereof described herein can share five of the six CDR sequences of siplizumab. In certain embodiments, one of the CDRs of the anti-CD2 antibody or antigen-binding fragment thereof described herein is different from the counterpart CDR of siplizumab and that the CDR sequence is different by one amino acid. In certain embodiments, the difference between the CDR sequence of the anti-CD2 antibody or antigen-binding fragment thereof described herein and the counterpart CDR of siplizumab is a conservative amino acid substitution. For example, an anti-CD2 antibody or antigen-binding fragment thereof can have one, two, three, four, five, or six conservative amino acid substitutions relative to the set of six CDRs of siplizumab, or relative to the set of three CDRs in the heavy chain of siplizumab, or relative to the set of three CDRs in the light chain of siplizumab.

In certain embodiments, the VL CDR1, VL CDR2, or VL CDR3 of the anti-CD2 antibody or antigen-binding fragment thereof described herein is the CDR that differs from its siplizumab counterpart. In other embodiments, the VH CDR1, VH CDR2, or VH CDR3 of the anti-CD2 antibody or antigen-binding fragment thereof described herein is the CDR that differs from its siplizumab counterpart. In certain embodiments, the different CDR of the anti-CD2 antibody or antigen-binding fragment thereof described herein is longer than the sequence of its siplizumab counterpart. In other embodiments, the different CDR of the anti-CD2 antibody or antigen-binding fragment thereof described herein is shorter than the sequence of its siplizumab counterpart.

In certain embodiments, the sequence of the heavy chain constant region (CH) of the anti-CD2 antibody or antigen-binding fragment thereof described herein is about or at least about 80%, 85%, 90%, 91%, 92%, 93%, 94%, 95%, 96%, 97%, 98%, 99%, or 100% identical to SEQ ID NO: 9. In certain embodiments, the sequence of the heavy chain constant region of the anti-CD2 antibody or antigen-binding fragment thereof described herein is about or at least about 80%, 85%, 90%, 91%, 92%, 93%, 94%, 95%, 96%, 97%, 98%, 99%, or 100% identical to SEQ ID NO: 10. In certain embodiments, the sequence of the heavy chain constant region of the anti-CD2 antibody or antigen-binding fragment thereof described herein is about or at least about 80%, 85%, 90%, 91%, 92%, 93%, 94%, 95%, 96%, 97%, 98%, 99%, or 100% identical to SEQ ID NO: 11. In certain embodiments, the sequence of the heavy chain constant region of the anti-CD2 antibody or antigen-binding fragment thereof described herein is about or at least about 80%, 85%, 90%, 91%, 92%, 93%, 94%, 95%, 96%, 97%, 98%, 99%, or 100% identical to SEQ ID NO: 12. In certain embodiments, the sequence of the heavy chain constant region of the anti-CD2 antibody or antigen-binding fragment thereof described herein is about or at least about 80%, 85%, 90%, 91%, 92%, 93%, 94%, 95%, 96%, 97%, 98%, 99%, or 100% identical to SEQ ID NO: 13. In certain embodiments, the sequence of the light chain constant region (CL) of the anti-CD2 antibody or antigen-binding fragment thereof described herein is about or at least about 80%, 85%, 90%, 91%, 92%, 93%, 94%, 95%, 96%, 97%, 98%, 99%, or 100% identical to SEQ ID NO: 14. In certain embodiments, the sequence of the light chain constant region of the anti-CD2 antibody or antigen-binding fragment thereof described herein is about or at least about 80%, 85%, 90%, 91%, 92%, 93%, 94%, 95%, 96%, 97%, 98%, 99%, or 100% identical to SEQ ID NO: 15.

In certain embodiments, the anti-CD2 antibody or antigen-binding fragment thereof described herein can be directed to three regions of human CD2 known in the art as T11₁, T11₂, and T11₃ (Peterson, A., Seed, B., 1987. Nature 329, 842-846; Branco et al., 1999. Transplantation 68, 1588-1596; Arulanandam, A. R., et al., 1993. Proc. Natl. Acad. Sci. U.S.A. 90, 11613-11617; Damschroder et al., 2004. Molecular Immunology 41, 985-1000). Three residues in the adhesion domain of human CD2 that are critical for its binding to siplizumab are N18, K55, and T5 (Damschroder et al., 2004. Molecular Immunology 41, 985-1000). In certain embodiments, the residues N18, K55, and T59 in the extracellular CD2 domain are critical residues in the binding affinity of the anti-CD2 antibody or antigen-binding fragment thereof described herein to human CD2. In certain embodiments, the anti-CD2 antibody or antigen-binding fragment thereof described herein binds the same epitope as siplizumab. In certain embodiments, the anti-CD2 antibody or antigen-binding fragment thereof described herein binds competitively with siplizumab to human CD2.

In certain embodiments, the DNA sequence of the anti-CD2 antibody or antigen-binding fragment thereof can be modified to optimize the product yield during manufacture. The sequence optimization can increase product yield while not impacting product quality secretion of the molecule during production since the amino acid sequence generated can be the same as described in Table 1. The optimized DNA sequence for the anti-CD2 antibody or antigen-binding fragment thereof heavy chain and light chain are presented in Table 2.

TABLE 2
Optimized sequences of an anti-CD2 antibody
or antigen-binding fragment thereof

| Description | Sequence |
|---|---|
| Optimized | CAAGTGCAGCTGGTGCAGAGCGGAGCTGAGGT |
| Heavy | GCAGAGACCCGGCGCCAGCGTCAAGGTGAGCT |
| chain | GTAAGGCCAGCGGCTACATCTTCACAGAATAC |
| sequence | TACATGTACTGGGTGAGGCAAGCCCCCGGCCA |
| (SEQ ID | AGGACTGGAGCTGGTGGGCAGAATCGATCCAG |
| NO: 16) | AGGATGGCAGCATCGACTACGTGGAGAAGTTC |
| | AAGAAGAAGGTGACTCTGACAGCCGACACAAG |
| | CAGCAGCACTGCTTACATGGAGCTGAGCTCTC |
| | TGACTAGCGATGACACTGCCGTGTACTACTGT |
| | GCTAGGGGCAAGTTCAACTATAGGTTCGCCTA |
| | CTGGGGCCAAGGCACTCTGGTGACAGTCAGCA |
| | GCGCTAGCACCAAGGGCCCATCGGTCTTCCCC |
| | CTGGCACCCTCCTCCAAGAGCACCTCTGGGGG |
| | CACAGCGGCCCTGGGCTGCCTGGTCAAGGACT |
| | ACTTCCCCGAACCGGTGACGGTGTCGTGGAAC |
| | TCAGGCGCCCTGACCAGCGGCGTGCACACCTT |
| | CCCGGCCGTCCTACAGTCCTCAGGACTCTACT |
| | CCCTCAGCAGCGTGGTGACCGTGCCCTCCAGC |
| | AGCTTGGGCACCCAGACCTACATCTGCAACGT |
| | GAATCACAAGCCCAGCAACACCAAGGTGGACA |
| | AGAAGGTTGAGCCCAAATCTTGTGACAAAACT |
| | CACACATGCCCACCGTGCCCAGCACCTGAACT |
| | CCTGGGGGGACCGTCAGTCTTCCTCTTCCCCC |
| | CAAAACCCAAGGACACCCTCATGATCTCCCGG |
| | ACCCCTGAGGTCACATGCGTGGTGGTGGACGT |
| | GAGCCACGAAGACCCTGAGGTCAAGTTCAACT |
| | GGTACGTGGACGGCGTGGAGGTGCATAATGCC |
| | AAGACAAAGCCGCGGGAGGAGCAGTACAACAG |
| | CACGTACCGTGTGGTCAGCGTCCTCACCGTCC |
| | TGCACCAGGACTGGCTGAATGGCAAGGAGTAC |
| | AAGTGCAAGGTCTCCAACAAAGCCCTCCCAGC |
| | CCCCATCGAGAAAACCATCTCCAAAGCCAAAG |
| | GGCAGCCCCGAGAACCACAGGTGTACACCCTG |
| | CCCCCATCCCGGGACGAGCTGACCAAGAACCA |
| | GGTCAGCCTGACCTGCCTGGTCAAAGGCTTCT |
| | ATCCCAGCGACATCGCCGTGGAGTGGGAGAGC |
| | AATGGGCAGCCGGAGAACAACTACAAGACCAC |
| | GCCTCCCGTGCTGGACTCCGACGGCTCCTTCT |
| | TCCTCTACAGCAAGCTCACCGTGGACAAGAGC |
| | AGGTGGCAGCAGGGGAACGTCTTCTCATGCTC |
| | CGTGATGCATGAGGCTCTGCACAACCACTACA |
| | CGCAGAAGAGCCTCTCCCTGTCTCCGGGTAAA |
| | TGATGA |
| Optimized | GACGTGGTGATGACTCAGAGCCCTCCTTCTCT |
| Light | GCTGGTGACTCTGGGCCAGCCAGCCAGCATCA |
| chain | GCTGTAGGAGCAGCCAGTCTCTGCTGCACTCC |
| sequence | AGCGGCAACACTTATCTGAACTGGCTGCTGCA |
| (SEQ ID | GAGACCCGGCCAGAGCCCTCAGCCTCTGATCT |
| NO: 17) | ACCTCGTGAGCAAGCTGGAGAGCGGCGTGCCA |
| | GATAGGTTTAGCGGCAGCGGAAGCGGCACTGA |
| | CTTCACTCTGAAGATCAGCGGCGTGGAAGCTG |
| | AGGATGTGGGCGTCTACTACTGCATGCAGTTC |
| | ACACACTACCCATACACTTTCGGCCAAGGCAC |
| | AAAGCTGGAAATCAAGCGTACGGTGGCTGCAC |
| | CATCTGTCTTCATCTTCCCGCCATCTGATGAG |
| | CAGTTGAAATCTGGAACTGCCTCTGTTGTGTG |
| | CCTGCTGAATAACTTCTATCCCAGAGAGGCCA |
| | AAGTACAGTGGAAGGTGGATAACGCCCTCCAA |
| | TCGGGTAACTCCCAGGAGAGTGTCACAGAGCA |
| | GGACAGCAAGGACAGCACCTACAGCCTCAGCA |
| | GCACCCTGACGCTGAGCAAAGCAGACTACGAG |
| | AAACACAAAGTCTACGCCTGCGAAGTCACCCA |
| | TCAGGGCCTGAGCTCGCCCGTCACAAAGAGCT |
| | TCAACAGGGGAGAGTGTTGATGA |

In certain embodiments, the sequence of the heavy chain of the anti-CD2 antibody or antigen-binding fragment thereof described herein is about or at least about: 80%, 85%, 90%, 91%, 92%, 93%, 94%, 95%, 96%, 97%, 98%, 99%, or 100% identical to SEQ ID NO: 16. In certain embodiments, the sequence of the light chain of the anti-CD2 antibody or antigen-binding fragment thereof described herein is about or at least about: 80%, 85%, 90%, 91%, 92%, 93%, 94%, 95%, 96%, 97%, 98%, 99%, or 100% identical to SEQ ID NO: 17.

In certain embodiments, the anti-CD2 antibody or antigen-binding fragment thereof interferes with the CD58/CD2 signaling cascade. In certain embodiments, the anti-CD2 antibody or antigen-binding fragment thereof blocks CD58. An example of a compound that inhibits the CD58/CD2 pathway is alefacept.

In certain embodiments, the depleting version of siplizumab can be used for the treatment of an autoimmune disease (e.g., developed as a result of organ or tissue transplant) or used in a subject that has or will undergo organ or tissue transplant.

In certain embodiments, an anti-CD2 antibody or an antigen binding fragment thereof described herein has no or has reduced antibody-dependent cellular cytotoxicity (“ADCC”). In certain embodiments, an anti-CD2 antibody or an antigen binding fragment thereof can be generated as to exhibit reduced or absent ADCC using methods including, but not limited to, Fc silencing, subclass switching, deglycosylation, and other mutations or modifications of the Fc region. These methods are described, for example, in U.S. Provisional Application No. 63/135,381, which is incorporated herein by reference in its entirety for non-limiting examples of an anti-CD2 antibody or an antigen binding fragment thereof that may be used in the methods described herein.

ADCC activity can be determined by any commercially available kit (see, e.g. Promega ADCC Reporter Bioassay, Core Kit (Cat. #G7010, G7018), or any appropriate assay. Such assays can include, but are not limited to, a flow cytometry-based assay, a fluorometric assay, or a bioluminescent reporter assay.

In certain embodiments, the anti-CD2 antibody or an antigen binding fragment thereof described herein exhibits at most 90% of the ADCC activity of siplizumab in an in vitro assay. An example of such an assay is described in the methods of Golay et al., Haematologica. January 2003; 88:1002-1012. Specifically, the anti-CD2 antibody or an antigen binding fragment thereof provided herein exhibits at most 0%, 5%, 10%, 20%, 30%, 40%, 50%, 60%, 70%, 80%, or at most 90% of the ADCC activity as compared to siplizumab.

In certain embodiments, in vivo administration of the anti-CD2 antibody or an antigen binding fragment thereof) provided herein exhibits at most 90% of the ADCC activity as compared to in vivo administration of siplizumab in a humanized mouse model or a human subject in a clinical setting. Specifically, in vivo administration of the CD2-binding molecule (or an anti-CD2 antibody or an antigen binding fragment thereof) provided herein exhibits at most 0%, 5%, 10%, 20%, 30%, 40%, 50%, 60%, 70%, 80%, or at most 90% of the ADCC activity as in vivo administration of siplizumab in a humanized mouse model or a human subject in a clinical setting.

Immunomodulatory activities exhibited by the anti-CD2 antibody or antigen-binding fragment thereof described herein can include, but are not limited to, the inhibition of the activation and proliferation of CD4+/CD25+ T cells, increasing percentage of FOXP3⁺ regulatory T cells, and the suppression of CD69+ NK cells. In certain embodiments, the anti-CD2 antibody or antigen-binding fragment thereof described herein exhibits the same immunomodulatory activity (e.g., the same type and at the same level) as siplizumab. The determination of immunomodulatory activity can be achieved by any appropriate method known in the art. These methods can include, but are not limited to, cell proliferation assays, T cell activation functional assays, ELISPOT assays, intracellular staining, cytokine capture, tetramer staining, spectra-typing assays, and biosensor assays. As an example, T cell activation can be determined by any commercially available assay kit (see, e.g. Promega T Cell Activation Bioassay (NFAT or IL-2) (Cat. #J1621 or J1651)), or any appropriate assay, wherein briefly an assay plate is coated with anti-CD3 antibodies, human PBMCs or mouse peripheral target cells are added, anti-CD28 is added to the cells, and proliferation is quantified.

In certain embodiments, the anti-CD2 antibody or antigen-binding fragment thereof described herein can inhibit the activation and proliferation of CD4+/CD25+ T cells to a level that is about or at least about 50% of the level achieved by siplizumab in an in vitro assay. An example of such an assay is described in the methods of Ng et al., Blood. 2001; 98:2736-2744. Specifically, the anti-CD2 antibody or antigen-binding fragment thereof described herein can inhibit the activation and proliferation of CD4+/CD25+ T cells in vitro to a level of about or at least about 50%, 55%, 60%, 65%, 70%, 75%, 80%, 85%, 90%, 95%, or 100% of the level achieved by siplizumab.

In certain embodiments, in vivo administration of the anti-CD2 antibody or antigen-binding fragment thereof provided herein exhibits about or at least about 50% of the level of CD4+/CD25+ T cell activation/proliferation as compared to in vivo administration of siplizumab in a humanized mouse model or a human subject in a clinical setting. Specifically, in vivo administration of the anti-CD2 antibody or antigen-binding fragment thereof provided herein exhibits about or at least about 50%, 55%, 60%, 65%, 70%, 75%, 80%, 85%, 90%, 95%, or 100% of the level of CD4+/CD25+ T cell activation/proliferation as compared to in vivo administration of siplizumab in a humanized mouse model or a human subject in a clinical setting.

In certain embodiments, the anti-CD2 antibody or antigen-binding fragment thereof described herein can increase the amount of FOXP3⁺ regulatory T cells as compared to siplizumab. In certain embodiments, the anti-CD2 antibody or antigen-binding fragment thereof described herein can increase the amount of FOXP3⁺ regulatory T cells to a level of about or at least about 50% of the amount achieved by siplizumab in an in vitro assay. An example of such an assay is described in the methods of Sambucci et al., Scientific Reports. 8: 3674 (2018). Specifically, the anti-CD2 antibody or antigen-binding fragment thereof described herein can increase the amount of FOXP3⁺ regulatory T cells in vitro to a level about or at least about 50%, 55%, 60%, 65%, 70%, 75%, 80%, 85%, 90%, 95%, or 100% of the amount of FOXP3⁺ regulatory T cells achieved by siplizumab.

In certain embodiments, in vivo administration of the anti-CD2 antibody or antigen-binding fragment thereof described herein exhibits about or at least about 50% of the level of FOXP3⁺ regulatory T cells as compared to in vivo administration of siplizumab in a humanized mouse model or a human subject in a clinical setting. Specifically, in vivo administration of the anti-CD2 antibody or antigen-binding fragment thereof described herein exhibits about or at least about 50%, 55%, 60%, 65%, 70%, 75%, 80%, 85%, 90%, 95%, or 100% of the level of FOXP3⁺ regulatory T cells as compared to in vivo administration of siplizumab in a humanized mouse model or a human subject in a clinical setting.

In certain embodiments, the anti-CD2 antibody or antigen-binding fragment thereof described herein can inhibit the expression of CD69+ NK cells as compared to siplizumab. In certain embodiments, the anti-CD2 antibody or antigen-binding fragment thereof provided herein exhibits about or at most about 50% of the expression of CD69+ NK cells as compared to siplizumab in an in vitro assay. An example of such an assay is described in the methods of Thum et el., Human Reproduction. 19:10, pp. 2395-2400, 2004. Specifically, the anti-CD2 antibody or antigen-binding fragment thereof described herein exhibits at most 10%, 20%, 30%, 40%, 50%, 60%, 70%, 80%, or at most 90% of the expression of CD69+ NK cells in vitro as compared to siplizumab.

In certain embodiments, in vivo administration of the anti-CD2 antibody or antigen-binding fragment thereof provided herein exhibits about or at most about 50% of the expression of CD69+ NK cells as compared to in vivo administration of siplizumab in a humanized mouse model or a human subject in a clinical setting. Specifically, in vivo administration of the anti-CD2 antibody or antigen-binding fragment thereof provided herein exhibits about 0 at most about 10%, 20%, 30%, 40%, 50%, 60%, 70%, 80%, or about or at most about 90% of the expression of CD69+ NK cells as compared to in vivo administration of siplizumab in a humanized mouse model or a human subject in a clinical setting.

In some embodiments, administration of the anti-CD2 antibody or antigen-binding fragment thereof described herein completely abrogates ADCC activity in a recipient while retaining immunomodulatory activity. In certain embodiments, an anti-CD2 antibody or antigen binding fragment thereof described herein is an IgG1 antibody and has a modification in the Fc region. In certain embodiments, an anti-CD2 antibody or antigen binding fragment thereof described herein is an IgG2 antibody and has a modification in the Fc region. In certain embodiments, an anti-CD2 antibody or antigen binding fragment thereof described herein is an IgG3 antibody and has a modification in the Fc region. In certain embodiments, an anti-CD2 antibody or antigen binding fragment thereof described herein is an IgG4 antibody and has a modification in the Fc region. In certain embodiments, an anti-CD2 antibody or antigen binding fragment thereof described herein comprises at least one mutation (e.g., about or at least one, about or at least two, about or at least three, about or at least least four, about or at least least five, about or at least least six, about or at least least seven, about or at least least eight, about or at least least nine, about or at least least ten, or more than about ten mutations) in comparison to siplizumab. In certain embodiments, an anti-CD2 antibody or antigen binding fragment thereof described herein comprises about one mutation, about two mutations, about three mutations, about four mutations, about five mutations, about six mutations, about seven mutations, about eight mutations, about nine mutations, about ten mutations, or more than about ten mutations in relation to siplizumab (e.g., mutation in the Fc region of siplizumab). In certain embodiments, an anti-CD2 antibody or antigen binding fragment thereof described herein comprises siplizumab CDRs in IgG1 heavy chain and at least one mutation (e.g., about or at least one, about or at least two, about or at least three, about or at least least four, about or at least least five, about or at least least six, about or at least least seven, about or at least least eight, about or at least least nine, about or at least least ten, or more than about ten mutations). In certain embodiments, an anti-CD2 antibody or antigen binding fragment thereof described herein comprises siplizumab CDRs in IgG2 heavy chain and at least one mutation (e.g., about or at least one, about or at least two, about or at least three, about or at least least four, about or at least least five, about or at least least six, about or at least least seven, about or at least least eight, about or at least least nine, about or at least least ten, or more than about ten mutations). In certain embodiments, an anti-CD2 antibody or antigen binding fragment thereof described herein comprises siplizumab CDRs in IgG3 heavy chain and at least one mutation (e.g., about or at least one, about or at least two, about or at least three, about or at least least four, about or at least least five, about or at least least six, about or at least least seven, about or at least least eight, about or at least least nine, about or at least least ten, or more than about ten mutations). In certain embodiments, an anti-CD2 antibody or antigen binding fragment thereof described herein comprises siplizumab CDRs in IgG4 heavy chain and at least one mutation (e.g., about or at least one, about or at least two, about or at least three, about or at least least four, about or at least least five, about or at least least six, about or at least least seven, about or at least least eight, about or at least least nine, about or at least least ten, or more than about ten mutations). The mutation can comprise of at least one alteration in the amino acid sequence of an anti-CD2 antibody or antigen binding fragment thereof as compared to the wild-type counterpart wherein the alteration results in the reduction or elimination of the binding of the Fc region to its cognate receptor. In some embodiments, a mutation is one or more mutation and includes a mutation in amino acid position L234, L235, P329, V234, G237, P238, H268, V309, A330, P331, and/or S228 (e.g., based on Edelman (EU) numbering). In some embodiments, a mutation is one or more mutation and includes a mutation in amino acid position L234 and/or L235. In some embodiments, a mutation is one or more mutation and includes a mutation in amino acid position L234, L235, and/or P329. In some embodiments, a mutation is one or more mutation and includes a mutation in amino acid position V234, G237, P238, H268, V309, A330, and/or P331. In some embodiments, a mutation is one or more mutation and includes a mutation in amino acid position S228. In some embodiments, a mutation is one or more mutation and includes a mutation in amino acid position S228, P329, and/or L235. In some embodiments, a mutation is one or more mutation and includes L234A (or another conservative amino acid mutation). In some embodiments, a mutation is one or more mutation and includes L235A (or another conservative amino acid mutation). In some embodiments, a mutation is one or more mutation and includes P329G (or another conservative amino acid mutation). In some embodiments, a mutation is one or more mutation and includes V234A (or another conservative amino acid mutation). In some embodiments, a mutation is one or more mutation and includes G237A (or another conservative amino acid mutation). In some embodiments, a mutation is one or more mutation and includes P238S (or another conservative amino acid mutation). In some embodiments, a mutation is one or more mutation and includes H268A (or another conservative amino acid mutation). In some embodiments, a mutation is one or more mutation and includes V309L (or another conservative amino acid mutation). In some embodiments, a mutation is one or more mutation and includes A330S (or another conservative amino acid mutation). In some embodiments, a mutation is one or more mutation and includes P331S (or another conservative amino acid mutation). In some embodiments, a mutation is one or more mutation and includes S228P (or another conservative amino acid mutation). In some embodiments, a mutation is one or more mutation and includes L235E (or another conservative amino acid mutation). In some embodiments, the mutation includes L234A and L235A (or other conservative amino acid mutations). In some embodiments, the mutation includes P329G, L234A and L235A (or other conservative amino acid mutations). In some embodiments, the mutation includes V234A, G237A, P238S, H268A, V309L, A330S, and P331S (or other conservative amino acid mutations) (e.g., for IgG2). In some embodiments, a mutation is one or more mutation and includes S228P (or another conservative amino acid mutation) (e.g., for IgG4). In some embodiments, a mutation is one or more mutation and includes P329G, S228P, and L235E (or another conservative amino acid mutations) (e.g., for IgG4). In some embodiments, the amino acid position is a position based on any antibody numbering scheme (e.g., Edelman (EU) numbering). In some embodiments, the amino acid position is a position based on Edelman (EU) numbering. In some embodiments, the amino acid position is a position based on Kabat numbering scheme. In some embodiments, the amino acid position is a position based on Clothia numbering scheme. In some embodiments, the amino acid position is a position based on IMGT numbering scheme.

The modification can comprise of at least one alteration in the amino acid sequence of the anti-CD2 antibody or antigen-binding fragment thereof Fc region as compared to the wild-type Fc region wherein the alteration results in the reduction or elimination of the binding of the Fc region to its cognate receptor. The Fc receptor is located on immune effectors cells including B cells, NK cells, macrophages, and neutrophils. Without being bound by theory, in wild type IgG, the Fc interaction with the Fc receptor (FcR) leads to downstream effector cell functions including stimulating the phagocytic or cytotoxic activities of the immune cell. Reduction or elimination of Fc/FcR interaction results in elimination of effector functions.

In certain embodiments, the anti-CD2 antibody or antigen binding fragment thereof exhibits reduced binding to the FcγRIIIA receptor as compared to siplizumab. In certain embodiments, an anti-CD2 antibody or antigen binding fragment thereof exhibits reduced binding to the FcγRIIA receptor as compared to siplizumab. In certain embodiments, an anti-CD2 antibody or antigen binding fragment thereof exhibits reduced binding to the FcγRI receptor as compared to siplizumab. In certain embodiments, an anti-CD2 antibody or antigen binding fragment thereof) exhibits increased binding to the FcγRIIIA receptor as compared to siplizumab. In certain embodiments, an anti-CD2 antibody or antigen binding fragment thereof exhibits increased binding to the FcγRIIA receptor as compared to siplizumab. In certain embodiments, an anti-CD2 antibody or antigen binding fragment thereof exhibits increased binding to the FcγRI receptor as compared to siplizumab. In certain embodiments, an anti-CD2 antibody or antigen binding fragment thereof as described herein binds at about, at least about, or at most about 0%, 5%, 10%, 15%, 20%, 25%, 30%, 35%, 40%, 45%, 50%, 55%, 60%, 65%, 70%, 75%, 80%, 85%, 90%, or 95% of the binding ability exhibited by siplizumab. In certain embodiments, an anti-CD2 antibody or antigen binding fragment thereof as described herein has about or at least about 5%, 10%, 15%, 20%, 25%, 30%, 35%, 40%, 45%, 50%, 55%, 60%, 65%, 70%, 75%, 80%, 85%, 90%, or 95% higher binding ability (e.g., to FcγRIIA, FcγRIIIA, and/or FcγRI) as compared to the binding ability exhibited by siplizumab (e.g., to FcγRIIA, FcγRIIIA, and/or FcγRI). In certain embodiments, an anti-CD2 antibody or antigen binding fragment thereof as described herein has about or at least about 5%, 10%, 15%, 20%, 25%, 30%, 35%, 40%, 45%, 50%, 55%, 60%, 65%, 70%, 75%, 80%, 85%, 90%, or 95% lower binding ability (e.g., to FcγRIIA, FcγRIIIA, and/or FcγRI) as compared to the binding ability exhibited by siplizumab (e.g., to FcγRIIA, FcγRIIIA, and/or FcγRI). Assays to detect binding events can include, but are not limited to, Enzyme-Linked Immunosorbent Assays (ELISAs) and/or surface plasmon resonance (SPR) methods such as the Biacore system.

In certain embodiments, the Fc region can be modified by any appropriate method known in the art. In certain embodiments, a modification can result in Fc silencing. In certain embodiments, the modification can include the mutation of the amino acid sequence of the IgG Fc. In certain embodiments, the modification can include the mutation of the glycosylation site (N297) or of the consensus sequence comprising N297. In certain embodiments, modifications can include mutations that inhibit FcγR and C1q binding. In certain embodiments, these mutations can include any or all of the mutations K322A, L234A and L235A. In certain embodiments, an anti-CD2 antibody or antigen binding fragment thereof described herein can be a Fab, wherein no Fc is present. In certain embodiments, an anti-CD2 antibody or antigen binding fragment thereof described herein is an IgG4 antibody. Without being bound by theory, the IgG4 subclass is desirable for therapeutic purposes due to the lack of effector functions including ADCC (Davies and Sutton, Immunol Rev. 2015 November; 268(1): 139-15). In certain embodiments, an anti-CD2 antibody or antigen binding fragment thereof as described herein is an IgG2 antibody. Without being bound by theory, therapeutically beneficial characteristics of the IgG2 subclass include that IgG2 does not cross placenta (Einarsdottir et al., PLOS One. 2014 Sep. 24; 9(9):e108319) and that IgG2 has very low/no Fc receptor binding capacity (Vidarsson et al., Front Immunol. 2014; 5:520).

In certain embodiments, the anti-CD2 antibody or antigen-binding fragment thereof described herein can have an antigen binding variable region and a Fc region. In certain embodiments, the Fc region of the anti-CD2 antibody or antigen-binding fragment thereof described herein contains a glycosylation consensus sequence in each of the heavy chains of the antibody. In certain embodiments, the glycosylation consensus sequence is Asn-X-Ser. In certain embodiments, the glycosylation consensus sequence is Asn-X-Thr. In certain embodiments, the anti-CD2 antibody or antigen-binding fragment thereof described herein is glycosylated at Asn297. In certain embodiments, the anti-CD2 antibody or antigen-binding fragment thereof described herein has been deglycosylated. In certain embodiments, the glycan attached to the asparagine residue can be an N-linked glycan. In certain embodiments, the glycan attached to the asparagine residue can be an O-linked glycan. In a specific embodiment, the anti-CD2 antibody or antigen-binding fragment thereof described herein can contain a single N-linked glycosylation site on Asn297 of the heavy chain.

In certain embodiments, the anti-CD2 antibody or antigen-binding fragment thereof described herein can be glycosylated at the Fc glycosylation consensus sequence. In certain embodiments, the anti-CD2 antibody or antigen-binding fragment thereof described herein is deglycosylated. In a certain embodiment, the deglycosylation of the anti-CD2 antibody or antigen-binding fragment thereof described herein is achieved by modifying the Fc region, specifically by introducing a point mutation at position N297. In a certain embodiment, the mutation introduced at position 297 (N297) can be, but is not limited to, N297G, N297Q, or N297A. Without being bound by theory, the N297 point mutation can result in the lack of glycosylation and silencing of Fc signaling. In a certain embodiment, the deglycosylation of the anti-CD2 antibody or antigen-binding fragment thereof described herein is achieved by chemical or enzymatic degradation of the anti-CD2 antibody or antigen-binding fragment thereof glycan structures. In a specific embodiment, the chemical or enzymatic methods of glycan degradation preserves the Fc amino acid sequence.

In certain embodiments, the constant region of the anti-CD2 antibody or antigen-binding fragment thereof described herein can be switched with the constant region of an antibody of a different subclass. Without being bound by theory, while the variable region does not change, a subclass-switched antibody retains its specific affinity while interacting with different effector molecules (Valenzuela and Schaub, Transplantation. 2018 January; 102(1S Suppl 1):S7-S13). In a certain embodiment, the constant region of the anti-CD2 antibody or antigen-binding fragment thereof described herein can be switched with a constant region of a different antibody. In a certain embodiment, this switch can result in the anti-CD2 antibody or antigen-binding fragment thereof becoming a different subclass of antibody than it was originally. In certain embodiments, the constant region of the anti-CD2 antibody or antigen-binding fragment thereof can be switched with a different antibody while preserving the specific binding of the variable regions. In certain embodiments, the constant region of the anti-CD2 antibody or antigen-binding fragment thereof can be switched with a different antibody and the specific variable region is preserved, wherein the VH region of the anti-CD2 antibody or antigen-binding fragment thereof is about or at least about 80%, 85%, 90%, 91%, 92%, 93%, 94%, 95%, 96%, 97%, 98%, 99%, or 100% identical to SEQ ID NO: 1, and the sequence of the VL region of the anti-CD2 antibody or antigen-binding fragment thereof is about or at least about 80%, 85%, 90%, 91%, 92%, 93%, 94%, 95%, 96%, 97%, 98%, 99%, or 100% identical to SEQ ID NO: 2.

In certain embodiments, the anti-CD2 antibody or antigen binding fragment thereof binds specifically to the same epitope in human CD2 as siplizumab. In certain embodiments, the anti-CD2 antibody or antigen binding fragment thereof can be an animal-specific antibody, a human-specific antibody, a chimeric antibody, a humanized antibody, a full length antibody, an antibody fragment, a single chain variable fragment (scFv), a naturally occurring antibody, a synthetic antibody, an engineered antibody, enlarged anti-CD2 variants wherein additional components are added to the Fc region (e.g., a component can include an scFv, a CH2 domain, and/or a CH3 domain), or a combination thereof. In certain embodiments, the antibody Fc region has a point mutation (e.g., in N297) resulting in Fc silencing. In certain embodiments, the antibody is an IgG1. In certain embodiments, the antibody is an IgG2. In certain embodiments, the antibody is an IgG4. In certain embodiments, the antibody has a different native constant region than siplizumab. In certain embodiments, the antibody has a different native constant region than siplizumab and a point mutation in the Fc region resulting in Fc silencing. In some embodiments, the anti-CD2 antibody or antigen binding fragment thereof is a humanized anti-CD2 monoclonal antibody. In a specific embodiment, the anti-CD2 antibody or antigen binding fragment thereof is siplizumab. In certain embodiments, the anti-CD2 antibody or antigen binding fragment thereof as described herein can have a modified Fc region wherein the modification can include, but is not limited to, a point mutation resulting in Fc silencing, a switched native constant region, or an Fc silenced switched new native constant region. These combinations of different antibody subclasses and modifications produce different versions of the anti-CD2 antibody or antigen binding fragment thereof which are outlined in Table 3. In certain embodiments, the anti-CD2 antibody or antigen binding fragment thereof as described herein can be an IgG1, an IgG2, or an IgG4 subclass of antibody.

TABLE 3
Examples of versions of anti-CD2 antibody
or antigen-binding fragment thereof

| IgG | Modification of CD2-binding molecule (or anti-CD2 |
|---|---|
| subclass | antibody or antigen-binding fragment thereof) |
| IgG1 | A point mutation resulting in silenced Fc |
| IgG1 | same variable region but combined with a different native |
| | constant region |
| IgG1 | same variable region but combined with a Fc-silenced new |
| | native constant region |
| IgG2 | same variable region but combined with a different native |
| | constant region |
| IgG2 | same variable region but combined with a Fc-silenced new |
| | native constant region |
| IgG4 | same variable region but combined with a different native |
| | constant region |
| IgG4 | same variable region but combined with a Fc-silenced new |
| | native constant region |

Comparisons of anti-CD2 antibody or antigen binding fragment thereof are outlined in Table 4.

TABLE 4
Comparisons of anti-CD2 antibody or
antigen-binding fragment thereof

| | VH | VL | Deglycosyli- | Fc | IgG |
|---|---|---|---|---|---|
| Molecule | region: | region: | ated | modification | subclass |
| CD2-binding | SEQ ID | SEQ ID | Yes | None | IgG1 |
| molecule 1 | NO: 1 | NO: 2 | | | |
| CD2-binding | SEQ ID | SEQ ID | No | LALA-PG | IgG1 |
| molecule 2 | NO: 1 | NO: 2 | | mutations | |
| CD2-binding | SEQ ID | SEQ ID | No | V234A, | IgG2 |
| molecule 3 | NO: 1 | NO: 2 | | G237A, | |
| | | | | P238S, | |
| | | | | H268A, | |
| | | | | V309L, | |
| | | | | A330S, P331S | |
| CD2-binding | SEQ ID | SEQ ID | No | S228P, L235E, | IgG4 |
| molecule 4 | NO: 1 | NO: 2 | | P329G | |

In certain embodiments, the anti-CD2 antibody or antigen binding fragment thereof described herein, including examples presented above, have little to no ADCC activity compared to siplizumab, yet retain the immunomodulatory effects exhibited by siplizumab. In certain embodiments, the anti-CD2 antibody or antigen-binding fragment thereof provided herein can have a molecular weight that is higher than the molecular weight of an unmodified IgG1 antibody. In certain embodiments, this increase of molecular weight can be accomplished by the addition of duplicate regions of the molecule. In certain embodiments, the regions can be attached to the anti-CD2 antibody or antigen-binding fragment thereof through such means known in the art such as, but not limited to, chemical conjugation, recombinant fusion, and covalent attachment.

In certain embodiments, the additional regions to be attached to the anti-CD2 antibody or antigen-binding fragment thereof, can include, but are not limited to, duplicate regions of the molecule such as an additional variable heavy chain (VH), an additional variable light chain (VL), a scFv comprising the fusion of an additional VH and an additional VL, an additional CH2 domain, or an additional CH3 domain. In certain embodiments, the additional regions can be attached to the Fc region of the anti-CD2 antibody or antigen-binding fragment thereof.

In certain embodiments, an scFv can be attached to the Fc region of the anti-CD2 antibody or antigen binding fragment thereof. In certain embodiments the scFv can comprise a fusion of the VH and the VL of siplizumab. In certain embodiments, the scFv can comprise a fusion of a VH, wherein the VH can comprise an amino acid sequence of SEQ ID NO: 1, and a VL, wherein the VL can comprise an amino acid sequence of SEQ ID NO: 2. In a specific embodiment, the scFv binds to CD2. In a certain embodiment, the CDRs of the scFv are the same as the CDRs of siplizumab. In certain embodiments, the scFv attached to the Fc region of the anti-CD2 antibody or antigen-binding fragment thereof described herein binds to an epitope on CD2. In certain embodiments, the scFv attached to the Fc region of the anti-CD2 antibody or antigen binding fragment thereof described herein binds to the same epitope of CD2 as siplizumab. In certain embodiments, the scFv can be generated from an IgG2 antibody or an IgG4 antibody.

In certain embodiments, an additional CH2 domain can be attached to the Fc. In certain embodiments, the CH2 domain can comprise an amino acid sequence that is about or at least about 80%, 85%, 90%, 95%, 98%, or 99% identical to the amino acid sequence of SEQ ID NO: 18 or 19. In certain embodiments, the additional CH2 domain can be generated from an IgG2 antibody or an IgG4 antibody. In certain embodiments, an additional CH3 domain can be attached to the Fc. In certain embodiments, the CH3 domain can comprise an amino acid sequence that is about or at least about 80%, 85%, 90%, 95%, 98%, or 99% identical to the amino acid sequence of SEQ ID NO:20 or 21. In certain embodiments, the additional CH3 domain can be generated from an IgG2 antibody or an IgG4 antibody. In a certain embodiment, the additional domains can be attached to the C-terminal end of the Fc. In certain embodiments, the domains can be attached to the anti-CD2 antibody or antigen-binding fragment thereof through such means known in the art such as, but not limited to, chemical conjugation, recombinant fusion, and covalent attachment. In certain embodiments, the domains can be attached to the anti-CD2 antibody or antigen-binding fragment thereof through such means known in the art such as, but not limited to, chemical conjugation, recombinant fusion, and covalent attachment. Examples of CD2 antibody or antigen-binding fragment thereof that can be used in disclosure and production thereof are described, for example, in U.S. Provisional Application No. 63/042,844, which is incorporated herein by reference in its entirety

In certain embodiments, the anti-CD2 antibody or antigen-binding fragment thereof described herein can comprise a VH of SEQ ID NO: 1, a VL of SEQ ID NO: 2, a CH of SEQ ID NO: 9, and a CL of SEQ ID NO: 14; a VH of SEQ ID NO: 1, a VL of SEQ ID NO: 2, a CH of SEQ ID NO: 10, and a CL of SEQ ID NO: 14; a VH of SEQ ID NO: 1, a VL of SEQ ID NO: 2, a CH of SEQ ID NO: 11, and a CL of SEQ ID NO: 14; a VH of SEQ ID NO: 1, a VL of SEQ ID NO: 2, a CH of SEQ ID NO: 12, and a CL of SEQ ID NO: 14; or a VH of SEQ ID NO: 1, a VL of SEQ ID NO: 2, a CH of SEQ ID NO: 13, and a CL of SEQ ID NO: 14. In certain embodiments, the anti-CD2 antibody or antigen-binding fragment thereof described herein can comprise a VH of SEQ ID NO: 1, a VL of SEQ ID NO: 2, a CH of SEQ ID NO: 9, and a CL of SEQ ID NO: 15; a VH of SEQ ID NO: 1, a VL of SEQ ID NO: 2, a CH of SEQ ID NO: 10, and a CL of SEQ ID NO: 15; a VH of SEQ ID NO: 1, a VL of SEQ ID NO: 2, a CH of SEQ ID NO: 11, and a CL of SEQ ID NO: 15; a VH of SEQ ID NO: 1, a VL of SEQ ID NO: 2, a CH of SEQ ID NO: 12, and a CL of SEQ ID NO: 15; or a VH of SEQ ID NO: 1, a VL of SEQ ID NO: 2, a CH of SEQ ID NO: 13, and a CL of SEQ ID NO: 15.

In certain embodiments, the anti-CD2 antibody or antigen-binding fragment thereof described herein can comprise a heavy chain of SEQ ID NO: 16; a VL of SEQ ID NO: 2, and a CL of SEQ ID NO: 14; a heavy chain of SEQ ID NO: 16; a VL of SEQ ID NO: 2, and a CL of SEQ ID NO: 15; a VH of SEQ ID NO: 1; a CH of SEQ ID NO: 9; and a light chain of SEQ ID NO: 17; a VH of SEQ ID NO: 1; a CH of SEQ ID NO: 10; and a light chain of SEQ ID NO: 17; a VH of SEQ ID NO: 1; a CH of SEQ ID NO: 11; and a light chain of SEQ ID NO: 17; a VH of SEQ ID NO: 1; a CH of SEQ ID NO: 12; and a light chain of SEQ ID NO: 17; a VH of SEQ ID NO: 1; a CH of SEQ ID NO: 13; and a light chain of SEQ ID NO: 17; or a heavy chain of SEQ ID NO: 16 and a light chain of SEQ ID NO: 17.

In certain embodiments, the anti-CD2 antibody or antigen-binding fragment thereof may comprise enlarged variants. In certain embodiments, the Fc of the anti-CD2 antibody or antigen-binding fragment thereof described herein comprises an additional region such that the molecular weight of the anti-CD2 antibody or antigen-binding fragment thereof is increased by 10 kDa, 11 kDa, 12 kDa, 13 kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa, or 60 kDa. In certain embodiments, the Fc of the anti-CD2 antibody or antigen-binding fragment thereof described herein comprises an additional region such that the molecular weight of the anti-CD2 antibody or antigen-binding fragment thereof is increased by a range of 10 kDa to 15 kDa, 11 kDa to 16 kDa, 12 kDa to 17 kDa, 13 kDa to 18 kDa, 14 kDa to 19 kDa, 15 kDa to 20 kDa, 16 kDa to 21 kDa, 17 kDa to 22 kDa, 18 kDa to 23 kDa, 19 kDa to 24 kDa, 20 kDa to 25 kDa, 21 kDa to 26 kDa, 22 kDa to 27 kDa, 23 kDa to 28 kDa, 24 kDa to 29 kDa, 25 kDa to 30 kDa, 26 kDa to 31 kDa, 27 kDa to 32 kDa, 28 kDa to 33 kDa, 29 kDa to 34 kDa, 30 kDa to 35 kDa, 31 kDa to 36 kDa, 32 kDa to 37 kDa, 33 kDa to 38 kDa, 34 kDa to 39 kDa, 35 kDa to 40 kDa, 36 kDa to 41 kDa, 37 kDa to 42 kDa, 38 kDa to 43 kDa, 39 kDa to 44 kDa, 40 kDa to 45 kDa, 41 kDa to 46 kDa, 42 kDa to 47 kDa, 43 kDa to 48 kDa, 44 kDa to 49 kDa, 45 kDa to 50 kDa, 46 kDa to 51 kDa, 47 kDa to 52 kDa, 48 kDa to 53 kDa, 49 kDa to 54 kDa, 50 kDa to 55 kDa, 51 kDa to 56 kDa, 52 kDa to 57 kDa, 53 kDa to 58 kDa, 54 kDa to 59 kDa, or 55 kDa to 60 kDa.

In certain embodiments, duplicate regions of the anti-CD2 antibody or antigen-binding fragment thereof can be attached to the molecule to produce enlarged variants. In certain embodiments, these regions can be attached to the Fc region of the anti-CD2 antibody or antigen-binding fragment thereof through such means known in the art such as, but not limited to, chemical conjugation, recombinant fusion, and covalent attachment.

In certain embodiments, the enlarged variants of the anti-CD2 antibody or antigen-binding fragment thereof can be derived from an IgG1, IgG 2, or IgG4. In certain embodiments, an additional scFv can be attached to the Fc of the anti-CD2 antibody or antigen-binding fragment thereof to produce an enlarged variant. In certain embodiments, the scFv can comprise a VH and VL wherein the VH can comprise an amino acid sequence that is about or at least about 80%, 85%, 90%, 95%, 98%, or 99% identical to the amino acid sequence of SEQ ID NO:1, and wherein the VL can comprise an amino acid sequence that is about or at least about 80%, 85%, 90%, 95%, 98%, or 99% identical to the amino acid sequence of SEQ ID NO:2.

In certain embodiments, an additional CH2 domain can be attached to the Fc of the anti-CD2 antibody or antigen-binding fragment thereof to produce an enlarged variant. In certain embodiments, the additional CH2 domain can comprise an amino acid sequence that is about or at least about 80%, 85%, 90%, 95%, 98%, or 99% identical to the amino acid sequence of SEQ ID NO:18 or 19. In certain embodiments, an additional CH3 domain can be attached to the Fc of the anti-CD2 antibody or antigen-binding fragment thereof to produce an enlarged variant. In certain embodiments, the additional CH3 domain can comprise an amino acid sequence that is about or at least about 80%, 85%, 90%, 95%, 98%, or 99% identical to the amino acid sequence of SEQ ID NO:20 or 21. In some embodiments, the additional fragment or domains can be attached to the C-terminal end of the Fc. In certain embodiments, the enlarged anti-CD2 variant can be an IgG2 antibody. In certain embodiments, the enlarged anti-CD2 variant can be an IgG4 antibody. The details of enlarged anti-CD2 variants are shown in Table 5.

In certain embodiments, the additional region attached to the anti-CD2 antibody or antigen-binding fragment thereof can comprise an unrelated or artificial amino acid sequence. In certain embodiments, the additional region attached to the anti-CD2 antibody or antigen-binding fragment thereof can comprise a fragment, such as a structural domain, from another human protein. In certain embodiments, the additional region attached to the anti-CD2 antibody or antigen-binding fragment thereof can comprise a fragment of a domain from another human protein. In certain embodiments, the addition of such an additional region does not interfere with the binding activity of the parent anti-CD2 antibody or antigen-binding fragment thereof and/or does not increase the immunogenicity of the resulting anti-CD2 binder relative to the parent anti-CD2 antibody or antigen-binding fragment thereof in humans.

TABLE 5
Enlarged anti-CD2 variants

| | | Additional | Theoretical |
|---|---|---|---|
| | IgG | antibody | protein |
| Description | subclass | component | weight |
| Molecule 5A | IgG2 | scFv | 199.4 kDa |
| Molecule 5B | IgG4 | scFv | 199.6 kDa |
| Molecule 6A | IgG2 | CH2 domain | 170.1 kDa |
| Molecule 6B | IgG4 | CH2 domain | 170.3 kDa |
| Molecule 7A | IgG2 | CH3 domain | 171.2 kDa |
| Molecule 7B | IgG4 | CH3 domain | 171.4 kDa |

In certain embodiments, the conditioning regimen provided herein comprises administering an anti-CD2 antibody or antigen binding fragment thereof to a transplant recipient prior to transplant and/or cell infusion. In some embodiments, an anti-CD2 antibody or antigen binding fragment thereof is administered to a recipient 1 day prior to transplant and/or cell infusion, 2 days prior to transplant and/or cell infusion, 3 days prior to transplant and/or cell infusion, 4 days prior to transplant and/or cell infusion, 5 days prior to transplant and/or cell infusion, 6 days prior to transplant and/or cell infusion, 7 days prior to transplant and/or cell infusion, 8 days prior to transplant and/or cell infusion, 9 days prior to transplant and/or cell infusion, 10 days prior to transplant and/or cell infusion, or more than 10 days prior to transplant and/or cell infusion. In some embodiments, an anti-CD2 antibody or antigen binding fragment thereof is administered to a recipient 1 day after the transplant and/or cell infusion, 2 days after the transplant and/or cell infusion, 3 days after the transplant and/or cell infusion, 4 days after the transplant and/or cell infusion, 5 days after the transplant and/or cell infusion, 6 days after the transplant and/or cell infusion, 7 days after the transplant and/or cell infusion, 8 days after the transplant and/or cell infusion, 9 days after the transplant and/or cell infusion, 10 days after the transplant and/or cell infusion, or more than 10 days after the transplant and/or cell infusion. In some embodiments, an anti-CD2 antibody or antigen binding fragment thereof is administered to a recipient on the same day as the transplant and/or cell infusion. In some embodiments, an anti-CD2 antibody or antigen binding fragment thereof is administered to a recipient six days prior to transplant and/or cell infusion, 1 day prior to transplant and/or cell infusion, on the same day as the transplant and/or cell infusion, and 1 day after the transplant and/or cell infusion; six days prior to transplant and/or cell infusion, 1 day prior to transplant and/or cell infusion, on the same day as the transplant and/or cell infusion, 1 day after the transplant and/or cell infusion, and six days after the transplant and/or cell infusion; 1 day prior to transplant and/or cell infusion; 1 day after transplant and/or cell infusion; on the same day as the transplant and/or cell infusion; six days prior to transplant and/or cell infusion; six days after transplant and/or cell infusion; 1 day prior to transplant and/or cell infusion and on the same day as the transplant and/or cell infusion; 1 day after the transplant and/or cell infusion and on the same day as the transplant and/or cell infusion; 1 day prior to transplant and/or cell infusion and 1 day after transplant and/or cell infusion; 1 day and/or 2 days prior to transplant and/or cell infusion; 2 days and/or 3 days prior to transplant and/or cell infusion; 3 days prior and/or 4 days prior to transplant and/or cell infusion; 1 day prior and/or 2 days prior and/or 3 days prior to transplant and/or cell infusion; 1 day prior and/or 2 days prior and/or 3 days prior and/or 4 days prior to transplant and/or cell infusion; 1 day prior and/or 2 days prior and/or 3 days prior and/or 4 days prior to transplant and/or cell infusion, and/or 5 days prior and/or 6 days prior to transplant and/or cell infusion; 1 day and/or 2 days after the transplant and/or cell infusion; 2 days and/or 3 days after the transplant and/or cell infusion; 3 days and/or 4 days after the transplant and/or cell infusion; 1 day after and/or 2 days after and/or 3 days after the transplant and/or cell infusion; 1 day after and/or 2 days after and/or 3 days after and/or 4 days after the transplant and/or cell infusion; 1 day after and/or 2 days after and/or 3 days after and/or 4 days after, and/or 5 days after and/or 6 days after the transplant and/or cell infusion. In some embodiments, an anti-CD2 antibody or antigen binding fragment thereof is not administered to a recipient 2 days prior to transplant and/or cell infusion. In some embodiments, an anti-CD2 antibody or antigen binding fragment thereof is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) prior to transplant and/or cell infusion. In some embodiments, an anti-CD2 antibody or antigen binding fragment thereof is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) after the transplant and/or cell infusion. In a specific embodiment, the anti-CD2 antibody or antigen binding fragment thereof is administered to a recipient 1 day prior and 6 days prior to transplant and/or cell infusion. In a specific embodiment, the anti-CD2 antibody or antigen binding fragment thereof is administered to a recipient 1 day prior, 6 days prior to transplant and/or cell infusion, on the day of the transplant and/or cell infusion, 1 day after, and/or 6 days after the transplant and/or cell infusion. In certain embodiments, a test dose of the anti-CD2 antibody or antigen binding fragment thereof can be administered. In some embodiments, the anti-CD2 antibody or antigen binding fragment thereof is administered to the subject after the transplant. In some embodiments, the anti-CD2 antibody or antigen binding fragment thereof is administered to the subject after about or after at least about 1 day, 2 days, 3 days, 4 days, 5 days, 6 days, 7 days, 8 days, 9 days, 10 days, 11 days, 12, days, 13 days, 14 days, 21 days, 30 days, 1 month, 2 months, 3 months, 4 months, 5 months, 6 months, 7 months, 8 months, 9 months, 10 months, 11 months, 12 months, or more than 12 months after the transplant. In certain embodiments, the administration of the test dose is optional. In some embodiments, if the anti-CD2 antibody or antigen binding fragment thereof is administered to a transplant recipient more than once or more than one day, the dosage is the same on all days and/or the same per administration. In some embodiments, if the anti-CD2 antibody or antigen binding fragment thereof is administered to a transplant recipient more than once or more than one day, the dosage is not the same on all days and/or not the same per administration. In some embodiments, the anti-CD2 antibody or antigen binding fragment thereof is administered to a transplant recipient at a dose of 0.6 mg/kg. In some embodiments, the anti-CD2 antibody or antigen binding fragment thereof is not administered to a transplant recipient at a dose of 0.1 mg/kg. In some embodiments, the anti-CD2 antibody or antigen binding fragment thereof is not administered to a transplant recipient at a dose of 0.1 mg/kg two days prior to transplant and/or cell infusion.

In certain embodiments, the dose amount of anti-CD2 antibody or antigen binding fragment thereof administered to the recipient in the postoperative regimen is the same as the dose amount administered in the conditioning regimen. In certain embodiments, the dose amount of anti-CD2 antibody or antigen binding fragment thereof administered to the recipient in the postoperative regimen is different than the dose amount administered in the conditioning regimen.

In certain embodiments, the anti-CD2 antibody or antigen binding fragment thereof is administered to a transplant recipient at a dose amount of about, at least about, or at most about: 0.05 mg/kg/dose, 0.1 mg/kg/dose, 0.15 mg/kg/dose, 0.2 mg/kg/dose, 0.25 mg/kg/dose, 0.3 mg/kg/dose, 0.35 mg/kg/dose, 0.4 mg/kg/dose, 0.45 mg/kg/dose, 0.5 mg/kg/dose, 0.55 mg/kg/dose, 0.6 mg/kg/dose, 0.65 mg/kg/dose, 0.7 mg/kg/dose, 0.75 mg/kg/dose, 0.8 mg/kg/dose, 0.85 mg/kg/dose, 0.9 mg/kg/dose, 0.95 mg/kg/dose, or 1.0 mg/kg/dose. In certain embodiments, the anti-CD2 antibody or antigen binding fragment thereof is administered to a transplant recipient at a dose amount of about 0.6 mg/kg. In certain embodiments, the anti-CD2 antibody is administered to a transplant recipient at dose ranges of about, at least about, or at most about: 0.1-0.3 mg/kg/dose, 0.2-0.4 mg/kg/dose, 0.3-0.5 mg/kg/dose, 0.4-0.6 mg/kg/dose, 0.45-0.65 mg/kg/dose, 0.5-0.7 mg/kg, 0.55-0.75 mg/kg/dose, 0.6-0.8 mg/kg/dose, 0.65-0.85 mg/kg/dose, 0.7-0.9 mg/kg/dose, or 0.8-1.0 mg/kg/dose. In a specific embodiment, the anti-CD2 antibody or antigen binding fragment thereof is administered to a transplant recipient at a dose amount of 0.1 mg/kg/dose. In a specific embodiment, the anti-CD2 antibody or antigen binding fragment thereof is to a transplant recipient at a dose amount of 0.6 mg/kg/dose.

In some embodiments, an anti-CD2 antibody or antigen-binding fragment thereof described herein is administered to a subject in need at a dose amount of about, at least about, or at most about: 0.01 mg/kg/dose, 0.02 mg/kg/dose, 0.03 mg/kg/dose, 0.04 mg/kg/dose, 0.05 mg/kg/dose, 0.1 mg/kg/dose, 0.15 mg/kg/dose, 0.2 mg/kg/dose, 0.25 mg/kg/dose, 0.3 mg/kg/dose, 0.35 mg/kg/dose, 0.4 mg/kg/dose, 0.45 mg/kg/dose, 0.5 mg/kg/dose, 0.55 mg/kg/dose, 0.6 mg/kg/dose, 0.65 mg/kg/dose, 0.7 mg/kg/dose, 0.75 mg/kg/dose, 0.8 mg/kg/dose, 0.85 mg/kg/dose, 0.9 mg/kg/dose, 0.95 mg/kg/dose, 1.0 mg/kg/dose, 2.0 mg/kg/dose, 3.0 mg/kg/dose, 4.0 mg/kg/dose, 5.0 mg/kg/dose, 6.0 mg/kg/dose, 7.0 mg/kg/dose, 8.0 mg/kg/dose, 9.0 mg/kg/dose, 10 mg/kg/dose, 11 mg/kg/dose, 12 mg/kg/dose, 13 mg/kg/dose, 14 mg/kg/dose, 15 mg/kg/dose, 16 mg/kg/dose, 17 mg/kg/dose, 18 mg/kg/dose, 19 mg/kg/dose, 20 mg/kg/dose, 21 mg/kg/dose, 22 mg/kg/dose, 23 mg/kg/dose, 24 mg/kg/dose, 25 mg/kg/dose, 26 mg/kg/dose, 27 mg/kg/dose, 28 mg/kg/dose, 29 mg/kg/dose, 30 mg/kg/dose, 31 mg/kg/dose, 32 mg/kg/dose, 33 mg/kg/dose, 34 mg/kg/dose, 35 mg/kg/dose, 36 mg/kg/dose, 37 mg/kg/dose, 38 mg/kg/dose, 39 mg/kg/dose, 40 mg/kg/dose, 41 mg/kg/dose, 42 mg/kg/dose, 43 mg/kg/dose, 44 mg/kg/dose, 45 mg/kg/dose, 46 mg/kg/dose, 47 mg/kg/dose, 48 mg/kg/dose, 49 mg/kg/dose, or 50 mg/kg/dose. In some embodiments, an anti-CD2 antibody or antigen-binding fragment thereof described herein is administered to a subject in need at dose ranges of about 0.1-0.3 mg/kg/dose, 0.2-0.4 mg/kg/dose, 0.3-0.5 mg/kg/dose, 0.4-0.6 mg/kg/dose, 0.45-0.65 mg/kg/dose, 0.5-0.7 mg/kg, 0.55-0.75 mg/kg/dose, 0.6-0.8 mg/kg/dose, 0.65-0.85 mg/kg/dose, 0.7-0.9 mg/kg/dose, 0.8-1.0 mg/kg/dose, between 1.0 mg/kg/dose and 6.0 mg/kg/dose, between 5.0 mg/kg/dose and 10 mg/kg/dose, between 9.0 mg/kg/dose and 15 mg/kg/dose, between 14 mg/kg/dose and 20 mg/kg/dose, between 19 mg/kg/dose and 25 mg/kg/dose, between 24 mg/kg/dose and 30 mg/kg/dose, between 29 mg/kg/dose and 35 mg/kg/dose, between 34 mg/kg/dose and 40 mg/kg/dose, between 39 mg/kg/dose and 45 mg/kg/dose, or between 44 mg/kg/dose and 50 mg/kg/dose.

In some embodiments, the anti-CD2 antibody or antigen binding fragment thereof is administered to a transplant recipient at an amount of about, at least about, or at most about: 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, between 1000 mg and 1600 mg, between 1500 mg and 2100 mg, between 2000 mg and 2600 mg, between 2500 mg and 3100 mg, between 3000 mg and 3600 mg, between 3500 mg and 4100 mg, between 4000 mg and 4600 mg, or between 4500 mg and 5000 mg. In some embodiments, an anti-CD2 antibody or antigen-binding fragment thereof described herein can be administered to a subject in need thereof at an amount of 2400 mg.

In certain embodiments, the anti-CD2 antibody or antigen binding fragment thereof can be administered to a transplant recipient in a convenient manner known in the art including subcutaneously, intravenously, intravascularly, topically, intra-arterially, intra-cranially, intramuscularly, orally, intra-orbitally, by inhalation, transdermally, intra-peritonially, or through a route of administration which allows for the depletion of T-cells in the recipient. In a specific embodiment, the anti-CD2 antibody or antigen binding fragment thereof is administered intravenously.

In certain embodiments, the anti-CD2 antibody or antigen binding fragment thereof is a humanized monoclonal antibody. In a specific embodiment, the anti-CD2 antibody or antigen binding fragment thereof can be Siplizumab (MEDI-507). In certain embodiments, the administration of the anti-CD2 antibody or antigen binding fragment thereof can be modified as described herein to achieve and/or maintained mixed chimerism.

Without being bound by theory, the anti-CD2 antibody or antigen binding fragment thereof increases the level of regulatory T cells in the recipient. Specifically, the anti-CD2 antibody or antigen binding fragment thereof can increase the level of FOXP3⁺ regulatory T cells in the recipient, e.g., by about or at least about 10%, 20%, 30%, 40%, 50%, 60%, 70%, 80%, 90%, 100%, 125%, 150%, 175%, 200%, or by at least 250% relative to FOXP3⁺ regulatory T cells without treatment with the anti-CD2 antibody or antigen binding fragment thereof.

In some embodiments, a method described herein comprises administration of the anti-CD2 antibody or antigen-binding fragment thereof and one or more immunosuppressants. Without being bound by theory, immunosuppressants that can be combined with administration of the anti-CD2 antibody or antigen-binding fragment thereof described herein include, but are not limited to, tacrolimus, cyclosporine, belatacept, anti-CD40 antibodies, anti-CD40L (CD154), anti-OX40, anti-OX40L antibodies, anti-CD28 antibodies, anti-CD27 antibodies, anti-ICOS antibodies, anti- or agonistic 4-1BB (CD137) antibodies, BCL-2 inhibitors, mycophenolate mofetil, mycophenolic acid derivatives such as Myfortic (enteric coated mycophenolate sodium), sirolimus, everolimus, anti-thymocyte globulin, basiliximab, prednisone, cyclophosphamide, fludarabine, and rituximab (Adams et al., J Immunol, 2016, 197 (6) 2045-2050; Kinnear et al., Transplantation. 2013 Feb. 27; 95(4): 527-535; Zhang and Vignali, Immunity. 2016 May 17; 44(5):1034-51).

### 5.3.3 Cyclophosphamide

Cyclophosphamide, as used herein, is a compound administered to the recipient to suppress the immune system. Cyclophosphamide (2-[bis(2-chloroethyl)amino]tetrahydro-2H-1,3,2-oxazaphosphorine 2-oxide monohydrate) is administered to induce tolerance towards the transplanted organ and brand names of cyclophosphamide include Cytoxan®, Neosar®, and Endoxan®.

In certain embodiments, the conditioning regimen provided herein comprises administering cyclophosphamide to a transplant recipient prior to transplant. In some embodiments, the cyclophosphamide can be administered to the recipient three days prior to transplant, four days prior to transplant, five days prior to transplant, six days prior to transplant, three days and four days prior to transplant, four days and five days prior to transplant, five days and six days prior to transplant, three days and four days and five days prior to transplant, or four days and five days and six days prior to transplant. In a specific embodiment, cyclophosphamide can be administered to a recipient five days and six days prior to transplant. In a specific embodiment, cyclophosphamide can be administered to a recipient four days and five days prior to transplant.

In certain embodiments, the conditioning regimen provided herein comprises administering cyclophosphamide to a transplant recipient prior to transplant and/or cell infusion. In some embodiments, cyclophosphamide is administered to a recipient 1 day prior to transplant and/or cell infusion, 2 days prior to transplant and/or cell infusion, 3 days prior to transplant and/or cell infusion, 4 days prior to transplant and/or cell infusion, 5 days prior to transplant and/or cell infusion, 6 days prior to transplant and/or cell infusion, 7 days prior to transplant and/or cell infusion, 8 days prior to transplant and/or cell infusion, 9 days prior to transplant and/or cell infusion, 10 days prior to transplant and/or cell infusion, or more than 10 days prior to transplant and/or cell infusion. In some embodiments, cyclophosphamide is administered to a recipient 1 day after the transplant and/or cell infusion, 2 days after the transplant and/or cell infusion, 3 days after the transplant and/or cell infusion, 4 days after the transplant and/or cell infusion, 5 days after the transplant and/or cell infusion, 6 days after the transplant and/or cell infusion, 7 days after the transplant and/or cell infusion, 8 days after the transplant and/or cell infusion, 9 days after the transplant and/or cell infusion, 10 days after the transplant and/or cell infusion, or more than 10 days after the transplant and/or cell infusion. In some embodiments, cyclophosphamide is administered to a recipient on the same day as the transplant and/or cell infusion. In some embodiments, cyclophosphamide is administered to a recipient four days prior to transplant and/or cell infusion and/or five days prior to transplant and/or cell infusion; on the same day as the transplant and/or cell infusion; four days prior to transplant and/or cell infusion; five days prior to transplant and/or cell infusion; 1 day prior to transplant and/or cell infusion; 1 day after transplant and/or cell infusion; on the same day as the transplant and/or cell infusion; 1 day and/or 2 days prior to transplant and/or cell infusion; 2 days and/or 3 days prior to transplant and/or cell infusion; 3 days prior and/or 4 days prior to transplant and/or cell infusion; 4 days prior and/or 5 days prior to transplant and/or cell infusion; 1 day prior and/or 2 days prior and/or 3 days prior to transplant and/or cell infusion; 1 day prior and/or 2 days prior and/or 3 days prior and/or 4 days prior to transplant and/or cell infusion; 1 day prior and/or 2 days prior and/or 3 days prior and/or 4 days prior, and/or 5 days prior to transplant and/or cell infusion; 1 day prior and/or 2 days prior and/or 3 days prior and/or 4 days prior, and/or 5 days prior, and/or 6 days prior to transplant and/or cell infusion; 1 day and/or 2 days after the transplant and/or cell infusion; 2 days and/or 3 days after the transplant and/or cell infusion; 3 days and/or 4 days after the transplant and/or cell infusion; 1 day after and/or 2 days after and/or 3 days after the transplant and/or cell infusion; 1 day after and/or 2 days after and/or 3 days after and/or 4 days after the transplant and/or cell infusion; 1 day after and/or 2 days after and/or 3 days after and/or 4 days after, and/or 5 days after the transplant and/or cell infusion; 1 day after and/or 2 days after and/or 3 days after and/or 4 days after, and/or 5 days after and/or 6 days after the transplant and/or cell infusion. In some embodiments, cyclophosphamide is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) prior to transplant and/or cell infusion. In some embodiments, cyclophosphamide is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) after the transplant and/or cell infusion. In a specific embodiment, cyclophosphamide is administered to a recipient 4 days prior and 5 days prior to transplant and/or cell infusion. In certain embodiments, a test dose of cyclophosphamide can be administered. In certain embodiments, the administration of the test dose is optional. In some embodiments, if the cyclophosphamide is administered to a transplant recipient more than once or more than one day, the dosage is the same on all days and/or the same per administration. In some embodiments, if the cyclophosphamide is administered to a transplant recipient more than once or more than one day, the dosage is not the same on all days and/or not the same per administration. In certain embodiments, the dose amount of cyclophosphamide administered to the recipient in the postoperative regimen is the same as the dose amount administered in the conditioning regimen. In certain embodiments, the dose amount of cyclophosphamide administered to the recipient in the postoperative regimen is different than the dose amount administered in the conditioning regimen. In some embodiments, cyclophosphamide is administered prophylactically.

In certain embodiments, the postoperative treatment regimen provided herein comprises administering cyclophosphamide to a transplant recipient. In certain embodiments, cyclophosphamide can be administered on the day of the transplant, 1 day after, 2 days after, 3 days after, 4 days after, 5 days after, 6 days after, 7 days after, 8 days after, 9 days after, or 10 days after the transplant surgery. In certain embodiments, cyclophosphamide can be administered on the day of the transplant surgery through 2 days after, on the day of the transplant surgery through 4 days after, on the day of the transplant surgery through 6 days after, on the day of the transplant surgery through 7 days after, on the day of the transplant surgery through 8 days after, on the day of the transplant surgery through 9 days after, or on the day of the transplant surgery through 10 days after the transplant surgery. In a specific embodiment, cyclophosphamide can be administered on the day of the transplant surgery through 7 days after the surgery.

In certain embodiments, the cyclophosphamide can be administered to a transplant recipient at a dose amount of about, at least about, or at most about: 5 mg/kg/dose, 10 mg/kg/dose, 11 mg/kg/dose, 12 mg/kg/dose, 13 mg/kg/dose, 13.5 mg/kg/dose, 14 mg/kg/dose, 14.5 mg/kg/dose, 15 mg/kg/dose, 15.5 mg/kg/dose, 16 mg/kg/dose, 17 mg/kg/dose, 18 mg/kg/dose, 19 mg/kg/dose, 20 mg/kg/dose, 22.5 mg/kg/dose, 25 mg/kg/dose, 30 mg/kg/dose, 35 mg/kg/dose, 40 mg/kg/dose, 45 mg/kg/dose, 50 mg/kg/dose, 55 mg/kg/dose, 56 mg/kg/dose, 57 mg/kg/dose, 58 mg/kg/dose, 59 mg/kg/dose, 60 mg/kg/dose, 61 mg/kg/dose, 62 mg/kg/dose, 63 mg/kg/dose, 64 mg/kg/dose, 65 mg/kg/dose, 70 mg/kg/dose, 75 mg/kg/dose, 80 mg/kg/dose, 85 mg/kg/dose, or 90 mg/kg/dose. In certain embodiments, the cyclophosphamide can be administered to a transplant recipient at dose of about 60 mg/kg. In certain embodiments, the cyclophosphamide can be administered to a transplant recipient at dose of about 22.5 mg/kg. In certain embodiments, the cyclophosphamide can be administered to a transplant recipient at dose of at least about or at most about 60 mg/kg. In certain embodiments, the cyclophosphamide can be administered to a transplant recipient at dose of at least about or at most about 22.5 mg/kg. In certain embodiments, the cyclophosphamide can be administered to a transplant recipient at dose ranges of 5-15 mg/kg/dose, 10-20 mg/kg/dose, 15-25 mg/kg/dose, 15-65 mg/kg/dose, 20-30 mg/kg/dose, 25-35 mg/kg/dose, 30-40 mg/kg/dose, 35-45 mg/kg/dose, 40-50 mg/kg/dose, 45-55 mg/kg/dose, 50-60 mg/kg, 55-65 mg/kg/dose, 60-70 mg/kg/dose, 65-75 mg/kg/dose, 70-80 mg/kg/dose, 75-85 mg/kg/dose, or 80-90 mg/kg/dose. In a specific embodiment, the cyclophosphamide can be administered to a transplant recipient at a dose amount of 60 mg/kg/dose. In a specific embodiment, the cyclophosphamide can be administered to a transplant recipient at a dose amount of 50 mg/kg/dose. In a specific embodiment, the cyclophosphamide can be administered to a transplant recipient at a dose amount of 22.5 mg/kg/dose. In a specific embodiment, the cyclophosphamide can be administered to a transplant recipient at a dose amount of 14.5 mg/kg/dose.

In a certain embodiment, the conditioning regimen can include cyclophosphamide and fludarabine administration to the patient. In certain embodiments, if fludarabine is administered to a patient, without being bound by theory, cyclophosphamide can be administered at a lower dose than if the patient did not receive fludarabine. When fludarabine is administered as a component of the conditioning regimen, the concentration of cyclophosphamide administration can include 5-15 mg/kg/dose, 10-20 mg/kg/dose, 15-25 mg/kg/dose, 20-30 mg/kg/dose, 25-35 mg/kg/dose, 30-40 mg/kg/dose, 35-45 mg/kg/dose, and 40-50 mg/kg/dose. In some embodiments, fludarabine is administered at 10 mg/m² and cyclophosphamide is administered to a subject at 22.5 mg/kg.

In certain embodiments, the cyclophosphamide can be administered to a transplant recipient in a convenient manner known in the art including subcutaneously, intravenously, intravascularly, topically, intraarterially, intracranially, intramuscularly, orally, intraorbitally, by inhalation, transdermally, intraperitonially, or through a route of administration which allows for the proper action of the cyclophosphamide by the recipient. In a specific embodiment, the cyclophosphamide is administration intravenously.

In certain embodiments, total body irradiation can be used in the place of cyclophosphamide. In certain embodiments, the administration of the cyclophosphamide can be modified as described herein to achieve and/or maintained mixed chimerism in the recipient.

### 5.3.4 B-Cell Depleting Antibody (e.g., Rituximab)

Rituximab, as used herein, is an anti-CD20 chimeric monoclonal antibody administered to the recipient to suppress the immune system. Rituximab has murine variable regions and human constant k region and Fc region. The Fc region can bind to human complement, initiate antibody dependent cellular cytotoxicity (ADCC), and complement dependent cytotoxicity (CDC). Administration of rituximab results in depletion of B cells.

In certain embodiments, the conditioning regimen provided herein comprises administering a B-cell depleting antibody (e.g., rituximab) to a transplant recipient. In some embodiments, one dose of a B-cell depleting antibody (e.g., rituximab) can be administered before the transplant. In some embodiments, two doses of a B-cell depleting antibody (e.g., rituximab) can be administered before the transplant. In some embodiments, more than two doses of a B-cell depleting antibody (e.g., rituximab) can be administered before the transplant.

In some embodiments, the B-cell depleting antibody (e.g., rituximab) can be administered to the recipient 1 day before the transplant, 2 days before the transplant, 3 days before the transplant, 4 days before the transplant, 5 days before the transplant, 6 days before the transplant, 7 days before the transplant, 8 days before the transplant, 9 days before the transplant, 10 days before the transplant, 9 days and 2 days before the transplant, 1 day and 4 days before transplant, 1 day and 5 days before transplant, 1 day and 6 days before transplant, 2 days and 5 days before the transplant, 2 days and 6 days before the transplant, 2 days and 7 days before the transplant, 3 days and 6 days before the transplant, 3 days and 7 days before the transplant, 3 days and 8 days before the transplant, 1 day and 3 days and 5 days before transplant, 2 days and 4 days and 6 days before transplant, 3 days and 5 days and 7 days before transplant, or 4 days and 6 days and 8 days before the transplant. In a specific embodiment, a B-cell depleting antibody (e.g., rituximab) can be administered to a recipient 2 days before and 7 days before the transplant. In a specific embodiment, a B-cell depleting antibody (e.g., rituximab) is not administered to a recipient 7 days before the transplant.

In certain embodiments, the conditioning regimen provided herein comprises administering a B-cell depleting antibody (e.g., rituximab) to a transplant recipient prior to transplant and/or cell infusion. In some embodiments, a B-cell depleting antibody (e.g., rituximab) is administered to a recipient 1 day prior to transplant and/or cell infusion, 2 days prior to transplant and/or cell infusion, 3 days prior to transplant and/or cell infusion, 4 days prior to transplant and/or cell infusion, 5 days prior to transplant and/or cell infusion, 6 days prior to transplant and/or cell infusion, 7 days prior to transplant and/or cell infusion, 8 days prior to transplant and/or cell infusion, 9 days prior to transplant and/or cell infusion, 10 days prior to transplant and/or cell infusion, or more than 10 days prior to transplant and/or cell infusion. In some embodiments, a B-cell depleting antibody (e.g., rituximab) is administered to the subject after the transplant and/or cell infusion. In some embodiments, a B-cell depleting antibody (e.g., rituximab) is administered to a recipient 1 day after the transplant and/or cell infusion, 2 days after the transplant and/or cell infusion, 3 days after the transplant and/or cell infusion, 4 days after the transplant and/or cell infusion, 5 days after the transplant and/or cell infusion, 6 days after the transplant and/or cell infusion, 7 days after the transplant and/or cell infusion, 8 days after the transplant and/or cell infusion, 9 days after the transplant and/or cell infusion, 10 days after the transplant and/or cell infusion, 11 days after the transplant and/or cell infusion, 12 days after the transplant and/or cell infusion, or more than 12 days after the transplant and/or cell infusion. In some embodiments, a B-cell depleting antibody (e.g., rituximab) is administered to a recipient on the same day as the transplant and/or cell infusion. In some embodiments, a B-cell depleting antibody (e.g., rituximab) is administered to a recipient nine days prior to transplant and/or cell infusion and/or two days prior to transplant and/or cell infusion; nine days prior to transplant and/or cell infusion, and/or two days prior to transplant and/or cell infusion, and/or five days after transplant and/or cell infusion, and/or twelve days after the transplant and/or cell infusion; nine days prior to transplant and/or cell infusion; two days prior to transplant and/or cell infusion; 1 day prior to transplant and/or cell infusion; 1 day after transplant and/or cell infusion; on the same day as the transplant and/or cell infusion; 5 days after transplant and/or cell infusion; 12 days after transplant and/or cell infusion; 1 day and/or 2 days prior to transplant and/or cell infusion; 2 days and/or 3 days prior to transplant and/or cell infusion; 3 days prior and/or 4 days prior to transplant and/or cell infusion; 9 days prior and/or 2 days prior to transplant and/or cell infusion; 1 day prior and/or 2 days prior and/or 3 days prior to transplant and/or cell infusion; 1 day prior and/or 2 days prior and/or 3 days prior and/or 4 days prior to transplant and/or cell infusion; 1 day prior and/or 2 days prior and/or 3 days prior and/or 4 days prior and/or 5 days prior to transplant and/or cell infusion; 1 day prior and/or 2 days prior and/or 3 days prior and/or 4 days prior and/or 5 days prior and/or 6 days prior to transplant and/or cell infusion; 1 day prior and/or 2 days prior and/or 3 days prior and/or 4 days prior and/or 5 days prior and/or 6 days prior and/or 7 days prior and/or 8 days prior and/or 9 days prior to transplant and/or cell infusion; 1 day and/or 2 days after the transplant and/or cell infusion; 2 days and/or 3 days after the transplant and/or cell infusion; 3 days and/or 4 days after the transplant and/or cell infusion; 1 day after and/or 2 days after and/or 3 days after the transplant and/or cell infusion; 1 day after and/or 2 days after and/or 3 days after and/or 4 days after the transplant and/or cell infusion; 1 day after and/or 2 days after and/or 3 days after and/or 4 days after, and/or 5 days after the transplant and/or cell infusion; 1 day after and/or 2 days after and/or 3 days after and/or 4 days after, and/or 5 days after and/or 6 days after the transplant and/or cell infusion; 1 day after and/or 2 days after and/or 3 days after and/or 4 days after, and/or 5 days after and/or 6 days after and/or 7 days after the transplant and/or cell infusion; 1 day after and/or 2 days after and/or 3 days after and/or 4 days after, and/or 5 days after and/or 6 days after and/or 7 days after, and/or 8 days after and/or 9 days after and/or 10 days after and/or 11 days after and/or 12 days after the transplant and/or cell infusion. In some embodiments, a B-cell depleting antibody (e.g., rituximab) is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) prior to transplant and/or cell infusion. In some embodiments, a B-cell depleting antibody (e.g., rituximab) is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) after the transplant and/or cell infusion. In a specific embodiment, a B-cell depleting antibody (e.g., rituximab) is administered to a recipient 9 days prior and 2 days prior to transplant and/or cell infusion. In a specific embodiment, a B-cell depleting antibody (e.g., rituximab) is administered to a recipient 5 days after and 12 days after the transplant and/or cell infusion. In a specific embodiment, a B-cell depleting antibody (e.g., rituximab) is administered to a recipient 9 days prior and 2 days prior to transplant and/or cell infusion 5 days after and 12 days after the transplant and/or cell infusion. In some embodiments, a B-cell depleting antibody (e.g., rituximab) is administered to the subject after about or after at least about 1 day, 2 days, 3 days, 4 days, 5 days, 6 days, 7 days, 8 days, 9 days, 10 days, 11 days, 12, days, 13 days, 14 days, 21 days, 30 days, 1 month, 2 months, 3 months, 4 months, 5 months, 6 months, 7 months, 8 months, 9 months, 10 months, 11 months, 12 months, or more than 12 months after the transplanting. In certain embodiments, a test dose of a B-cell depleting antibody (e.g., rituximab) can be administered. In certain embodiments, the administration of the test dose is optional. In some embodiments, if the B-cell depleting antibody (e.g., rituximab) is administered to a transplant recipient more than once or more than one day, the dosage is the same on all days and/or the same per administration. In some embodiments, if a B-cell depleting antibody (e.g., rituximab) is administered to a transplant recipient more than once or more than one day, the dosage is not the same on all days and/or not the same per administration. In certain embodiments, the dose amount of a B-cell depleting antibody (e.g., rituximab) administered to the recipient in the postoperative regimen is the same as the dose amount administered in the conditioning regimen. In certain embodiments, the dose amount of a BB-cell depleting antibody (e.g., rituximab) administered to the recipient in the postoperative regimen is different than the dose amount administered in the conditioning regimen. In some embodiments, cyclophosphamide is administered prophylactically.

In certain embodiments, the postoperative treatment regimen provided herein comprises administering a BB-cell depleting antibody (e.g., rituximab) to a transplant recipient. In some embodiments, one dose of a BB-cell depleting antibody (e.g., rituximab) can be administered postoperatively. In some embodiments, two doses of a B-cell depleting antibody (e.g., rituximab) can be administered postoperatively. In some embodiments, more than two doses of a BB-cell depleting antibody (e.g., rituximab) can be administered postoperatively.

In some embodiments, a B-cell depleting antibody (e.g., rituximab) can be administered to the recipient 3 days after the transplant, 4 days after the transplant, 5 days after the transplant, 6 days after the transplant, 7 days after the transplant, 8 days after the transplant, 9 days after the transplant, 10 days after the transplant, 11 days after the transplant, 12 days after the transplant, 13 days after the transplant, 3 days and 9 days after transplant, 3 days and 10 days after transplant, 4 days and 10 days after transplant, 4 days and 11 days after transplant, 5 days and 11 days after transplant, 5 days and 12 days after transplant, 6 days and 12 days after transplant, 6 days and 13 days after transplant, 3 days and 6 days and 9 days after transplant, 4 days and 7 days and 10 days after transplant, 5 days and 8 days and 12 days after transplant, 6 days and 9 days and 12 days after transplant, or 7 days and 10 days and 12 days after transplant. In a specific embodiment, rituximab can be administered to a recipient 5 days after and 12 days after the transplant.

In certain embodiments, a BB-cell depleting antibody (e.g., rituximab) can be administered to a transplant recipient at a dose amount of about, at least about, or at most about: 200 mg/m²/dose, 225 mg/m²/dose, 250 mg/m²/dose, 275 mg/m²/dose, 300 mg/m²/dose, 325 mg/m²/dose, 350 mg/m²/dose, 355 mg/m²/dose, 360 mg/m²/dose, 365 mg/m²/dose, 366 mg/m²/dose, 367 mg/m²/dose, 368 mg/m²/dose, 369 mg/m²/dose, 370 mg/m²/dose, 371 mg/m²/dose, 372 mg/m²/dose, 373 mg/m²/dose, 374 mg/m²/dose, 375 mg/m²/dose, 380 mg/m²/dose, 385 mg/m²/dose, 390 mg/m²/dose, 395 mg/m²/dose, 400 mg/m²/dose, 425 mg/m²/dose, 450 mg/m²/dose, 475 mg/m²/dose, 500 mg/m²/dose, or more than 500 mg/m²/dose. In a specific embodiment, rituximab can be administered to a recipient at a dose amount of 375 mg/m²/dose.

In certain embodiments, a B-cell depleting antibody (e.g., rituximab) can be administered to a transplant recipient at a dose range of about, at least about, or at most about: 200-250 mg/m²/dose, 225-275 mg/m²/dose, 250-300 mg/m²/dose, 275-325 mg/m²/dose, 300-350 mg/m²/dose, 325-375 mg/m²/dose, 350-400 mg/m²/dose, 375-425 mg/m²/dose, 400-450 mg/m²/dose, 425-475 mg/m²/dose, or 450-500 mg/m²/dose.

In certain embodiments, a B-cell depleting antibody (e.g., rituximab) can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritonially, or through a route of administration which allows for the proper action of a B-cell depleting antibody (e.g., rituximab) by the recipient. In a specific embodiment, a B-cell depleting antibody (e.g., rituximab) is administration intravenously. In certain embodiments, the administration of a B-cell depleting antibody (e.g., rituximab) can be modified as described herein to achieve and/or maintained mixed chimerism in the recipient.

In certain embodiments, substitute compounds can be used in the place of rituximab. These compounds can include plasmapheresis, Alemtuzumab (Lemtrada®), IdeS (Imflidase®), and anti-CD19 directed treatments.

### 5.3.5 Fludarabine

Fludarabine, as used herein, is a purine analogue administered to the recipient to suppress the immune system. Fludarabine (9H-Purin-6-amine, 2-fluoro-9-(5-O-phosphono-0-D-arabino-furanosyl) (2-fluoro-ara-AMP) is also recognized by the brand names Oforta® and Fludara®. In some embodiments, a comparable agent or another (e.g., equivalent) anti-neoplastic agent can be administered to a subject instead of fludarabine or in addition to fludarabine. In some embodiments, a comparable agent or another (e.g., equivalent) anti-neoplastic agent is administered using the same dosage and/or on the same day(s) as provided herein for fludarabine. In some embodiments, a comparable agent or another (e.g., equivalent) anti-neoplastic agent is administered using a dosage according to a product label of the anti-neoplastic agent or known/determined by one skilled in the art.

In certain embodiments, the conditioning regimen provided herein comprises administering fludarabine to a transplant recipient prior to transplant and/or cell infusion. In some embodiments, fludarabine is administered to a recipient 1 day prior to transplant and/or cell infusion, 2 days prior to transplant and/or cell infusion, 3 days prior to transplant and/or cell infusion, 4 days prior to transplant and/or cell infusion, 5 days prior to transplant and/or cell infusion, 6 days prior to transplant and/or cell infusion, 7 days prior to transplant and/or cell infusion, 8 days prior to transplant and/or cell infusion, 9 days prior to transplant and/or cell infusion, 10 days prior to transplant and/or cell infusion, or more than 10 days prior to transplant and/or cell infusion. In some embodiments, fludarabine is administered to a recipient 1 day after the transplant and/or cell infusion, 2 days after the transplant and/or cell infusion, 3 days after the transplant and/or cell infusion, 4 days after the transplant and/or cell infusion, 5 days after the transplant and/or cell infusion, 6 days after the transplant and/or cell infusion, 7 days after the transplant and/or cell infusion, 8 days after the transplant and/or cell infusion, 9 days after the transplant and/or cell infusion, 10 days after the transplant and/or cell infusion, 11 days after the transplant and/or cell infusion, 12 days after the transplant and/or cell infusion, or more than 12 days after the transplant and/or cell infusion. In some embodiments, fludarabine is administered to a recipient on the same day as the transplant and/or cell infusion. In some embodiments, fludarabine is administered to a recipient three days prior to transplant and/or cell infusion and/or four days prior to transplant and/or cell infusion and/or five days prior to transplant and/or cell infusion and/or six days prior to transplant and/or cell infusion; two days prior to transplant and/or cell infusion and/or three days prior to transplant and/or cell infusion, and/or four days after transplant and/or cell infusion, and/or five days after the transplant and/or cell infusion, and/or six days after the transplant and/or cell infusion; six days prior to transplant and/or cell infusion; five days prior to transplant and/or cell infusion; four days prior to transplant and/or cell infusion; three days prior to transplant and/or cell infusion; 1 day prior to transplant and/or cell infusion; 1 day after transplant and/or cell infusion; on the same day as the transplant and/or cell infusion; 2 days after transplant and/or cell infusion; 3 days after transplant and/or cell infusion; 4 days after transplant and/or cell infusion; 5 days after transplant and/or cell infusion; 12 days after transplant and/or cell infusion; 1 day and/or 2 days prior to transplant and/or cell infusion; 2 days and/or 3 days prior to transplant and/or cell infusion; 3 days prior and/or 4 days prior to transplant and/or cell infusion; 9 days prior and/or 2 days prior to transplant and/or cell infusion; 1 day prior and/or 2 days prior and/or 3 days prior to transplant and/or cell infusion; 1 day prior and/or 2 days prior and/or 3 days prior and/or 4 days prior to transplant and/or cell infusion; 1 day prior and/or 2 days prior and/or 3 days prior and/or 4 days prior and/or 5 days prior to transplant and/or cell infusion; 1 day prior and/or 2 days prior and/or 3 days prior and/or 4 days prior and/or 5 days prior and/or 6 days prior to transplant and/or cell infusion; 1 day prior and/or 2 days prior and/or 3 days prior and/or 4 days prior and/or 5 days prior and/or 6 days prior and/or 7 days prior and/or 8 days prior and/or 9 days prior to transplant and/or cell infusion; 1 day and/or 2 days after the transplant and/or cell infusion; 2 days and/or 3 days after the transplant and/or cell infusion; 3 days and/or 4 days after the transplant and/or cell infusion; 1 day after and/or 2 days after and/or 3 days after the transplant and/or cell infusion; 1 day after and/or 2 days after and/or 3 days after and/or 4 days after the transplant and/or cell infusion; 1 day after and/or 2 days after and/or 3 days after and/or 4 days after, and/or 5 days after the transplant and/or cell infusion; 1 day after and/or 2 days after and/or 3 days after and/or 4 days after, and/or 5 days after and/or 6 days after the transplant and/or cell infusion; 1 day after and/or 2 days after and/or 3 days after and/or 4 days after, and/or 5 days after and/or 6 days after and/or 7 days after the transplant and/or cell infusion; 1 day after and/or 2 days after and/or 3 days after and/or 4 days after, and/or 5 days after and/or 6 days after and/or 7 days after, and/or 8 days after and/or 9 days after and/or 10 days after and/or 11 days after and/or 12 days after the transplant and/or cell infusion. In some embodiments, fludarabine is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) prior to transplant and/or cell infusion. In some embodiments, fludarabine is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) after the transplant and/or cell infusion. In a specific embodiment, fludarabine is administered to a recipient 3 days prior, 4 days prior, 5 days prior, and 6 days prior to transplant and/or cell infusion. In certain embodiments, a test dose of fludarabine can be administered. In certain embodiments, the administration of the test dose is optional. In some embodiments, if fludarabine is administered to a transplant recipient more than once or more than one day, the dosage is the same on all days and/or the same per administration. In some embodiments, if a fludarabine is administered to a transplant recipient more than once or more than one day, the dosage is not the same on all days and/or not the same per administration. In certain embodiments, the dose amount of fludarabine administered to the recipient in the postoperative regimen is the same as the dose amount administered in the conditioning regimen. In certain embodiments, the dose amount of fludarabine administered to the recipient in the postoperative regimen is different than the dose amount administered in the conditioning regimen. In some embodiments, fludarabine is administered prophylactically.

In certain embodiments, the postoperative treatment regimen provided herein comprises administering fludarabine to a transplant recipient. In some embodiments, one dose of fludarabine can be administered postoperatively. In some embodiments, two doses of fludarabine can be administered postoperatively. In some embodiments, more than two doses of fludarabine can be administered postoperatively.

In certain embodiments, the conditioning regimen provided herein comprises administering fludarabine to a transplant recipient prior to transplant. In certain embodiments, the conditioning regimen comprises fludarabine administration and cyclophosphamide administration. In certain embodiments, the conditioning regimen comprises fludarabine administration and low dose cyclophosphamide administration, wherein the dose of cyclophosphamide can include 5-15 mg/kg/dose, 10-20 mg/kg/dose, 15-25 mg/kg/dose, 20-30 mg/kg/dose, 25-35 mg/kg/dose, 30-40 mg/kg/dose, 35-45 mg/kg/dose, and 40-50 mg/kg/dose.

In some embodiments, the fludarabine can be administered to the recipient one day prior to transplant, two days prior to transplant, three days prior to transplant, four days prior to transplant, five days prior to transplant, six days prior to transplant, one day and two days prior to transplant, two days and three days prior to transplant, three days and four days prior to transplant, four days and five days prior to transplant, one day and two days and three days prior to transplant, two days and three days and four days prior to transplant, three days and four days and five days prior to transplant, or three days and four days and five days and six days prior to transplant. In a specific embodiment, cyclophosphamide can be administered to a recipient five days and four days prior to transplant and fludarabine can be administered to a recipient three days, four days, five days, and six days prior to transplant. In a specific embodiment, fludarabine can be administered to a recipient two days, three days, and four days prior to transplant. In a specific embodiment, cyclophosphamide can be administered to a recipient five days and six days prior to transplant and fludarabine can be administered to a recipient two days, three days, and four days prior to transplant. In a specific embodiment, cyclophosphamide can be administered to a recipient three days prior to transplant and fludarabine can be administered to a recipient two days, three days, and four days prior to transplant.

In certain embodiments, the fludarabine can be administered to a transplant recipient at a dose amount of about, at least about, or at most about: 5 mg/m²/dose, 6 mg/m²/dose, 7 mg/m²/dose, 8 mg/m²/dose, 9 mg/m²/dose, 10 mg/m²/dose, 11 mg/m²/dose, 12 mg/m²/dose, 13 mg/m²/dose, 14 mg/m²/dose, 15 mg/m²/dose, 20 mg/m²/dose, 21 mg/m²/dose, 22 mg/m²/dose, 23 mg/m²/dose, 24 mg/m²/dose, 25 mg/m²/dose, 26 mg/m²/dose, 27 mg/m²/dose, 28 mg/m²/dose, 29 mg/m²/dose, 30 mg/m²/dose, 31 mg/m²/dose, 32 mg/m²/dose, 33 mg/m²/dose, 34 mg/m²/dose, 35 mg/m²/dose, 40 mg/m²/dose, 45 mg/m²/dose, or 50 mg/m²/dose. In certain embodiments, the fludarabine can be administered to a transplant recipient at a dose amount of about 10 mg/m²/dose. In certain embodiments, the fludarabine can be administered to a transplant recipient at dose ranges of about, at least about, or at most about: 5-15 mg/m²/dose, 10-15 mg/m²/dose, 20-30 mg/m²/dose, 25-35 mg/m²/dose, 30-40 mg/m²/dose, 35-45 mg/m²/dose, or 40-50 mg/m²/dose. In a specific embodiment, the fludarabine can be administered to a transplant recipient at a dose amount of about, at least about, or at most about 24 mg/m²/dose. In a specific embodiment, the fludarabine can be administered to a transplant recipient at a dose amount of about 10 mg/m²/dose and cyclophosphamide can be administered at a range of 5-15 mg/kg/dose, 10-20 mg/kg/dose, 15-25 mg/kg/dose, 20-30 mg/kg/dose, or 25-35 mg/kg/dose. In a specific embodiment, the fludarabine can be administered to a transplant recipient at a dose amount of 24 mg/m²/dose and cyclophosphamide can be administered at a range of 5-15 mg/kg/dose, 10-20 mg/kg/dose, 15-25 mg/kg/dose, 20-30 mg/kg/dose, or 25-35 mg/kg/dose. In a specific embodiment, the fludarabine can be administered to a transplant recipient at a dose amount of 24 mg/m²/dose and cyclophosphamide can be administered at a dose amount of 14.5 mg/kg/dose.

In certain embodiments, the fludarabine can be administered to a transplant recipient at a dose amount of 5 mg/sqm, 10 mg/sqm, 15 mg/sqm, 20 mg/sqm, 21 mg/sqm, 22 mg/sqm, 23 mg/sqm, 24 mg/sqm, 25 mg/sqm, 26 mg/sqm, 27 mg/sqm, 28 mg/sqm, 29 mg/sqm, 30 mg/sqm, 31 mg/sqm, 32 mg/sqm, 33 mg/sqm, 34 mg/sqm, 35 mg/sqm, 40 mg/sqm, 45 mg/sqm, or 50 mg/sqm. In certain embodiments, the fludarabine can be administered to a transplant recipient at dose ranges of about, at least about, or at most about: 5-15 mg/sqm, 10-15 mg/sqm, 20-30 mg/sqm, 25-35 mg/sqm, 30-40 mg/sqm, 35-45 mg/sqm, or 40-50 mg/sqm. In a specific embodiment, the fludarabine can be administered to a transplant recipient at a dose amount of about 10 mg/sqm. In a specific embodiment, the fludarabine can be administered to a transplant recipient at a dose amount of about, at least about, or at most about 30 mg/sqm. In a specific embodiment, the fludarabine can be administered to a transplant recipient at a dose amount of about 10 mg/sqm and cyclophosphamide can be administered at a range of about 15-25 mg/kg/dose, 20-25 mg/kg/dose, 20-30 mg/kg/dose, 40-50 mg/kg/dose, 45-55 mg/kg/dose, 50-60 mg/kg/dose, or 55-65 mg/kg/dose. In a specific embodiment, the fludarabine can be administered to a transplant recipient at a dose amount of 30 mg/sqm and cyclophosphamide can be administered at a range of about 15-25 mg/kg/dose, 20-25 mg/kg/dose, 20-30 mg/kg/dose, 40-50 mg/kg/dose, 45-55 mg/kg/dose, 50-60 mg/kg/dose, or 55-65 mg/kg/dose. In a specific embodiment, the fludarabine can be administered to a transplant recipient at a dose amount of 10 mg/sqm and cyclophosphamide can be administered at a dose amount of 22.5 mg/kg/dose. In a specific embodiment, the fludarabine can be administered to a transplant recipient at a dose amount of 30 mg/sqm and cyclophosphamide can be administered at a dose amount of 50 mg/kg/dose.

In certain embodiments, the fludarabine can be administered to a transplant recipient in a convenient manner known in the art including subcutaneously, intravenously, intravascularly, topically, intraarterially, intracranially, intramuscularly, orally, intraorbitally, by inhalation, transdermally, intraperitonially, or through a route of administration which allows for the proper action of the fludarabine by the recipient. In a specific embodiment, the fludarabine is administration intravenously. In a specific embodiment, the fludarabine is administration orally.

### 5.3.6 Thymic Irradiation

In certain embodiments, the conditioning regimen provided herein comprises the recipient undergoing thymic irradiation before the transplant surgery. In some embodiments, the thymic irradiation can be performed 1 day before, 2 days before, more than 2 days before, or 1 and 2 days before the transplant surgery. In certain embodiments, the dosage of thymic irradiation is such as to be sufficient to deplete intrathymic T-cells. In certain embodiments, the dosage of thymic irradiation can be 100-1000 cGy (centigray). In certain embodiments, the dosage of thymic irradiation is about, at least about, or at most about 100 cGy, 200 cGy, 300 cGy, 400 cGy, 500 cGy, 600 cGy, 700 cGy, 800 cGy, 900 cGy, or 1000 cGy. In certain embodiments, the dosage of thymic irradiation is about, at least about, or at most about 7 Gy. In a specific embodiment, the recipient can undergo a thymic irradiation of about 7 Gy on the day before the transplant surgery. In a specific embodiment, the recipient can undergo a thymic irradiation of 700 cGy on the day before the transplant surgery. In certain embodiments, the timing and dosing of the thymic irradiation can be modified as described herein to achieve and/or maintained mixed chimerism in the recipient.

### 5.3.7 Total Body Irradiation

In certain embodiments, the conditioning regimen provided herein comprises the recipient undergoing total body irradiation before the transplant surgery. In some embodiments, the total body irradiation can be performed 3 days before the transplant, 4 days before the transplant, 5 days before the transplant, 6 days before the transplant, 7 days before the transplant, 3 and 4 days before the transplant, 3 and 5 days before the transplant, 4 and 5 days before the transplant, 4 and 6 days before the transplant, 5 and 6 days before the transplant, 5 and 7 days before the transplant, or 6 and 7 days before the transplant surgery. In certain embodiments, the dosage of total body irradiation can be 0.5-2.5 Gy. In certain embodiments, the dosage of total body irradiation can be about, at least about, or at most about: 0.5 Gy, 0.75 Gy, 1.0 Gy, 1.25 Gy, 1.50 Gy, 1.75 Gy, 2.0 Gy, 2.25 Gy, 2.50 Gy, 0.5-1.5 Gy, 0.75-1.25 Gy, 1.0-2.0 Gy, 1.25-2.25 Gy, or 1.50-2.50 Gy. In certain embodiments, the recipient can undergo total body irradiation once a day. In certain embodiments, the recipient can undergo total body irradiation twice a day. In a specific embodiment, the recipient can undergo a total body irradiation of 1.5 Gy, twice a day, 5 days and 4 days before the transplant surgery. In a specific embodiment, the recipient can undergo a total body irradiation of 1.5 Gy, twice a day, 6 days and 5 days before the transplant surgery. In certain embodiments, the timing and dosing of the total body irradiation can be modified as described herein to achieve and/or maintained mixed chimerism in the recipient.

### 5.3.8 Tacrolimus

Tacrolimus, as used herein, is a macrolide antibiotic administered to a recipient to suppress the immune system. Tacrolimus has a mode of action similar to CyA (calceinurin inhibitor), and brand names of tacrolimus include Prograf®, Adport®, Advagraf®, Protopic®, Astagraf XL®, Modigraf®, and Envarsus XR®.

In certain embodiments, the conditioning regimen provided herein comprises administering tacrolimus to a transplant recipient. In certain embodiments, tacrolimus can be administered 1 day before the transplant, 2 days before the transplant, 3 days before the transplant, 1 day and 2 days before the transplant, 1 day and 3 days before transplant, or 1 day and 2 days and 3 days before the transplant. In a specific embodiment, tacrolimus can be administered to a recipient 1 day before the transplant. In some embodiments, tacrolimus is administered on the same day as the transplant.

Tacrolimus can be included in the postoperative treatment regimen to suppress the immune system and inhibit the development of Graft versus Host disease in the recipient. The postoperative treatment regimen can include a constant course followed by a tapering course of tacrolimus administration to the recipient.

In certain embodiments, the postoperative treatment regimen provided herein comprises administering Tacrolimus to a transplant recipient. In certain embodiments, Tacrolimus can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, 30 days after, 1 month after, 2 months after, 3 months after, 4 months after, 5 months after, 6 months after, 7 months after, 8 months after, 9 months after, 10 months after, 11 months after, 12 months after, 13 months after, 14 months after, 15 months after, 16 months after, 17 months after, or 18 months after the transplant surgery. In some embodiments, Tacrolimus is administered to the subject after the transplant. In some embodiments, Tacrolimus is administered to the subject after about or after at least about 1 day, 2 days, 3 days, 4 days, 5 days, 6 days, 7 days, 8 days, 9 days, 10 days, 11 days, 12, days, 13 days, 14 days, 21 days, 30 days, 1 month, 2 months, 3 months, 4 months, 5 months, 6 months, 7 months, 8 months, 9 months, 10 months, 11 months, 12 months, 18 months, 20 months, 24 months, or more than 24 months after the transplant. In some embodiments, Tacrolimus is administered to the subject after about or after at least about 1-2 months, 2-3 months, 3-4 months, 4-5 months, 5-6 months, 6-7 months, 7-8 months, 8-9 months, 9-10 months, 11-12 months, 1-6 months, 2-6 months, 3-6 months, 4-6 months, 6-12 months, or more than 6-12 months after the transplant. In some embodiments, tacrolimus is administered for a continuous amount of time (e.g., from the day of the transplant to about 1 month, or to about 9 months, or to about 12 months after the transplant or after the start of treatment). In some embodiments, tacrolimus is administered at an amount of about 8-10 ng/ml. In some embodiments, tacrolimus is administered at an amount of about 4-11 ng/ml.

In certain embodiments, a single dose amount of tacrolimus can be administered. In certain embodiments, multiple dose amounts of tacrolimus can be administered. In certain embodiments, a constant dose of tacrolimus can be administered. In certain embodiments, a tapering course of tacrolimus can be administered. In certain embodiments, a constant dose of tacrolimus followed by a tapering course of tacrolimus can be administered postoperatively.

In certain embodiments, tacrolimus can be administered to a transplant recipient at a frequency of once a day or twice a day. In certain embodiments, tacrolimus can be administered twice a day at a dose amount of about, at least about, or at most about: 0.01 mg/kg/dose, 0.02 mg/kg/dose, 0.03 mg/kg/dose, 0.04 mg/kg/dose, 0.05 mg/kg/dose, 0.06 mg/kg/dose, 0.07 mg/kg/dose, 0.08 mg/kg/dose, 0.09 mg/kg/dose, 0.1 mg/kg/dose, 0.01-0.05 mg/kg/dose, 0.05-0.1 mg/kg/dose, 0.02-0.06 mg/kg/dose, 0.03-0.07 mg/kg/dose, 0.04-0.08 mg/kg/dose, or 0.01-0.1 mg/kg/dose. In a specific embodiment, tacrolimus can be administered to a transplant recipient twice a day at a dose amount of 0.05 mg/kg/dose.

In certain embodiments, tacrolimus can be administered postoperatively to a recipient at a sufficient dose amount to obtain the target trough blood levels of 1-5 ng/ml, 5-10 ng/ml, 8-10 ng/ml, 4-11 ng/ml, 10-15 ng/ml, 1-11 ng/ml, 2-12 ng/ml, 3-13 ng/ml, 4-14 ng/ml, 5-15 ng/ml, 6-16 ng/ml, 7-17 ng/ml, 8-18 ng/ml, 9-19 ng/ml, 10-20 ng/ml, or 15-20 ng/ml. In a specific embodiment, the target trough blood levels can be 10-15 ng/ml.

In some embodiments, tacrolimus is administered to a subject at a dose of about, at least about, or at most about 1-5 ng/ml, 5-10 ng/ml, 8-10 ng/ml, 4-11 ng/ml, 10-15 ng/ml, 1-11 ng/ml, 2-12 ng/ml, 3-13 ng/ml, 4-14 ng/ml, 5-15 ng/ml, 6-16 ng/ml, 7-17 ng/ml, 8-18 ng/ml, 9-19 ng/ml, 10-20 ng/ml, or 15-20 ng/ml. In some embodiments, tacrolimus is administered to a subject at a dose of about, at least about, or at most about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, 22 ng/ml, 25 ng/ml, 30 ng/ml, or more than 30 ng/ml.

In certain embodiments, tacrolimus can be administered to a transplant recipient at a constant dose. The duration of time a constant dose is administered can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, 90 days, 120 days, 150 days, 6 months, 7 months, 8 months, 9 months, 10 months, 11 months, 12 months, or more than 12 months after the transplant. In certain embodiments, tacrolimus administered to a transplant recipient can be tapered to discontinuation. In certain embodiments, this tapering course can take place over 1 month, 2 months, 3 months, 4 months, 5 months, 6 months, 7 months, 8 months, 9 months, 10 months, 11 months, 12 months, 13 months, 14 months, 15 months, 16 months, 17 months, or 18 months after the transplant.

In certain embodiments, tacrolimus can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritonially, or through a route of administration which allows for the proper action of the tacrolimus by the recipient. In a specific embodiment, tacrolimus can be administered orally. In a specific embodiment, the tacrolimus can be administered intravenously. In certain embodiments, the administration of tacrolimus can be modified as described herein to achieve and/or maintained mixed chimerism in the recipient.

In certain embodiments, potential equivalents of tacrolimus can be substituted in the postoperative treatment regimen. These substitutes can include cyclosporine (Gengraf®, Neoral®, and Sandimmune®), Belatacept (Nulojix), sirolumus, and everolimus. In specific embodiments, Belatacept can be used to suppress the immune system in the recipient.

In certain embodiments, the postoperative treatment regimen provided herein comprises administering Belatacept to a transplant recipient. In certain embodiments, Belatacept can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, 30 days after, 31 days after, 32 days after, 33 days after, 34 days after, 35 days after, 36 days after, 37 days after, 38 days after, 39 days after, 40 days, 60 days, 90 days, 120 days, 150 days, 6 months, 7 months, 8 months, 9 months, 10 months, 11 months, 12 months, or more than 12 months after the transplant surgery. In certain embodiments, Belatacept can be administered a single time. In certain embodiments, Belatacept can be administered multiple times. In a specific embodiment, Belatacept can be administered 5 times after the transplant surgery. In certain embodiments, Belatacept can be administered 1 day after the transplant surgery. In certain embodiments, Belatacept can be administered 5 days after the transplant surgery. In certain embodiments, Belatacept can be administered 12 days after the transplant surgery. In certain embodiments, Belatacept can be administered 19 days after the transplant surgery. In certain embodiments, Belatacept can be administered 33 days after the transplant surgery. In a specific embodiment, Belatacept can be administered 1 day and 5 days and 12 days and 19 days and 33 days after the transplant surgery.

In certain embodiments, Belatacept or tacrolimus can be administered to a transplant recipient at a dose amount of about, at least about, or at most about: 2 mg/kg/day, 2.5 mg/kg/day, 3 mg/kg/day, 3.5 mg/kg/day, 4 mg/kg/day, 4.5 mg/kg/day, 5 mg/kg/day, 5.5 mg/kg/day, 6 mg/kg/day, 6.5 mg/kg/day, 7 mg/kg/day, 7.5 mg/kg/day, 8 mg/kg/day, 8.5 mg/kg/day, 9 mg/kg/day, 9.5 mg/kg/day, 10 mg/kg/day, 10.5 mg/kg/day, 11 mg/kg/day, 11.5 mg/kg/day, 12 mg/kg/day, 12.5 mg/kg/day, 13 mg/kg/day, 13.5 mg/kg/day, 14 mg/kg/day, 14.5 mg/kg/day, 15 mg/kg/day, 15.5 mg/kg/day, 16 mg/kg/day, 16.5 mg/kg/day, 17 mg/kg/day, 17.5 mg/kg/day, 18 mg/kg/day, 2-6 mg/kg/day, 3-7 mg/kg/day, 4-8 mg/kg/day, 5-9 mg/kg/day, 6-10 mg/kg/day, 7-11 mg/kg/day, 8-12 mg/kg/day, 9-13 mg/kg/day, 10-14 mg/kg/day, 11-15 mg/kg/day, 12-16 mg/kg/day, 13-17 mg/kg/day, or 14-18 mg/kg/day. In a specific embodiment, Belatacept or tacrolimus can be administered to a transplant recipient at a dose amount of 10 mg/kg/day.

In certain embodiments, a single dose amount of tacrolimus can be administered. In certain embodiments, multiple dose amounts of the tacrolimus can be administered. In certain embodiments, a constant dose of tacrolimus can be administered. In certain embodiments, a tapering course of tacrolimus can be administered. In certain embodiments, a constant dose of tacrolimus followed by a tapering course of tacrolimus can be administered.

In certain embodiments, tacrolimus can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritonially, or through a route of administration which allows for the proper action of the tacrolimus by the recipient. In a specific embodiment, tacrolimus can be administered orally. In a specific embodiment, the tacrolimus can be administered intravenously. In certain embodiments, the administration of tacrolimus can be modified as described herein to achieve and/or maintained mixed chimerism in the recipient

In certain embodiments, the conditioning regimen provided herein comprises administering tacrolimus to a transplant recipient prior to transplant and/or cell infusion. In some embodiments, tacrolimus is administered to a recipient 1 day prior to transplant and/or cell infusion, 2 days prior to transplant and/or cell infusion, 3 days prior to transplant and/or cell infusion, 4 days prior to transplant and/or cell infusion, 5 days prior to transplant and/or cell infusion, 6 days prior to transplant and/or cell infusion, 7 days prior to transplant and/or cell infusion, 8 days prior to transplant and/or cell infusion, 9 days prior to transplant and/or cell infusion, 10 days prior to transplant and/or cell infusion, or more than 10 days prior to transplant and/or cell infusion. In some embodiments, tacrolimus is administered to a recipient 1 day after the transplant and/or cell infusion, 2 days after the transplant and/or cell infusion, 3 days after the transplant and/or cell infusion, 4 days after the transplant and/or cell infusion, 5 days after the transplant and/or cell infusion, 6 days after the transplant and/or cell infusion, 7 days after the transplant and/or cell infusion, 8 days after the transplant and/or cell infusion, 9 days after the transplant and/or cell infusion, 10 days after the transplant and/or cell infusion, 11 days after the transplant and/or cell infusion, 12 days after the transplant and/or cell infusion, or more than 12 days after the transplant and/or cell infusion. In some embodiments, tacrolimus is administered to a recipient on the same day as the transplant and/or cell infusion (e.g., and for a period of time thereafter (e.g., 1 month, or 9-12 months)). In some embodiments, tacrolimus is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) prior to transplant and/or cell infusion. In some embodiments, tacrolimus is administered to a recipient about, at least about, or at most about: 1 time (or on 1 day), 2 times (or on 2 different days), 3 times (or on 3 different days), 4 times (or on 4 different days), 5 times (or on 5 different days), 6 times (or on 6 different days), 7 times (or on 7 different days), 8 times (or on 8 different days), 9 times (or on 9 different days), 10 times (or on 10 different days), or more than 10 times (or on 10 different days) after the transplant and/or cell infusion. In some embodiments, tacrolimus is administered to a recipient for about 1 day, 10 days, 15 days, 20 days, 25 days, 30 days, 1 month, 2 months, 3 months, 4 months, 5 months, 6 months, 7 months, 8 months, 9 months, 10 months, 11 months, 12 months, or more than 12 months after transplant. In some embodiments, if tacrolimus is administered to a transplant recipient more than once or more than one day, the dosage is the same on all days and/or the same per administration. In some embodiments, if tacrolimus is administered to a transplant recipient more than once or more than one day, the dosage is not the same on all days and/or not the same per administration. In certain embodiments, the dose amount of tacrolimus administered to the recipient in the postoperative regimen is the same as the dose amount administered in the conditioning regimen. In certain embodiments, the dose amount of tacrolimus administered to the recipient in the postoperative regimen is different than the dose amount administered in the conditioning regimen. In some embodiments, tacrolimus is administered prophylactically.

In certain embodiments, the postoperative treatment regimen provided herein comprises administering tacrolimus to a transplant recipient. In some embodiments, one dose of tacrolimus can be administered postoperatively. In some embodiments, two doses of tacrolimus can be administered postoperatively. In some embodiments, more than two doses of tacrolimus can be administered postoperatively.

### 5.3.9 Anti-IL6R Antibody

In certain embodiments, the methods described herein can include a therapeutic blockage of IL-6 cytokine signaling. In certain embodiments, IL-6 signaling can be blocked by targeting IL-6. In certain embodiments, IL-6 signaling can be blocked by targeting the IL6R. In certain embodiments, the IL6R can be targeted by a small molecule inhibitor (Hong et al., Immunol. 2015, 195(1) 237-245). In certain embodiments, the IL6R can be targeted by an antibody. In specific embodiments, an anti-IL6R treatment can be a humanized monoclonal antibody. In a specific embodiment, an anti-IL6R receptor can be tocilizumab. In certain embodiments, treatment may be administered to a transplant recipient to mitigate acute kidney injury (AKI, also known as engraftment syndrome) while at the same time likely further increasing the Treg levels.

In certain embodiments, an anti-IL6R antibody for use with the present methods and compositions comprises 1, 2, or 3 of the heavy chain CDRs of tocilizumab. In certain embodiments, an anti-IL6R antibody for use with the present methods and compositions comprises 1, 2, or 3 of the light chain CDRs of tocilizumab. In certain embodiments, an anti-IL6R antibody for use with the present methods and compositions comprises 1, 2, 3, 4, 5, or all 6 of the CDRs of tocilizumab.

TABLE 6
CDR sequences of tocilizumab

| CDR | | |
|---|---|---|
| Number | Heavy Chain | Light Chain |
| 1 | SDHAWS | RASQDISSYLN |
| | (SEQ ID NO: 22) | (SEQ ID NO: 25) |
| 2 | YISYSGITTYNPSLKS | YTSRLHS |
| | (SEQ ID NO: 23) | (SEQ ID NO: 26) |
| 3 | SLARTTAMDY | QQGNTLPYT |
| | (SEQ ID NO: 24) | (SEQ ID NO: 27) |

In certain embodiments, the anti-IL6R antibody for use with the present methods and compositions comprises 1, 2, 3, 4, 5, or all 6 of the CDRs set forth in Table 6. In certain embodiment, 1, 2, 3, 4, 5, or all 6 have 1, 2, 3, 4, 5, 6, 7, 8, 9, or 10 amino acid substitutions. In a more specific embodiment, such an amino acid substitution is a conservative amino acid substitution.

In certain embodiments, the anti-IL6R antibody for use with the methods and compositions provided herein has a heavy chain variable region comprising an amino acid sequence of about or at least about 80%, 85%, 90%, 95%, 98%, at least 99% or 100% identity to SEQ ID NO:28. In certain embodiments, the anti-IL6R antibody for use with the methods and compositions provided herein has a light chain variable region comprising an amino acid sequence of about or at least about 80%, 85%, 90%, 95%, 98%, at least 99% or 100% identity to SEQ ID NO:29.

In certain embodiments, the anti-IL6R antibody binds immunospecifically to the same epitope in human IL6R as tocilizumb.

In certain embodiments, the anti-IL6R antibody can be an animal-specific antibody, a human-specific antibody, a chimeric antibody, a humanized antibody, a be full length antibody, an antibody fragment, a single chain variable fragment (scFv), a naturally occurring antibody, a synthetic antibody, an engineered antibody, or a combination of these. In a specific embodiment, the anti-IL6R antibody can be a humanized anti-IL6R monoclonal antibody.

In certain embodiments, the postoperative treatment regimen provided herein comprises administering anti-IL6R antibody or tocilizumab to a transplant recipient. In certain embodiments, anti-IL6R antibody or tocilizumab can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 7 days after and 14 days after, on the day of and 1 day after, on the day of and 1 day after and 2 days after, on the day of and 1 day after and 2 days after and 3 days after, on the day of and 1 day after and 2 days after and 3 days after and 4 days after the transplant surgery, 1 days after and 2 days after and 3 days after, 2 days after and 3 days after and 4 days after, 3 days after and 4 days after and 5 days after, 4 days after and 5 days after and 6 days after, 5 days after and 6 days after and 7 days after, 6 days after and 7 days after and 8 days after, 7 days after and 8 days after and 9 days after, 8 days after and 9 days after and 10 days after, 9 days after and 10 days after and 11 days after, 10 days after and 11 days after and 12 days after, 12 days after and 13 days after and 14 days after, 13 days after and 14 days after and 15 days after, 8 days after and 9 days after and 10 days after, 8 days after and 9 days after and 10 days after, 16 days after and 17 days after and 18 days after, 17 days after and 18 days after and 19 days after, or 18 days after and 19 days after and 20 days after the day of the transplant surgery. In some embodiments, anti-IL6R antibody or tocilizumab is administered to the subject after the transplant. In some embodiments, anti-IL6R antibody or tocilizumab is administered to the subject after about or after at least about 1 day, 2 days, 3 days, 4 days, 5 days, 6 days, 7 days, 8 days, 9 days, 10 days, 11 days, 12, days, 13 days, 14 days, 21 days, 30 days, 1 month, 2 months, 3 months, 4 months, 5 months, 6 months, 7 months, 8 months, 9 months, 10 months, 11 months, 12 months, 18 months, 20 months, 24 months, or more than 24 months after the transplant. In some embodiments, anti-IL6R antibody or tocilizumab is administered to the subject after about or after at least about 1-2 months, 2-3 months, 3-4 months, 4-5 months, 5-6 months, 6-7 months, 7-8 months, 8-9 months, 9-10 months, 11-12 months, 1-6 months, 2-6 months, 3-6 months, 4-6 months, 6-12 months, or more than 6-12 months after the transplant.

In a certain embodiment, the method further comprises the administration of tocilizumab to the patient post-transplant surgery. In a certain specific embodiment, tocilizumab can be administered if the patient presents with early post-transplant chimeric transition syndrome (CTS). In a certain specific embodiment, tocilizumab can be administered to a subject if CTS occurs. In a certain specific embodiment, tocilizumab is administered to a subject at a dose of about 8 mg/kg.

In certain embodiments, tocilizumab can be administered to patients at a dose amount of about, at least about, or at most about: 2.0 mg/kg/dose, 3.0 mg/kg/dose, 4.0 mg/kg/dose, 5.0 mg/kg/dose, 6.0 mg/kg/dose, 6.25 mg/kg/dose, 6.5 mg/kg/dose, 6.75 mg/kg/dose, 7.0 mg/kg/dose, 7.25 mg/kg/dose, 7.5 mg/kg/dose, 7.75 mg/kg/dose, 8.0 mg/kg/dose, 8.25 mg/kg/dose, 8.5 mg/kg/dose, 8.75 mg/kg/dose, 9.0 mg/kg/dose, 9.25 mg/kg/dose, 9.5 mg/kg/dose, 9.75 mg/kg/dose, 10.0 mg/kg/dose, 10.25 mg/kg/dose, 10.5 mg/kg/dose, 10.75 mg/kg/dose, 11.0 mg/kg/dose, 11.25 mg/kg/dose, 11.5 mg/kg/dose, 11.75 mg/kg/dose, 12.0 mg/kg/dose, 12.25 mg/kg/dose, 12.5 mg/kg/dose, 12.75 mg/kg/dose, 13.0 mg/kg/dose, 14.0 mg/kg/dose, 15.0 mg/kg/dose, 16.0 mg/kg/dose, 17.0 mg/kg/dose, 18.0 mg/kg/dose, 6.0-10.0 mg/kg/dose, 6.25-10.25 mg/kg/dose, 6.5-10.5 mg/kg/dose, 6.75-10.75 mg/kg/dose, 7.0-11.0 mg/kg/dose, 7.25-11.25 mg/kg/dose, 7.5-11.5 mg/kg/dose, 7.75-11.75 mg/kg/dose, 8.0-12.0 mg/kg/dose, 8.25-12.25 mg/kg/dose, 8.5-12.5 mg/kg/dose, 8.75-12.75 mg/kg/dose, or 9.0-13.0 mg/kg/dose. In a specific embodiment, tocilizumab can be administered to patients at a dose amount of 8.0 mg/kg/dose. In a specific embodiment, tocilizumab can be administered to patients at a dose amount of 12.0 mg/kg/dose. In a specific embodiment, if a patient weighs over 30 mg/kg, the tocilizumab will be administered at a concentration of 8.0 mg/kg. In a specific embodiment, if a patient weighs less than 30 mg/kg, the tocilizumab can be administered at a concentration of 12.0 mg/kg. In a specific embodiment, the administration of tocilizumab will occur over the span of 1 hour, up to a total of 4 doses (if insufficient response was on the first dose), at least 8 hours apart between the doses.

In certain embodiments, tocilizumab can be administered if the patient presents with early post-transplant acute kidney injury (AKI). In specific embodiments, creatinine levels can be used to determine AKI. In certain embodiments, tocilizumab can be administered alone. In a specific embodiment, tocilizumab can be administered in combination with corticosteroids. If insufficient clinical improvement in the signs and symptoms of AKI occurs after the first dose, additional doses of tocilizumab may be administered. In some embodiments, patients can be administered one additional dose of tocilizumab, two additional doses of tocilizumab, or three additional doses of tocilizumab. If additional doses of tocilizumab are administered, in a specific embodiment, at least 8 hours must elapse in between consecutive doses.

In some embodiments, patients can be administered one dose of tocilizumab daily. In some embodiments, patients can be administered multiple doses of tocilizumab daily. In some embodiments, patients can be administered one dose of tocilizumab. In some embodiments, patients can be administered 2 doses of tocilizumab. In some embodiments, patients can be administered 3 doses of tocilizumab. In some embodiments, patients can be administered 4 doses tocilizumab. In a specific embodiment, one dose of tocilizumab can be administered, followed by additional doses until clinical signs and symptoms of AKI improve.

In a specific embodiment, tocilizumab can be administered intravenously or subcutaneously.

In certain embodiments, an alternate antagonist of IL6 signaling can be administered. In certain embodiments, these alternate antagonists of IL6 signaling can include, but are not limited to, soluble gp130, CS-IVa-Be, Bazedoxifene, and LMT-28. In a certain embodiment, an alternate antagonist of IL6 signaling can be administered to the patient post-transplant surgery. In a certain specific embodiment, an alternate antagonist of IL6 signaling can be administered if the patient presents with early post-transplant acute kidney injury (AKI).

In certain embodiments, the anti-IL-6 treatment can be an anti-IL6R such as, but not limited to ACTEMRA®, RoActemra®, sarilumab, and ALX-0061. In certain embodiments, the anti-IL-6 treatment can be an anti-IL6 mAb such as, but not limited to, siltuximab, sirukumab, clazakizumab, MEDI5117, and olokizumab.

### 5.3.10 Steroids

A steroid, as used herein, is a compound administered to the recipient of an organ transplant to suppress the immune system. Prednisone is a corticosteroid, chemical name 17,21-dihydroxypregna-1,4-dienne-3,11,20-trione (C21H2605). Brand names of prednisone include, but is not limited to, Deltasone®, Sterapred®, Rayos®, Prednicot®, and Meticorten®. In some embodiments, a steroid is a corticosteroid.

In certain embodiments, the postoperative treatment regimen provided herein comprises administering steroids to a transplant recipient. In certain embodiments, steroids can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, or 30 days after the transplant surgery. In certain embodiments, steroids can be administered for about 10 days, 15 days, 20 days, 30 days, 1 month, 2 months, 3 months, 4 months, 5 months, 6 months, 7 months, 8 months, 9 months, 10 months, 11 months, 12 months, or more than 12 months after transplant. In some embodiments, a steroid is administered to the subject after the transplant. In some embodiments, a steroid is administered to the subject after about or after at least about 1 day, 2 days, 3 days, 4 days, 5 days, 6 days, 7 days, 8 days, 9 days, 10 days, 11 days, 12, days, 13 days, 14 days, 21 days, 30 days, 1 month, 2 months, 3 months, 4 months, 5 months, 6 months, 7 months, 8 months, 9 months, 10 months, 11 months, 12 months, 18 months, 20 months, 24 months, or more than 24 months after the transplant. In some embodiments a steroid is administered to the subject after about or after at least about 1-2 months, 2-3 months, 3-4 months, 4-5 months, 5-6 months, 6-7 months, 7-8 months, 8-9 months, 9-10 months, 11-12 months, 1-6 months, 2-6 months, 3-6 months, 4-6 months, 6-12 months, or more than 6-12 months after the transplant.

In certain embodiments, a steroid can be administered on the day of the transplant surgery through 5 days after, on the day of the transplant surgery through 10 days after, on the day of the transplant surgery through 15 days after, on the day of the transplant surgery through 20 days after, on the day of the transplant surgery through 25 days after, or on the day of the transplant surgery through 30 days after, on the day of the transplant surgery through 6 months after the transplant surgery.

In certain embodiments, a single dose of the steroid can be administered. In certain embodiments, multiple doses of the steroid can be administered. In certain embodiments, a constant dose of steroids can be administered. In certain embodiments, a pulse of steroids can be administered. In certain embodiments, a tapering course of steroids can be administered. In certain embodiments, a constant dose of steroids followed by a tapering course of steroids can be administered. In certain embodiments, a constant dose of steroids, with pulses of steroids, and a tapering course of steroids can be administered.

In certain embodiments, the steroid can be administered to a transplant recipient at a dose amount of about, at least about, or at most about: 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, or 3.0 mg/kg. In a specific embodiment, the steroid can be administered to a transplant recipient at a dose amount of 2 mg/kg.

In certain embodiments, the steroid can be administered to a transplant recipient at a pulsed dose amount of 100 mg/dose, 200 mg/dose, 250 mg/dose, 300 mg/dose, 400 mg/dose, 500 mg/dose, 600 mg/dose, 700 mg/dose, 800 mg/dose, 900 mg/dose, or 1000 mg/dose. In certain embodiments, the steroid can be administered to a transplant recipient at a dose amount of about 250 mg/dose. In a specific embodiment, the steroid can be administered at pulsed dose amount of 500 mg/dose. In certain embodiments, a pulse of the steroid can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, the day of the transplant and for about 20 days, the day of the transplant and for about 6 months, the day of and 1 day and 2 days after, 3 days and 4 days and 5 days after, 6 days and 7 days and 8 days after, 9 days and 10 days and 11 days after, 10 days and 11 days and 12 days after, 11 days and 12 days and 13 days after, or 13 days and 14 days and 15 days after the transplant. In a specific embodiment, a pulse of the steroid is administered the day of the transplant. In a specific embodiment, a pulse of steroid is administered 10 days and 11 days and 12 days after the transplant.

In certain embodiments, the steroid can be administered to a transplant recipient at a constant dose. The duration of time a constant dose is administered can be 1 day, 2 days, 3 days, 4 days, 5 days, 6 days, 7 days, 8 days, 9 days, 10 days, 15 days, 20 days, 30 days, 1 month, 2 months, 3 months, 4 months, 5 months, 6 months, 7 months, 8 months, 9 months, or longer than 9 months. In certain embodiments, the steroids administered to a transplant recipient can be tapered to discontinuation. In certain embodiments, this tapering course can take place over 5 days, 6 days, 7 days, 8 days, 9 days, 10 days, 11 days, 12 days, 13 days, 14 days 15 days, 16 days, 17 days, 18 days, 19 days, 20 days, 21 days, 22 days, 23 days, 24 days 25 days, 26 days, 27 days, 28 days, 29 days, 30 days, 1 month, 2 months, 3 months, 4 months, 5 months, 6 months, 7 months, 8 months, 9 months, or longer than 9 months. In a specific embodiment, the tapering course can take place over 10 days. In a specific embodiment, the tapering course can take place over 20 days. In a specific embodiment, the tapering course can take place over 6 months.

In certain embodiments, for the tapering course, the steroids administered to the recipient can be reduced by about, at least about, or at most about: 0.01 mg/kg per day, 0.02 mg/kg per day, 0.03 mg/kg per day, 0.04 mg/kg per day, 0.05 mg/kg per day, 0.06 mg/kg per day, 0.07 mg/kg per day, 0.08 mg/kg per day, 0.09 mg/kg per day, 0.1 mg/kg per day, 0.2 mg/kg per day, 0.3 mg/kg per day, 0.4 mg/kg per day, 0.5 mg/kg per day, 0.6 mg/kg per day, 0.7 mg/kg per day, 0.8 mg/kg per day, 0.9 mg/kg per day, 1.0 mg/kg per day, 1.1 mg/kg per day, 1.2 mg/kg per day, 1.3 mg/kg per day, 1.4 mg/kg per day, 1.5 mg/kg per day, 1.6 mg/kg per day, 1.7 mg/kg per day, 1.8 mg/kg per day, 1.9 mg/kg per day, 2.0 mg/kg per day, or more than 2.0 mg/kg per day.

In certain embodiments, the steroid can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritonially, or through a route of administration which allows for the proper action of the steroid by the recipient. In a specific embodiment, the steroid can be administered orally. In a specific embodiment, the steroid can be administered intravenously.

In a specific embodiment, the steroid administered in the postoperative treatment regimen can be prednisone. In a specific embodiment, the pulsed steroid administered in the postoperative treatment regimen can be methylprednisone. In certain embodiments, the administration of steroids can be modified as described herein to achieve and/or maintained mixed chimerism in the recipient.

### 5.3.11 Cyclosporine

Cyclosporine A (CyA), as used herein, is a compound administered to a recipient to suppress the immune system, with a specific action on T cells. CyA (C62HinN1012) can be administered to a transplant recipient to inhibit the development of Graft versus Host disease. Brand names of CyA include Gengraf®, Neoral®, and Sandimmune®.

In certain embodiments, the conditioning regimen provided herein comprises administering CyA to a transplant recipient. In certain embodiments, CyA can be administered 1 day before the transplant, 2 days before the transplant, 3 days before the transplant, 1 day and 2 days before the transplant, 1 day and 3 days before transplant, or 1 day and 2 days and 3 days before the transplant. In a specific embodiment, CyA can be administered to a recipient 1 day before the transplant.

Cyclosporine (CyA) can be included in the postoperative treatment regimen to suppress the immune system and inhibit the development of Graft versus Host disease in the recipient. The postoperative treatment regimen can include a constant course followed by a tapering course of CyA administration to the recipient.

In certain embodiments, the postoperative treatment regimen provided herein comprises administering CyA to a transplant recipient. In certain embodiments, CyA can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, 30 days after, 1 month after, 2 months after, 3 months after, 4 months after, 5 months after, 6 months after, 7 months after, 8 months after, 9 months after, 10 months after, 11 months after, 12 months after, 13 months after, 14 months after, 15 months after, 16 months after, 17 months after, or 18 months after the transplant surgery. In some embodiments, CyA is administered to the subject after the transplant. In some embodiments, CyA is administered to the subject after about or after at least about 1 day, 2 days, 3 days, 4 days, 5 days, 6 days, 7 days, 8 days, 9 days, 10 days, 11 days, 12, days, 13 days, 14 days, 21 days, 30 days, 1 month, 2 months, 3 months, 4 months, 5 months, 6 months, 7 months, 8 months, 9 months, 10 months, 11 months, 12 months, 18 months, 20 months, 24 months, or more than 24 months after the transplant. In some embodiments CyA is administered to the subject after about or after at least about 1-2 months, 2-3 months, 3-4 months, 4-5 months, 5-6 months, 6-7 months, 7-8 months, 8-9 months, 9-10 months, 11-12 months, 1-6 months, 2-6 months, 3-6 months, 4-6 months, 6-12 months, or more than 6-12 months after the transplant.

In certain embodiments, a single dose amount of CyA can be administered. In certain embodiments, multiple dose amounts of the CyA can be administered. In certain embodiments, a constant dose of CyA can be administered. In certain embodiments, a tapering course of CyA can be administered. In certain embodiments, a constant dose of CyA followed by a tapering course of CyA can be administered postoperatively.

In certain embodiments, CyA can be administered to a transplant recipient at a dose amount of 2 mg/kg/day, 2.5 mg/kg/day, 3 mg/kg/day, 3.5 mg/kg/day, 4 mg/kg/day, 4.5 mg/kg/day, 5 mg/kg/day, 5.5 mg/kg/day, 6 mg/kg/day, 6.5 mg/kg/day, 7 mg/kg/day, 7.5 mg/kg/day, 8 mg/kg/day, 8.5 mg/kg/day, 9 mg/kg/day, 9.5 mg/kg/day, 10 mg/kg/day, 10.5 mg/kg/day, 11 mg/kg/day, 11.5 mg/kg/day, 12 mg/kg/day, 12.5 mg/kg/day, 13 mg/kg/day, 13.5 mg/kg/day, 14 mg/kg/day, 14.5 mg/kg/day, 15 mg/kg/day, 15.5 mg/kg/day, 16 mg/kg/day, 16.5 mg/kg/day, 17 mg/kg/day, 17.5 mg/kg/day, 18 mg/kg/day, 2-6 mg/kg/day, 3-7 mg/kg/day, 4-8 mg/kg/day, 5-9 mg/kg/day, 6-10 mg/kg/day, 7-11 mg/kg/day, 8-12 mg/kg/day, 9-13 mg/kg/day, 10-14 mg/kg/day, 11-15 mg/kg/day, 12-16 mg/kg/day, 13-17 mg/kg/day, or 14-18 mg/kg/day. In a specific embodiment, CyA can be administered to a transplant recipient at a dose amount of 8 mg/kg/day. In a specific embodiment, CyA can be administered to a transplant recipient at a dose amount of 9 mg/kg/day. In a specific embodiment, CyA can be administered to a transplant recipient at a dose amount of 10 mg/kg/day. In a specific embodiment, CyA can be administered to a transplant recipient at a dose amount of 11 mg/kg/day. In a specific embodiment, CyA can be administered to a transplant recipient at a dose amount of 12 mg/kg/day. In a specific embodiment, CyA can be administered to a transplant recipient at a dose range of 8-12 mg/kg/day.

In certain embodiments, CyA can be administered postoperatively to a recipient at a sufficient dose amount to obtain the target trough blood levels of 100-200 ng/ml, 125-225 ng/ml, 150-250 ng/ml, 175-275 ng/ml, 200-300 ng/ml, 225-325 ng/ml, 250-350 ng/ml, 275-375 ng/ml, 300-400 ng/ml, 325-425 ng/ml, 350-450 ng/ml, 375-475 ng/ml, or 400-500 ng/ml. In a specific embodiment, the target trough blood levels can be 250-350 ng/ml.

In certain embodiments, CyA can be administered to a transplant recipient at a constant dose. The duration of time a constant dose is administered can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the transplant. In certain embodiments, CyA is administered to a transplant recipient can be tapered to discontinuation. In certain embodiments, this tapering course can take place over 1 month, 2 months, 3 months, 4 months, 5 months, 6 months, 7 months, 8 months, 9 months, 10 months, 11 months, 12 months, 13 months, 14 months, 15 months, 16 months, 17 months, or 18 months after the transplant.

In certain embodiments, CyA can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritonially, or through a route of administration which allows for the proper action of the CyA by the recipient. In a specific embodiment, CyA can be administered orally. In a specific embodiment, the CyA can be administered intravenously. In certain embodiments, the administration of CyA can be modified as described herein to achieve and/or maintained mixed chimerism in the recipient.

In certain embodiments, substitute compounds can be used in the place of CyA. These compounds can include tacrolimus (Prograf®, Adport®, Advagraf®, Envarsus®, Modigraf®, Astagraf®), Belatacept (Nulojix®), sirolimus, and everolimus. In specific embodiments, Belatacept can be used to suppress the immune system in the recipient.

In certain embodiments, the postoperative treatment regimen provided herein comprises administering Belatacept to a transplant recipient. In certain embodiments, Belatacept can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, 30 days after, 31 days after, 32 days after, 33 days after, 34 days after, 35 days after, 36 days after, 37 days after, 38 days after, 39 days after, or 40 days after the transplant surgery. In certain embodiments, Belatacept can be administered a single time. In certain embodiments, Belatacept can be administered multiple times. In a specific embodiment, Belatacept can be administered 5 times after the transplant surgery. In certain embodiments, Belatacept can be administered 1 day after the transplant surgery. In certain embodiments, Belatacept can be administered 5 days after the transplant surgery. In certain embodiments, Belatacept can be administered 12 days after the transplant surgery. In certain embodiments, Belatacept can be administered 19 days after the transplant surgery. In certain embodiments, Belatacept can be administered 33 days after the transplant surgery. In a specific embodiment, Belatacept can be administered 1 day and 5 days and 12 days and 19 days and 33 days after the transplant surgery.

In certain embodiments, Belatacept can be administered to a transplant recipient at a dose amount of 2 mg/kg/day, 2.5 mg/kg/day, 3 mg/kg/day, 3.5 mg/kg/day, 4 mg/kg/day, 4.5 mg/kg/day, 5 mg/kg/day, 5.5 mg/kg/day, 6 mg/kg/day, 6.5 mg/kg/day, 7 mg/kg/day, 7.5 mg/kg/day, 8 mg/kg/day, 8.5 mg/kg/day, 9 mg/kg/day, 9.5 mg/kg/day, 10 mg/kg/day, 10.5 mg/kg/day, 11 mg/kg/day, 11.5 mg/kg/day, 12 mg/kg/day, 12.5 mg/kg/day, 13 mg/kg/day, 13.5 mg/kg/day, 14 mg/kg/day, 14.5 mg/kg/day, 15 mg/kg/day, 15.5 mg/kg/day, 16 mg/kg/day, 16.5 mg/kg/day, 17 mg/kg/day, 17.5 mg/kg/day, 18 mg/kg/day, 2-6 mg/kg/day, 3-7 mg/kg/day, 4-8 mg/kg/day, 5-9 mg/kg/day, 6-10 mg/kg/day, 7-11 mg/kg/day, 8-12 mg/kg/day, 9-13 mg/kg/day, 10-14 mg/kg/day, 11-15 mg/kg/day, 12-16 mg/kg/day, 13-17 mg/kg/day, or 14-18 mg/kg/day. In a specific embodiment, Belatacept can be administered to a transplant recipient at a dose amount of 10 mg/kg/day. In certain embodiments, the administration of Belatacept can be modified as described herein to achieve and/or maintained mixed chimerism in the recipient.

### 5.3.12 Mycophenolate Mofetil

Mycophenolate mofetil (MMF), as used herein, can be included in the postoperative treatment regimen to suppress the immune system and inhibit the development of Graft versus Host disease in the recipient. Brand names of MMF can include CellCept® and Myfortic®. The postoperative treatment regimen can include a constant course followed by a tapering course of MMF administration to the recipient.

In certain embodiments, the postoperative treatment regimen provided herein comprises administering MMF to a transplant recipient. In certain embodiments, MMF can be administered on the day of the transplant, on the day of the transplant and for about 2 months, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, 30 days after, 1 month after, 2 months after, 3 months after, 4 months after, 5 months after, 6 months after, 7 months after, 8 months after, 9 months after, 10 months after, 11 months after, 12 months after, 13 months after, 14 months after, 15 months after, 16 months after, 17 months after, or 18 months after the transplant surgery. In some embodiments, MMF is administered to the subject after the transplant. In some embodiments, MMF is administered to the subject after about or after at least about 1 day, 2 days, 3 days, 4 days, 5 days, 6 days, 7 days, 8 days, 9 days, 10 days, 11 days, 12, days, 13 days, 14 days, 21 days, 30 days, 1 month, 2 months, 3 months, 4 months, 5 months, 6 months, 7 months, 8 months, 9 months, 10 months, 11 months, 12 months, 18 months, 20 months, 24 months, or more than 24 months after the transplant. In some embodiments MMF is administered to the subject after about or after at least about 1-2 months, 2-3 months, 3-4 months, 4-5 months, 5-6 months, 6-7 months, 7-8 months, 8-9 months, 9-10 months, 11-12 months, 1-6 months, 2-6 months, 3-6 months, 4-6 months, 6-12 months, or more than 6-12 months after the transplant.

In certain embodiments, a single dose amount of MMF can be administered. In certain embodiments, multiple dose amounts of MMF can be administered. In certain embodiments, a constant dose of MMF can be administered. In certain embodiments, a tapering course of MMF can be administered. In certain embodiments, a constant dose of MMF followed by a tapering course of MMF can be administered postoperatively.

In certain embodiments, MMF can be administered to a transplant recipient at a dose amount of about, at least about, or at most about: 100 mg/dose, 200 mg/dose, 300 mg/dose, 400 mg/dose, 500 mg/dose, 600 mg/dose, 700 mg/dose, 800 mg/dose, 900 mg/dose, 1000 mg/dose, 1100 mg/dose, 1200 mg/dose, 1300 mg/dose, 1400 mg/dose, 1500 mg/day, 1600 mg/dose, 1700 mg/dose, 1800 mg/dose, 1900 mg/dose, 2000 mg/dose, 100-1000 mg/dose, 200-1200 mg/dose, 300-1300 mg/dose, 400-1400 mg/dose, 500-1500 mg/dose, 600-1600 mg/dose, 700-1700 mg/dose, 800-1800 mg/dose, 900-1900 mg/dose, or 1000-2000 mg/dose. In a specific embodiment, MMF can be administered to a transplant recipient at a dose of about 2 g/day. In a specific embodiment, MMF can be administered to a transplant recipient at a dose range 500-1500 mg/dose. In a specific embodiment, MMF is administered to a transplant recipient at a dose range 500-1500 mg/day once per day. In a specific embodiment, MMF is administered to a transplant recipient at a dose range 500-1500 mg/day twice per day.

In certain embodiments, MMF can be administered to a transplant recipient at a constant dose. The duration of time a constant dose is administered can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 4 months, 5 months, 6 months, 7 months, 8 months, 9 months, 10 months, or more than 10 months after the transplant. In certain embodiments, MMF is administered to a transplant recipient can be tapered to discontinuation. In certain embodiments, this tapering course can take place over 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, or more than 10 months after the transplant.

In certain embodiments, MMF can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritonially, or through a route of administration which allows for the proper action of the MMF by the recipient. In a specific embodiment, MMF can be administered orally. In a specific embodiment, the MMF can be administered intravenously. In certain embodiments, the administration of MMF can be modified as described herein to achieve and/or maintained mixed chimerism in the recipient.

5.3.13 mTOR Inhibitor

In certain embodiments, the methods described herein include administering an mTOR inhibitor to the recipient. Without being bound by theory, the mTOR inhibitor can be administered to the recipient to inhibit T-cell and B-cell activation to prevent transplant rejection. In certain embodiments, an mTOR inhibitor can be used in combination with tacrolimus or can be used instead of tacrolimus. In specific embodiments, the mTOR inhibitor described in the methods presented herein can be rapamycin (e.g., sirolimus) or everolimus.

In some embodiments, mTOR inhibitor is administered to the subject after the transplant. In some embodiments, mTOR inhibitor is administered to the subject after about or after at least about 1 day, 2 days, 3 days, 4 days, 5 days, 6 days, 7 days, 8 days, 9 days, 10 days, 11 days, 12, days, 13 days, 14 days, 21 days, 30 days, 1 month, 2 months, 3 months, 4 months, 5 months, 6 months, 7 months, 8 months, 9 months, 10 months, 11 months, 12 months, 18 months, 20 months, 24 months, or more than 24 months after the transplant. In some embodiments mTOR inhibitor is administered to the subject after about or after at least about 1-2 months, 2-3 months, 3-4 months, 4-5 months, 5-6 months, 6-7 months, 7-8 months, 8-9 months, 9-10 months, 11-12 months, 1-6 months, 2-6 months, 3-6 months, 4-6 months, 6-12 months, or more than 6-12 months after the transplant.

In certain embodiments, the postoperative regimen provided herein comprises administering rapamycin (or mTOR inhibitor) to a transplant recipient after the transplant. In certain embodiments, rapamycin or mTOR inhibitor is administered daily. In certain embodiments, administration of rapamycin or mTOR inhibitor can be initiated immediately after the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, 30 days after, or more than 30 days after the transplant surgery. In certain embodiments, administration of rapamycin or mTOR inhibitor starts about 1 month after transplant to about 12 months after transplant. In certain embodiments, rapamycin is administered for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, or at least 12 months. In certain embodiments, rapamycin is administered in 1, 2, 3, 4, or 5 daily doses.

In certain embodiments, the first dose of rapamycin or mTOR inhibitor is a loading dose and can be higher than the subsequent daily doses. In certain embodiments, a dose of rapamycin or mTOR inhibitor is about 5-8 ng/ml. In certain embodiments, the first dose can be 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 1-4 mg/kg, 2-6 mg/kg, 4-8 mg/kg, 6-10 mg/kg, or 8-12 mg/kg. In certain embodiments, the daily dose can be 0.5 mg/kg, 1 mg/kg, 1.5 mg/kg, 2 mg/kg, 2.5 mg/kg, 3 mg/kg, 3.5 mg/kg, 4 mg/kg, 1-3 mg/kg, or 1.5-2.5 mg/kg. In a specific embodiment, the rapamycin is administered at an initial dose of 6 mg/kg and followed by a daily dose of 2 mg/kg. In a specific embodiment, rapamycin (or mTOR inhibitor) is administered orally.

In certain embodiments, rapamycin or mTOR inhibitor can be administered postoperatively to a recipient at a sufficient dose amount to obtain the target whole blood levels of 1-5 ng/ml, 2-10 ng/ml, 5-8 ng/ml, 4-12 ng/ml, 6-14 ng/ml, 8-16 ng/ml, 10-18 ng/ml, 12-20 ng/ml, 14-22 ng/ml, or 16-24 ng/ml. In a specific embodiment, the target whole blood levels can be 4-12 ng/ml.

In some embodiments, mTOR inhibitor is administered to a subject at a dose of about, at least about, or at most about 1-5 ng/ml, 5-10 ng/ml, 8-10 ng/ml, 4-11 ng/ml, 10-15 ng/ml, 1-11 ng/ml, 2-12 ng/ml, 3-13 ng/ml, 4-14 ng/ml, 5-15 ng/ml, 6-16 ng/ml, 7-17 ng/ml, 8-18 ng/ml, 9-19 ng/ml, 10-20 ng/ml, or 15-20 ng/ml. In some embodiments, tacrolimus is administered to a subject at a dose of about, at least about, or at most about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, 22 ng/ml, 25 ng/ml, 30 ng/ml, or more than 30 ng/ml.

In certain embodiments, rapamycin or mTOR inhibitor can be administered to a transplant recipient in a convenient manner known in the art including subcutaneously, intravenously, intravascularly, topically, intra-arterially, intra-cranially, intramuscularly, orally, intra-orbitally, by inhalation, transdermally, intra-peritonially, or through a route of administration which allows for the appropriate action of rapamycin or mTOR inhibitor to occur in the recipient. In a specific embodiment, the rapamycin or mTOR inhibitor is administered orally. In certain embodiments, the administration of rapamycin or mTOR inhibitor can be modified as described herein to achieve and/or maintained mixed chimerism in the recipient.

In certain embodiments, the mTOR inhibitor can be everolimus. In certain embodiments, everolimus or mTOR inhibitor is administered twice daily. In certain embodiments, administration of everolimus or mTOR inhibitor can be initiated immediately after the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, or 30 days after the transplant surgery. In certain embodiments, everolimus or mTOR inhibitor is administered for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, or at least 12 months.

In certain embodiments, the dose range of everolimus or mTOR inhibitor administered can be 0.25 mg/kg/dose, 0.5 mg/kg/dose, 0.75 mg/kg/dose, 1.0 mg/kg/dose, 1.25 mg/kg/dose, 1.5 mg/kg/dose, 1.75 mg/kg/dose, 2.0 mg/kg/dose, 0.25-0.5 mg/kg/dose, 0.5-0.75 mg/kg/dose, 0.75-1.0 mg/kg/dose, 1.0-1.25 mg/kg/dose, 1.25-1.50 mg/kg/dose, 1.50-1.75 mg/kg/dose, or 1.75-2.0 mg/kg/dose. In a specific embodiment, the everolimus or mTOR inhibitor is administered twice daily at a dose range of 0.75-1.0 mg/kg/dose. In a specific embodiment, everolimus or mTOR inhibitor is administered orally.

In certain embodiments, everolimus or mTOR inhibitor can be administered postoperatively to a recipient at a sufficient dose amount to obtain the target whole blood levels of 0.1-5 ng/ml, 1-6 ng/ml, 2-7 ng/ml, 3-8 ng/ml, 4-9 ng/ml, 5-10 ng/ml, 6-11 ng/ml, 7-12 ng/ml, or 8-13 ng/ml. In a specific embodiment, the target whole blood levels can be 3-8 ng/ml. In certain embodiments, the administration of everolimus or mTOR inhibitor can be modified as described herein to achieve and/or maintained mixed chimerism in the recipient.

In certain embodiments, the mTOR inhibitor can be temsirolimus, everolimus, sirolimus (rapamycin), ridaforolimus, non-rapamycin analog mTOR inhibiting compounds including, but not limited to, LY294002, wortmannin, quercetin, myricentin, staurosporine, or ATP competitive inhibitors.

### 5.4 Transplantation

The procedure for obtaining and implanting the organ or tissue is well-known to the skilled artisan. Any procedure for the surgical removal from the donor and the surgical implantation in the recipient can be used with the methods provided herein. In certain embodiments, the organ or tissue can be treated between removal and implantation. In a specific embodiment, the organ or tissue is treated between removal and implantation as described in Section 5.3.1.

### 5.5 Bone Marrow Infusion

The procedures for obtaining and infusing bone marrow are well-known to the skilled artisan. In certain embodiments, the donor bone marrow is unprocessed. In certain embodiments, at least 1×, 2×, 3×, 4×, 5×, 6×, 7×, 8×, or at least 9×10⁷ cells/kg are infused. In certain embodiments, at least 1×, 2×, 3×, 4×, 5×, 6×, 7×, 8×, or at least 9×10⁸ cells/kg are infused. In certain more specific embodiments, 2-3×10⁸ cells/kg are infused. In certain embodiments, the procedure and amount of the bone marrow infusion can be modified as described herein to achieve and/or prolonged mixed chimerism in the recipient.

### 5.6 Outcome Assessment

In certain embodiments, the efficacy of the conditioning regimen to achieve chimerism can be assessed. Flow cytometric phenotyping can be used to determine achievement of mixed chimerism (percentage of donor cells in lymphohematopoetic system) and the duration of mixed chimerism. Chimerism can be assessed using of class I, HLA-specific monoclonal antibodies to detect donor and recipient leukocytes and also using polymerase-chain-reaction (PCR) assays of variable short tandem repeats.

In certain embodiments, without being bound by theory, the combination of an anti-CD2 antibody or antigen binding fragment thereof, tocilizumab (e.g., if CTS occurs or administered in specific days), cyclophosphamide (such as 60 mg/kg), rituximab, and/or thymus irradiation minimizes the risk of, duration of, or severity of CTS. In certain embodiments, without being bound by theory, the combination of an anti-CD2 antibody or antigen binding fragment thereof, cyclophosphamide (such as 22.5 mg/kg), rituximab, fludarabine (e.g., 10 mg/m²), and/or thymus irradiation minimizes the risk of, duration of, or severity of CTS. In certain embodiments, without being bound by theory, the combination of low dose cyclophosphamide (such as 22 mg/kg) and fludarabine (such as 10 mg/m²) minimizes the risk of, duration of, or severity of CTS. In certain embodiments, the duration or severity of CTS can be measured as an assessment of clinical outcome. In certain embodiments, any of the assays or experiments described herein (e.g., in the Examples Section 6)

In certain embodiments, functional assays can be used to detect biomarkers which are predictive of transplant rejection including the presence of circulating donor-specific antibodies (DSA) and levels of B-cell activating factor (BAFF) in the serum of the recipient which can be determined by ELISA. In certain embodiments, flow cytometric analyses on circulating lymphocytes can be performed to determine status of immune system reconstitution of the recipient. In certain embodiments, mixed lymphocyte reaction (MLR) of the recipient's peripheral blood mononuclear cells can be performed to assess the response of the recipient's cells to the donor cells and if the response changes after transplant surgery. In certain embodiments, functional assays can be used to determine if induction of tolerance to the transplanted organ was achieved. These assays can include flow cytometric analysis to determine FoxP3+ T cells: CD4+ T cells ratio as an indicator for the presence of regulatory T-cells.

In certain embodiments, a biopsy of the transplanted graft can be performed to examine the health of the transplanted organ and determine induction of tolerance or evidence of graft rejection. Such evidence visualized by biopsy can include proteinuria, infiltration mononuclear cells, red cell fragments, arterial fibrinoid necrosis, or deposition of C4d. Tissue biopsies can be examined using routine light microscopy, immunofluorescence, and electron microscopy therapy (Panel B). In certain embodiments, total RNA can be isolated from the biopsy and mRNA levels for markers of interest can be determined. These markers of interest can include transcription factor FOXP3 and granzyme B. Biopsy of the transplant can occur 6 months after, 12 months after, 2 years after, 3 years after, 4 years after, 5 years after, 6 years after, 7 years after, 8 years after, 9 years after, or years after the transplant surgery.

In certain embodiments, assessments of the outcome of the transplant surgery can include the monitoring of the function of the transplanted organ in the recipient. For example, if a kidney is the transplanted organ, the glomerular filtration rate (GFR) of the kidney can be monitored as an assessment of the outcome of the transplant surgery. In certain embodiments, other assessments of the outcome of the transplant surgery can include the monitoring of the incidence of infection, the incidence of opportunistic infection, the onset of any treatment-related adverse events, and the patient's post-transplant quality of life.

In certain embodiments, the efficacy of a method of treatment described herein can be assessed by determining the survival of the transplanted graft (e.g., liver or kidney). In certain embodiments, a biopsy of the transplanted graft (e.g., liver or kidney) can be performed to examine the health of the transplanted organ (e.g., liver or kidney) and determine induction of tolerance or evidence of graft rejection. Tissue biopsies can be examined using routine light microscopy, immunofluorescence, and electron microscopy.

In certain embodiments, the efficacy of a method of treatment described herein can be determined by detecting treated biopsy-proven acute rejection (tBPAR) in a recipient. In specific embodiments, no tBPAR is detected in a recipient at 10 months, 12 months, 15 months, 18 months, 20 months, 25 months, 30 months, 35 months, 40 months, 45 months, 50 months, 55 months, or 60 months post-transplant.

In certain embodiments, assessments of the outcome of the transplant surgery can include the monitoring of the function of the transplanted organ (e.g., liver or kidney) in the recipient. For example, the efficacy of a method of treatment described herein may be assessed by measuring organ function at 1 month, 2 months, 3 months, 4 months, 5 months, 6 months, 7.5 months, 9 months, 10.5 months, 12 months, 13.5 months, 15 months, 16.5 months, 18 months, 19.5 months, 21 months, 22.5 months, 24 months, 27 months, 30 months, 36 months, 42 months, 48 months, or, 54 months post-transplant, or at 1-5 months, 5-10 months, 10-15 months, 15-20 months, 20-25 months, 25-30 months, 30-35 months, 35-40 months, 40-45 months, 45-50 months, 50-55 months, or 55-60 months post-transplant. Liver function may be determined, for example, by conducting liver function tests, such as measurements of alanine transaminase (ALT), aspartate transaminase (AST), alkaline phosphatase (ALP), gamma-glutamyl transferase (GGT), serum bilirubin, prothrombin time (PT), the international normalized ratio (INR) and/or albumin. In particular embodiments, a method of treatment described herein results in a recipient having normal organ function (as determined by organ function tests) for at least 10 months, at least 15 months, at least 20 months, at least 25 months, at least 30 months, at least 35 months, at least 40 months, at least 45 months, at least 50 months, at least 55 months, or at least 60 months post-transplant. In particular embodiments, a method of treatment described herein results in a recipient having better organ function (as determined by organ function tests) for at least 10 months, at least 15 months, at least 20 months, at least 25 months, at least 30 months, at least 35 months, at least 40 months, at least 45 months, at least 50 months, at least 55 months, or at least 60 months post-transplant as compared to a patient undergoing standard of care post-organ transplant therapy.

In specific embodiments, a method of treatment described herein results in a recipient having ALT values of 5-60 U/L, 7-55 U/L, 10-55 U/L, 15-50 U/L, 20-40 U/L or 25-35 U/L for at least 10 months, at least 15 months, at least 20 months, at least 25 months, at least 30 months, at least 35 months, at least 40 months, at least 45 months, at least 50 months, at least 55 months, or at least 60 months post-transplant.

In specific embodiments, a method of treatment described herein results in a recipient having AST values of 5-50 U/L, 8-48 U/L, 10-45 U/L, 15-40 U/L, or 20-30 U/L for at least 10 months, at least 15 months, at least 20 months, at least 25 months, at least 30 months, at least 35 months, at least 40 months, at least 45 months, at least 50 months, at least 55 months, or at least 60 months post-transplant.

In specific embodiments, a method of treatment described herein results in a recipient having ALP values of 30-150 U/L, 40-129 U/L, 50-120 U/L, 60-110 U/L, 70-100 U/L, or 80-90 U/L for at least 10 months, at least 15 months, at least 20 months, at least 25 months, at least 30 months, at least 35 months, at least 40 months, at least 45 months, at least 50 months, at least 55 months, or at least 60 months post-transplant.

In specific embodiments, a method of treatment described herein results in a recipient having GGT values of 5-70 U/L, 8-61 U/L, 10-50 U/L, 15-45 U/L, 20-40 U/L, or 25-35 U/L for at least 10 months, at least 15 months, at least 20 months, at least 25 months, at least 30 months, at least 35 months, at least 40 months, at least 45 months, at least 50 months, at least 55 months, or at least 60 months post-transplant.

In specific embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 10 months, at least 15 months, at least 20 months, at least 25 months, at least 30 months, at least 35 months, at least 40 months, at least 45 months, at least 50 months, at least 55 months, or at least 60 months post-transplant.

In specific embodiments, a method of treatment described herein results in a recipient having a PT of 7-15 seconds, 8-14 seconds, 9-13 seconds, 9.4-12.5 seconds, or 10-12 seconds for at least 10 months, at least 15 months, at least 20 months, at least 25 months, at least 30 months, at least 35 months, at least 40 months, at least 45 months, at least 50 months, at least 55 months, or at least 60 months post-transplant.

In specific embodiments, a method of treatment described herein results in a recipient having albumin values of 2-6 g/dL, 2.5-5.5 g/dL, 3-5 g/dL, 3.5-5 g/dL, or 4-4.5 g/dL for at least 10 months, at least 15 months, at least 20 months, at least 25 months, at least 30 months, at least 35 months, at least 40 months, at least 45 months, at least 50 months, at least 55 months, or at least 60 months post-transplant.

In specific embodiments, a method of treatment described herein results in a recipient having an INR 1-3, 1.5-2.5, 1.5-2 or below 1.1 for at least 10 months, at least 15 months, at least 20 months, at least 25 months, at least 30 months, at least 35 months, at least 40 months, at least 45 months, at least 50 months, at least 55 months, or at least 60 months post-transplant.

For example, the efficacy of a method of treatment described herein may be assessed by measuring renal function at 10 months, 15 months, 20 months, 25 months, 30 months, 35 months, 40 months, 45 months, 50 months, 55 months, or 60 months post-transplant. Renal function may be determined, for example, by measuring serum creatinine and/or glomerular filtration rate. In particular embodiments, a method described herein can result in a recipient having normal kidney function (as determined by serum creatinine and/or glomerular filtration rate (GFR)). In particular embodiments, a method described herein can result in a recipient having better kidney function (as determined by serum creatinine and/or glomerular filtration rate) as compared to a patient undergoing standard of care post-liver transplant therapy.

In specific embodiments, a method of treatment described herein results in a recipient having serum creatinine values of 0.5-1.5 mg/mL, 0.6-1.4 mg/mL, 0.7-1.3 mg/mL, 0.8-1.2 mg/mL, or 0.9-1.1 mg/mL for at least 10 months, at least 15 months, at least 20 months, at least 25 months, at least 30 months, at least 35 months, at least 40 months, at least 45 months, at least 50 months, at least 55 months, or at least 60 months post-transplant.

In specific embodiments, a method of treatment described herein results in a recipient having a GFR of 40-90, 45-85, 50-80, 55-75, 60-70, or above 90 for at least 10 months, at least 15 months, at least 20 months, at least 25 months, at least 30 months, at least 35 months, at least 40 months, at least 45 months, at least 50 months, at least 55 months, or at least 60 months post-transplant.

### 5.7 Pharmaceutical Compositions

Provided herein are pharmaceutical compositions comprising a pharmaceutically effective amount of an anti-CD2 antibody or antigen-binding fragment thereof as described herein. Also provided herein are pharmaceutical compositions comprising one or more than one of a second agent (e.g., rituximab, cyclophosphamide, tocilizumab, corticosteroid, tacrolimus, MMF, antiproliferative agent, steroid, sirolimus, polyclonal rabbit anti-thymocyte globulin (rATG), fludarabine, B-cell depleting antibody, immunosuppressive agent, chemotherapeutic agent, antiproliferative agent, anti-IL6R antibody, antineoplastic agent, and/or irradiation) as described herein. In some embodiments, a pharmaceutical composition is administered with, prior to, or after thymic (or thymus) irradiation, transplant, and/or cell (e.g., bone marrow) infusion. In some embodiments, a pharmaceutical composition is administered prophylactically (e.g., tocilizumab).

In some embodiments, a pharmaceutical composition is administered to a subject prior to transplant. In some embodiments, a pharmaceutical composition is administered to a subject prior to cell infusion (e.g., bone marrow cell infusion). In some embodiments, a pharmaceutical composition is administered to a subject after a transplant. In some embodiments, a pharmaceutical composition is administered to a subject after cell infusion (e.g., bone marrow cell infusion). In some embodiments, a pharmaceutical composition is administered to a subject on the same day as the day of the transplant. In some embodiments, a pharmaceutical composition is administered to a subject on the same day as the day of cell infusion (e.g., bone marrow cell infusion). In some embodiments, a pharmaceutical composition is administered to a subject 20, 19, 18, 17, 16, 15, 14, 13, 12, 11, 10, 9, 8, 7, 6, 5, 4, 3, 2, and/or 1 day(s) prior to transplant. In some embodiments, a pharmaceutical composition is administered to a subject 20, 19, 18, 17, 16, 15, 14, 13, 12, 11, 10, 9, 8, 7, 6, 5, 4, 3, 2, and/or 1 day(s) prior to cell infusion. In some embodiments, a pharmaceutical composition is administered to a subject 20, 19, 18, 17, 16, 15, 14, 13, 12, 11, 10, 9, 8, 7, 6, 5, 4, 3, 2, and/or 1 day(s) after the transplant. In some embodiments, a pharmaceutical composition is administered to a subject 20, 19, 18, 17, 16, 15, 14, 13, 12, 11, 10, 9, 8, 7, 6, 5, 4, 3, 2, and/or 1 day(s) after cell infusion. In some embodiments, a pharmaceutical composition is administered to a subject 9 days prior to transplant. In some embodiments, a pharmaceutical composition is administered to a subject 9 days prior to cell infusion. In some embodiments, a pharmaceutical composition is administered to a subject 6 days prior to transplant. In some embodiments, a pharmaceutical composition is administered to a subject 6 days prior to cell infusion. In some embodiments, a pharmaceutical composition is administered to a subject 5 days prior to transplant. In some embodiments, a pharmaceutical composition is administered to a subject 5 days prior to cell infusion. In some embodiments, a pharmaceutical composition is administered to a subject 4 days prior to transplant. In some embodiments, a pharmaceutical composition is administered to a subject 4 days prior to cell infusion. In some embodiments, a pharmaceutical composition is administered to a subject 3 days prior to transplant. In some embodiments, a pharmaceutical composition is administered to a subject 3 days prior to cell infusion. In some embodiments, a pharmaceutical composition is administered to a subject 2 days prior to transplant. In some embodiments, a pharmaceutical composition is administered to a subject 2 days prior to cell infusion. In some embodiments, a pharmaceutical composition is administered to a subject 1 day prior to transplant. In some embodiments, a pharmaceutical composition is administered to a subject 1 day prior to cell infusion. In some embodiments, a pharmaceutical composition is administered to a subject 1 day after the transplant. In some embodiments, a pharmaceutical composition is administered to a subject 1 day after cell infusion. In some embodiments, a pharmaceutical composition is administered to a subject at least one, at least two, at least three, at least four, at least five, at least six, at least seven, at least eight, at least nine, at least ten, or more than at least ten times prior to a transplant. In some embodiments, a pharmaceutical composition is administered to a subject at least one, at least two, at least three, at least four, at least five, at least six, at least seven, at least eight, at least nine, at least ten, or more than at least ten times prior to cell infusion. In some embodiments, a pharmaceutical composition is administered to a subject at least one, at least two, at least three, at least four, at least five, at least six, at least seven, at least eight, at least nine, at least ten, or more than at least ten days prior to a transplant. In some embodiments, a pharmaceutical composition is administered to a subject at least one, at least two, at least three, at least four, at least five, at least six, at least seven, at least eight, at least nine, at least ten, or more than at least ten days prior to cell infusion. In some embodiments, a pharmaceutical composition is administered to a subject at least one, at least two, at least three, at least four, at least five, at least six, at least seven, at least eight, at least nine, at least ten, or more than at least ten times after a transplant. In some embodiments, a pharmaceutical composition is administered to a subject at least one, at least two, at least three, at least four, at least five, at least six, at least seven, at least eight, at least nine, at least ten, or more than at least ten times after cell infusion. In some embodiments, a pharmaceutical composition is administered to a subject at least one, at least two, at least three, at least four, at least five, at least six, at least seven, at least eight, at least nine, at least ten, or more than at least ten days after a transplant. In some embodiments, a pharmaceutical composition is administered to a subject at least one, at least two, at least three, at least four, at least five, at least six, at least seven, at least eight, at least nine, at least ten, or more than at least ten days after cell infusion.

In some embodiments, a pharmaceutical composition is administered to a subject for a continuous amount of time/days prior to a transplant. In some embodiments, a pharmaceutical composition is administered to a subject for a continuous amount of time/days prior to cell infusion. In some embodiments, a pharmaceutical composition is administered to a subject for a continuous amount of time/days after a transplant. In some embodiments, a pharmaceutical composition is administered to a subject for a continuous amount of time/days after cell infusion. In some embodiments, a pharmaceutical composition is administered to a subject for about, at least about, or at most about 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 12 months, or more than 12 months after a transplant. In some embodiments, a pharmaceutical composition is administered to a subject for about, at least about, or at most about 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 12 months, or more than 12 months after cell infusion.

In some embodiments, a pharmaceutical composition is administered to a subject 9 and/or 2 days prior to transplant. In some embodiments, a pharmaceutical composition is administered to a subject 9 and/or 2 days prior to cell infusion. In some embodiments, a pharmaceutical composition is administered to a subject 6, 5, 4 and/or 3 days prior to transplant. In some embodiments, a pharmaceutical composition is administered to a subject 6, 5, 4 and/or 3 days prior to cell infusion. In some embodiments, a pharmaceutical composition is administered to a subject one day prior to transplant, on the same day as the transplant, and/or on the day after the transplant. In some embodiments, a pharmaceutical composition is administered to a subject one day prior to cell infusion, on the same day as cell infusion, and/or on the day after cell infusion. In some embodiments, a pharmaceutical composition is administered to a subject six days prior to transplant, one day prior to transplant, on the same day as the transplant, on the day after the transplant, and/or six days after the transplant. In some embodiments, a pharmaceutical composition is administered to a subject six days prior to cell infusion, one day prior to cell infusion, on the same day as cell infusion, on the day after cell infusion, and/or six days after cell infusion. In some embodiments, a pharmaceutical composition is administered to a subject on the same day as the transplant. In some embodiments, a pharmaceutical composition is administered to a subject on the same day as cell infusion. In some embodiments, a pharmaceutical composition is administered to a subject one day after the transplant. In some embodiments, a pharmaceutical composition is administered to a subject one day after cell infusion.

In some embodiments, a pharmaceutical composition is administered to the subject after the transplant. In some embodiments, a pharmaceutical composition is administered to the subject after about or after at least about 1 day, 2 days, 3 days, 4 days, 5 days, 6 days, 7 days, 8 days, 9 days, 10 days, 11 days, 12, days, 13 days, 14 days, 21 days, 30 days, 1 month, 2 months, 3 months, 4 months, 5 months, 6 months, 7 months, 8 months, 9 months, 10 months, 11 months, 12 months, 18 months, 20 months, 24 months, or more than 24 months after the transplant. In some embodiments a pharmaceutical composition is administered to the subject after about or after at least about 1-2 months, 2-3 months, 3-4 months, 4-5 months, 5-6 months, 6-7 months, 7-8 months, 8-9 months, 9-10 months, 11-12 months, 1-6 months, 2-6 months, 3-6 months, 4-6 months, 6-12 months, or more than 6-12 months after the transplant.

In some embodiments, a pharmaceutical composition comprising a B-cell depleting antibody (e.g., rituximab at, e.g., 375 mg/m²) is administered to a subject (e.g., on days −9 and/or −2 prior to transplant/cell infusion). In some embodiments, a pharmaceutical composition comprising a B-cell depleting antibody (e.g., rituximab at, e.g., 375 mg/m²) is administered to a subject (e.g., on days −9 and/or −2 prior to transplant/cell infusion and/or five days after transplant/cell infusion, and/or twelve days after transplant/cell infusion). In some embodiments, a pharmaceutical composition comprising an anti-neoplastic agent (e.g., fludarabine at, e.g., 10 mg/m²) is administered to a subject (e.g., on days −6, −5, −4, and/or −3 prior to transplant/cell infusion). In some embodiments, a pharmaceutical composition comprising an anti-CD2 antibody or an antigen-binding fragment thereof (e.g., siplizumab at 0.6 mg/kg) is administered to a subject (e.g., on the day prior to transplant/cell infusion (day-1), on the day of the transplant/cell infusion (day 0), and/or on the day after the transplant/cell infusion (day 1)). In some embodiments, a pharmaceutical composition comprising an anti-CD2 antibody or an antigen-binding fragment thereof (e.g., siplizumab at 0.6 mg/kg) is administered to a subject (e.g., six days prior to transplant/cell infusion, on the day prior to transplant/cell infusion (day −1), on the day of the transplant/cell infusion (day 0), and/or on the day after the transplant/cell infusion (day 1)). In some embodiments, a pharmaceutical composition comprising an anti-CD2 antibody or an antigen-binding fragment thereof (e.g., siplizumab at 0.6 mg/kg) is administered to a subject (e.g., six days prior to transplant/cell infusion, on the day prior to transplant/cell infusion (day −1), on the day of the transplant/cell infusion (day 0), on the day after the transplant/cell infusion (day 1), and/or six days after the transplant (day 6)). In some embodiments, a pharmaceutical composition comprising a chemotherapy agent (e.g., cyclophosphamide, e.g., at 22.5 mg/kg) is administered to a subject (e.g., on days −5 and/or −4 prior to transplant). In some embodiments, irradiation (e.g., thymus irradiation, 7 Gy) is given to the subject on the day prior to transplant. In some embodiments, a pharmaceutical composition comprising an immunosuppressive agent (e.g., tacrolimus, e.g., at 8-10 ng/mL) is administered to the subject (e.g., on the day of transplant and/or for about 1 month). In some embodiments, a pharmaceutical composition comprising an immunosuppressive agent (e.g., tacrolimus, e.g., at 4-11 ng/ml) is administered to the subject (e.g., on the day of transplant and/or for about 9-12 months). In some embodiments, a pharmaceutical composition comprising an immunosuppressive agent (e.g., tacrolimus, e.g., at 4-11 ng/ml) is administered to a subject after the transplant (e.g., after about 1 month, 2 months, 3 months, 4 months, 5 months, 6 months, 7 months, 8 months, 9 months, 10 months, 11 months, 12 months, 18 months, 24 months, or more than 24 months after the transplant). In some embodiments, a pharmaceutical composition comprising an antiproliferative agent (e.g., MMF, e.g., at about 1 g/day) is administered to the subject (e.g., on the day of transplant and/or for about 1-2 months). In some embodiments, a pharmaceutical composition comprising an antiproliferative agent (e.g., MMF, e.g., at about 1 g/day) is administered to a subject after the transplant (e.g., after about 1 month, 2 months, 3 months, 4 months, 5 months, 6 months, 7 months, 8 months, 9 months, 10 months, 11 months, 12 months, 18 months, 24 months, or more than 24 months after the transplant). In some embodiments, a pharmaceutical composition comprising an immunosuppressive agent (e.g., corticosteroid) is administered to the subject (e.g., on the day of transplant and/or for about 6 months). In some embodiments, a pharmaceutical composition comprising an immunosuppressive agent (e.g., corticosteroid) is administered to the subject (e.g., on the day of transplant and/or for about 20 days). In some embodiments, a pharmaceutical composition comprising an immunosuppressive agent (e.g., corticosteroid) is administered to a subject after the transplant (e.g., after about 1 month, 2 months, 3 months, 4 months, 5 months, 6 months, 7 months, 8 months, 9 months, 10 months, 11 months, 12 months, 18 months, 24 months, or more than 24 months after the transplant). In some embodiments, a pharmaceutical composition comprising an immunosuppressive agent (e.g., sirolimus, e.g., initial trough 5-8 ng/mL) is administered to the subject (e.g., after the transplant (e.g., after about 1 month, 2 months, 3 months, 4 months, 5 months, 6 months, 7 months, 8 months, 9 months, 10 months, 11 months, 12 months, 18 months, 24 months, or more than 24 months after the transplant, and/or one month after the day of transplant and/or for about 12 months). In some embodiments, a pharmaceutical composition comprising an anti-IL6R antibody (e.g., tocilizumab) is administered to a subject (e.g., if CTS occurs, seven days after transplant or cell infusion, and/or fourteen days after transplant or cell infusion). In some embodiments, a pharmaceutical composition comprising an anti-IL6R antibody (e.g., tocilizumab) is administered to a subject after the transplant (e.g., after about 1 month, 2 months, 3 months, 4 months, 5 months, 6 months, 7 months, 8 months, 9 months, 10 months, 11 months, 12 months, 18 months, 24 months, or more than 24 months after the transplant).

In certain embodiments, pharmaceutical compositions described herein can be formulated for subcutaneous, intravenous, intravascular, topical, intrarticular, intraarterial, intracranial, intramuscular, oral, intraorbital, inhalation, intraperitonial, intraosseous, endotracheal, sublingual, buccal, rectal, intradermal, intrathecal, intramedullary, or transdermal routes of administration. In some embodiments, a pharmaceutical composition described herein is formulated for intravenous administration. In some embodiments, a pharmaceutical composition described herein is formulated for subcutaneous administration.

In certain embodiments, the composition formulated for intravenous or subcutaneous administration can be a solution, suspension, or an emulsion. In certain embodiments, an agent (e.g., an anti-CD2 antibody or antigen binding fragment thereof) described herein is formulated for intravenous or subcutaneous administration by combining the agent with a pharmaceutically appropriate vehicle. In certain embodiments, vehicles used can be, but are not limited to, water, saline, Ringer's solution, dextrose solution, glycerol, ethanol, 1-10% human serum albumin, 5% dextrose in water, N-methylpyrrolidone, dimethyl sulfoxide, N,N-dimethylacetamide, propylene glycol, polyethylene glycol 400, diethylene glycol monoethyl ether, TWEEN 80, TWEEN 20, polyoxyl-35 castor oil, polyoxyl 40 hydrogenated castor oil, caprylocaproyl macrogol-8-glycerides, soybean oil, polyxoyethyllated oleic glycerides, and medium chain mono- and diglycerides. In certain embodiments, liposomes and non-aqueous vehicles such as fixed oils can also be used to administer an agent (e.g., an anti-CD2 antibody or antigen binding fragment thereof) as described herein.

In certain embodiments, the vehicle can contain additives that maintain isotonicity (e.g., sodium chloride, mannitol). In certain embodiments the vehicle can contain additives to maintain chemical stability. These additives can include, but are not limited to, buffers (e.g. maleic acid, tartaric acid, lactic acid, citric acid, acetic acid, sodium bicarbonate, and sodium phosphate), and preservatives (e.g. detergents, oxidizing agents, and ionic buffers.) The resulting pharmaceutical formulation is sterilized by known or suitable techniques.

In certain embodiments, the intravenous administration of the pharmaceutical composition described herein can be administered as a bolus injection, a slow intravenous injection, or a continuous intravenous infusion. In certain embodiments, the subcutaneous administration of the pharmaceutical composition described herein can be administered as a bolus injection.

### 5.8 Kits

Provided herein is a kit comprising a pharmaceutical composition described herein, contained in one or more containers. Provided herein is a kit containing an anti-CD2 antibody or an antigen-binding fragment thereof and/or one or more than one second agent (e.g., rituximab, cyclophosphamide, tocilizumab, corticosteroid, tacrolimus, MMF, antiproliferative agent, steroid, sirolimus, fludarabine, chemotherapy agent, anti-IL6R antibody, a B-cell depleting antibody, immunosuppressive agent, anti-neoplastic agent, and/or antiproliferative agent) as described herein. In some embodiments, a kit comprises a pharmaceutical composition described herein, contained in one or more containers. In some embodiments, a kit includes one or more anti-CD2 antibody or an antigen-binding fragment thereof and/or a second agent of the disclosure or a composition thereof in the same or a different container. In some embodiments, a kit includes at least one anti-CD2 antibody or an antigen-binding fragment thereof in the same or a different container. In some embodiments, a kit contains a library of anti-CD2 antibody or an antigen-binding fragment thereof. In some embodiments, a kit includes another active agent/second agent. In some embodiments, a kit includes a control and/or a reference (e.g., a reference antibody). In some embodiments, a kit may further include reagents and/or instructions for creating and/or synthesizing compounds and/or compositions described herein. In some embodiments, a kit may also include one or more buffers. In some embodiments, a kit includes another agent (e.g., another active agent) in the same or a different container. In some embodiments, a container with an agent (e.g., anti-CD2 antibody or an antigen-binding fragment thereof) is provided for a single dose administration or multiple dose administrations. In some embodiments, an agent (e.g., anti-CD2 antibody or an antigen-binding fragment thereof) is present in a container in a kit in an amount sufficient for multiple dosages, usages, or administration. In some embodiments, a kit includes other components necessary for administration of an agent (e.g., anti-CD2 antibody or an antigen-binding fragment thereof) (e.g., a kit includes a syringe, a catheter, a cannula, a pump, or any injection device). In certain embodiments, the kit comprises devices that can be used to administer the pharmaceutical composition described herein, including, but not limited to, syringes, needle-less injectors, drip bags, perfusion pumps, pumps, patches, and inhalers. In some embodiments, a kit includes a pharmaceutically acceptable carrier, diluent, excipient, and/or buffer, in the same or separate container as the container holding one or more of agent as described herein. In some embodiments, a kit includes components and/or instructions for thymic irradiation.

Components of a kit can be in separate containers or can be combined in a single container. In some embodiments, kit components may be packaged either in aqueous media, in powder form, in crystal form, or in lyophilized form. The containers that the pharmaceutical composition can be packaged in can include, but are not limited to, bottles, packets, ampoules, tubes, inhalers, bags, vials, and containers. The container means of the kits can include at least one of a vial, test tube, flask, bottle, needle-less injectors, drip bags, perfusion pumps, pumps, patches, an inhaler, ampoules, syringe or other container means, into which a component may be placed. In some embodiments, a component is suitably aliquoted. In some embodiments, where there is more than one kit component, a reagent and the corresponding label can be packaged together. In some embodiments, a kit contains second, third or other additional containers into which additional components may be separately placed. In some embodiments, a kit includes a second container means for containing sterile, pharmaceutically acceptable buffers and/or other diluents. In some embodiments, various combinations of components are included in one or more vial. In some embodiments, a kit includes a means for containing antibody and/or compounds and/or compositions of the disclosure in close confinement for commercial sale. Such containers may include injection or blow-molded plastic containers into which desired vials are retained.

In some embodiments, kit components are provided in one and/or more liquid solutions. In some embodiments, a liquid solution is an aqueous solution or a sterile aqueous solution. In some embodiments, kit components are provided as dried powder(s). When reagents and/or components are provided as dry powders, such powders may be reconstituted by the addition of suitable volumes of a solvent. In some embodiments, the solvents are provided in another container means. In some embodiments, labeling dyes are provided as dried powders. In some embodiments, labeling dyes are provided in an amount of about, at least about, or at most about 10, 20, 30, 40, 50, 60, 70, 80, 90, 100, 120, 120, 130, 140, 150, 160, 170, 180, 190, 200, 300, 400, 500, 600, 700, 800, 900, 1000 micrograms. In some embodiments, dye can be re-suspended in any suitable solvent, such as DMSO.

In some embodiments, a kit include instructions for using the components of the kit. In some embodiments, a kit contains instructions related to dosage, administration, applications, storage conditions, a list of diseases that can be treated or prevented by using one or more of the kit components, and/or use of the components. In some embodiments, instructions are recorded on a suitable recording medium. For example, instructions can be printed on a substrate, such as a paper, a tag (e.g., adhesive tag), or plastic. In some embodiments, instructions are present in a kit as a package insert or on a label attached to a container or components of a kit. In some embodiments, instructions are provided as electronic storage data file present on a suitable computer readable storage medium, e.g. CD-ROM, diskette, USB storage device, or flash drive. In some embodiments, instructions are not present in a kit but are present in a remote source, e.g. via the internet.

### 5.9 Equivalents and Incorporation by Reference

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

### 6. EXAMPLES

### 6.1 Example 1. A Safety and Tolerability Study of an Anti-CD2 Antibody or Antigen Binding Fragment Thereof (e.g., Siplizumab) Combined with Donor Bone Marrow Cell Infusion and Non-Myeloblative Conditioning, Including Fludarabine and Cyclophosphamide, for Tolerance Induction in De Novo Living Donor Renal Transplantation

In this study, an anti-CD2 antibody or antigen binding fragment thereof (e.g. siplizumab), is combined with donor bone marrow cells and non-myeloablative conditioning using fludarabine and cyclophosphamide, in de novo living donor renal transplant recipients to determined allogeneic tolerance.

Primary objective: to induce renal allograft tolerance (proportion of recipients off immunosuppression with maintenance of good renal function at month 24 post-transplant).

Key secondary objectives: To determine the composite incidence of biopsy proven acute rejection (BPAR), death and graft loss (with and without loss to follow up) at month 24 post-transplant and to determine the incidence and mean fluorescence intensity (MFI) of de novo DSA (donor specific antibodies) at month 24 post-transplant.

Secondary objectives:
To evaluate the safety, tolerability and activity of an anti-CD2 antibody or antigen binding fragment thereof (e.g., siplizumab) and fludarabine based conditioning regimen over time
Incidence and grade of CTS
Incidence of death and graft loss
Incidence of biopsy proven acute rejection (BPAR) and treated BPAR (with Banff classification)
Renal function (estimated glomerular filtration rate (eGFR)) and change of renal function over time (e.g., evaluated at Months 3, 6, 12, 24 and then yearly thereafter through Month 60)
Incidence of serious adverse events and adverse events
Incidence of severe or clinically significant opportunistic infections
Incidence of BK viremia, infection and nephropathy
Incidence of malignancies.
Incidence and severity of GvHD
Incidence of new onset diabetes post-transplant (NODAT)
Incidence of chronic rejection

TABLE 7
Primary and Secondary Objectives and Related Endpoints

| Objective(s) | Endpoints |
|---|---|
| Primary objective | Endpoints for primary objective |
| To induce renal allograft tolerance | Immunosuppression dosing |
| (proportion of recipients off | Serum creatinine (Estimated |
| immunosuppression with | Glomerular Filtration Rate; eGFR) |
| maintenance of good renal | AE/SAEs |
| function at Month 24 post- | Renal biopsy |
| transplant) | |
| Key Secondary Objectives | Endpoints for key secondary |
| To determine the composite | objective |
| incidence of biopsy proven acute | Incidence of Biopsy Proven Acute |
| rejection (BPAR), death and graft | Rejection (BPAR) |
| loss (with and without loss to | Patient/graft survival and |
| follow-up) at Month 24 post- | disposition |
| transplant | Incidence of de novo Donor |
| To determine the incidence and | Specific Antibodies (DSA) |
| mean fluorescence intensity | |
| (MFI) of de novo donor-specific | |
| antibody (DSA) at Month 24 | |
| post-transplant | |
| Secondary Objectives | Endpoints for secondary objectives |
| To evaluate the safety, tolerability | Adverse Event (AE)/Serious |
| and activity of an anti-CD2 | Adverse Event (SAEs), clinical |
| antibody or antigen binding | laboratory data |
| fragment thereof (e.g., siplizumab) | |
| and fludarabine-based | |
| conditioning regimen over time | |
| Objective(s) | Endpoints |
| Incidence and grade of CTS | Serum creatinine, AEs |
| Incidence of death and graft loss | Patient/graft survival |
| Incidence of BPAR and treated | Renal biopsy, concomitant |
| BPAR (with Banff | medications, immunosuppression |
| classification) | dosing, concentrations |
| Renal function (eGFR) and change | eGFR and change in eGFR |
| of eGFR over time | AE/SAE |
| Incidence of serious adverse | AE/SAE infections System Organ |
| events and adverse events | Class (SOC) |
| Incidence of severe and clinically | Incidence of BK AE events, biopsy |
| significant opportunistic infections | AE/SAE malignancy/neoplasms |
| Incidence of BK viremia, infection | SOC |
| and nephropathy | GvHD assessment, AE/SAEs |
| Incidence of malignancies | Diabetes AE, lab data, concomitant |
| Incidence and severity of GvHD | use of anti-diabetic medications |
| Incidence of new onset diabetes | AE/SAEs, biopsy, Incidence of |
| post-transplant (NODAT) | chronic rejection |
| Incidence of chronic rejection | |

### Exploratory Objectives:

Assessment of lymphocyte depletion and recovery, including Tregs and T effector/memory cells, quantified by flow cytometry
Time to neutrophil recovery following transplant (ANC >500/mm3)
Time to platelet recovery following transplant (platelets >20,000/mm3 and transfusion independent)
Characterize the pharmacokinetics of an anti-CD2 antibody or antigen binding fragment thereof (e.g., siplizumab)
Assessment of donor-specific lymphoid and myeloid chimerism by:
   Flow cytometry
   Variable short tandem repeats (VNTR) PCR-based DNA microsatellite analyses
Incidence or progression of abnormal histologic findings of cellular or antibody mediated rejection (i.e., TG, IF/TA, C4d, BK/Polyoma virus nephropathy)
Follow-up assessment and safety monitoring of the donor for hematologic and renal function, and other complications of living donation
Evaluation of biomarkers of tolerance (e.g., FoxP3 on biopsy)
Evaluation of cytokine panel over time
Evaluation of HLA eplet mismatch load and relationship to outcomes

TABLE 8
Exploratory Objectives and Related Endpoints

| Exploratory Objectives | Endpoints for exploratory objectives |
|---|---|
| Assessment of lymphocyte | FACS analysis of lymphocyte |
| depletion and recovery, including | subsets |
| Tregs and T effector/memory | ANC in hematology |
| cells, quantified by flow | Platelets in hematology |
| cytometry | |
| Time to neutrophil recovery | |
| following transplant (ANC > | |
| 500/mm3) | |
| Time to platelet recovery | |
| following transplant (platelets > | |
| 20,000/mm3 and transfusion | |
| independent) | |
| Characterize the pharmacokinetics | an anti-CD2 antibody or antigen |
| of an anti-CD2 antibody or | binding fragment thereof (e.g., |
| antigen binding fragment thereof | siplizumab) PK/Adenosine |
| (e.g., siplizumab) | deaminase (ADA) Analysis |
| Assessment of donor-specific | Chimerism by flow and PCR |
| lymphoid and myeloid chimerism | Renal biopsy data and histology |
| by: | |
| Flow cytometry | |
| VNTR PCR-based DNA | |
| microsatellite analyses | |
| Incidence or progression of | |
| abnormal histologic findings of | |
| cellular or antibody mediated | |
| rejection (e.g., TG, IF/TA, C4d, | |
| BK/Polyoma virus nephropathy | |
| Follow-up assessment and safety | Donor follow-up data |
| monitoring of the donor for | |
| hematologic and renal function, | |
| and other complications of living | |
| donation | |
| Evaluation of biomarkers of | Histopathology |
| tolerance (e.g., FoxP3 on biopsy) | |
| Evaluation of cytokine panel over | Cytokine panel by visit |
| time | |
| Evaluation of HLA eplet | HLA typing and analysis by |
| mismatch load and relationship to | eplet mismatch. Outcomes; |
| outcomes | BPAR, Off |
| | immunosuppression, patient |
| | and graft survival, DSA |

### Study Design:

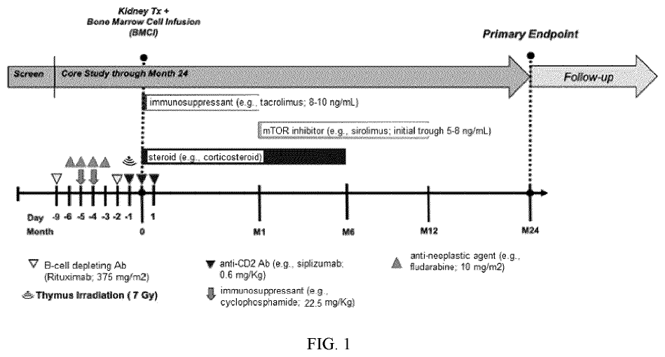

Approximately 12 subjects are enrolled in this study. The primary objective is assessed at Month 24, the recipients remain in study up to 5 years post-transplant for follow-up (refer to FIG. 1). The study evaluates an anti-CD2 antibody or antigen binding fragment thereof (e.g., siplizumab) based treatment regimen in 12 patients. Based on the primary objective outcome (i.e., proportion of patients off immunosuppression with good renal function and an acceptable safety profile at Month 24), the protocol can be amended to allow for the enrollment of additional patients.

During the screening period (from Day −37 to Day −10) and after informed consent is signed, baseline subject information is obtained from the donor and recipient, including date of birth, age, sex, race and ethnicity. In addition, relevant medical history, including current medical conditions, cause of end stage renal disease (ESRD), dialysis and medication history is reviewed. A full physical examination, vital signs, laboratory assessments, and pregnancy testing are performed.

Pre-transplant screening procedures include donor/recipient HLA typing (molecular), complement-dependent cytotoxicity (CDC) lymphocyte cross-match (or virtual cross-match), qualitative DSA by single antigen bead (SAB) assay and donor/recipient viral serology. Standard of care procedures performed prior to consent can be considered in the determination of subject eligibility (e.g., HLA/ABO typing, specific laboratory results). Recipients, and their respective donors who complete the screening period and meet all inclusion criteria are enrolled into the study.

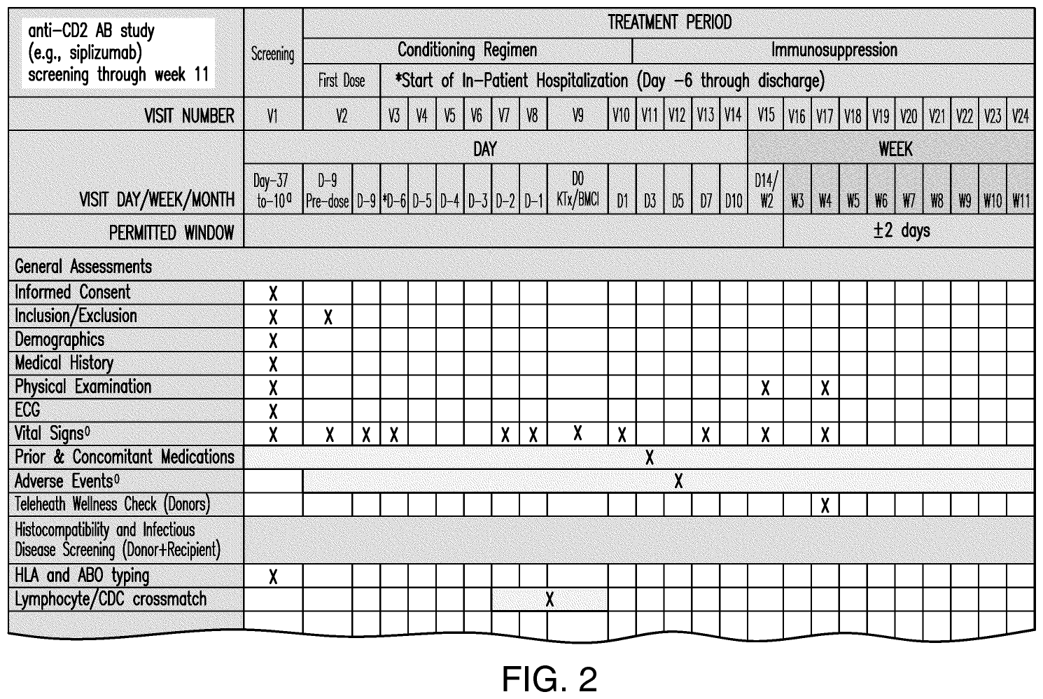

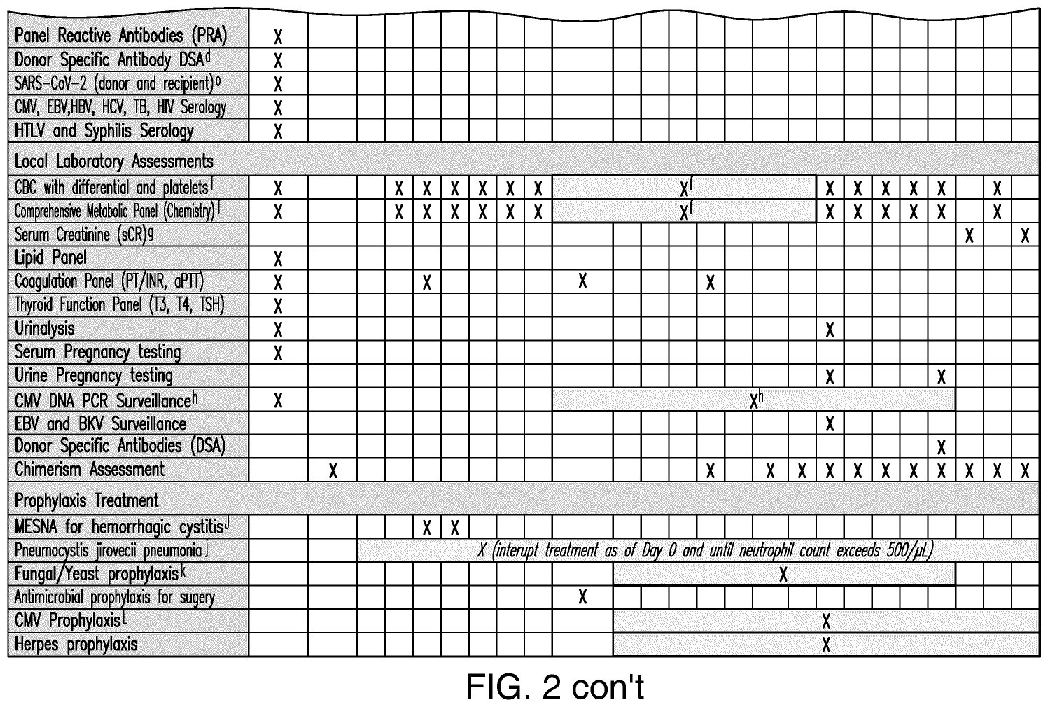

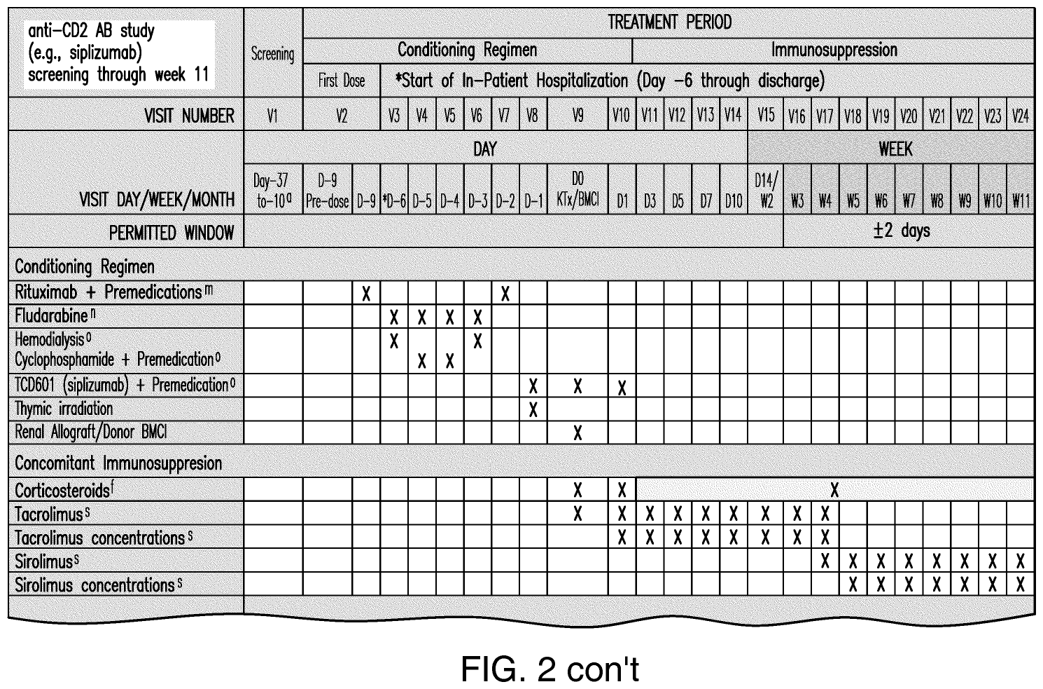

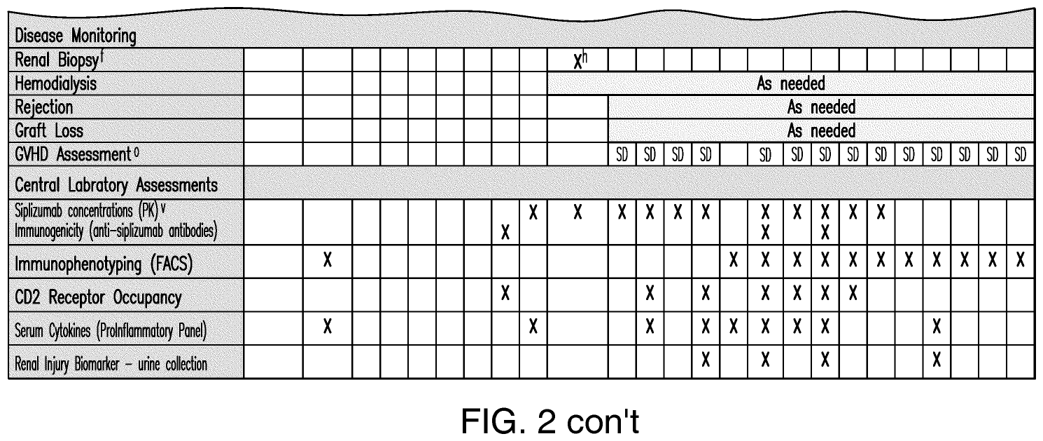

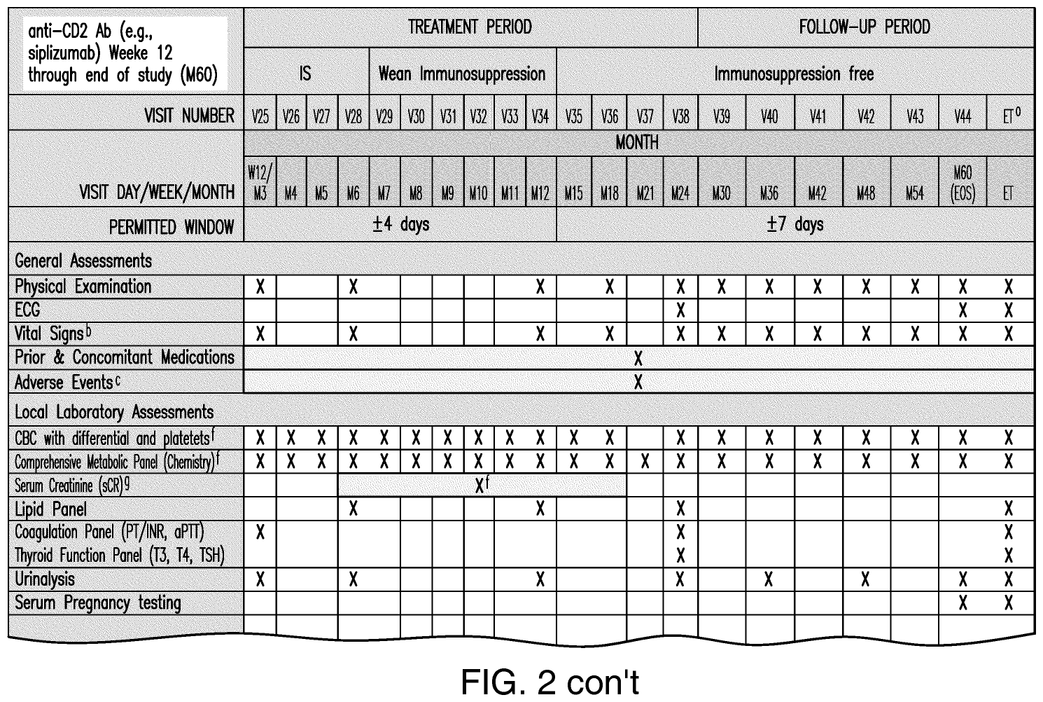

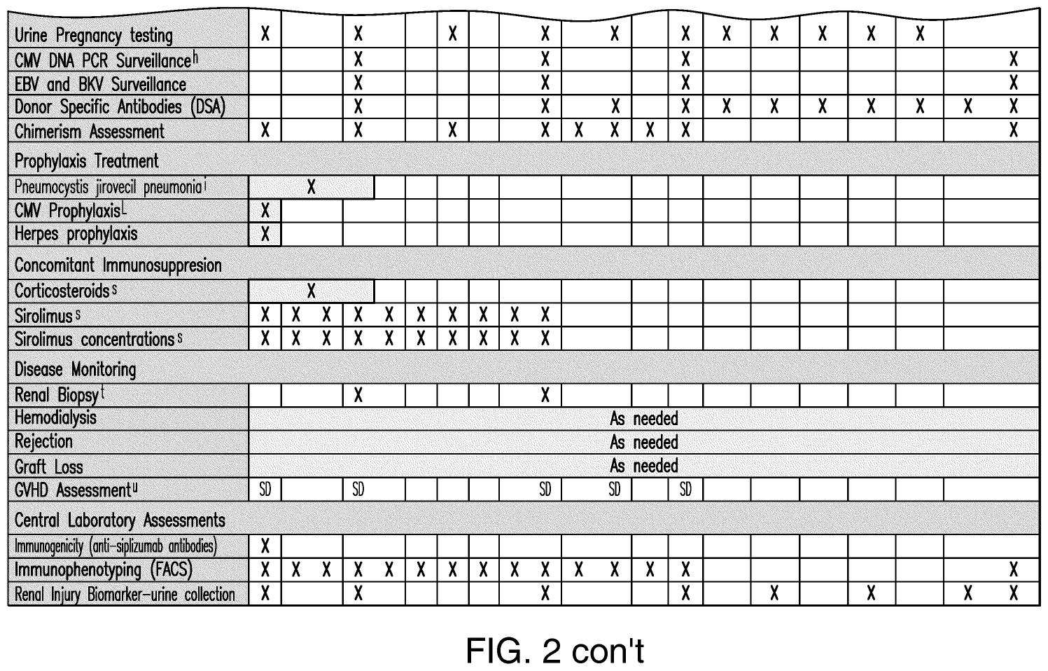

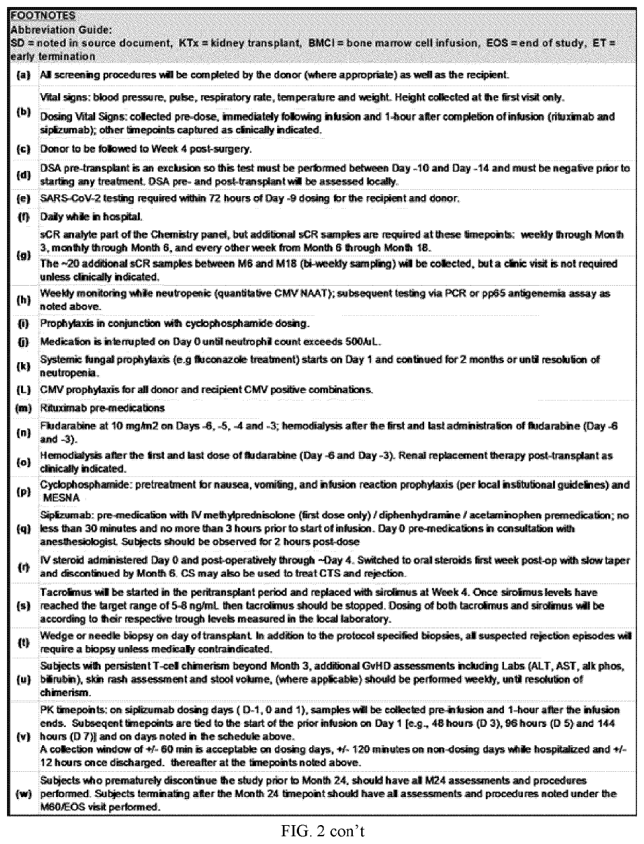

Day −9 is defined as the start of the Study Treatment Period (Conditioning Regimen) and Day 0 as the day of transplant surgery. The first study treatment visit (Day −9) is administered on an outpatient basis. Recipients are admitted to the hospital as of Day −6, prior to the next study treatment administration, and continue the study as an inpatient until discharge from the hospital post-transplant. Discharge from the hospital vary based on the clinical status of the donor and recipient and are determined at the discretion and judgment of the clinical team and physicians. Following discharge from the hospital, subjects return to the clinic for weekly visits through Week 11, monthly visits from Month 3 through Month 12, quarterly visits up until Month 24 and then visits every 6 months until the end of study (EOS)/Month 60. Throughout these visits safety and PK/PD assessments are collected according to FIG. 2.

Recipients initiate pre-transplant conditioning with an infusion of rituximab (375 mg/m²) on Day −9. Fludarabine (10 mg/m²) is administered on Days −6, −5, −4, −3 with cyclophosphamide (22.5 mg/kg) on Days −5 and −4. A second dose of rituximab is administered on Day −2 and thymic irradiation 700 cGy on Day −1. The anti-CD2 antibody or antigen binding fragment thereof (e.g., siplizumab) is administered at a dose of 0.6 mg/kg on Days −1, 0 and 1. A combined renal transplant and donor bone marrow cell infusion is performed on Day 0.

IV steroids are administered on the Day of transplant and post-operatively through approximately Day 4 based on dose and tolerability. During the first week post-transplant subjects transition from IV to oral steroids with a slow taper through Month 6, at which time steroids are discontinued.

Concentration-controlled, background, immunosuppression start with tacrolimus on perioperative Day 0, (target trough 8-10 ng/mL) and then switched to sirolimus (SRL) at Week 4. Sirolimus (initial target trough 5-8 ng/mL) continue until Month 6. In some cases, no mycophenolate is used in this study. A per protocol biopsy is conducted at Month 6, and if the a priori defined immunosuppression weaning criteria are met, the SRL dose is reduced (e.g., to achieve a target trough concentration of 3-5 ng/mL). At Month 12 post-transplant, based on an assessment of a 1-year protocol biopsy and allograft function or renal function, SRL is withdrawn completely.

Standard safety assessments include vital signs, physical examinations, ECGs, clinical laboratory evaluations (hematology, blood chemistry and urinalysis), AE and SAE monitoring. In addition to standard clinical laboratory assessments, subjects are monitored regularly for coagulopathies as well as signs and symptoms of inflammatory, hematologic or renal toxicity/rejection. Subject's serum creatinine, serum cytokines and renal injury makers are measured frequently in the first few weeks post-transplant and thereafter with a focus on renal injury, dysfunction or acute rejection. Changes in renal function are assessed via serum creatinine and estimated glomerular filtration rates (eGFR) using the Chronic Kidney Disease Epidemiology Collaboration (CKD-EPI) and Modification of Diet in Renal Disease (MDRD) formula. All biopsies are histopathologically evaluated and graded in real-time, based on the Banff classification of renal allograft pathology guidelines by a trained renal pathologist. Exploratory urinary injury biomarker samples are collected for future analysis which can help to assess the type and gross location of renal injury, i.e., glomerular or tubular. Extensive immunophenotyping via fluorescence-activated cell sorter (FACS) analysis are conducted during the study to assess donor chimerism as well as changes in leukocyte subsets and recovery following conditioning and donor bone marrow cell infusion.

### Inclusion Criteria for Recipient Subjects:

1. Able to understand the study requirements and provide written informed consent before any study assessment is performed.
2. Male or female recipients aged 18-60 years.
3. Undergoing a first renal transplant.
4. Recipient of a renal transplant from a non-HLA identical but at least haploidentical, ABO compatible living donor.
5. Capable of complying with the schedule of study visits, especially after discontinuation of immunosuppression.
6. Stable cardio-pulmonary status per the judgement of the investigator, with ejection fraction >40% within 3 months of transplant, and eligible for transplantation.
7. EBV seropositive within 6 months of screening.
8. Negative SARS-CoV-2 nucleic acid amplification test (NAAT) within 72 hours of Day −9 (Start of Treatment Period).
9. Recipient free from any other locally endemic infections that can be contraindications to solid organ or BMT/HSCT transplantation according to applicable guidelines.
10. Male study subjects willing to maintain barrier contraception (condom) and agree not to father a child until 180 days after the last dose of fludarabine.

### Recipient: Histocompatibility Testing (HLA/ABO Typing):

High-resolution HLA typing pre-transplant and HLA class I and class II antigens (including HLA A, B, C, DR, DP and DQ) are performed. In addition, serological testing of blood groups as well as B and T-cell flow cytometry crossmatch tests are performed. In addition to providing information on the quality of the match, the high-resolution HLA results ensure capability of detecting hematopoietic cells of donor origin by FACS and by immunohistochemistry. Exploratory assessments of eplet mismatch load are calculated.

### Recipient: History of Sensitization (PRA, DSA):

The potential subject's serum is screened prior to transplant for reactivity against the proposed donor as well as against a panel of HLA specificities (PRA). The screening for anti-donor HLA antibodies (DSA) utilize single-antigen bead assays (e.g., Luminex).

### Recipient: Infectious Disease Screening:

Cytomegalovirus IgG Antibody
Cytomegalovirus IgM Antibody
Epstein-Barr Virus Nuclear Antigen IgG Antibodies
Hepatitis B Core Antibody
Hepatitis B Surface Antibody
Hepatitis B Surface Antigen
Hepatitis C Antibody
TB QuantiFERON
HIV I/II Screen
HTLV1 Antibody
Syphilis Serology
SARS-CoV-2 nucleic acid amplification testing (testing within 72 hours of Day −9)

### Recipient: Clinical Laboratory Testing:

Complete Blood Count with diff (CBC)
Coagulation Panel
Comprehensive Metabolic Panel (Chemistry)
Lipid Panel
T3/T4/TSH
Urinalysis
Serum Pregnancy test (for females of childbearing potential)
CMV DNA NAAT (baseline assessment; testing within 72 hours of transplant)

### Inclusion Criteria for Donor Subjects:

1. Donor subject willing and able to provide informed consent prior to any study assessments being performed.
2. Male or female not less than 18 or more than 65 years of age.
3. Be in excellent health per conventional pre-donor history (medical, laboratory and psychosocial evaluation).
4. Negative for HBsAg, HIV, HCV (RNA) and HTLV-1. Viral test results within 28 days of Study Day 0 (Day of Surgery) are acceptable.
5. Negative SARS-CoV-2 NAAT within 72 hours of Study Day −9 (Start of Conditioning Regimen for recipient).
6. Willingness to adhere to COVID protocols (e.g., social distancing, mask usage) and institutional guidelines from Day −9 through Day of Surgery.
7. Negative for latent TB infection as detected by Quantiferon Gold Plus IGRA (or current standard interferon gamma release assay for TB).
8. Free from any other locally endemic infections that are contraindications to solid organ or BMT/HSCT transplantation according to applicable guidelines.

### Exclusion Criteria for Recipient Subjects:

1. Use of other investigational drugs (or enrollment in another investigational drug study) within 30 days of screening or 5 half-lives of the medication, whichever is longer.
2. History of hypersensitivity to any of the study treatments or its excipients or to drugs of similar chemical classes (e.g., MEDI-507, tacrolimus, sirolimus, cyclophosphamide or rituximab).
3. Recipient with end stage renal disease due to focal segmental glomerulosclerosis (FSGS) or membranoproliferative glomerulonephritis (MPGN/C3 glomerulopathy).
4. Recipient with donor specific anti-HLA antibody (DSA) as measured by single antigen bead (e.g., Luminex) assay(s) within 28 days prior to transplant.
5. Recipient with a positive donor cross-match result (assayed according to local practice) within 28 days prior to transplant.
6. Recipient with any panel reactive antibodies (PRA >20%) within 2 months prior to transplant.
7. Subjects with leukopenia (WBC less than 2,000/mm3) or thrombocytopenia (platelet count <100,000/mm3) at baseline.
8. Sero-positive for HIV-1 or HBsAg. Subjects who are sero-positive for Hepatitis C virus are excluded without proof of sustained viral response (SVR) after anti-HCV treatment. Results within 6 months of transplant are acceptable.
9. Subjects with latent TB infection as detected by Quantiferon Gold Plus IGRA (or current standard interferon gamma release assay for TB).
10. Subjects with a history of cancer other than basal cell carcinoma of the skin or carcinoma in situ of the cervix.
11. Subjects with a clinically significant laboratory abnormality that would preclude participation in the study (e.g., >2.5×ULN values for (a) liver chemistries [(ALT, AST, ALP), (b) bilirubin, (c) coagulation studies (PT, aPTT)]).
12. Subjects who, in the opinion of the investigator, are not capable of giving informed consent for the study or who are unable or unwilling to adhere to the study requirement outlined in the protocol.
13. Subjects with any other clinically significant medical condition or laboratory abnormality that would, in the judgment of the investigator, interfere with the subject's ability to participate in the study.
14. Subjects who have received any live-attenuated vaccine within 2 months of planned transplant.
15. Pregnant or nursing (lactating) women, where pregnancy is defined as the state of a female after conception and until the termination of gestation, confirmed by a positive human chorionic gonadotropin (hCG) laboratory test.

### Investigational Product:

anti-CD2 antibody or antigen binding fragment thereof (e.g., siplizumab (TCD601)) is applied at a dose of 0.6 mg/kg per administration. Anti-CD2 antibody or antigen binding fragment thereof (e.g., siplizumab) is supplied in 6 mL vials containing 60 mg and supplied at a concentration of 10 mg/mL. The subject is weighed within 24 hours of each infusion administration and this weight serves as the basis for final dose calculations and compounding. The dose is calculated by the following formula: Dose (mg)=(patient weight (kg)×dose level (mg/kg)). For example, a subject whose weight is 70 kg receiving a dose of 0.6 mg/kg can receive 42 mg of anti-CD2 antibody or antigen binding fragment thereof (e.g., siplizumab) (70 kg×0.6 mg/kg=42 mg) and would require 1 vial of anti-CD2 antibody or antigen binding fragment thereof (e.g., siplizumab). The product is then administered to the subject via IV infusion using a syringe pump.

### Conditioning Regimen:

Anti-CD2 antibody (e.g., siplizumab, 0.6 mg/kg): Days −1, 0, +1
Rituximab, 375 mg/m²: Days −9, −2
Fludarabine, 10 mg/m²: Days −6, −5, −4, −3
Cyclophosphamide, 22.5 mg/kg: Days −5, −4
Thymic Irradiation, 700 cGy: Day −1

Recipients receive a conditioning regimen starting with rituximab on Days −9 and −2, an anti-CD2 antibody or antigen binding fragment thereof (e.g., siplizumab) on Days −1, 0, 1, fludarabine (10 mg/m²) on Days −6, −5, −4, −3, cyclophosphamide (22.5 mg/kg) on Days −5 and −4, and thymic irradiation 700 cGy on Day −1. Methylprednisolone 500 mg IV bolus are given on Day 0 and 1 with a switch from IV to oral steroids during the first week post-operatively, with a slow taper and then discontinuation of oral steroid at Month 6.

For prophylaxis of the risk of hemorrhagic cystitis and hematuria due to cyclophosphamide, MESNA (2-mercaptoethane sulfonate sodium) is given and patients undergo hemodialysis before and after cyclophosphamide administration. Combined kidney transplantation with donor bone marrow cell infusion is performed on day 0. Tacrolimus (trough 8-10 ng/mL) starts at time of transplant and given until Week 4, when patients are switched to sirolimus (initial target trough 5-8 ng/mL).

### Anti-CD2 Antibody or Antigen Binding Fragment Thereof Premedication:

prior to each infusion of an anti-CD2 antibody or antigen binding fragment thereof (e.g., siplizumab), subjects can receive premedication with 650-1000 mg acetaminophen (or paracetamol) and an H1-antagonist (antihistamine, e.g. 25 mg diphenhydramine or 4 mg chlorpheniramine) to minimize signs and symptoms of an infusion reaction. Additionally, for the first dose of an anti-CD2 antibody or antigen binding fragment thereof (e.g., siplizumab) (Day −1), 8 mg/kg methylprednisolone sodium succinate can be given prior to starting the infusion. Administration of required pre-medications can occur no less than 30 minutes and no more than 3 hours priors to the start of the infusion.

### Anti-CD2 Antibody or Antigen Binding Fragment Thereof Administration (Days −1, 0 and 1):

The first dose of an anti-CD2 antibody or antigen binding fragment thereof (e.g., siplizumab; 0.6 mg/kg) is administered on Day −1. The anti-CD2 antibody or antigen binding fragment thereof (e.g., siplizumab) doses can be administered approximately 20-24 hours apart. The an anti-CD2 antibody or antigen binding fragment thereof (e.g., siplizumab) solution is infused intravenously via syringe pump over a period of around 1 hour. Pre-medication precedes administration of an anti-CD2 antibody or antigen binding fragment thereof (e.g., siplizumab) on Days −1 and Day 1.

On Day 0, the infusion can be administered pre- or intra-operatively and timed so that the completion of the infusion is no earlier than 4 hours prior to revascularization and perfusion of the allograft. Pre-medication can be coordinated with any pre-operative medications to avoid therapeutic duplication in consultation with the anesthesiologist. When administered intra-operatively, the infusion must be completed prior to revascularization and administration of the bone marrow cell infusion (BMCI). This ensures both donor and alloreactive T-lymphocytes are suppressed and depletion is initiated in the hours and days post-transplant. Subjects are carefully monitored (a minimum of 2-hours) after each dose for infusion reactions.

The infusions can be given directly into a peripheral vein or a separate lumen in an indwelling, multi-lumen, central catheter (e.g., and not administered concurrently with other medications since compatibility with other IV medications is not known). The date, start time, completion time and total dose of an anti-CD2 antibody or antigen binding fragment thereof (e.g., siplizumab) administration is recorded.

In case of notable AEs and/or SAEs, including loss of efficacy and/or associated Pharmacokinetic (PK)/Pharmacodynamic (PD) data collected during the study, changes to the next planned dose level across the study may be considered and implemented.

### Rituximab Premedication:

acetaminophen (650-1000 mg PO), an H1-antagonist (antihistamine, e.g. 25-50 mg diphenhydramine, 4 mg chlorpheniramine), and hydrocortisone sodium succinate (e.g., Solu-Cortef; 100 mg IV) can be administered to subjects 2 hours prior to each dose of rituximab.

### Rituximab Administration (Days −9 and −2):

Rituximab (375 mg/m²/dose) is administered on Days −9 and −2. In those subjects receiving ongoing renal replacement therapy, rituximab can be administered several hours after hemodialysis. The first rituximab solution for infusion can be administered intravenously at an initial rate of 50 milligrams/hour (mg/hr). The rate may be escalated by 50 mg/hr every 30 minutes to a maximum of 400 mg/hr. The second dose of rituximab is administered on Day −2. The second infusion may be started at 100 milligrams/hour (mg/hr) and titrated by 100 mg/hr every 30 minutes to a maximum of 400 mg/hr if the subject tolerated the first infusion.

Subjects are carefully monitored (a minimum of an hour) for infusion reactions. In the event the subject does not tolerate the first infusion, the initial infusion guidelines can be followed. If hypersensitivity or an infusion-related event develops, the infusion can be temporarily slowed or interrupted. The infusion can then be continued at one-half the previousrate upon improvement of symptoms. The start and stop times and total dose for rituximab administration can be recorded.

### Fludarabine Premedication/Treatment Hemodialysis:

subjects who were not on dialysis prior to transplant require hemodialysis treatment after the first and last dose of fludarabine, as prophylaxis against fludarabine toxicity. Recipients, with no existing vascular access, are seen by a nephrologist on or prior to Day −6 for vascular access and insertion of a catheter in preparation for hemodialysis during conditioning. Hemodialysis can be performed 5-7 hours after the first administration of fludarabine on Day −6, and 5-7 hours after the last administration of fludarabine on Day −3. The duration (or dose) of dialysis can be as close to 4 hours in duration or per local nephrologist recommendation.

Dialysis date, time and duration can be recorded. If the subject's eGFR is <10 mL/min and the subject is on hemodialysis or continuous ambulatory peritoneal dialysis (CAPD) at study entry, an additional hemodialysis is performed on Day −1.

### Fludarabine Administration (Days −6, −5, −4, and −3):

Fludarabine (10 mg/m²) can be administered intravenously over approximately 30 minutes on Days −6, −5, −4, −3. Use of anti-emetic therapy (e.g., granisetron or alternate) per local institutional standards to prevent nausea and vomiting is recommended while following fludarabine labeling and precautions.

### Cyclophosphamide Premedication/Treatment:

Prior to administration of cyclophosphamide on Days −5 and −4, the recipient can receive pretreatment for nausea, vomiting and infusion reaction prophylaxis (e.g., a glucocorticoid, antihistamine, anxiolytic and antiemetic). Subjects can undergo prophylaxis against hemorrhagic cystitis with MESNA as follows:
Oliguric subjects: MESNA, 15 mg/kg administered via 4 bolus intravenous injections 15 minutes before, and 3, 6 and 9 hours after cyclophosphamide administration.
Non-oliguric subjects: MESNA 15 mg/kg administered via 4 bolus intravenous injections 15 minutes before, and 3, 6 and 9 hours after cyclophosphamide administration, plus IV hydration (e.g. with 5% dextrose, 1500 mL/M2/24 hrs as tolerated) beginning 4 hours prior to cyclophosphamide. If the subject is oliguric, bladder washout can also be performed.

### Cyclophosphamide Administration (Days −5 and −4):

Subjects receive cyclophosphamide, 22.5 mg/kg/day (based on lesser of ideal or actual body weight). Cyclophosphamide is dissolved in 250 mL 5% dextrose and infused over 1 hour via intravenous infusion. Administration of cyclophosphamide can occur at least 20 hours following hemodialysis.

### Thymic Irradiation (Day −1):

7 Gy of thymic irradiation is administered in a single dose on Day −1. A field size and dose rate is done per institutional's standards.

### Donor Nephrectomy and Bone Marrow Procurement:

the donor undergoes nephrectomy and bone marrow cell procurement under general anesthesia on Day 0 (day of transplant). Unfractionated donor bone marrow cells (intended minimum of 2×10⁸ TNC/kg of recipient body weight) are prepared for infusion according to the below:
Prior to procurement, recipient weight is used to calculate a target number of cells based on 2×10⁸ TNC/kg for the donor collection. A midpoint collection evaluation of the number of TNCs collected can also be performed to assess the progress of collection.
Marrow are taken from each posterior iliac crest using needle/trocar sets and aspirating marrow into heparinized syringes with a maximum of 5-10 mL of marrow per aspiration. Repeated aspirations are then performed by reinserting the needle through the same skin puncture site into different bone sites until approximately 20-33% of the intended volume is collected. Skin puncture sites are then changed, and the aspirations are repeated. With this method, three to five skin puncture sites overlying each iliac crest are utilized.
Prior to completion of Bone Marrow Procurement a TNC count can be performed; if additional cells are required, they can be obtained, provided institutional guidelines for BM volume are not exceeded.
Once sufficient cells are obtained the Bone Marrow are filtered to remove fat, bone and clots.
Anticoagulant can be added to the Bone Marrow extract to prevent clotting (e.g., heparin or anticoagulant citrate-dextrose or according to local practice). In order to minimize the effect of the anticoagulant at the time of infusion (given the risk of intra- and post-operative bleeding), the marrow is plasma depleted and partially resuspended in saline.
The plasma is removed by transferring the marrow product into standard blood transfer pack containers, centrifuging, and expressing the supernatant plasma with a plasma extractor into another transfer pack container.
Intravenous 0.9% sodium chloride in a volume equivalent to approximately one half of the volume of the expressed plasma is then added to the Bone Marrow Cells. This also lowers the volume of the infusion.
The Bone Marrow cell sample are characterized as to the total volume, total nucleated cell content, total CD3+ T-cell content, and total CD34+ cell content.
The final Bone Marrow sample can be stored in a cooler or as appropriate for transport back to the operating room and infusion into the recipient.

### Recipient Transplantation and Bone Marrow Cell Infusion:

On study Day 0, transplant recipients receive the allograft. A wedge or needle baseline biopsy is obtained from the allograft pre-implantation. Following revascularization of the allograft and upon confirmation that no bleeding or leakage from the vascular anastomoses are present, the recipient receive the bone marrow cell infusion. The cells are infused intravenously, at a rate of approximately 300-500 mL/hr. The infusion can begin no later than 4 hours following reperfusion.

A partial thromboplastin time is measured half-way through the infusion (or if bleeding seems excessive) and following completion of the infusion. Protamine 25 mg can be given intravenously for a PTT of >60 seconds or for an elevated PTT if that is believed to be the cause of bleeding.

### Concomitant Immunosuppression Medication can Include:

Immediate release tacrolimus (TAC): 0.5 mg, 1.0 mg, or 5.0 mg capsules or IV; Sirolimus (SRL): 0.5, 1.0 or 2.0 mg tablets or oral solution; and Corticosteroids (CS): oral and IV administration.

### Tacrolimus Administration (Day 0 to Week 4):

TAC is administered as capsules or tablets orally (p.o.) twice a day (BID) and adjusted to maintain serum trough (C0) concentrations within the target range of 8-10 ng/mL. TAC is discontinued at Week 4, after subjects are switched to SRL and the SRL trough level is in the range 5-8 ng/ml. The switch can be performed earlier, if needed (e.g., BK infection). If oral administration is not feasible or practical, IV TAC containing the equivalent of 5 mg/mL tacrolimus administration by continuous intravenous infusion can be substituted per label. Once a day TAC is not permitted.

TAC can be started as soon as possible in the peri-transplant period and is initiated no later than 24 hours after reperfusion of the allograft. The lowest permitted dosing of TAC in this study is 0.5 mg BID or IV equivalent. Subjects who discontinue their study regimen are expected to remain in the study on the local standard of care until Month 12.

TAC dosing is modified as needed. In the event of TAC intolerance (e.g., nephrotoxicity, neurotoxicity) dose reduction of TAC can be necessary.

### Sirolimus Administration (Week 4 to Month 12):

Sirolimus is administered p.o. once per day from Week 4, replacing tacrolimus, with a starting dose of 3 mg/d, with dose adjustment and therapeutic drug monitoring with an initial target trough concentration of 5-8 ng/mL. During this period, daily troughs can be done for TAC and SRL and once the SRL target is reached, TAC can be stopped.

In case of severe SRL side effects (e.g., severe neutropenia, thrombocytopenia, oral ulcers) immunosuppression may be switched back to TAC and weaning can be carried out according to the schedule below as the target concentration ranges are the same for both TAC and SRL.

### Sirolimus Weaning (Starting at Month 6): Sirolimus Weaning Criteria

1. Stable renal function (sCr <2.0 mg/dL) under immunosuppression, unless a transient rise in creatinine is related to an alternative cause of renal dysfunction.
2. Detectable multilineage white blood cell chimerism (any level) in the early post-transplant period.
3. No current or prior DSA.
4. No current evidence of GvHD.
5. No evidence of antibody mediated rejection (ABMR) or Banff grade IA or greater T-cell mediated rejection (TCMR) on the Month 6 (or most recent) renal biopsy. History of borderline changes do not disqualify a participant from complete immunosuppression withdrawal, provided that the biopsy result is normal at Month 12.

A biopsy is repeated at Month 12 and if the weaning criteria continue to be met and there remains no evidence of rejection, SRL may be stopped completely.

### Sirolimus Weaning Schedule:

The schedule below is a guideline and dose reduction can be delayed or paused based on the overall clinical considerations; trough concentration measurement throughout SRL dose reduction.
Month 6-9: SRL trough level target of 3-5 ng/ml
Month 9-12: SRL trough level target of 1-3 ng/ml

The co-administration of drugs known to strongly inhibit or induce CYP3A4/P-gp are known to decrease or increase SRL concentrations and should be avoided if possible. If these drugs are required, the physician has to carefully monitor SRL trough concentrations and adjust them accordingly during and after the use of the interacting treatment.

### Corticosteroid Administration:

Corticosteroids (CS) are given at several times in the study:
Premedication: rituximab-hydrocortisone sodium succinate (SoluCortef; 100 mg IV) forpremedication prior to rituximab (Days −9 and −2)
Premedication: an anti-CD2 antibody or antigen binding fragment thereof (e.g., siplizumab)-methylprednisolone sodium succinate (SoluMedrol; 8 mg/kgIV) as first dose premedication for an anti-CD2 antibody or antigen binding fragment thereof (e.g., siplizumab) (Day −1)
Transplant immunosuppression:
   IV methylprednisolone sodium succinate (SoluMedrol): 500 mg Days 0 and +1 then taper from Day 2; 250 mg, Day 3; 125 mg, Day 4; 75 mg, Days 5-7; 60 mg.
   Steroid route of administration can be switched to from IV to oral on or around Day 4.
   16 mg po bid to Day 14
   8 mg po bid to Day 28
   4 mg po bid to Month 2
   4 mg po qd until Month 6 when steroids are stopped.

If CTS is observed steroids can be considered in first line treatment. If rejection is observed steroids can be used as first line treatment.

### Background IS

Immunosuppression
Tacrolimus, perioperative start Day 0 to Day 30; (target trough 8-10 ng/mL),
Sirolimus: Day 30 through Month 12 post-transplant (initial target trough 5-8 ng/ml) (Note: Transition may occur earlier based on BK viral load and/or serum creatinine)
Steroids: an IV bolus on the day of transplant (Day 0) and post-operative Day 1; tapered and withdrawn over 6 months with oral steroids.

### Safety Assessments:

AEs and SAEs
Clinical chemistry, hematology, vital signs, and serology
Renal function

### Data Analysis:

The primary objective is calculated based on incidence. Other efficacy objectives are similarly analyzed. AE/SAE data is coded and displayed by SoC and preferred term with incidence. Lab data is analyzed by visit with summary statistics and change from baseline.

### Sample Size Determination:

approximately 12 subjects/patients are enrolled. Additional patients can be enrolled depending on the primary objective and safety profile.

### Study Duration:

the primary endpoint is assessed at Month 24, with all recipients enrolled followed to Month 60.

### Additional Procedure: Renal Biopsies:

a wedge or needle biopsy is performed on the explanted allograft prior to transplant. All subsequent biopsies are performed percutaneously under ultrasound guidance at Month 6 and 12 and as needed in cases of suspected rejection. Biopsies are performed in subjects with platelet counts >50,000 mL.

### Additional Procedure: Hemodialysis:

hemodialysis is performed twice, e.g., on Days −6 and −3, to mitigate against the risks of fludarabine and cyclophosphamide toxicity

### Infectious Prophylaxis:

CMV Prophylaxis: Cytomegalovirus (CMV) prophylaxis is central to the successful outcome of BMCI and organ transplantation and CMV anti-viral prophylaxis is adjusted to each subject's expected risk of viral activation. All donor and recipient subjects have their pre-transplant CMV serologic status obtained at Screening. All subjects can receive sero-negative or CMV “Safe” blood (leukofiltered). The greatest risk for CMV infection in solid organ transplantation is for the donor seropositive (D+) into recipient seronegative (R−) combination. For CMV positive combinations (D+/R−, D−R+ and D+/R+) subjects:
have weekly monitoring by quantitative CMV DNA NAAT assay while neutropenic and following engraftment
receive ganciclovir, or the locally approved CMV prophylaxis regimen. This is initiated prior to renal transplantation (day −1) until 6 months post-transplant. During neutropenia, in CMV+ recipients, ganciclovir should not be used and letermovir should be considered for up to 100 days as available.

Provided CMV pedigreed blood products are utilized, there is essentially no risk of CMV disease in the D−R− population, other than via sexual contacts and no additional prophylaxis or treatment is required.

### Human Herpes Virus (HHV):

All subjects receive acyclovir or famciclovir for HHV infection prophylaxis for at least 3 months, or according to standard guidelines and local practice.

Pneumocystis jirovecii (Pneumocystis carinii) Pneumonia (PCP):

Transplant recipients receive levofloxacin, 500 mg daily, beginning on Day −9. This prophylaxis is interrupted, starting on Day 0 (day of transplant) and continue until the neutrophil count exceeds 500 cells/μl. This prophylaxis may be interrupted briefly in the peri-operative period if participants are unable to take oral medications.

After the first month, following normalization of hematologic status, subjects can be switched to sulfamethoxazole and trimethoprim (SMZ-TMP), one single strength tablet daily, and continue through 6 months post-transplant. Consideration is given to extending the period of prophylaxis for an additional 6 months if it is recommended. In the event of allergy or intolerance to the components of SMZ-TMP, atovaquone and/or inhaled pentamidine may be used in its place for PCP prophylaxis.

### Hepatitis B Virus (HBV):

Prophylaxis for HBV reactivation (e.g. Hepatitis B Immunoglobulin; HBIg) during the course of this study are administered at the discretion of the physician.

### Fungal/Yeast Infection Prophylaxis:

Fungal/yeast prophylaxis is followed. For example, itraconazole or fluconazole is started on transplant Day 1 and continued for two months or at least until resolution of neutropenia. For oral thrush (Candida), Nystatin can be used in a swish and swallow regimen; alternatively, clotrimazole (Mycelex®) lozenges/troches can be used. Attention should be paid to the interaction of azole antifungal agents which may increase blood concentrations of TAC and SRL.

### Immunization:

Immunization of transplant candidates for vaccine preventable diseases is recommended more than 2 weeks prior to transplantation or starting at 1-6 months after transplantation. If given prior to transplantation, the full immunization series should be completed before the transplant procedure. In certain situations, it may be appropriate to wait until 3 or more months after transplantation to vaccinate, such as following T- or B-cell depletion therapy.

### Treatment of Acute Rejection Episodes:

In all suspected acute rejection episodes, regardless of initiation of anti-rejection treatment, a renal biopsy is performed within 48 hours. Acute rejections are treated with bolus methylprednisolone (other CS are acceptable at an equivalent dose). Recommended treatment is at least 3 boluses of IV methylprednisolone with a minimal dose of 250 mg/bolus or at least 2 boluses of IV methylprednisolone with a minimal total dose of 750 mg.

Subjects who experience steroid-resistant rejections, vascular rejections, or rejections with a Banff grade 2′ 2B can be treated with other anti-rejection therapies (i.e., antibody therapy).

### Diagnosis, Treatment of Chimeric Transition Syndrome (CTS):

The diagnosis of CTS is made by the physician based on the overall clinical presentation of the subject which can include fluid retention, fever, loss of or reduction in chimerism, and increased serum creatinine.

A diagnosis of CTS is made following biopsy confirmation and the exclusion of alternative causalities for renal dysfunction. CTS is reported as a Serious Adverse Event, and designated per the Severity criteria outlined below:
Mild: elevation of up to 2× baseline sCr
Moderate: >2 and <4× baseline sCr
Severe: >4× baseline sCr OR need for any dialysis

Suspected CTS is any increase in sCr >2× baseline, where baseline is the lowest sCr observed post-transplant. In a case where renal function has not recovered (e.g. delayed graft function (DGF)) ultrasound is first used to evaluate blood flow to the graft with any DGF treated.

Treatment for CTS is initiated without biopsy per physician's judgment and may include pulse steroids, and dialysis. IV methylprednisolone sodium succinate at 500 mg for 2-3 days, depending on CTS symptoms (renal function) can be considered for first line therapy. When second-line therapy is required, 8 mg/kg of tocilizumab can be considered to mitigate inflammatory responses caused by IL-6, as employed in the setting of cell therapy and stem cell transplantation. In the case of rapid deterioration of renal function in combination of reduced renal blood flow, plasma exchange can be considered.

### Management of CMV:

The standard of care for treating symptomatic CMV disease is a minimum of 2-3 weeks of intravenous ganciclovir (with dosage adjustment recommended for renal dysfunction per Table 9), oral valganciclovir, or other approved antiviral medication per local practice. CMV IgG can be added for seronegative subjects with viral activation. Intravenous therapy is discontinued once clearance of the virus from the blood occurs, as demonstrated by CMV antigenemia assay (or quantitative assay).

In subjects with relapsing infection, CMV IgG can be administered in conjunction with the intravenous ganciclovir therapy in sero-negative recipients. Treatment is maintained in the setting of active GvHD.

TABLE 9
Ganciclovir dosing guidance in patients with reduced renal function

| CrCl(ml/min) | Intravenous dose (mg/kg) | Frequency |
|---|---|---|
| >70 | 5 | Q12 h |
| 50-69 | 2.5 | Q12 |
| 25-49 | 2.5 | Daily |
| 10-24 | 1.25 | Daily |
| <10 (Hemodialysis) | 1.25 three times a week | Post-dialysis |
| <10 (Peritoneal | *2.5 | Daily |
| dialysis) | | |

*After a loading dose of 5 mg/kg. All subjects (prophylaxis or therapeutic) receive leukocyte or CMV-negative blood; CMV-hyperimmune globulin 150 mg/kg IV for first dose and 100 mg/kg × 4 at the discretion of the physician.

### Management of BK Viremia:

BK Polyoma virus (BKV) screening utilizes plasma (or whole blood) viral load (VL) molecular assays. A urinary test for BKV (cytology for decoy cells or urine BKV loads over 7-log geq/mL) is adequate for screening; if negative, the risk for polyoma virus associated nephropathy (PVAN) is low.

Quantitative cutoffs for presumptive diagnosis of BKV nephropathy include plasma DNA VL >10,000 copies/mL (whole blood polymerase chain reaction (PCR) VL >1500-3500 copies/mL), urine VP1 mRNA load >6.5×10⁵ copies/ng total RNA, or urine DNA load >10e7 copies/mL; higher viral loads are increasingly predictive of PVAN. Biopsy is suggested for confirmation if creatinine is elevated. Renal histopathology provides definitive diagnosis of PVAN.

### Management of Delayed Graft Function (DGF):

DGF for this trial is defined as the need for dialysis performed within 7 days of transplant. In case a subject experiences DGF, the DGF is by definition starting at reperfusion after the transplantation procedure. If the graft dysfunction is starting later according to the investigator, then this condition is considered secondary graft dysfunction. DGF treatments maintain sufficient immunological coverage for the graft and can include maintaining, interrupting, or reducing the dose of study treatment and the use of ATG. If a polyclonal antibody or ATG is used prior to Day 4, the investigational product is discontinued, and the subject is placed on SoC.

### Primary Graft Non-Function (PGNF):

PGNF is defined as dialysis starting after transplantation with a continuous record of post-transplant dialysis until either transplantectomy, retransplantation, or death.

### EBV-PTLD:

In patients diagnosed with EBV-PLTD, therapy is individualized. An anti-CD2 antibody or antigen binding fragment thereof (e.g., siplizumab) doses planned for this trial can result in T- and NK-cell depletion and minimal to no modulation of the B-cell compartment, respectively. However, B-cell compartment coverage can be from concomitant, peri-transplant rituximab. Clinicians can refer to the anti-CD2 antibody or antigen binding fragment thereof (e.g., siplizumab) IB when considering therapeutic options for the treatment of EBV-PTLD.

### Treatment of GvHD (Acute or Chronic):

Acute GvHD is a serious possible complication following bone marrow cell infusion. In case of acute GvHD, the applicable treatment guidelines, typically using high dose steroids as first line therapy, can be followed. Calcineurin inhibitor therapy can continue if the patient is already receiving it. Second line treatment is variable and depends on the severity of the GvHD and organ(s) involved but may include ruxolitinib, mycophenolate mofetil and/or sirolimus. Treatment of chronic GvHD is also variable but is generally steroid based to which second line, glucocorticoid sparing agents such as ibrutinib or ruxolitinib may be added.

### Conditioning Regimen (Day −9 to Day 1) Day −9 (Outpatient)

Following confirmation of all eligibility criteria during screening, including a negative SARS-CoV-2 test performed within 72 hours of Day −9 (recipient and donor), the donor/recipient pair are enrolled into the study and the following completed for recipient subjects before their first conditioning regimen dose:
Vital signs
Review new or changes to existing medications and/or adverse events
Blood collection for local lab chimerism assessment
Central lab blood collection for immunophenotyping (FACS) and serum cytokines
Initiate levofloxacin (infection prophylaxis)

The conditioning regimen commences with the recipient receiving the first dose of rituximab (+premedication) on Day −9. Vital signs are captured pre-dose, immediately post-infusion and 1-hour following the end of infusion.

### Day −6:

Starting on Day −6, recipient subjects are hospitalized and remain domiciled for the remainder of the conditioning regimen, until discharge following transplantation. The following procedures are collected and/or performed on Day −6:
Vital signs
Review of new or changes to existing medications and/or adverse events.
Blood collection for CBC and chemistry
Confirmation and/or placement of vascular access for hemodialysis
Fludarabine administration
   Hemodialysis 5-7 hours after completion of fludarabine infusion and prior to cyclophosphamide administration on Day −5

### Day −5:

Review new or changes to existing medications and/or adverse events
Blood collection for CBC, chemistry, and coagulation panels
Initiation of PCP prophylaxis treatment
Fludarabine administration
MESNA for hemorrhagic cystitis prophylaxis (prn)
Cyclophosphamide pre-medication administration
Cyclophosphamide administration

### Day −4:

Review new or changes to existing medications and/or adverse events
Blood collection for CBC and chemistry
Fludarabine administration
MESNA for hemorrhagic cystitis prophylaxis (prn)
Cyclophosphamide pre-medication administration
Cyclophosphamide administration

### Day −3:

Review new or changes to existing medications and/or adverse events
Blood collection for CBC and chemistry
Fludarabine administration
   Hemodialysis 5-7 hours after completion of fludarabine infusion

### Day −2

Review new or changes to existing medications and/or adverse events
Lymphocyte/CDC crossmatch (completed prior to transplantation).

Blood collection for CBC and chemistry
Central Lab blood collection for immunogenicity (anti-CD2 antibodies) and CD2RO
Rituximab pre-medications
Rituximab infusion
   Vital signs captured pre-dose, immediately post-infusion and 1-hour following theend of infusion

### Day −1

Review new or changes to existing medications and/or adverse events
Lymphocyte/CDC crossmatch
Blood collection for CBC and chemistry
Central lab blood collection for serum cytokines and anti-CD2 antibody or antigen binding fragment thereof (e.g., siplizumab) concentrations (PK)
Thymic irradiation
Anti-CD2 antibody or antigen binding fragment thereof (e.g., siplizumab) administration
   Pre-medications administered 30 minutes to 3 hours prior to infusion
   PK sample collected pre-dose and 1-hour post end of an anti-CD2 antibody or antigen binding fragment thereof (e.g., siplizumab) infusion
   Vital signs are captured pre-dose, immediately post-infusion and 1-hour following the end of infusion

### Day 0 (Day of Transplant)

Review new or changes to existing medications and/or adverse events
Interrupt PCP prophylaxis as of Day 0
Blood collection for CBC and chemistry (daily while hospitalized; other timepoints asclinically indicated)
Blood collection for coagulation panel
CMV DNA NAAT (weekly monitoring while neutropenic)
Prophylactic antimicrobial treatment prior to surgery
Tacrolimus administration (initiated no later than 24 hours post-transplant)
Corticosteroid administration
Donor Surgical Procedures
   Nephrectomy
   Bone marrow procurement
   Preparation of donor cells
Recipient Surgical Procedures
   Allograft wedge/needle biopsy
   PK sample collected PRE-DOSE
   Anti-CD2 antibody or antigen binding fragment thereof (e.g., siplizumab) pre-medication administration (with anesthesiologist consultation)
   Anti-CD2 antibody or antigen binding fragment thereof (e.g., siplizumab) infusion
   Vital signs captured pre-dose, immediately post-infusion and 1-hour following the end of anti-CD2 antibody or antigen binding fragment thereof (e.g., siplizumab) infusion
   Renal transplant
   Infusion of donor bone marrow cells
   PK sample (collected pre-dose and 1-hour post end of anti-CD2 antibody or antigen binding fragment thereof (e.g., siplizumab) infusion)
   PTT (once infusion of cells is 50% complete)

### Day 1:

Review new and changes to medication and any adverse events
Vital signs
Blood collection for CBC, chemistry and coagulation panel
Corticosteroid administration
Anti-CD2 antibody or antigen binding fragment thereof (e.g., siplizumab) administration (final dose)
   Pre-medications administered 30 minutes to 3 hours prior to infusion
   PK sample collected pre-dose and 1-hr post end of anti-CD2 antibody or antigen binding fragment thereof (e.g., siplizumab) infusion
   Vital signs captured pre-dose, immediately post-infusion and 1-hour following the end of infusion

### Immunosuppression Period (Day 1 to Month 12):

The Conditioning Period effectively ends with the final administration of an anti-CD2 antibody or antigen binding fragment thereof (e.g., siplizumab) and when subjects initiate or continue immunosuppression treatment with tacrolimus (Day 0) and corticosteroids (Day 2).

In addition to the procedures noted above, and also starting on Day 1, subjects initiate broad infection prophylaxis against fungal/yeast, CMV and HSV infection. Also during this period, and while hospitalized, the subject has daily CBC and Comprehensive Metabolic Panel (Chemistry) assessments, weekly CMV DNA NAAT surveillance, and assessments for graft loss, rejection, and GvHD.

Subjects are administered tacrolimus through Month 1 at which time tacrolimus are stopped and sirolimus starts 12 hours later. Sirolimus and corticosteroids continue until Month 6 at which point weaning of immunosuppression commences. Corticosteroids are stopped at Month 6 and sirolimus are tapered over 6 months to complete withdrawal at Month 12 post-transplant.

Subjects remain hospitalized, treated, and subsequently discharged. Following discharge, recipient subjects return for weekly clinic visits through Week 12 (Month 3) and then monthly visits through Month 12. All donor subjects have a telehealth assessment at 4 weeks post-nephrectomy to assess general well-being and to assess for any adverse events.

### Immunosuppression Free Period (Month 12 to Month 60):

Following the Month 12 visit, subjects return for clinic visits on a quarterly basis through Month 24 for overall health and laboratory assessments and for evaluation of adverse events. Month 24 is the study endpoint and a critical visit. Following completion of the Month 24 visit, subjects enter a longer term follow-up period and have bi-yearly visits until they return for the final study visit at Month 60.

### Efficacy Assessments:

The assessment of success in withdrawal of immunosuppression as the primary endpoint relies on a composite of renal function as measured by the changes in serum creatinine and eGFR (MDRD and CKD-EPI) over time as well as incidence and severity of acute rejection on per protocol and for-cause biopsies. In addition to standard safety assessments, the incidence of graft loss, death and lost to follow-up are included as a key secondary composite endpoint

### Electrocardiogram (ECG):

A standard 12-lead ECG is conducted with the subject in a supine position at screening, Month 24 and end of study. The ECG is evaluated for potential contraindication for transplantation at screening and or changes from baseline at the Month 24 and Month 60 visits. The Fridericia QT correction formula (QTcF) can be used for clinical decisions.

### Laboratory Evaluations:

The following laboratory assessments are performed:

TABLE 10
Laboratory Assessments

| Laboratory Panel | Analytes |
|---|---|
| Complete Blood | RBC |
| Count (CBC) | Hemoglobin |
| with differential | Hematocrit |
| | MCV |
| | MCH |
| | WBC |
| | Differential (% and abs) |
| | Platelet Count |
| Coagulation | INR/prothrombin time (PT) |
| Studies | aPTT |
| Comprehensive | Albumin |
| Metabolic | BUN |
| Panel | Total protein |
| | Creatinine |
| | Sodium |
| | ALT/SGPT |
| | Potassium |
| | AST/SGOT |
| | Bicarbonate (C02) |
| | Alkaline Phosphatase |
| | Chloride |
| | Bilirubin |
| | Calcium |
| | Glucose |
| Lipid Panel | Cholesterol |
| | Triglycerides |
| | LDH/HDL |
| Thyroid Panel | T3/T4/TSH |
| Urinalysis | Leukocytes |
| | Specific Gravity |
| | Nitrates |
| | Ketones |
| | Urobilinogen |
| | Bilirubin |
| | Protein |
| | Glucose |
| | pH |
| | Microscopic, as indicated |
| | Blood |
| Pregnancy testing | A negative serum pregnancy test for females |
| | of childbearing potential required prior to |
| | Day −9. |
| Donor Specific | Blood samples (antibodies directed against |
| (DSA) Antibodies | antigens expressed on donor organs) |
| Immunogenicity | Blood samples to determine the presence of |
| | anti-CD2 antibodies, using a bridging |
| | Enzyme-linked Immunosorbent assay |
| | (ELISA)-based assay |

### CMV DNA NAAT:

All subjects have quantitative CMV viral load measured locally by a NAAT-based method, preferably calibrated in IU/mL. No specific viral load cutoffs are proposed for the initiation of antiviral therapy. However, persistent low-level viremia (<2500 IU) suggests excess immunosuppression or stimulation by other infections or processes (e.g. rejection).

### EBV DNA PCR:

All subjects have quantitative EBV viral load measured in serum (or whole blood) by a standardized PCR-based method. All measurements are conducted by a laboratory utilizing World Health Organization (WHO) EBV international reference standards. There are currently no consensus guidelines on thresholds for EBV DNAemia or viral load in adult renal transplant recipients; therefore, any PCR positive result that is increasing from baseline or a previous assessment should be flagged to the investigator for local follow-up per protocol, including a full physical and neurological exam with careful attention to the liver, spleen, allograft and lymph nodes. In addition, ad hoc abdominal and allograft ultrasound to screen for signs and symptoms of potential PTLD lesions are conducted.

### EBV-PTLD Surveillance:

Clinical manifestations of EBV infection range from asymptomatic infection to clinically significant and potentially life-threatening disease in transplant recipients. EBV infection can be either primary (new infection occurring in an immunologically naïve subject) or secondary due to either reactivation of latent EBV in the transplant recipient under the pressure of immune suppression or reinfection with a new EBV strain. In general, secondary infection tends to be mild or even asymptomatic. Histologic evaluation is important in defining disease status of a subject with suspected PTLD; manifestations can evolve in individual subjects. The World Health Organization has provided standardized criteria for the pathologic evaluation of lesions associated with EBV in solid organ transplant recipients.

The following signs and symptoms can guide the clinician in assessing the risk of, and for, EBV-PTLD.

TABLE 11
EBV Signs and Symptoms

| | |
|---|---|
| Signs: | Pallor |
| | Lymphadenopathy |
| | Subcutaneous nodules |
| | Tonsillar enlargement/inflammation |
| | Hepatosplenomegaly |
| | Focal neurologic signs |
| | Mass lesions found on imaging obtained for |
| | other reasons |
| Constitutional and | Unexplained fever or night sweats |
| Systemic | Malaise |
| Symptoms: | Weight loss and/or anorexia |
| | Sore throat |
| | Swollen glands |
| | Headache or focal neurological symptoms |
| Allograft-specific | Liver: jaundice, abdominal pain |
| symptoms: | Intestine: abdominal pain, gastrointestinal |
| | bleeding, nausea and vomiting |
| | Heart/Lung: shortness of breath, cough, |
| | decreased lung function (lung alone) |
| | Renal: kidney dysfunction |
| Lab Tests: | Serial increase in EBV viral load from |
| | peripheral blood |

TABLE 12
Diagnostic Evaluation

| | |
|---|---|
| Routine | CBC with |
| Laboratory | differential |
| Assessments: | Platelets |
| | Serum electrolytes (inc. |
| | BUN/creatinine, calcium) |
| | Liver Function Test |
| | Uric acid |
| | Lactate dehydrogenase |
| | Quantitative immunoglobulins |
| | EBV serologies (anti-Epstein-Barr nuclear antigen |
| | (EBNA),viral capsid antigen (VCA) and early |
| | antigen (EA) |
| | EBV viral load from peripheral blood |
| | Stool: occult bleeding |
| Routine | Chest radiograph (anteroposterior and lateral) |
| Procedures: | CT or PET scan of neck/chest/abdomen/pelvis |
| | Core needle or excisional biopsy of lesion(s) with flow |
| | cytometry of lymphocytes (when feasible) |
| | EBER, CD20 histochemistry studies of pathologic |
| | samples |
| Procedures | Bone marrow biopsy |
| based on | Bone scan |
| S/Sx: | Brain CT/MRI |
| | Gastrointestinal endoscopy |
| | Lumbar puncture |
| | Ultrasound (abdomen and allograft) |
| Lab Tests: | Serial increase in EBV viral load from peripheral blood |

### Imaging: PTLD Surveillance:

When warranted based on physical examination findings and/or EBV surveillance, subjects can have an ultrasound performed of the abdominal cavity and allograft to rule out nodal and/or extra-nodal EBV-LPD lesions. CT, MRI and/or PET imaging can be considered for staging and monitoring of biopsy confirmed PTLD.

### Renal Biopsy:

A renal biopsy is performed, on Day 0 and at the 6 and 12 Month Visits. Biopsies are read by the local pathologist according to local practice and the 2018 Banff criteria.

Additionally, for all suspected rejection episodes, regardless of initiation of anti-rejection treatment, a renal biopsy must be performed within 48 hrs. The results are used for subject management for acute rejection.

### Treated Biopsy Proven Acute Rejection:

Treated biopsy proven acute rejection (tBPAR) is any condition in which the subject receives anti-rejection treatment and is histologically diagnosed as acute rejection according to the 2018 Banff criteria, including borderline rejections.

### Graft-versus-Host Disease (GvHD):

GvHD assessments are carried out regularly according to the visit schedule and Acute GvHD Assessment. For surveillance for acute GvHD in patients with persistent chimerism (beyond Month 3) additional weekly visits should be performed to check LFTs (for liver GvHD), rash for skin GvHD and stool volume for GI GvHD, if diarrhea is present. Biopsies of skin, gut and/or liver is performed as clinically indicated to confirm a diagnosis of GvHD. Acute GvHD is diagnosed, Staged and Graded (I-IV) according to current hematology/oncology practice guidelines. Similarly, chronic GvHD is assessed according to applicable consensus guidelines for diagnosis and staging of chronic GvHD.

Organ Staging of Acute Graft-vs.-Host Disease

| | | Liver | | | |
|---|---|---|---|---|---|
| Stage | Skin | (bilirubin) | | Upper GI | Lower GI Gut |
| 0 | No active (erythematous) | <2 | mg/dL^{b} | No or intermittent | <500 mL/day |
| | GvHD rash | | | nausea, vomiting | or <3 |
| | | | | or anorexia | episodes/day |
| 1 | Maculopapular rash, <25% | 2-3 | mg/dL^{b} | Persistent | 500-999 mL |
| | BSA^{a} | | | nausea, | diarrhea/day^{c} |
| | | | | vomiting or | |
| | | | | anorexia | |
| 2 | Maculopapular rash, 25- | 3.1-6 | mg/dL^{b} | | 1000-1500 mL |
| | 50% BSA^{a} | | | | diarrhea/day^{c} |
| 3 | Maculopapular rash, > | 6.1-15 | mg/dL^{b} | | >1500 mL |
| | 50% BSA^{a} | | | | diarrhea/day^{c} |
| 4 | Generalized | >15 | mg/dL^{b} | | Severe abdominal pain |
| | erythroderma (>50% | | | | with or without ileus |
| | BSA) plus bullous | | | | |
| | formation and | | | | |
| | desquamation >5% BSA | | | | |

^{a}BSA = Body surface area, use “Rule of Nines” to determine extent of rash.
^{b}Range given as total bilirubin. Downgrade one stage if an additional cause of elevated bilirubin has been documented.
^{c}Downgrade one stage if an additional cause of elevated stool output has been documented.

Overall Clinical Grading of Acute GVHD

| Grade | Degree of Organ Involvement |
|---|---|
| 0 | No stage 1-4 of any organ |
| I | Stage 1-2 rash and no liver, lower GI or upper GI involvement |
| II | Stage 3 rash and/or stage 1 liver and/or stage 1 upper GI and/or |
| | stage 1 lower GI. |
| III | Stage 2-3 liver and/or stage 2-3 lower GI, with stage |
| | 0-3 skin and/or stage 0-1 upper GI. |
| IV | Stage 4 skin, liver, or lower GI involvement, with stage 0-1 |
| | upper GI. |

### Graft Loss:

The allograft is presumed to be lost on the day the subject starts dialysis and is not able to subsequently be removed from dialysis. If the subject undergoes allograft nephrectomy prior to starting permanent dialysis, then the day of nephrectomy is the day of graft loss.

### Pharmacodynamic (PD) Assessments:

All PD blood samples are taken by either direct venipuncture or an indwelling cannula.

### Immunophenotyping (FACS):

The effect of an anti-CD2 antibody or antigen binding fragment thereof (e.g., siplizumab) and conditioning on circulating leukocytes and T-cells are determined using flow cytometry FACS. T-cell recovery, including naive and memory-type CD4 and CD8 cells and other subsets, as well as B cell subsets, NK cell, dendritic cell subsets are monitored. Cell subpopulations that are analyzed may include, but are not limited to, those bearing one or more of the following cell surface or intracellular markers such as: CD2, CD3, CD4, CD5, CD8, CD19, CD25, CD27, CD38, CD45RA, CD45RO, CD59, CD127, CD138, CD154, FoxP3, and HLA-DR.

### CD2 Receptor Occupancy:

Peripheral CD2 receptor occupancy by an anti-CD2 antibody or antigen binding fragment thereof (e.g., siplizumab) is determined by flow cytometry analysis, measuring free or total CD2 receptors on T-cells.

### Chimerism Assessment:

The nature and level of donor hematopoietic chimerism in the recipient's bone marrow post-transplantation are monitored.

Flow cytometric analysis of blood after the donor cell infusion are performed for the assessment of chimerism. Testing of percent chimerism can be done for multiple lineages. These analyses can be performed to determine distinct donor/recipient profiles. The frequencies of recipient and donor-derived leukocytes and their subsets are measured by FACS, until donor chimerism (via donor MHC class I expression) becomes undetectable. The frequencies of recipient leukocytes and their subsets recovered in the blood of patients using FACS are evaluated every month after transplantation through Month 12 and then quarterly through Month 24.

Microsatellite analysis of recipient PBMC using PCR based genomic DNA amplification of variable short tandem repeats (VNTR) is used to distinguish donor/recipient profiles. This assay is generally capable of detecting approximately 0.1% donor-type cells. Assays on separated PBMC cell fractions (T-cells: when present at >30 cells/mm3, B-cells: when present at >30 mm3, granulocytes and monocytes) are used to assess chimerism in different lineages.

### Pharmacokinetic (PK) Assessments:

Pharmacokinetic (e.g., siplizumab and anti-siplizumab antibodies) samples are obtained and evaluated in all subjects at all dose levels. The timing of the PK sample collection can be altered based on emergent data.

Anti-CD2 antibody or antigen binding fragment thereof (e.g., siplizumab) concentrations are determined by a validated ELISA method; the anticipated Lower Limit of Quantification (LLOQ) is 10 ng/mL and concentrations are expressed in mass per volume units.

The following PK parameters are determined using the actual recorded sampling times and non-compartmental method(s): Cmax, Tmax, area under the curve (AUC) last, AUCinf, T1/2, Vz/F and CL/F from the plasma concentration-time data.

The linear trapezoidal method is used for AUC calculation. Regression analysis of the terminal plasma elimination phase for the determination of T1/2 includes at least 3 data points after Cmax. If the adjusted R2 value of the regression analysis of the terminal phase is less than 0.75, no values are reported for T1/2, AUCinf and CL.

### Assessment of Treatment Exposure and Compliance:

PK parameters (measures of treatment exposure) are determined in all subjects treated with study treatment. TAC and SRL trough concentrations are determined locally. The local trough values are used to adjust the TAC and SRL dosing as needed to achieve the time-dependent target concentrations.

### Exploratory Biomarker Assessments

### Renal Injury Biomarkers

Urine is collected, frozen, and banked pending a potential assessment of renal biomarkers including but not limited to KIM-1, NGAL/Lipocalin-2, a-GST, Cystatin C, Clusterin, total protein, albumin, and beta2-microglobulin.

### Cytokine Analysis:

Serum blood samples are collected during the study for cytokine assessment via multiplex cytokine (or similar) platform (e.g., IL-1b, IL-2, IL-4, IL-5, IL-6, IL-8, IL-10, IL-12, IL-13, IL-17, IFN-gamma, TGF-beta, TNF-alpha) and analyzed.

### Updated 2018 Banff Classification:

Category 1: Normal biopsy or nonspecific changes
Category 2: Antibody-mediated changes.
Active ABMR: all three features must be present for diagnosis. Biopsies showing histological features plus evidence of current/recent antibody interaction with vascular endothelium or DSA, but not both, may be designated as suspicious for acute/active ABMR. Lesions may be clinically acute or smoldering or may be subclinical; it should be noted if the lesion is C4d-positive or C4d-negative, based on the following criteria:
1. Histologic evidence of acute tissue injury, including one or more of the following:
   Microvascular inflammation (g >0 in the absence of recurrent or de novo glomerulonephritis, and/or ptc >0)
   Intimal or transmural arteritis (v>0)1 ¹ It should be noted that these arterial lesions may be indicative of ABMR, TCMR, or mixed ABMR/TCMR. The v lesions areonly scored in arteries having a continuous media with two or more smooth muscle layers.
   Acute thrombotic microangiopathy in the absence of any other cause
   Acute tubular injury in the absence of any other apparent cause
2. Evidence of current/recent antibody interaction with vascular endothelium, including at least one of the following:
   Linear C4d staining in peritubular capillaries (C4d2 or C4d3 by IF on frozen sections or C4d >0 by IHC on paraffin sections)
   At least moderate microvascular inflammation ([g+ptc] 2′2), although in the presence of acute TCMR, borderline infiltrate, or infection; ptc 2′2 alone is not sufficient, and g must be 2′1
   Increased expression of gene transcripts in the biopsy tissue strongly associated with ABMR
3. Serologic evidence of DSAs (HLA or other antigens)
C4d staining or expression of validated transcripts/classifiers as noted above in criterion 2 may substitute for DSA; however thorough DSA testing, including testing for non-HLA antibodies if HLA antibody testing is negative, is strongly advised whenever criteria 1 and 2 are met
Chronic active ABMR²: all three features must be present for diagnosis. As with active ABMR, biopsies showing histological features plus evidence of current/recent antibody interaction with vascular endothelium or DSA, but not both, may be designated as suspicious, and it should be noted if the lesion is C4d-positive or C4d-negative, based on the criteria listed: ² Lesions of chronic, active ABMR can range from primarily active lesions with early TG evident only by EM (cg1a) to those with advanced TG and other chronic changes in addition to active microvascular inflammation. In the absence of evidence of current/recent antibody interaction with the endothelium, the term “active” should be omitted; in such cases, DSAs may be present at the time of biopsy or at any previous time after transplantation.
1. Morphologic evidence of chronic tissue injury, including one or more of the following:
   TG (cg >0), if no evidence of chronic thrombotic microangiopathy or chronic recurrent/de novo glomerulonephritis; includes changes evident by electron microscopy (EM) alone (cg1a)
   Severe peritubular capillary basement membrane multilayering (requires EM)³ ³ Seven or more layers in one cortical peritubular capillary and five or more in two additional capillaries, avoiding portions cut tangentially.
   Arterial intimal fibrosis of new onset, excluding other causes; leukocytes within thesclerotic intima favor chronic ABMR if there is no prior history of biopsy-proven TCMR with arterial involvement but are not required
2. Identical to criterion 3 for active ABMR, above,
3. Identical to criterion 3 for active ABMR, above, including strong recommendation for DSA testing whenever criteria 1 and 2 are met
   C4d Staining without Evidence of Rejection
All three features must be present for diagnosis⁴ ⁴ The clinical significance of these findings may be quite different in grafts exposed to anti-blood group antibodies (ABO-incompatible allografts), in which they do not appear to be injurious to the graft and may represent accommodation; however, with anti-HLA antibodies, such lesions may progress to chronic ABMR and more outcome data are needed.
1. Linear C4d staining in peritubular capillaries (C4d2 or C4d3 by IF on frozen sections, or C4d >0 by IHC on paraffin sections)
2. Criterion 1 for active or chronic, active ABMR not met.
3. No molecular evidence for ABMR as in criterion 2 for active and chronic, active ABMR
4. No acute or chronic active TCMR, or borderline changes

### Category 3: Borderline Changes.

### Suspicious for Acute TCMR

Foci of tubulitis (t>0) with minor interstitial inflammation (i0 or i1) or interstitial inflammation (i2, i3) with mild (t1) tubulitis; retaining the i1 threshold for borderlinewith t>0 is permitted although this must be made transparent in reports and publications
No intimal or transmural arteritis (v=0)

### Category 4: TCMR

### Acute TCMR

Grade IA. Interstitial inflammation (>25% of nonsclerotic cortical parenchyma, i2 or i3) and foci of moderate tubulitis (t2) involving 1 or more tubules, not including tubules that are severely atrophic⁵ ⁵ A severely atrophic tubule is defined as one with each of the following 3 features: a diameter <25% of that of unaffected or minimally affected tubules on the biopsy, an undifferentiated-appearing, cuboidal or flattened epithelium, and pronounced wrinkling and/or thickening of the tubular basement membrane
Grade IB. Interstitial inflammation involving >25% of nonsclerotic cortical parenchyma (12 or i3) with severe tubulitis (t3) involving 1 or more tubules, not including tubules that are severely atrophic⁵
Grade IIA. Mild to moderate intimal arteritis (v1) with or without interstitial inflammation and tubulitis
Grade IIB. Severe intimal arteritis (v2), with or without interstitial inflammation and/or tubulitis
Grade III. Transmural arteritis and/or arterial fibrinoid necrosis of medial smooth muscle cells with accompanying mononuclear cell intimal arteritis (v3), with or without interstitial inflammation and/or tubulitis

### Chronic Active TCMR

Grade IA: Interstitial inflammation involving >25% of the total cortex (ti score 2 or3) and >25% of the sclerotic cortical parenchyma (i-IFTA score 2 or 3) with moderate tubulitis (t2) involving 1 or more tubules, not including severely atrophic tubules5; other known causes of i-IFTA should be ruled out
Grade IB: Interstitial inflammation involving >25% of the total cortex (ti score 2 or 3) and >25% of the sclerotic cortical parenchyma (i-IFTA score 2 or 3) with severe tubulitis (t3) involving 1 or more tubules, not including severely atrophic tubules5; other known causes of i-IFTA should be ruled out
Grade II: Chronic allograft arteriopathy (arterial intimal fibrosis with mononuclear cell inflammation in fibrosis and formation of neointima)

### Category 5: IFTA

Grade I (Mild): Banff Lesion Score ci1 OR Banff Lesion Score ct1
Grade II (Moderate) Banff Lesion Score ci2 OR Banff Lesion Score ct2
Grade III (Severe) Banff Lesion Score ci3 OR Banff Lesion Score ct3

### Category 6: Other Changes not Considered to be Caused by Acute or Chronic Rejection

BK-Virus Nephropathy
Posttransplant Lymphoproliferative Disorder
Calcineurin Inhibitor Toxicity
Acute Tubular Injury
Recurrent Disease
De Novo Glomerulopathy (Other Than TG)
Pyelonephritis
Drug-Induced Interstitial Nephritis

### Legend:

ABMR, antibody-mediated rejection; cg, glomerular double contours; DSA, donor-specific antibody; EM, electron microscopy; g, glomerulitis; i, inflammation; IF, immunofluorescence; IHC, immunohistochemistry; ptc, peritubular capillaritis; t, tubulitis; TCMR, T-cell mediated rejection; TG, transplant glomerulopathy; TMA, thrombotic microangiopathy; v, intimal arteritis.

### 6.2 Example 2. An Adaptive, Regimen Finding, Safety and Tolerability Study of an Anti-CD2 Antibody or Antigen Binding Fragment Thereof (e.g., Siplizumab), Combined with Donor Bone Marrow Cell Infusion and Non-Myeloablative Conditioning, for Tolerance Induction in De Novo Living Donor Renal Transplantation

This study was designed to identify and optimize an allogeneic tolerance-inducing regimen that mitigates Chimeric Transition Syndrome (CTS) symptoms while maintaining the benefits of complete immunosuppression withdrawal. CTS is an early event characterized by a rise in serum creatinine and transient renal dysfunction that has been associated with the rapid loss of donor chimerism.

### Primary Objective:

To determine the optimal regimen for renal allograft tolerance (proportion of recipients off immunosuppression with maintenance of good renal function at Month 24 post-transplant) while minimizing Chimeric Transition Syndrome (CTS).

### Key Secondary Objectives:

To determine the composite incidence of biopsy proven acute rejection (BPAR), death and graft loss (with and without loss to follow up) at Month 24 post-transplant and to determine the incidence and mean fluorescence intensity (MFI) of de novo DSA, at Month 24 post-transplant

### Secondary Objectives:

Incidence, duration, grade of Chimeric Transition Syndrome (CTS)
Proportion of recipients with transient mixed chimerism without CTS
Proportion of recipients off immunosuppression for at least 12 Months
Incidence of BPAR and treated BPAR (Banff classification)
Incidence of de-novo Donor-specific antibody
Incidence of death and graft loss
Incidence of chronic rejection
Renal function (eGFR) and rate of change of renal function over time
Incidence of serious adverse events and adverse events while on study
Incidence of severe or clinically significant opportunistic infections
Incidence of BK viremia, infection and nephropathy
Incidence of malignancies
Incidence and severity of GvHD
Incidence of new onset diabetes post-transplant (NODAT)

TABLE 13
Primary and Secondary Objectives and Related Endpoints

| Objective(s) | Endpoints |
|---|---|
| Primary objective | Endpoints for primary objective |
| To determine the optimal regimen | Immunosuppression dosing |
| for renal allograft tolerance | Serum creatinine (eGFR) |
| (proportion of recipients off | (Serius) adverse events (AE/SAEs) |
| immunosuppression with | Renal biopsy |
| maintenance of good renal | |
| function at Month 24 post- | |
| transplant) while minimizing | |
| chimeric transition syndrome | |
| Key Secondary Objectives | Endpoints for key secondary objective |
| To determine the composite | Incidence of BPAR |
| incidence of biopsy proven acute | Patient/graft survival and disposition |
| rejection (BPAR), death and graft | Incidence of de novo DSA |
| loss (with and without loss to | |
| follow-up) at Month 24 post- | |
| transplant | |
| To determine the incidence and | |
| mean fluorescence intensity | |
| (MFI) of de novo donor-specific | |
| antibody (DSA) at Month 24 | |
| post-transplant | |
| Secondary Objectives | Endpoints for secondary objectives |
| To evaluate the safety, tolerability | AE/SAEs, clinical laboratory data |
| and activity of an anti-CD2 | |
| antibody or antigen binding | |
| fragment thereof (e.g., siplizumab) | |
| and fludarabine-based | |
| conditioning regimen over time | |
| Secondary Objective(s) | Endpoints for secondary objectives |
| Incidence, duration, severity of | CTS (AE/SAE) |
| Chimeric Transition Syndrome | Serum creatine, AEs |
| CTS | Immunosuppression dosing |
| Proportion of recipients with | Patient/graft survival |
| transient mixed chimerism without | Renal biopsy, immunosuppression |
| CTS | dosing |
| Proportion of recipients off | Incidence of DSA |
| immunosuppression for at least 12 | Patient/graft survival |
| Months | AE/SAEs, biopsy, indicence of |
| Incidence of BPAR and treated | chronic rejection |
| BPAR (with Banff classification) | eGFR and change in eGFR |
| Incidence of de-novo donor- | AE/SAE |
| specific antibody | AE/SAE infections system organ |
| Incidence of death and graft loss | class (SOC) |
| Incidence of chronic rejection | Incidence of BK AE events, biopsy |
| Renal function (eGFR) and change | AE/SAE malignancy/neoplasms SOC |
| of eGFR over time | GvHD assessment, AE/SAEs |
| Incidence of serious adverse | Diabetes AE, lab data, concomitant |
| events and adverse events | use of |
| Incidence of severe and clinically | anti-diabetic medications |
| significant opportunistic infections | |
| Incidence of BK viremia, infection | |
| and nephropathy | |
| Incidence of malignancies | |
| Incidence and severity of GvHD | |
| Incidence of new onset diabetes | |
| post-transplant (NODAT) | |

### Exploratory Objectives:

Assessment of lymphocyte depletion and recovery, including Tregs and T effector/memory cells, quantified by flow cytometry
Time to neutrophil recovery following transplant (ANC >500/mm3)
Time to platelet recovery following transplant (platelets >20,000/mm3 and transfusion independent)
Characterize the pharmacokinetics of an anti-CD2 antibody or antigen binding fragment thereof (e.g., siplizumab)
Assessment of donor-specific lymphoid and myeloid chimerism by: Flow cytometry and VNTR PCR-based DNA microsatellite analyses
Incidence or progression of abnormal histologic findings of cellular or antibody mediated rejection (e.g., TG, IF/TA, C4d, BK/Polyoma virus nephropathy)
Assessment and safety monitoring of the donor for hematologic and renal function, and other complications of living donation
Evaluation of cytokine panel over time
Evaluation of HLA eplet mismatch load and relationship to outcomes

TABLE 14
Exploratory Objectives and Related Endpoints

| Exploratory Objectives | Endpoints for exploratory objectives |
|---|---|
| Assessment of lymphocyte | FACS analysis of lymphocyte |
| depletion and recovery, including | subsets |
| Tregs and T effector/memory | Absolute neutrophil count (ANC) in |
| cells, quantified by flow | hematology |
| cytometry) | Platelets in hematology |
| Time to neutrophil recovery | |
| following transplant (ANC > | |
| 500/mm3) | |
| Time to platelet recovery | |
| following transplant (platelets > | |
| 20,000/mm3 and transfusion | |
| independent) | |
| Characterize the pharmacokinetics | Anti-CD2 antibody or antigen |
| of an anti-CD2 antibody or | binding fragment thereof (c.g., |
| antigen binding fragment thereof | siplizumab) pharmacokinetic (PK)/ |
| (e.g., siplizumab) | anti-drug antibody (ADA) Analysis |
| Assessment of donor-specific | Chimerism by flow and polymerase |
| lymphoid and myeloid chimerism | chain reaction (PCR) |
| by: | |
| Flow cytometry | |
| Variable Short Tandem | |
| Repeats Polymerase Chain | |
| Reaction (VNTR PCR)- | |
| based DNA microsatellite | |
| analyses | |
| Incidence or progression of | Renal biopsy data and histology |
| abnormal histologic findings of | |
| cellular or antibody mediated | |
| rejection (e.g., TG, IF/TA, C4d, | |
| BK/Polyoma virus nephropathy | |
| Follow-up assessment and safety | Donor follow-up data |
| monitoring of the donor for | |
| hematologic and renal function, | |
| and other complications of living | |
| donation | |
| Evaluation of cytokine panel over | Cytokine panel by visit |
| time | |
| Evaluation of human leukocyte | HLA typing and analysis by |
| antigen (HLA) eplet mismatch | eplet mismatch. Outcomes; |
| load and relationship to outcomes | BPAR, Off |
| | immunosuppression, patient |
| | and graft survival, donor |
| | specific antibodies (DSA) |

### Study Design:

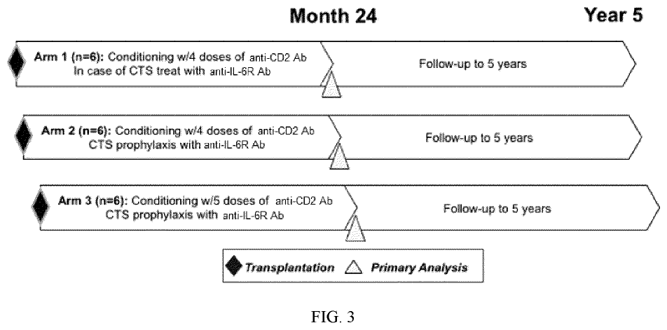

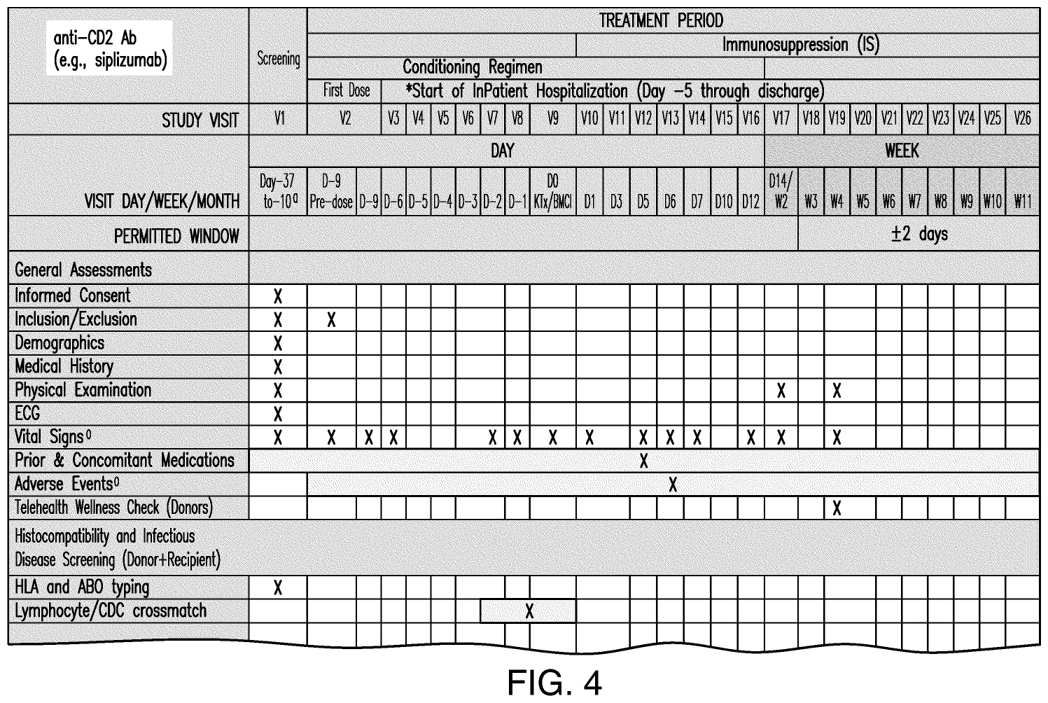

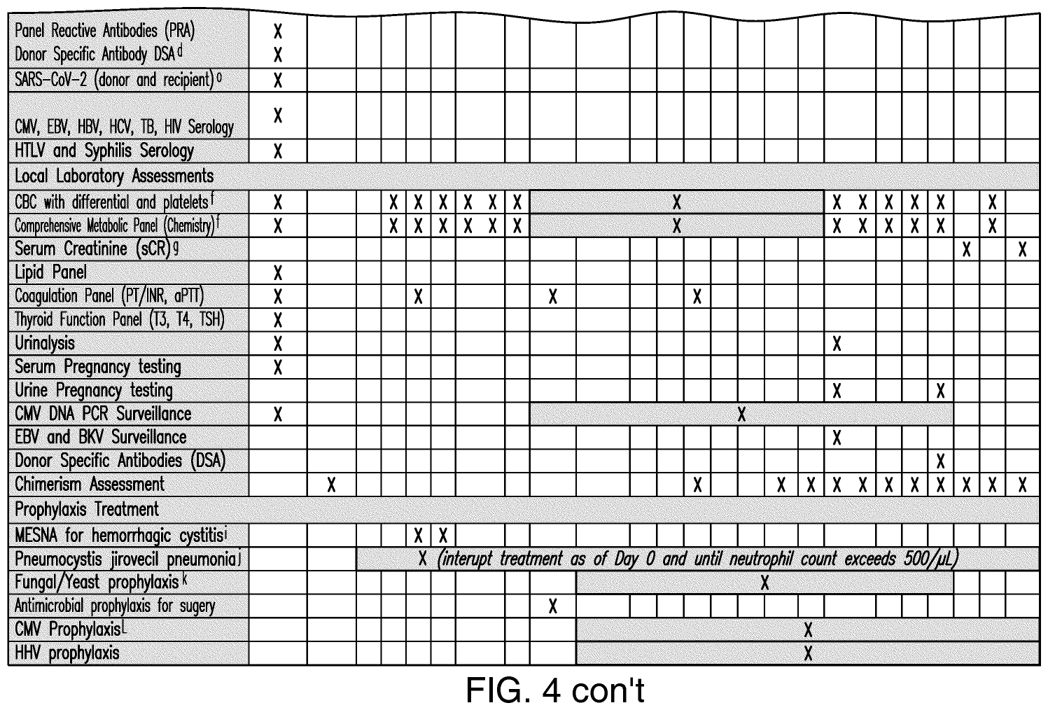

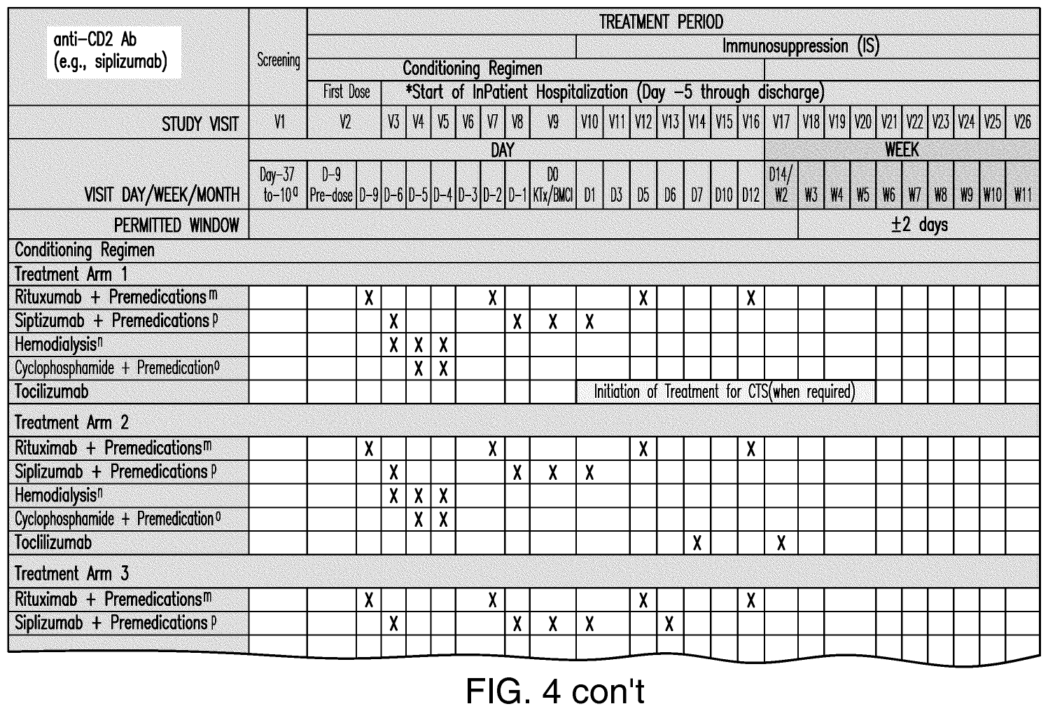

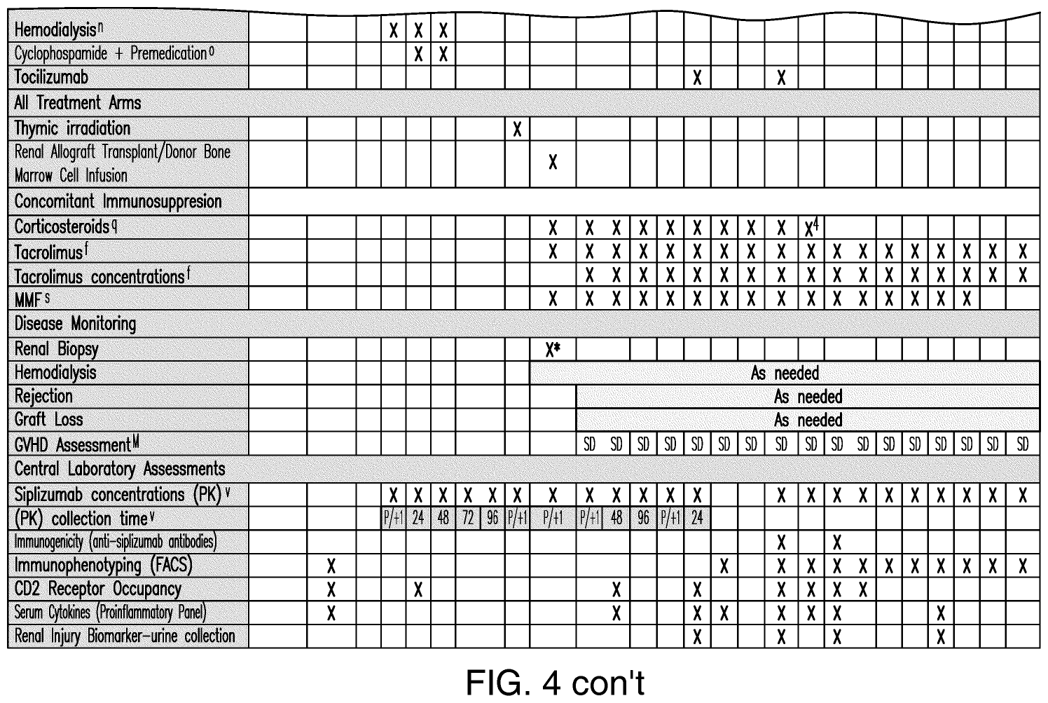

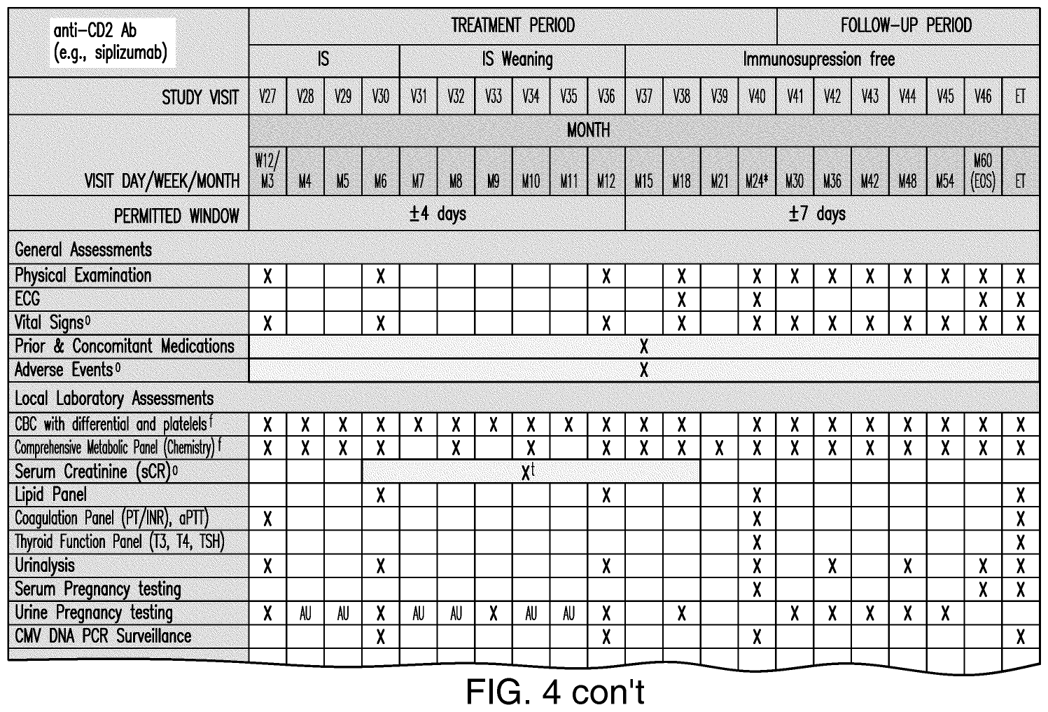

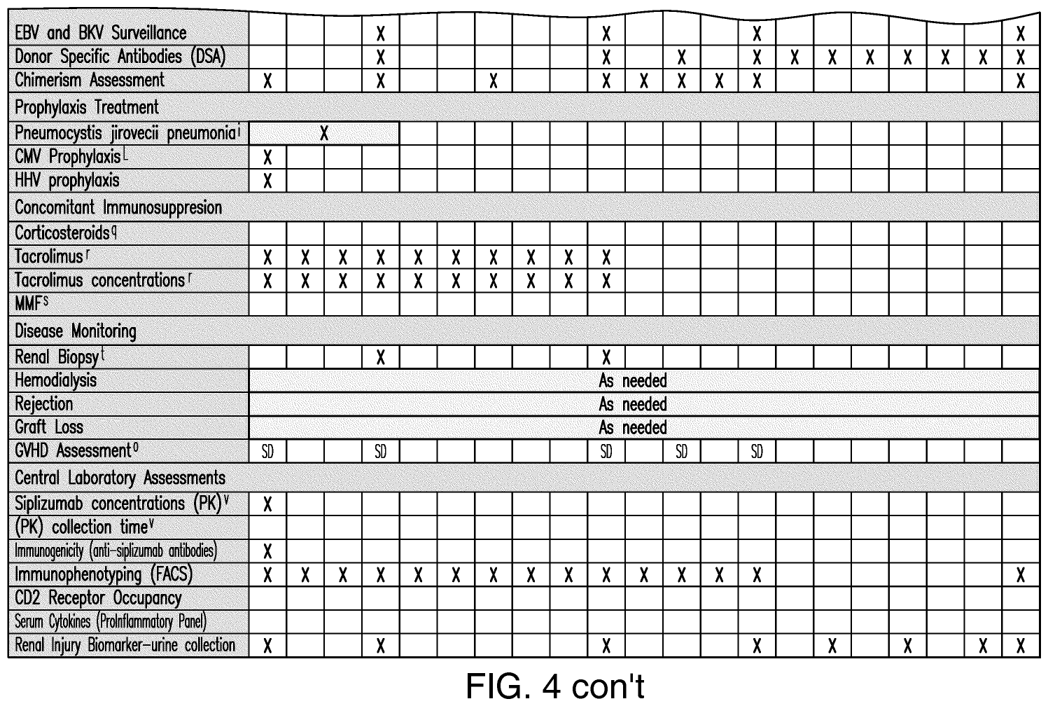

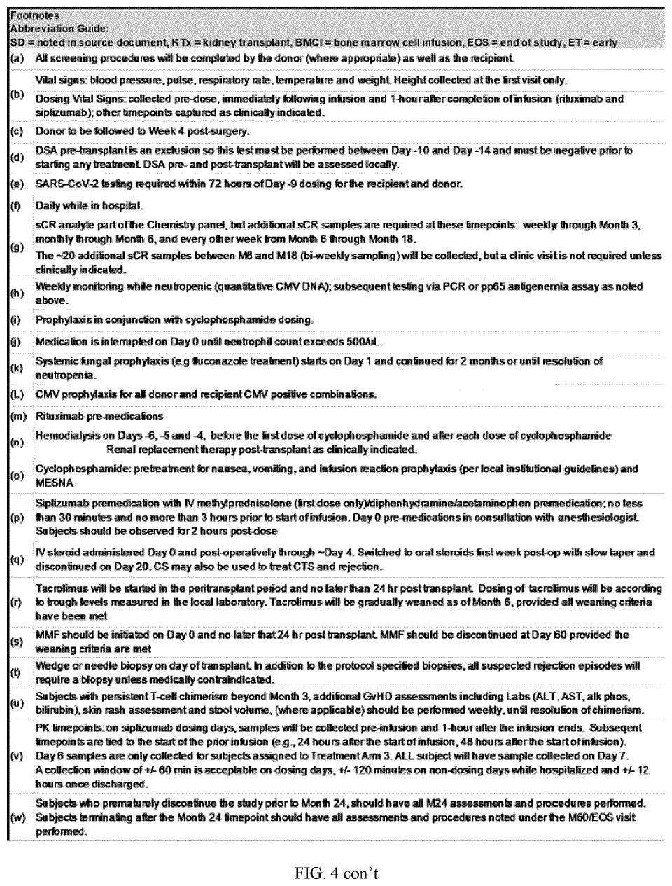

This 60-month adaptive study evaluates up to three anti-CD2 antibody or antigen binding fragment thereof (e.g., siplizumab)-based treatment regimens in a sequential manner. Up to 6 transplant recipients are planned in each arm of the study (FIG. 3). The study is designed with the primary endpoint at 24 months; thereafter recipients are followed with visits every 6 months until the end of study at 5 years post-transplant. The five-year study is designed with a primary endpoint at 24 months and twice-yearly assessments thereafter. Refer to FIG. 4 for the schedule of assessment.

Recipients and their respective donors who complete the screening period and meet all inclusion/exclusion criteria are enrolled in the study and begin the treatment period. Pre-transplant screening procedures include donor/recipient HLA typing (molecular), complement-dependent cytotoxicity (CDC) lymphocyte crossmatch (or virtual crossmatch), qualitative DSA by single antigen bead (SAB) assay, and donor/recipient viral serology. Recipient conditioning are initiated on Day −9 from their scheduled transplant date. The conditioning regimen includes rituximab (375 mg/m²) on Days −9, −2, 5 and 12; Cyclophosphamide (60 mg/kg) on Days −5, and −4; Thymic Irradiation (7 Gy) on Day −1; anti-CD2 antibody or antigen binding fragment thereof (e.g., siplizumab) (0.6 mg/kg) on Days −6, −1, 0 and 1 (and Day +6 for Arm 3 only); and combined kidney and unfractionated bone marrow cell infusion (2-3×10⁸ cells/kg) on Day 0. The donor undergoes simultaneous nephrectomy and iliac crest bone marrow aspiration under general anesthesia. Sufficient marrow is obtained to provide at least 2-3×10⁸ nucleated cells per kilogram weight of the recipient.

A short course of corticosteroids is administered initially as an IV bolus on Day 0, tapered down to oral dosing, and then stopped at Day 20.

Recipients in all treatment arms receive concentration-controlled background immunosuppression, peri-transplant, which includes, tacrolimus (4-11 ng/ml target trough) and mycophenolate mofetil at 2 g/d. These medications, including corticosteroid (CS) are weaned and stopped. MMF is discontinued at Week 8 without any down titration. Tacrolimus weaning is initiated between Month 6-9, provided the patient continues to meet the weaning criteria (including stable renal function with serum creatinine (sCr)<2.0 mg/dL and no rejection on the Month 6 protocol biopsy). It is intended that the discontinuation of tacrolimus (TAC) occurs between Month 9 and 12 at which time eligible subjects are immunosuppression-free.

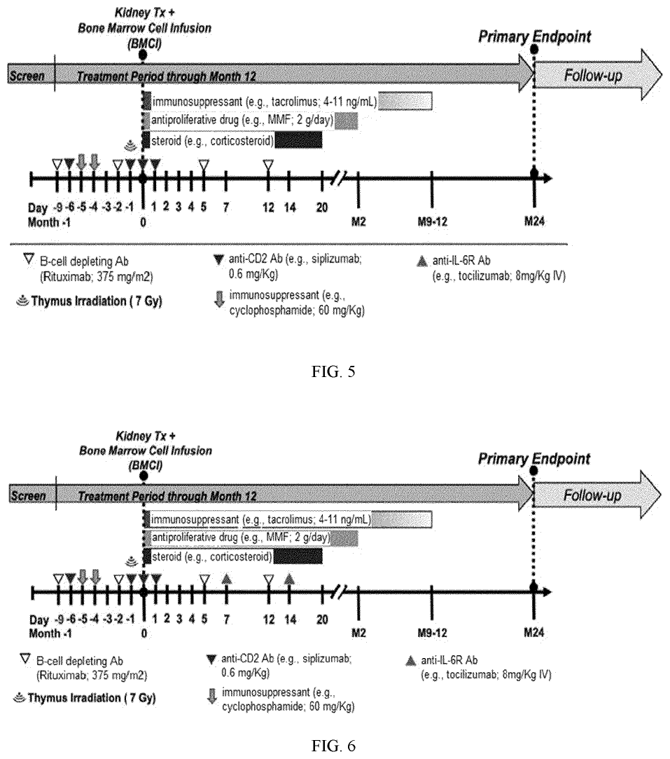

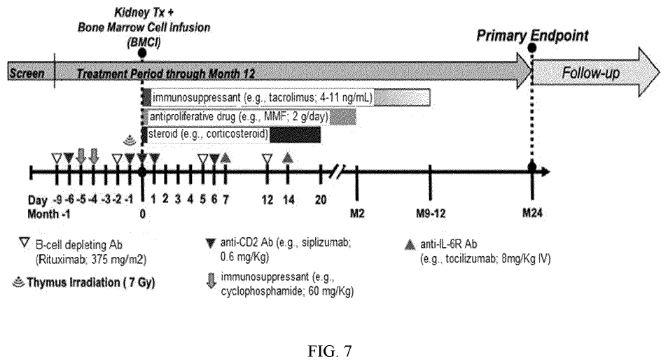

Tocilizumab is administered as treatment, or prophylaxis against CTS. Subjects across all 3 arms receive the same background conditioning and immunosuppression, with tocilizumab being administered in Arm 1 only if CTS presents and as prophylaxis in Arms 2 and 3. Additionally, subjects in Arm 3 receive one additional dose of an anti-CD2 antibody or antigen binding fragment thereof (e.g., siplizumab). Background immunosuppression in all three arms include concentration-controlled tacrolimus (trough target: 4-11 ng/ml) for the first 9-12 months and mycophenolate mofetil (MMF; 2 g/d) to Week 8. The study arms are as follows, all having the same background of conditioning and immunosuppression as described above:
Arm 1: anti-CD2 antibody or antigen binding fragment thereof (e.g., siplizumab): −6, −1, 0, 1; anti-IL-6R antibody (e.g., tocilizumab) treatment only If CTS occurs (FIG. 5)
Arm 2: anti-CD2 antibody or antigen binding fragment thereof (e.g., siplizumab): −6, −1, 0, 1; Prophylactic anti-IL-6R antibody (e.g., tocilizumab) day 7 and day 14 (FIG. 6)
Arm 3: anti-CD2 antibody or antigen binding fragment thereof (e.g., siplizumab): −6, −1, 0, 1, 6; Prophylactic anti-IL-6R antibody (e.g., tocilizumab) day 7 and day 14 (FIG. 7)

Subjects are enrolled following a 3+3 sequential Arm design scheme where the safety and tolerability of the regimen in one arm are assessed in 3 subjects and if acceptable an additional 3 subjects are enrolled. Starting with Arm 1, the first 3 treated patients are enrolled sequentially. Safety at Week 4, including the grade of CTS, must be acceptable prior to enrollment of the next subject. This time-lagged design continues through enrollment and treatment of the third subject in a given Arm. Once safety has been assessed as satisfactory 1-month post-transplant the other 3 subjects can be enrolled simultaneously: without any delay. Otherwise, the study proceed to the next treatment Arm where enrollment follows the same time-lagged 3+3 design.

If any patients in a given Arm present with moderate/severe CTS that Arm is closed to further recruitment and the study progresses to the next Arm. If all three Arms were required and each was completely enrolled, then 18 recipients and their respective donors, are enrolled.

Immunosuppression is weaned off according to per-protocol weaning criteria with MMF stopped at post-transplant Week 8. Tacrolimus weaning is started from Month 6-9, provided the subject continues to meet the weaning criteria (i.e., stable renal function with SCr <2.0 mg/dL and no BPAR on the Month 6 protocol biopsy). It is intended for subjects to completely discontinue tacrolimus between post-transplant Month 9 to 12. A further cohort of patients with higher levels of HLA mismatching than in these initial Arms, which will be at least haploidentical, can be implemented.

### Population:

up to 6 transplant recipients (and their respective donors) are enrolled in each arm of the study. If all three arms are required, then 18 recipients and 18 respective donors are enrolled

### Key Inclusion Criteria for Recipient:

1. Able to understand the study requirements and provide written informed consent before any study assessment performed
2. Recipients aged 18 to 65 years
3. Recipients undergoing their first renal transplant
4. Recipient of a renal transplant from a non-HLA identical, but at least haploidentical, ABO compatible living donor
5. Capable of complying with the schedule of study visits, especially after discontinuation of immunosuppression
6. Subjects with stable cardio-pulmonary status per the judgement of the investigator, with ejection fraction >40% within 3 months of transplant, and eligible for transplantation
7. EBV seropositive within 6 months of screening.
8. Negative SARS-CoV-2 nucleic acid amplification test (NAAT) within 72 hours of Day −9 (Start of Treatment Period).
9. Recipient free from any other locally endemic infections that would be contraindications to solid organ or BMT/HSCT (Bone Marrow Transplantation/Hematopoietic Stem Cell Transplantation) transplantation according toapplicable guidelines.
10. Male study subjects willing to maintain barrier contraception (condom) and agree not to father a child until 90 days after the last dose of MMF.

### Key Inclusion Criteria for Donor Subjects:

1. Donor subject willing and able to provide informed consent prior to any study assessments being performed
2. Subjects aged 18 to 65 years
3. Be in excellent health per conventional pre-donor history (medical, laboratory and psychosocial evaluation)
4. Negative for Hepatitis B Surface Antigen (HBsAg), Human Immunodeficiency Virus (HIV), Hepatitis C Virus (HCV) (RNA) and Human T-cell Leukemia Virus Type 1 (HTLV-1). Viral test results within 28 days of Day 0 (planned date of surgery) are acceptable.
5. Negative SARS-CoV-2 NAAT within 72 hours of Study Day −9 (Start of Conditioning regimen for recipient).
6. Willingness to adhere to COVID protocols (e.g., social distancing, mask usage) and institutional guidelines from Day −9 through Day of surgery.
7. Negative for latent tuberculosis (TB) infection as detected by Quantiferon Gold Plus Interferon Gamma Release Assay (IGRA) (or current standard interferon gamma release assay for TB).
8. Free from any other locally endemic infections that would be contraindications to solid organ or BMT/HSCT transplantation according to applicable guidelines.

### Key Exclusion Criteria for Recipient Subjects:

1. Use of other investigational drugs (or enrollment in another investigational drug study) within 30 days of screening or 5 half-lives of the medication, whichever is longer
2. History of hypersensitivity to any of the study treatments or its excipients or to drugs of similar chemical classes (e.g., MEDI-507, tacrolimus, MMF, cyclophosphamide or rituximab)
3. Recipient with end stage renal disease due to primary focal segmental glomerulosclerosis (FSGS) or membranoproliferative glomerulonephritis (MPGN/C3 glomerulopathy)
4. Recipient with any donor specific anti-HLA antibody (DSA) as measured by singleantigen bead (SAB) assay within 28 days prior to transplant
5. Recipient with a positive donor cross-match result (assayed according to local practice) within 28 days prior to transplant
6. Recipient with any panel reactive antibodies (PRA >20%) within two months prior to transplant.
7. Subjects with leukopenia (white blood cell (WBC) less than 2,000/mm3) or thrombocytopenia (platelet count <100,000/mm3) at baseline
8. Sero-positive for HIV-1 or HBsAg. Subjects who are sero-positive for Hepatitis C virus are excluded without proof of sustained viral response (SVR) after anti-HCV treatment (viral test results within 6 months of transplant are acceptable)
9. Subjects with latent TB infection as detected by Quantiferon Gold Plus IGRA (or current standard interferon gamma release assay for TB)
10. Subjects with a history of cancer other than basal cell carcinoma of the skin or carcinoma in situ of the cervix
11. Subjects with clinically significant laboratory abnormality that would preclude participation in the study [(e.g., >2.5× Upper Limit Normal (ULN) values for (a) liver chemistries (Alanine Aminotransferase (ALT), Aspartate Aminotransferase (AST), Alkaline Phosphatase (ALP)), (b) bilirubin, (c) coagulation studies (Prothrombin Time (PT), Activated Partial Thromboplastin Time (aPTT))].
12. Subjects who, in the opinion of the investigator/physician, are not capable of giving informed consent for the study or who are unable or unwilling to adhere to the study requirement outlined in the protocol
13. Subjects with any other clinically significant medical condition or laboratory abnormality that would, in the judgment of the investigator interfere with the subject's ability to participate in the study
14. Subjects who have received any live-attenuated vaccine within 2 months of planned transplant
15. Pregnant or nursing (lactating) women, where pregnancy is defined as the state of a female after conception and until the termination of gestation, confirmed by a positive human chorionic gonadotropin (hCG) laboratory test

Investigational Product: anti-CD2 antibody or antigen binding fragment thereof (e.g., siplizumab)

### Conditioning Regimen

Anti-CD2 antibody or antigen binding fragment thereof (e.g., siplizumab)
cyclophosphamide (60 mg/kg)
rituximab (375 mg/m²/dose)
thymic irradiation (TI, 7 Gy)
+/−tocilizumab (8 mg/kg IV)

### Background IS

tacrolimus (target trough 4-11 ng/mL)
mycophenolate mofetil (1.0 g by mouth (po) twice a day (BID))
corticosteroids

### Treatment Regimen:

The planned duration of treatment with an anti-CD2 antibody or antigen binding fragment thereof (e.g., siplizumab) is 4 doses over a period of 8 days for those assigned to Arm 1 or 2 and 5 doses over 13 days for subjects assigned to Arm 3. All subjects will receive an anti-CD2 antibody or antigen binding fragment thereof (e.g., siplizumab) on Days −6, −1, 0, and 1 with an additional dose provided to subjects in Arm 3 on Day 6. Prior to starting the conditioning regimen (Day −9) eligible subjects are assigned to one of 3 treatment groups as follows depending on the status of the study:
Arm 1: an anti-CD2 antibody or antigen binding fragment thereof (e.g., siplizumab) (Days −6, −1, 0, 1)+tocilizumab treatment if CTS occurs
Arm 2: an anti-CD2 antibody or antigen binding fragment thereof (e.g., siplizumab) (Days −6, −1, 0, 1)+prophylactic tocilizumab d7, d14
Arm 3: an anti-CD2 antibody or antigen binding fragment thereof (e.g., siplizumab) (Days −6, −1, 0, 1, 6)+prophylactic tocilizumab d7, d14

### Concomitant Treatments:

Conditioning (including TI and cyclophosphamide, with MESNA and dialysis) is as herein. Recipients receive a conditioning regimen starting with rituximab on Day −9 (and Days −2, 5, 12), an anti-CD2 antibody or antigen binding fragment thereof (e.g., siplizumab) [Days −6, −1, 0, 1 (plus Day 6 in Arm 3)], cyclophosphamide (60 mg/kg) on Days −5 and −4, followed by local thymic irradiation (7 Gy) on day −1. Methylprednisolone is given at time of transplant and daily through Day 3. Prednisone, 2 mg/kg/day starting on Day 4 through Day 12 is rapidly tapered and completely stopped on Day 20. For prophylaxis of hemorrhagic cystitis and hematuria due to cyclophosphamide, MESNA (2-mercaptoethane sulfonate sodium) is given and patients undergo hemodialysis before and after cyclophosphamide administration. Combined kidney transplantation with donor bone marrow cell infusion is performed on day 0. MMF (2 g/d) is started at time of transplant and is stopped at Week 8; if weaning criteria are met. Similarly, tacrolimus (target trough 4-11 ng/mL) is started at time of transplant and is down titrated after 6 months post-transplant, if weaning criteria are met. Infectious prophylaxis for viral and bacterial infections are given to the subjects as described herein (e.g., as described in Example 1). Premedication for an anti-CD2 antibody or antigen binding fragment thereof (e.g., siplizumab) and rituximab are given to subjects as described herein.

### Anti-CD2 Antibody or Antigen Binding Fragment Thereof (e.g., Siplizumab) Premedication:

Prior to each infusion of an anti-CD2 antibody or antigen binding fragment thereof (e.g., siplizumab), subjects can receive premedication with 650-1000 mg acetaminophen (or equivalent dose of paracetamol) and an H1-antagonist (antihistamine, e.g., 25 mg diphenhydramine or 4 mg chlorpheniramine) to minimize signs and symptoms of an infusion reaction. Additionally, prior to the first dose of an anti-CD2 antibody or antigen binding fragment thereof (e.g., siplizumab) on Day −6, 8 mg/kg methylprednisolone can be given prior to starting the infusion. Administration of required pre-medications should occur no less than 30 minutes and no more than 3 hours prior to the start of the infusion.

### Anti-CD2 Antibody or Antigen Binding Fragment Thereof (e.g., Siplizumab) Administration:

The first dose of an anti-CD2 antibody or antigen binding fragment thereof (e.g., siplizumab) (0.6 mg/kg) is administered on Day −6. An anti-CD2 antibody or antigen binding fragment thereof (e.g., siplizumab) concentrate is diluted in 0.9% normal saline and infused intravenously via syringe pump over a period of around 1 hour. Pre-medication must precede administration of an anti-CD2 antibody or antigen binding fragment thereof (e.g., siplizumab). This procedure should be followed for each of the 4 siplizumab infusions on Days −6, −1, 0, and 1. Anti-CD2 antibody or antigen binding fragment thereof (e.g., siplizumab) doses on Days −1, 0 and 1 can be administered approximately 20-24 hours apart. For subjects assigned to Arm 3, an additional dose of an anti-CD2 antibody or antigen binding fragment thereof (e.g., siplizumab) on Day +6 is administered. On Day 0, the infusion is administered pre- or intra-operatively. Anti-CD2 antibody or antigen binding fragment thereof (e.g., siplizumab) is administered pre-operatively, the infusion is timed so that completion is no earlier than 4 hours prior to revascularization and perfusion of the allograft. When administered intra-operatively, the infusion is completed prior to revascularization and administration of the bone marrow cell infusion (BMCI). This ensures both donor and recipient alloreactive T-lymphocytes are suppressed and depletion is initiated in the hours and days post-transplant. The infusions can be given directly into a peripheral vein or a separate lumen in an indwelling, multi-lumen, central catheter and not administered concurrently with other medications.

### Rituximab Premedication:

Acetaminophen (650-1000 mg PO), an H1-antagonist (antihistamine, e.g., 25-50 mg diphenhydramine or 4 mg chlorpheniramine), and hydrocortisone sodium succinate (e.g., Solu-Cortef; 100 mg IV) can administered to subjects 2 hours prior to each dose of rituximab

### Rituximab Administration (Days −9, −2, +5 and +12):

Rituximab (375 mg/m²/dose) is administered on Days −9, −2, +5 and +12. In those subjects receiving ongoing renal replacement therapy, rituximab can be administered several hours after hemodialysis. The first rituximab solution for infusion can be administered intravenously at an initial rate of 50 milligrams/hour (mg/hr). The rate may be escalated by 50 mg/hr every 30 minutes to a maximum of 400 mg/hr. Subsequent infusions may be started at 100 milligrams/hour (mg/hr) and titrated by 100 mg/hr every 30 minutes to a maximum of 400 mg/hr if the subject tolerated the first infusion.

### Cyclophosphamide (Days −5 and −4) Pretreatment (Hemodyalisis):

Within 24 hours prior to the first administration of cyclophosphamide subjects undergo hemodialysis as prophylaxis against toxicity. Recipients, with no existing vascular access, are seen by a nephrologist on or prior to Day −5 for vascular access and insertion of a catheter in preparation for hemodialysis during conditioning. Hemodialysis can be performed within 24 hours of the first administration of cyclophosphamide on Day −5. The duration (or dose) of dialysis can be as close to 4 hours in duration or per local nephrologist recommendation. If the subject's eGFR was <10 mL/min and the subject was on hemodialysis or continuous ambulatory peritoneal dialysis (CAPD) at study entry, an additional hemodialysis is performed on Day −1.

### Cyclophosphamide Premedication:

Prior to administration of cyclophosphamide on Days −5 and −4, the recipient can receive pretreatment for nausea, vomiting and infusion reaction prophylaxis (e.g., a glucocorticoid, antihistamine, anxiolytic and antiemetic) per institutional standards. Subjects undergo prophylaxis against hemorrhagic cystitis with MESNA as follows:
Oliguric subjects: MESNA, 15 mg/kg administered via 4 bolus intravenous injections 15 minutes before, and 3, 6 and 9 hours after cyclophosphamide administration.
Non-oliguric subjects: MESNA as above, plus IV hydration (e.g., with 5% dextrose, 1500 mL/M2/24 hrs as tolerated) beginning 4 hours prior to cyclophosphamide. If the subject is oliguric, bladder washout may be performed as well.

### Cyclophosphamide Administration (Days −5 and −4):

Subjects receive cyclophosphamide, 60 mg/kg/day (based on lesser of ideal or actual body weight). Cyclophosphamide is prepared per label for IV infusion with 250 mL sterile 5% dextrose and infused over 1 hour via intravenous infusion. Administration of cyclophosphamide can occur at least 20 hours following hemodialysis on Day −5.

### Post-Treatment (Hemodialysis):

Subjects require hemodialysis treatment 14 hours (+/−2 hrs) after each dose of cyclophosphamide. The duration (or dose) of dialysis can be as close to 4 hours in duration or per standard procedure.

### Thymic Irradiation (Day −1):

The 7 Gy of thymic irradiation is administered in a single dose on Day −1. The field size, shielding and dosimetry is calculated and administered per standard procedure.

### Donor Nephrectomy and Bone Marrow Procurement:

The donor undergoes nephrectomy and bone marrow cell procurement under general anesthesia on Day 0 (day of transplant). Unfractionated donor bone marrow cells (intended minimum of 2×10⁸ TNC/kg of recipient body weight) are prepared for infusion according to standard procedures.

### Recipient Transplantation and Bone Marrow Cell Infusion:

On study Day 0, transplant recipients receive the allograft. A wedge or needle baseline biopsy is obtained from the allograft pre-implantation. Following revascularization of the allograft and upon confirmation that no bleeding or leakage from the vascular anastomoses are present, the recipient receive the bone marrow cell infusion. The cells are infused intravenously, at a rate of approximately 300-500 mL/hr. The infusion can begin no later than 4 hours following reperfusion. A partial thromboplastin time can be measured half-way through the infusion (or if bleeding seems excessive) and following completion of the infusion. Protamine 25 mg may be given intravenously for a PTT of >60 seconds or for an elevated PTT if that is believed to be the cause of bleeding.

### Preparation and Administration of Tocilizumab:

Administration of tocilizumab varies by study arm. In Arm 1, tocilizumab use is only permissible as treatment in the presentation of CTS. If CTS occurs, tocilizumab may be initiated at any time and, if warranted, a further dose is given approximately one week later. Subjects assigned to treatment Arms 2 or 3 receives tocilizumab on Days 7 and 14 as prophylaxis against CTS. Tocilizumab can be prepared and administered according to applicable local labeling at a dosage of 8 mg/kg IV over 60 min.

Management of first dose (infusion) reactions: First dose or infusion reactions may occur upon the initial administration of rituximab, an anti-CD2 antibody or antigen binding fragment thereof (e.g., siplizumab), or tocilizumab. Although pre-medications are given prophylactically prior to administration of rituximab and an anti-CD2 antibody or antigen binding fragment thereof (e.g., siplizumab), further doses of acetaminophen and antihistamine may be administered if fever or chills occur post-study drug administration. Corticosteroids (e.g., 250 mg IV methylprednisolone) may be administered if a severe reaction occurs.

### Concomitant Immunosuppression:

The use of immediate release generic tacrolimus (TAC) and mycophenolate mofetil (MMF) are allowed in this study. Concomitant immunosuppression medications can include:
Immediate release TAC as 0.5 mg, 1.0 mg, or 5.0 mg capsules
MMF 250 mg or 500 mg film-coated tablets, or 250 mg capsules, or 500 mg vial for IV
CS for oral and IV administration

MMF Administration (Day 0 to Day 60): MMF (2 g/day) is administered orally (p.o) as two 500 mg tablets or four 250 mg capsules twice per day (BID). Intravenous administration (1000 mg; two 500 mg vials) may be considered for subjects who remain intubated >24 hours post-transplant and/or who are otherwise unable to swallow oral medications, until oral conversion is possible. The first dose of MMF is administered at the time of transplant and no later than 24 hours after reperfusion of the allograft.

MMF Discontinuation Criteria: Once the below weaning criteria are met, MMF can be discontinued at Week 8.
Stable renal function (sCr <2.0 mg/dL) with immunosuppression, unless a transient rise in creatinine is related to an alternative cause of renal dysfunction.
No evidence of antibody mediated rejection (ABMR) or Banff grade IA or greater T-cell mediated rejection (TCMR).

If renal function criteria are not met at Week 8 due to an alternative cause, MMF discontinuation may be delayed until the excursion in serum creatine is corrected and renal function is considered stable.

### Guidelines for MMF Dose Reduction:

Implementation of dose reduction is based on thrombocytopenia, leukopenia, neutropenia, or other AEs which are suspected to be related to study medication. The following guidelines should be used for both dose reduction and, once the event has resolved, restarting or increasing the dose of MMF back to original levels.

### Dose Reduction Guidelines:

### Platelets

platelet count <100,000/mm³ (dose may be reduced at the discretion of the Investigator/physician)
platelet count <75,000/mm³ (a second dose reduction can be considered
platelet count <50,000/mm³ (mandatory interruption of medication)

### WBC

WBC <3500/mm³ dose may be reduced at the discretion of the Investigator/physician
WBC <2500/mm³ a second dose reduction can be considered
WBC <2000/mm³ mandatory interruption of medication

### Tac Administration (Day 0 Wean Post Month 6, if Criteria are Met):

TAC is administered as capsules or tablets orally (p.o.) twice a day (BID) and adjusted to maintain serum trough (C0) concentrations within the target range of 4-11 ng/ml. If oral administration is not feasible or practical, IV administration of TAC by continuous intravenous infusion can be substituted per label. TAC can be started as soon as possible in the peri-transplant period and may follow local practice but should be initiated no later than 24 hours after reperfusion of the allograft. The lowest permitted dosing of TAC in this study is 0.5 mg BID or IV equivalent.

### Tacrolimus Weaning Criteria:

Serum TAC trough concentrations (4-11 ng/mL) is down-titrated slowly over a 3 to 6 month period starting at Month 6 provided all of the weaning criteria 1-5 below are met.
1. Stable renal function (sCr <2.0 mg/dL) with immunosuppression, unless a transient rise in creatinine is related to an alternative cause of renal dysfunction.
2. There has been detectable multilineage white blood cell chimerism (any level) in the early post-transplant period.
3. No current (or prior) DSA.
4. No current evidence of GvHD.
5. No evidence of antibody mediated rejection (ABMR) or Banff grade IA or greater T-cell mediated rejection (TCMR) on the Month 6 (or most recent) renal biopsy. History of borderline changes do not disqualify a participant from immunosuppression withdrawal, provided that the biopsy result is normal by Week 52.

### Tacrolimus Weaning Schedule (Starting at Month 6-9):

Once the above weaning criteria are met, tacrolimus doses can be reduced proportionately over approximately 6 months, as described below.
75% of Month 6 dose for the first month (e.g., Month 6-7)
50% of Month 6 dose for second two months (e.g., Months 7-9)
25% of Month 6 dose for third two months (e.g., Months 9-11)
12.5% of Month 6 dose for the last month (e.g., 11th month before Month 12 biopsy)

In the event a dose reduction alone results in an over-proportional reduction in TAC exposure, i.e., trough concentrations decrease more than anticipated, the same titration scheme can be applied to a reduction in exposure and doses can be adjusted accordingly. When TAC trough concentrations decrease below the assay limit of quantification, a reduction in the daily dose can be used to complete the tapering. TAC can be stopped completely after a second biopsy (nominally at Month 12 but may be earlier or later depending on the schedule and subject's clinical presentation) demonstrates the allograft is rejection free (e.g., as described in criterion 5 above).

### Corticosteroid Administration:

Corticosteroids (CS) are given at several times in the study for different purposes and the prespecified dosing is as follows:
Premedication for rituximab: hydrocortisone sodium succinate (SoluCortef; 100 mg IV) for premedication prior to rituximab (Days, −9, −2, 5 and 12)
Premedication for an anti-CD2 antibody or antigen binding fragment thereof (e.g., siplizumab): methylprednisolone sodium succinate (SoluMedrol; 8 mg/kg IV) as first dose premedication for an anti-CD2 antibody or antigen binding fragment thereof (e.g., siplizumab) (Day −6)
Transplant immunosuppression:
   IV methylprednisolone: 250 mg on Day 0; 125 mg on Day 1, 100 mg on Day 2, 80 mg on Day 3
   Prednisone 2 mg/kg/day starting Day 4 through Day 12 and rapidly tapering off completely by Day 20.
   Steroid route of administration can be switched from IV to oral with equipotent glucocorticoid dose on or around Day 4 per standard procedure and patient status.

If CTS or rejection is observed, steroid bolus can be considered in first line treatment.

### Conditioning Regimen:

The conditioning regimen begins with the recipient receiving their first dose of rituximab (+premedication) on Day −9 and ends when they receive their last dose of rituximab on Day 12.

### Day −9 (Outpatient):

Following confirmation of all eligibility criteria during screening, including a negative SARS-CoV-2 test performed within 72 hours of Day −9, the donor/recipient pair are enrolled into the study and the recipient subjects are subjected to the following before their first conditioning regimen dose:
Review new or changes to existing medications and/or adverse events
Blood collection for local lab chimerism assessment
Central lab blood collection for immunophenotyping (FACS), CD2RO, and serumcytokines
Initiate levofloxacin (PCP infection prophylaxis)
Rituximab pre-medications
Rituximab infusion
   Vital signs are captured pre-dose, immediately post-infusion and 1-hour following the end of infusion

### Day −6 (Outpatient)

Review of new or changes to existing medications and/or adverse events
Blood collection for local lab CBC and chemistry
Central Lab blood collection for immunogenicity (anti-CD2 antibodies) and CD2RO
Confirmation and/or placement of vascular access for hemodialysis
Hemodialysis must be completed within 24 hrs. prior to cyclophosphamide administration on Day −5
Anti-CD2 antibody or antigen binding fragment thereof (e.g., siplizumab) administration
   Pre-medications administered 30 minutes to 3 hours prior to infusion
   PK sample collected pre-dose and 1-hour following the end of infusion
   Vital signs are captured pre-dose, immediately post-infusion and 1-hour following the end of infusion

### Day −5

Review new or changes to existing medications and/or adverse events
Blood collection for local lab CBC, chemistry, and coagulation panels
MESNA for hemorrhagic cystitis prophylaxis (prn)
Cyclophosphamide pre-medication administration
Cyclophosphamide administration
Hemodialysis 14 hours post cyclophosphamide administration.
Central lab blood collection for PK and CD2RO

### Day −4

Review new or changes to existing medications and/or adverse events
Blood collection for local lab CBC and chemistry
MESNA for hemorrhagic cystitis prophylaxis (prn)
Cyclophosphamide pre-medication administration
Cyclophosphamide administration
Hemodialysis 14 hr post cyclophosphamide administration
Central lab blood collection for PK

### Day −3

Review new or changes to existing medications and/or adverse events
Ensure SARS-CoV-2 testing for donor (test performed 72 hours prior to Day 0)
Blood collection for local lab CBC and chemistry
Central lab blood collection for PK

### Day −2

Review new or changes to existing medications and/or adverse events
Ensure SARS-CoV-2 testing for donor if not previously completed (test performed 72 hours prior to Day 0)
Lymphocyte/CDC crossmatch (completed prior to transplantation)
Blood collection for local lab CBC and chemistry
Rituximab pre-medications
Rituximab infusion
Vital signs captured pre-dose, immediately post-infusion and 1-hour following the end of infusion
Central lab blood collection for PK

### Day −1

Review new or changes to existing medications and/or adverse events
Ensure SARS-CoV-2 testing for donor if not previously completed (test performed 72 hours prior to Day 0)
Lymphocyte/CDC crossmatch (if not previously completed; must have prior to transplantation)
Blood collection for local lab CBC and chemistry
Central lab blood collection for serum cytokines and anti-CD2 antibody or antigen binding fragment thereof (e.g., siplizumab) concentrations (PK)
Thymic irradiation
Anti-CD2 antibody or antigen binding fragment thereof (e.g., siplizumab) administration
   Pre-medications administered 30 minutes to 3 hours prior to infusion
   PK sample collected pre-dose and 1-hour following the end of infusion
   Vital signs are captured pre-dose, immediately post-infusion and 1-hour following the end of infusion

### Day 0 (Day of Transplant)

On the day of combined renal allograft and bone marrow cell infusion, the procedures listed below occurs. The donor nephrectomy/bone marrow procurement and recipient renal transplant are performed.
Review new or changes to existing medications and/or adverse events
Confirmation of negative SARS-CoV-2 result for donor recipient
Interrupt PCP prophylaxis as of Day 0
Blood collection for local lab CBC and chemistry (daily while hospitalized; other timepoints as clinically indicated)
Blood collection for local lab coagulation panel
Cytomegalovirus (CMV) DNA PCR (weekly monitoring while neutropenic)
Prophylactic antimicrobial treatment prior to surgery
Donor Surgical Procedures
   Nephrectomy
   Bone marrow procurement
   Preparation of donor cells
Recipient Surgical Procedures
   Allograft wedge/needle biopsy
   Renal transplant
   Infusion of donor bone marrow cells
PTT (once infusion of done bone marrow cells is 50% complete)
Anti-CD2 antibody or antigen binding fragment thereof (e.g., siplizumab) administration
   Pre-medications administered 30 minutes to 3 hours prior to infusion (with anesthesiologist consultation)
   PK sample collected pre-dose and 1-hour following the end of infusion
   Vital signs are captured pre-dose, immediately post-infusion and 1-hour following the end of infusion
Initiation of concomitant immunosuppression (TAC/MMF) within 24 hours post-transplant
Corticosteroid administration

### Day 1

Review new and changes to medication and any adverse events
Blood collection for local lab CBC and chemistry
Prophylaxis treatment for PCP, fungal/yeast, CMV, and HHV
Anti-CD2 antibody or antigen binding fragment thereof (e.g., siplizumab) administration
   Pre-medications administered 30 minutes to 3 hours prior to infusion
   PK sample collected pre-dose and 1-hour following the end of infusion
   Vital signs captured pre-dose, immediately post-infusion and 1-hour following theend of infusion
Corticosteroid administration
Concomitant IS (TAC/MMF) and TAC trough concentrations

### Day 3

Review new or changes to existing medications and/or adverse events
Blood collection for CBC and chemistry
CMV DNA PCR surveillance
Prophylaxis treatment for PCP, fungal/yeast, CMV, and HHV
Corticosteroid administration
Concomitant IS (TAC/MMF) and TAC trough concentrations
Central lab blood collection for anti-CD2 antibody or antigen binding fragment thereof (e.g., siplizumab) concentrations (PK), CD2, and serum cytokines

### Day 5

Review new or changes to existing medications and/or adverse events
Blood collection for CBC and chemistry
CMV DNA PCR surveillance
Prophylaxis treatment for PCP, fungal/yeast, CMV, and HHV
Rituximab infusion
   Vital signs captured pre-dose, immediately post-infusion and 1-hour following the end of infusion
Corticosteroid administration
Concomitant IS (TAC/MMF) and TAC trough concentrations
Central lab blood collection for anti-CD2 antibody or antigen binding fragment thereof (e.g., siplizumab) concentrations (PK)

### Day 6

Review new or changes to existing medications and/or adverse events
Blood collection for complete blood count (CBC) and chemistry
CMV DNA PCR surveillance.
Prophylaxis treatment for PCP, fungal/yeast, CMV, and HHV
Central lab blood collection for anti-CD2 antibody or antigen binding fragment thereof (e.g., siplizumab) concentrations (PK)
Treatment Arm 3 ONLY—an anti-CD2 antibody or antigen binding fragment thereof (e.g., siplizumab) administration
   Pre-medications administered 30 minutes to 3 hours prior to infusion
   PK sample collected pre-dose and 1-hour following the end of infusion
Vital signs captured pre-dose, immediately post-infusion and 1-hour following the end of infusion
Corticosteroid administration
Concomitant IS (TAC/MMF) and TAC trough concentrations

### Day 7

Review new or changes to existing medications and/or adverse events
Vital Signs captured
Blood collection for CBC, chemistry and coagulation panel
CMV DNA PCR surveillance
Blood collection for local lab chimerism assessment.
Prophylaxis treatment for PCP, fungal/yeast, CMV, and HHV
Treatment Arms 2 and 3 Only: Tocilizumab administration (Days 7 and 14)
Corticosteroid administration
Concomitant IS (TAC/MMF) and TAC trough concentrations
Central lab blood collection for anti-CD2 antibody or antigen binding fragment thereof (e.g., siplizumab) concentrations (PK), CD2, and serum cytokines
Central lab urine collection for renal injury markers

### Day 10

Review new or changes to existing medications and/or adverse events.
Blood collection for CBC and chemistry
Prophylaxis treatment for PCP, fungal/yeast, CMV, and HHV
Corticosteroid administration
Concomitant IS (TAC/MMF) and TAC trough concentrations
Central lab blood collection for FACS and serum cytokines

### Day 12

Review new or changes to existing medications and/or adverse events.
Blood collection for CBC and chemistry
Prophylaxis treatment for PCP, fungal/yeast, CMV, and HHV
Rituximab pre-medications
Rituximab infusion
   Vital signs captured pre-dose, immediately post-infusion and 1-hour following the end of infusion
Corticosteroid administration
Concomitant IS (TAC/MMF) and TAC trough concentrations

### Additional Assessments:

In addition to the assessments noted above, and also during the conditioning period, recipients are evaluated for signs/symptoms of rejection, graft loss, GvHD, and CTS as shown in FIG. 4.

### Immunosuppression Period (Post-Conditioning to Month 12):

The Conditioning Period of the protocol effectively ends with the final administration of rituximab on Day 12 and when subjects initiate or continue immunosuppression treatment with tacrolimus, MMF (Day 1) and corticosteroids. Tacrolimus can be started in the perioperative period on Day 0 but should be started within 24 hours of transplant surgery.

Subjects initiate broad infection prophylaxis against fungal/yeast, CMV and HSVinfection (See Example 6.1). During this period, and while hospitalized, the subject will have daily CBC and Comprehensive Metabolic Panel (Chemistry) assessments, weekly CMV DNAPCR surveillance, and assessments for graft loss, rejection, and GvHD (See Example 6.1). Corticosteroids continue until Study Day 20 and MMF continue through Week 8. Tacrolimus weaning starts at Month 6 and are tapered over 3-6 months to complete withdrawal at Month 9-12 post-transplant.

### Immunosuppression Free Period (Month 12 to Month 60):

Following the Month 12 visit, subjects return for clinic visits on a quarterly basis through Month 24 for overall health and laboratory assessments and for evaluation of adverse events. Month 24 is the study endpoint. Following completion of the Month 24 visit, subjects enter a longer-term follow-up period and have bi-yearly visits until they return for the final study visit at Month 60. FIG. 4 shows a comprehensive list of assessments and procedures to be completed at all study visits.

### Safety Assessments:

AEs and SAEs
Clinical chemistry, hematology, vital signs, and serology
Renal function

### Data Analysis:

The primary objective is assessed based on incidence. Other efficacy objectives are analyzed similarly. AE/SAE data is coded and displayed by SoC and preferred term with incidence. Lab data are analyzed by visit with summary statistics and change from baseline.

### Sample Size Determination:

A sample size of 6 subjects for each arm was chosen based on practical considerations, including the need to characterize the incidence and severity of CTS in the immediate post-transplant time period while balancing the overall risk-benefit in a regimen-finding study.

### Study Duration:

The primary endpoint is evaluated at 24 months post-transplant, while all recipients enrolled are followed to 5 years for outcomes.

TABLE 15
SEQUENCES

| SEQ | | |
|---|---|---|
| ID NO | DESCRIPTION | SEQUENCE |
| 1 | heavy chain | QVQLVQSGAEVQRPGASVKVSCKASGYIFTEYYMYWVRQAPGQG |
| | variable region | LELVGRIDPEDGSIDYVEKFKKKVTLTADTSSSTAYMELSSLTS |
| | | DDTAVYYCARGKFNYRFAYWGQGTLVTVSS |
| 2 | light chain | DVVMTQSPPSLLVTLGQPASISCRSSQSLLHSSGNTYLNWLLQR |
| | variable region | PGQSPQPLIYLVSKLESGVPDRESGSGSGTDFTLKISGVEAEDV |
| | | GVYYCMQFTHYPYTFGQGTKLEIK |
| 3 | heavy chain | EYYMY |
| | variable region | |
| | CDR 1 | |
| 4 | heavy chain | RIDPEDGSIDYVEKFKK |
| | variable region | |
| | CDR 2 | |
| 5 | heavy chain | GKFNYRFAY |
| | variable region | |
| | CDR 3 | |
| 6 | light chain | RSSQSLLHSSGNTYLN |
| | variable region | |
| | CDR 1 | |
| 7 | light chain | LVSKLES |
| | variable region | |
| | CDR 2 | |
| 8 | light chain | MQFTHYPYT |
| | variable region | |
| | CDR 3 | |
| 9 | heavy chain | ASTKGPSVFPLAPSSKSTSGGTAALGCLVKDYFPEPVTVSWNSG |
| | constant region | ALTSGVHTFPAVLQSSGLYSLSSVVTVPSSSLGTQTYICNVNHK |
| | | PSNTKVDKRVEPKSCDKTHTCPPCPAPELLGGPSVFLFPPKPKD |
| | | TLMISRTPEVTCVVVDVSHEDPEVKFNWYVDGVEVHNAKTKPRE |
| | | EQYNSTYRVVSVLTVLHQDWLNGKEYKCKVSNKALPAPIEKTIS |
| | | KAKGQPREPQVYTLPPSREEMTKNQVSLTCLVKGFYPSDIAVEW |
| | | ESNGQPENNYKTTPPVLDSDGSFFLYSKLTVDKSRWQQGNVFSC |
| | | SVMHEALHNHYTQKSLSLSPGK |
| 10 | heavy chain | ASTKGPSVFPLAPSSKSTSGGTAALGCLVKDYFPEPVTVSWNSG |
| | constant region | ALTSGVHTFPAVLQSSGLHSLSSVVTVPSSSLGTQTYICNVNHK |
| | | PSNTKVDKKVEPKSCDKTHTCPPCPAPELLGGPSVFLFPPKPKD |
| | | TLMISRTPEVTCVVVDVSHEDPEVKFNWYVDGVEVHNAKTKPRE |
| | | EQYNSTYRVVSVLTVLHQDWLNGKEYKCKVSNKALPAPIEKTIS |
| | | KAKGQPREPQVYTLPPSRDELTKNQVSLTCLVKGFYPSDIAVEW |
| | | ESNGQPENNYKTTPPVLDSDGSFFLYSKLTVDKSRWQQGNVFSC |
| | | SVMHEALHNHYTQKSLSLSPGK |
| 11 | heavy chain | ASTKGPSVFPLAPSSKSTSGGTAALGCLVKDYFPEPVTVSWNSG |
| | constant region | ALTSGVHTFPAVLQSSGLYSLSSVVTVPSSSLGTQTYICNVNHK |
| | | PSNTKVDKKVEPKSCDKTHTCPPCPAPELLGGPSVFLFPPKPKD |
| | | TLMISRTPEVTCVVVDVSHEDPEVKFNWYVDGVEVHNAKTKPRE |
| | | EQYNSTYRVVSVLTVLHQDWLNGKEYKCKVSNKALPAPIEKTIS |
| | | KAKGQPREPQVYTLPPSRDELTKNQVSLTCLVKGFYPSDIAVEW |
| | | ESNGQPENNYKTTPPVLDSDGSFFLYSKLTVDKSRWQQGNIFSC |
| | | SVMHEALHNHYTQKSLSLSPGK |
| 12 | heavy chain | ASTKGPSVFPLAPSSKSTSGGTAALGCLVKDYFPEPVTVSWNSG |
| | constant region | ALTSGVHTFPAVLQSSGLYSLSSVVTVPSSSLGTQTYICNVNHK |
| | | PSNTKVDKKVEPKSCDKTHTCPPCPAPELLGGPSVELFPPKPKD |
| | | TLMISRTPEVTCVVVDVSHEDPEVKFNWYVDGVEVHNAKTKPRE |
| | | EQYNSTYRVVSVLTVLHQDWLNGKEYKCKVSNKALPAPIEKTIS |
| | | KAKGQPREPQVYTLPPSRDELTKNQVSLTCLVKGFYPSDIAVEW |
| | | ESNGQPENNYKTTPPVLDSDGSFFLYSKLTVDKSRWQQGNVFSC |
| | | SVMHEALHNHYTQKSLSLSPGK |
| 13 | heavy chain | ASTKGPSVFPLAPSSKSTSGGTAALGCLVKDYFPEPVTVSWNSG |
| | constant region | ALTSGVHTFPAVLQSSGLYSLSSVVTVPSSSLGTQTYICNVNHK |
| | | PSNTKVDKKVEPKSCDKTHTCPPCPAPELLGGPSVFLFPPKPKD |
| | | TLMISRTPEVTCVVVDVSHEDPEVKFNWYVDGVEVHNAKTKPRE |
| | | EQYNSTYRVVSVLTVLHQDWLSGKEYKCKVSNKALPAPIEKTIS |
| | | KAKGQPREPQVYTLPPSRDELTKNQVSLTCLVKGFYPSDIAVEW |
| | | ESNGQPENNYKTTPPVLDSDGSFFLYSKLTVDKSRWQQGNVFSC |
| | | SVMHEALHNHYTQKSLSLSPGK |
| 14 | light chain | RTVAAPSVFIFPPSDEQLKSGTASVVCLLNNFYPREAKVQWKVD |
| | constant region | NALQSGNSQESVTEQDSKDSTYSLSSTLTLSKADYEKHKLYACE |
| | | VTHQGLSSPVTKSFNRGEC |
| 15 | light chain | RTVAAPSVFIFPPSDEQLKSGTASVVCLLNNFYPREAKVQWKVD |
| | constant region | NALQSGNSQESVTEQDSKDSTYSLSSTLTLSKADYEKHKVYACE |
| | | VTHQGLSSPVTKSFNRGEC |
| 16 | optimized heavy | CAAGTGCAGCTGGTGCAGAGCGGAGCTGAGGTGCAGAGACCCGG |
| | chain sequence | CGCCAGCGTCAAGGTGAGCTGTAAGGCCAGCGGCTACATCTTCA |
| | | CAGAATACTACATGTACTGGGTGAGGCAAGCCCCCGGCCAAGGA |
| | | CTGGAGCTGGTGGGCAGAATCGATCCAGAGGATGGCAGCATCGA |
| | | CTACGTGGAGAAGTTCAAGAAGAAGGTGACTCTGACAGCCGACA |
| | | CAAGCAGCAGCACTGCTTACATGGAGCTGAGCTCTCTGACTAGC |
| | | GATGACACTGCCGTGTACTACTGTGCTAGGGGCAAGTTCAACTA |
| | | TAGGTTCGCCTACTGGGGCCAAGGCACTCTGGTGACAGTCAGCA |
| | | GCGCTAGCACCAAGGGCCCATCGGTCTTCCCCCTGGCACCCTCC |
| | | TCCAAGAGCACCTCTGGGGGCACAGCGGCCCTGGGCTGCCTGGT |
| | | CAAGGACTACTTCCCCGAACCGGTGACGGTGTCGTGGAACTCAG |
| | | GCGCCCTGACCAGCGGCGTGCACACCTTCCCGGCCGTCCTACAG |
| | | TCCTCAGGACTCTACTCCCTCAGCAGCGTGGTGACCGTGCCCTC |
| | | CAGCAGCTTGGGCACCCAGACCTACATCTGCAACGTGAATCACA |
| | | AGCCCAGCAACACCAAGGTGGACAAGAAGGTTGAGCCCAAATCT |
| | | TGTGACAAAACTCACACATGCCCACCGTGCCCAGCACCTGAACT |
| | | CCTGGGGGGACCGTCAGTCTTCCTCTTCCCCCCAAAACCCAAGG |
| | | ACACCCTCATGATCTCCCGGACCCCTGAGGTCACATGCGTGGTG |
| | | GTGGACGTGAGCCACGAAGACCCTGAGGTCAAGTTCAACTGGTA |
| | | CGTGGACGGCGTGGAGGTGCATAATGCCAAGACAAAGCCGCGGG |
| | | AGGAGCAGTACAACAGCACGTACCGTGTGGTCAGCGTCCTCACC |
| | | GTCCTGCACCAGGACTGGCTGAATGGCAAGGAGTACAAGTGCAA |
| | | GGTCTCCAACAAAGCCCTCCCAGCCCCCATCGAGAAAACCATCT |
| | | CCAAAGCCAAAGGGCAGCCCCGAGAACCACAGGTGTACACCCTG |
| | | CCCCCATCCCGGGACGAGCTGACCAAGAACCAGGTCAGCCTGAC |
| | | CTGCCTGGTCAAAGGCTTCTATCCCAGCGACATCGCCGTGGAGT |
| | | GGGAGAGCAATGGGCAGCCGGAGAACAACTACAAGACCACGCCT |
| | | CCCGTGCTGGACTCCGACGGCTCCTTCTTCCTCTACAGCAAGCT |
| | | CACCGTGGACAAGAGCAGGTGGCAGCAGGGGAACGTCTTCTCAT |
| | | GCTCCGTGATGCATGAGGCTCTGCACAACCACTACACGCAGAAG |
| | | AGCCTCTCCCTGTCTCCGGGTAAATGATGA |
| 17 | optimized Light | GACGTGGTGATGACTCAGAGCCCTCCTTCTCTGCTGGTGACTCT |
| | chain sequence | GGGCCAGCCAGCCAGCATCAGCTGTAGGAGCAGCCAGTCTCTGC |
| | | TGCACTCCAGCGGCAACACTTATCTGAACTGGCTGCTGCAGAGA |
| | | CCCGGCCAGAGCCCTCAGCCTCTGATCTACCTCGTGAGCAAGCT |
| | | GGAGAGCGGCGTGCCAGATAGGTTTAGCGGCAGCGGAAGCGGCA |
| | | CTGACTTCACTCTGAAGATCAGCGGCGTGGAAGCTGAGGATGTG |
| | | GGCGTCTACTACTGCATGCAGTTCACACACTACCCATACACTTT |
| | | CGGCCAAGGCACAAAGCTGGAAATCAAGCGTACGGTGGCTGCAC |
| | | CATCTGTCTTCATCTTCCCGCCATCTGATGAGCAGTTGAAATCT |
| | | GGAACTGCCTCTGTTGTGTGCCTGCTGAATAACTTCTATCCCAG |
| | | AGAGGCCAAAGTACAGTGGAAGGTGGATAACGCCCTCCAATCGG |
| | | GTAACTCCCAGGAGAGTGTCACAGAGCAGGACAGCAAGGACAGC |
| | | ACCTACAGCCTCAGCAGCACCCTGACGCTGAGCAAAGCAGACTA |
| | | CGAGAAACACAAAGTCTACGCCTGCGAAGTCACCCATCAGGGCC |
| | | TGAGCTCGCCCGTCACAAAGAGCTTCAACAGGGGAGAGTGTTGA |
| | | TGA |
| 18 | CH2 domain | SVELFPPKPKDTLMISRTPEVTCVVVDVSAEDPEVQFNWYVDGV |
| | | EVHNAKTKPREEQFNSTFRVVSVLTVLHQDWLNGKEYKCKVSNK |
| | | GLPSSIEKTISKTK |
| 19 | CH2 domain | SVFLFPPKPKDTLMISRTPEVTCVVVDVSQEDPEVQFNWYVDGV |
| | | EVHNAKTKPREEQFNSTYRVVSVLTVLHQDWLNGKEYKCKVSNK |
| | | GLGSSIEKTISKAK |
| 20 | CH3 domain | GQPREPQVYTLPPSREEMTKNQVSLTCLVKGFYPSDIAVEWESN |
| | | GQPENNYKTTPPMLDSDGSFFLYSKLTVDKSRWQQGNVFSCSVM |
| | | HEALHNHYTQKSLSLSPGK |
| 21 | CH3 domain | GQPREPQVYTLPPSQEEMTKNQVSLTCLVKGFYPSDIAVEWESN |
| | | GQPENNYKTTPPVLDSDGSFFLYSRLTVDKSRWQEGNVFSCSVM |
| | | HEALHNHYTQKSLSLSLGK |
| 22 | tocilizumab | SDHAWS |
| | heavy chain | |
| | variable region | |
| | CDR 1 | |
| 23 | tocilizumab | YISYSGITTYNPSLKS |
| | heavy chain | |
| | variable region | |
| | CDR 2 | |
| 24 | tocilizumab | SLARTTAMDY |
| | heavy chain | |
| | variable region | |
| | CDR 3 | |
| 25 | tocilizumab | RASQDISSYLN |
| | light chain | |
| | variable region | |
| | CDR 1 | |
| 26 | tocilizumab | YTSRLHS |
| | light chain | |
| | variable region | |
| | CDR 2 | |
| 27 | tocilizumab | QQGNTLPYT |
| | light chain | |
| | variable region | |
| | CDR 3 | |
| 28 | tocilizumab | QVQLQESGPGLVRPSQTLSLTCTVSGYSITSDHAWSWVRQPPGR |
| | heavy chain | GLEWIGYISYSGITTYNPSLKSRVTMLRDTSKNQFSLRLSSVTA |
| | variable region | ADTAVYYCARSLARTTAMDYWGQGSLVTVSS |
| 29 | tocilizumab | DIQMTQSPSSLSASVGDRVTITCRASQDISSYLNWYQQKPGKAP |
| | light chain | KLLIYYTSRLHSGVPSRFSGSGSGTDFTFTISSLQPEDIATYYC |
| | variable region | QQGNTLPYTFGQGTKVEIK |

1. A method of transplanting an organ or a tissue from a donor to a subject, wherein the method comprises:
a) administering a B-cell depleting antibody to the subject;
b) administering an anti-CD2 antibody or antigen binding fragment thereof to the subject;
c) administering fludarabine to the subject;
d) transplanting the organ or the tissue into the subject; and
e) infusing bone marrow cells from the donor to the subject.

2. A method of transplanting an organ or a tissue from a donor to a subject, wherein the method comprises:
a) administering a B-cell depleting antibody to the subject;
b) administering an anti-CD2 antibody or antigen binding fragment thereof to the subject;
c) transplanting the organ or the tissue into the subject; and
d) infusing bone marrow cells from the donor to the subject;
wherein the B-cell depleting antibody is administered to the subject more than 7 days prior to the transplanting.

3. A method of transplanting an organ or a tissue from a donor to a subject, wherein the method comprises:
a) administering a B-cell depleting antibody to the subject;
b) administering an anti-CD2 antibody or antigen binding fragment thereof to the subject;
c) transplanting the organ or the tissue into the subject; and
d) infusing bone marrow cells from the donor to the subject;
wherein a first dose of the anti-CD2 antibody or antigen binding fragment thereof is 1 day prior to the transplanting.

4. A method of transplanting an organ or a tissue from a donor to a subject, wherein the method comprises:
a) administering a B-cell depleting antibody to the subject;
b) administering an anti-CD2 antibody or antigen binding fragment thereof to the subject;
c) transplanting the organ or the tissue into the subject; and
d) infusing bone marrow cells from the donor to the subject;
wherein the anti-CD2 antibody or antigen binding fragment thereof is administered to the subject more than 2 days prior to the transplanting.

5. A method of minimizing chimeric transition syndrome in a subject in need of an organ or a tissue transplant, wherein the method comprises:
a) administering a B-cell depleting antibody to the subject;
b) administering an anti-CD2 antibody or antigen binding fragment thereof to the subject;
c) administering fludarabine to the subject;
d) transplanting the organ or the tissue into the subject; and
e) infusing bone marrow cells from the donor to the subject.

6. A method of minimizing chimeric transition syndrome in a subject in need of an organ or a tissue transplant, wherein the method comprises:
a) administering a B-cell depleting antibody to the subject;
b) administering an anti-CD2 antibody or antigen binding fragment thereof to the subject;
c) transplanting the organ or the tissue into the subject; and
d) infusing bone marrow cells from the donor to the subject;
wherein the B-cell depleting antibody is administered to the subject more than 7 days prior to the transplanting.

7. A method of minimizing chimeric transition syndrome in a subject in need of an organ or a tissue transplant, wherein the method comprises:
a) administering a B-cell depleting antibody to the subject;
b) administering an anti-CD2 antibody or antigen binding fragment thereof to the subject;
c) transplanting the organ or the tissue into the subject; and
d) infusing bone marrow cells from the donor to the subject;
wherein a first dose of the anti-CD2 antibody or antigen binding fragment thereof is 1 day prior to the transplanting.

8. A method of minimizing chimeric transition syndrome in a subject in need of an organ or a tissue transplant, wherein the method comprises:
a) administering a B-cell depleting antibody to the subject;
b) administering an anti-CD2 antibody or antigen binding fragment thereof to the subject;
c) transplanting the organ or the tissue into the subject; and
d) infusing bone marrow cells from the donor to the subject;
wherein the anti-CD2 antibody or antigen binding fragment thereof is administered to the subject more than 2 days prior to the transplanting.

9. The method of any one of claims 1-8, wherein the B-cell depleting antibody is administered to the subject 9 days prior to the transplanting.

10. The method of any one of claims 1-9, wherein the B-cell depleting antibody is administered to the subject 9 days prior to and 2 days prior to the transplanting.

11. The method of any one of claims 1-10, wherein the B-cell depleting antibody is administered to the subject 9 days prior to and 2 days prior to the transplanting and 5 days post and 12 days post the transplanting.

12. The method of any one of claims 1-11, wherein the B-cell depleting antibody is rituximab.

13. The method of any one of claims 1-12, wherein the B-cell depleting antibody is administered to the subject at a dose of about 375 mg/m².

14. The method of any one of claims 1-13, wherein the anti-CD2 antibody or antigen binding fragment thereof is administered to the subject 1 day prior to the transplanting, on the day of the transplanting, and 1 day post the transplanting.

15. The method of any one of claims 1-14, wherein the anti-CD2 antibody or antigen binding fragment thereof is administered to the subject 6 days prior to the transplanting, 1 day prior to the transplanting, on the day of the transplanting, and 1 day post the transplanting.

16. The method of any one of claims 1-15, wherein the anti-CD2 antibody or antigen binding fragment thereof is administered to the subject 6 days prior to the transplanting, 1 day prior to the transplanting, on the day of the transplanting, 1 day post the transplanting, and 6 days post the transplanting.

17. The method of any one of claims 1-16, wherein the anti-CD2 antibody or antigen binding fragment thereof is siplizumab.

18. The method of any one of claims 1-17, wherein the anti-CD2 antibody or antigen binding fragment thereof is administered to the subject at a dose of about 0.6 mg/kg.

19. The method of any one of claims 1-18, wherein the organ or the tissue is kidney.

20. The method of any one of claims 1-19, wherein the subject is a human.

21. The method of any one of claims 1-20, wherein the donor is a human.

22. The method of any one of claims 1-21, wherein the method further comprises administering a non-myeloablative conditioning agent to the subject.

23. The method of claim 22, wherein the non-myeloablative conditioning agent comprises an immunosuppressive agent.

24. The method of claim 23, wherein the immunosuppressive agent is cyclophosphamide.

25. The method of claim 23 or 24, wherein the immunosuppressive agent is administered to the subject 5 days prior to and 4 days prior to the transplanting.

26. The method of any one of claims 23-25, wherein the immunosuppressive agent is administered to the subject at a dose of about 60 mg/kg.

27. The method of any one of claims 23-25, wherein the immunosuppressive agent is administered to the subject at a dose of about 22.5 mg/kg.

28. The method of any one of claims 22-27, wherein the non-myeloablative conditioning agent comprises an anti-neoplastic agent.

29. The method of claim 28, wherein the anti-neoplastic agent is fludarabine.

30. The method of claim 28 or 29, wherein the anti-neoplastic agent is administered to the subject 6 days prior to, 5 days prior to, 4 days prior to, and 3 days prior to the transplanting.

31. The method of any one of claims 1, 5, and 29, wherein the fludarabine is administered to the subject 6 days prior to, 5 days prior to, 4 days prior to, and 3 days prior to the transplanting

32. The method of any one of claims 28-30, wherein the anti-neoplastic agent is administered to the subject at a dose of about 10 mg/m².

33. The method of any one of claims 1, 5, 29, and 31, wherein the fludarabine is administered to the subject at a dose of about 10 mg/m².

34. The method of any one of claims 1-33, wherein the infusing bone marrow cells is on the same day as the transplanting.

35. The method of any one of claims 1-34, wherein the method further comprises providing thymus irradiation to the subject.

36. The method of claim 35, wherein the thymus irradiation is provided to the subject 1 day prior to the transplanting.

37. The method of claim 35 or 36, wherein the thymus irradiation is provided to the subject at about 7 Gy.

38. The method of any one of claims 1-37, wherein the method further comprises administering an anti-IL6R antibody to the subject.

39. The method of claim 38, wherein the anti-IL6R antibody is tocilizumab.

40. The method of claim 38 or 39, wherein the anti-IL6R antibody is administered to the subject at a dose of about 8 mg/kg.

41. The method of any one of claims 38-40, wherein the anti-IL6R antibody is administered to the subject 7 days and 14 days post the transplanting.

42. The method of any one of claims 1-41, wherein the method further comprises administering a steroid to the subject.

43. The method of claim 42, wherein the steroid is administered to the subject on the day of the transplanting.

44. The method of claim 42 or 43, wherein the steroid is administered to the subject for about 20 days after the transplanting.

45. The method of any one of claims 42-44, wherein the steroid is administered to the subject for about 6 months after the transplanting.

46. The method of any one of claims 42-45, wherein the steroid is corticosteroid.

47. The method of any one of claims 1-46, wherein the method further comprises administering tacrolimus to the subject.

48. The method of claim 47, wherein the tacrolimus is administered to the subject at a dose of about 4-11 ng/mL.

49. The method of claim 48, wherein the tacrolimus is administered to the subject at a dose of about 8-10 ng/mL.

50. The method of any one of claims 47-49, wherein the tacrolimus is administered to the subject on the day of the transplanting.

51. The method of any one of claims 47-50, wherein the tacrolimus is administered to the subject for about 9-12 months post the transplanting.

52. The method of any one of claims 47-50, wherein the tacrolimus is administered to the subject for about 1 month post the transplanting.

53. The method of any one of claims 1-52, wherein the method further comprises administering mycophenolate mofetil to the subject.

54. The method of claim 53, wherein the mycophenolate mofetil is administered to the subject at a dose of about 2 g/day.

55. The method of claim 53 or 54, wherein the mycophenolate mofetil is administered to the subject on the day of the transplanting.

56. The method of any one of claims 53-55, wherein the mycophenolate mofetil is administered to the subject for about 2 months post the transplanting.

57. The method of any one of claims 1-56, wherein the method further comprises administering sirolimus to the subject.

58. The method of claim 57, wherein the sirolimus is administered to the subject at a dose of about 5-8 ng/mL.

59. The method of claim 57 or 58, wherein the sirolimus is administered to the subject at about or after about 1 month post the transplanting.

60. The method of any one of claims 57-59, wherein the sirolimus is administered to the subject for up to about 12 months after the transplanting.

61. The method of any one of claims 1-60, wherein the B-cell depleting antibody is not administered 7 days prior to the transplanting.

62. The method of any one of claims 1-61, wherein the method induces mixed chimerism in the subject.

63. The method of claim 62, wherein the mixed chimerism is characterized by a percentage of donor cells in the lymphohematopoietic system of the subject of at least about 5%, 10%, 15%, 20%, 25%, 30%, 35%, 40%, 45%, 50%, 55%, 60%, 65%, 70%, 75%, 80%, 85%, or at least about 90%.

64. The method of claim 62 or 63, wherein the mixed chimerism persists for about or at most about 1 month, 2 months, 3 months, 4 months, 5 months, 6 months, 7 months, 8 months, 9 months, 10 months, 11 months, or at most 12 months.

65. The method of any one of claims 1-64, wherein the ratio of FoxP3+ T cells to CD4+ T cells is increased in the subject relative to the ratio of FoxP3+ T cells to CD4+ T cells in the absence of the anti-CD2 antibody or antigen binding fragment thereof.

66. The method of claim 65, wherein the FoxP3+ expression persists in actively proliferating T cells after the last administration of the anti-CD2 antibody or antigen binding fragment thereof.

67. The method of any one of claims 1-66, wherein the method reduces the risk of Graft versus Host Disease in the subject.

68. The method of any one of claims 1-67, wherein the anti-CD2 antibody or antigen binding fragment thereof comprises:
a. a heavy chain variable region CDR 1 of SEQ ID NO:3; and
b. a heavy chain variable region CDR 2 of SEQ ID NO:4; and
c. a heavy chain variable region CDR 3 of SEQ ID NO:5; and
d. a light chain variable region CDR 1 of SEQ ID NO:6; and
e. a light chain variable region CDR 2 of SEQ ID NO:7; and
f. a light chain variable region CDR 3 of SEQ ID NO:8.

69. The method of any one of claims 1-68, wherein the anti-CD2 antibody or antigen binding fragment thereof is a humanized antibody.

70. The method of any one of claims 1-69, wherein the anti-CD2 antibody or antigen binding fragment thereof is administered to the subject at a dose of more than about 0.1 mg/kg.

71. The method of any one of claims 1-69, wherein the anti-CD2 antibody or antigen binding fragment thereof is administered to the subject at a dose of about 0.6 mg/kg.

72. The method of any one of claims 1-71, wherein each dose of the anti-CD2 antibody or antigen binding fragment thereof that is administered to the subject comprises the same dose.

73. The method of any one of claims 1-72, wherein the method induces tolerance in the subject to the transplanted organ or tissue.

74. The method of any one of claims 47-73, wherein the tacrolimus is administered to the subject after the transplanting.

75. The method of any one of claims 47-74, wherein the tacrolimus is administered to the subject after about or after at least about 1 month, 2 months, 3 months, 4 months, 5 months, 6 months, 7 months, 8 months, 9 months, 10 months, 11 months, 12 months, or more than 12 months after the transplanting.

76. The method of any one of claims 47-75, wherein the tacrolimus is administered to the subject after about 3-4 months after the transplanting.

77. The method of any one of claims 53-76, wherein the mycophenolate mofetil is administered to the subject after the transplanting.

78. The method of any one of claims 53-77, wherein the mycophenolate mofetil is administered to the subject after about or after at least about 1 month, 2 months, 3 months, 4 months, 5 months, 6 months, 7 months, 8 months, 9 months, 10 months, 11 months, 12 months, or more than 12 months after the transplanting.

79. The method of any one of claims 53-78, wherein the mycophenolate mofetil is administered to the subject after about 3-4 months after the transplanting.

80. The method of any one of claims 42-79, wherein the steroid is administered to the subject after the transplanting.

81. The method of any one of claims 46-80, wherein the corticosteroid is administered to the subject after the transplanting.

82. The method of any one of claims 42-81, wherein the steroid is administered to the subject after about or after at least about 1 month, 2 months, 3 months, 4 months, 5 months, 6 months, 7 months, 8 months, 9 months, 10 months, 11 months, 12 months, or more than 12 months after the transplanting.

83. The method of any one of claims 46-82, wherein the corticosteroid is administered to the subject after about or after at least about 1 month, 2 months, 3 months, 4 months, 5 months, 6 months, 7 months, 8 months, 9 months, 10 months, 11 months, 12 months, or more than 12 months after the transplanting.

84. The method of any one of claims 42-83, wherein the steroid is administered to the subject after about 3-4 months after the transplanting.

85. The method of any one of claims 46-84, wherein the corticosteroid is administered to the subject after about 3-4 months after the transplanting.

86. The method of any one of claims 38-85, wherein the anti-IL6R antibody is administered to the subject after the transplanting.

87. The method of any one of claims 38-86, wherein the anti-IL6R antibody is administered to the subject after about or after at least about 1 month, 2 months, 3 months, 4 months, 5 months, 6 months, 7 months, 8 months, 9 months, 10 months, 11 months, 12 months, or more than 12 months after the transplanting.

88. The method of any one of claims 38-87, wherein the anti-IL6R antibody is administered to the subject after about 3-4 months after the transplanting.

89. The method of any one of claims 57-88, wherein the sirolimus is administered to the subject after the transplanting.

90. The method of any one of claims 57-89, wherein the sirolimus is administered to the subject after about or after at least about 1 month, 2 months, 3 months, 4 months, 5 months, 6 months, 7 months, 8 months, 9 months, 10 months, 11 months, 12 months, or more than 12 months after the transplanting.

91. The method of any one of claims 57-90, wherein the sirolimus is administered to the subject after about 3-4 months after the transplanting.
